SEQUENCE LISTING (939 sequences)

SEQ ID NO: 1 - protein, 1345 aa, Streptococcus mutans
  REGION 10..21: note = misc_feature - N-terminal RuvC-like domain
  REGION 759..766: note = misc_feature - RuvC-like domain
  REGION 837..863: note = misc_feature - HNH-like domain
  REGION 982..989: note = misc_feature - RuvC-like domain
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1368 aa, Streptococcus pyogenes [sequence omitted]

SEQ ID NO: 3 - DNA, 4107 bp, Streptococcus pyogenes
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 4 - protein, 1388 aa, Streptococcus thermophilus
  REGION 10..21: note = misc_feature - N-terminal RuvC-like domain
  REGION 760..767: note = misc_feature - RuvC-like domain
  REGION 844..870: note = misc_feature - HNH-like domain
  REGION 989..996: note = misc_feature - RuvC-like domain
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1334 aa, Listeria innocua
  REGION 10..21: note = misc_feature - N-terminal RuvC-like domain
  REGION 762..769: note = misc_feature - RuvC-like domain
  REGION 840..866: note = misc_feature - HNH-like domain
  REGION 985..992: note = misc_feature - RuvC-like domain
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 6 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - DNA, 3159 bp, Staphylococcus aureus
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 8 - DNA, 3159 bp, Staphylococcus aureus
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 9 - DNA, 3159 bp, Staphylococcus aureus
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 10 - DNA, 3159 bp, Staphylococcus aureus
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 11 - DNA, 3159 bp, Staphylococcus aureus
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 12 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 13 - DNA, 3249 bp, Neisseria meningitidis
  misc_feature 1..3249: note = Exemplary codon optimized Cas9
  source 1..3249: mol_type = other DNA
atggccgccttcaagcccaaccccatcaactacatcctgggcctggacatcggcatcgccagcgtgggctgggccatggtggagatcgacgaggacgagaaccccatctgcctgatcgacctgggtgtgcgcgtgttcgagcgcgctgaggtgcccaagactggtgacagtctggctatggctcgccggcttgctcgctctgttcggcgccttactcgccggcgcgctcaccgccttctgcgcgctcgccgcctgctgaagcgcgagggtgtgctgcaggctgccgacttcgacgagaacggcctgatcaagagcctgcccaacactccttggcagctgcgcgctgccgctctggaccgcaagctgactcctctggagtggagcgccgtgctgctgcacctgatcaagcaccgcggctacctgagccagcgcaagaacgagggcgagaccgccgacaaggagctgggtgctctgctgaagggcgtggccgacaacgcccacgccctgcagactggtgacttccgcactcctgctgagctggccctgaacaagttcgagaaggagagcggccacatccgcaaccagcgcggcgactacagccacaccttcagccgcaaggacctgcaggccgagctgatcctgctgttcgagaagcagaaggagttcggcaacccccacgtgagcggcggcctgaaggagggcatcgagaccctgctgatgacccagcgccccgccctgagcggcgacgccgtgcagaagatgctgggccactgcaccttcgagccagccgagcccaaggccgccaagaacacctacaccgccgagcgcttcatctggctgaccaagctgaacaacctgcgcatcctggagcagggcagcgagcgccccctgaccgacaccgagcgcgccaccctgatggacgagccctaccgcaagagcaagctgacctacgcccaggcccgcaagctgctgggtctggaggacaccgccttcttcaagggcctgcgctacggcaaggacaacgccgaggccagcaccctgatggagatgaaggcctaccacgccatcagccgcgccctggagaaggagggcctgaaggacaagaagagtcctctgaacctgagccccgagctgcaggacgagatcggcaccgccttcagcctgttcaagaccgacgaggacatcaccggccgcctgaaggaccgcatccagcccgagatcctggaggccctgctgaagcacatcagcttcgacaagttcgtgcagatcagcctgaaggccctgcgccgcatcgtgcccctgatggagcagggcaagcgctacgacgaggcctgcgccgagatctacggcgaccactacggcaagaagaacaccgaggagaagatctacctgcctcctatccccgccgacgagatccgcaaccccgtggtgctgcgcgccctgagccaggcccgcaaggtgatcaacggcgtggtgcgccgctacggcagccccgcccgcatccacatcgagaccgcccgcgaggtgggcaagagcttcaaggaccgcaaggagatcgagaagcgccaggaggagaaccgcaaggaccgcgagaaggccgccgccaagttccgcgagtacttccccaacttcgtgggcgagcccaagagcaaggacatcctgaagctgcgcctgtacgagcagcagcacggcaagtgcctgtacagcggcaaggagatcaacctgggccgcctgaacgagaagggctacgtggagatcgaccacgccctgcccttcagccgcacctgggacgacagcttcaacaacaaggtgctggtgctgggcagcgagaaccagaacaagggcaaccagaccccctacgagtacttcaacggcaaggacaacagccgcgagtggcaggagttcaaggcccgcgtggagaccagccgcttcccccgcagcaagaagcagcgcatcctgctgcagaagttcgacgaggacggcttcaaggagcgcaacctgaacgacacccgctacgtgaaccgcttcctgtgccagttcgtggccgaccgcatgcgcctgaccggcaagggcaagaagcgcgtgttcgccagcaacggccagatcaccaacctgctgcgcggcttctggggcctgcgcaaggtgcgcgccgagaacgaccgccaccacgccctggacgccgtggtggtggcctgcagcaccgtggccatgcagcagaagatcacccgcttcgtgcgctacaaggagatgaacgccttcgacggtaaaaccatcgacaaggagaccggcgaggtgctgcaccagaagacccacttcccccagccctgggagttcttcgcccaggaggtgatgatccgcgtgttcggcaagcccgacggcaagcccgagttcgaggaggccgacacccccgagaagctgcgcaccctgctggccgagaagctgagcagccgccctgaggccgtgcacgagtacgtgactcctctgttcgtgagccgcgcccccaaccgcaagatgagcggtcagggtcacatggagaccgtgaagagcgccaagcgcctggacgagggcgtgagcgtgctgcgcgtgcccctgacccagctgaagctgaaggacctggagaagatggtgaaccgcgagcgcgagcccaagctgtacgaggccctgaaggcccgcctggaggcccacaaggacgaccccgccaaggccttcgccgagcccttctacaagtacgacaaggccggcaaccgcacccagcaggtgaaggccgtgcgcgtggagcaggtgcagaagaccggcgtgtgggtgcgcaaccacaacggcatcgccgacaacgccaccatggtgcgcgtggacgtgttcgagaagggcgacaagtactacctggtgcccatctacagctggcaggtggccaagggcatcctgcccgaccgcgccgtggtgcagggcaaggacgaggaggactggcagctgatcgacgacagcttcaacttcaagttcagcctgcaccccaacgacctggtggaggtgatcaccaagaaggcccgcatgttcggctacttcgccagctgccaccgcggcaccggcaacatcaacatccgcatccacgacctggaccacaagatcggcaagaacggcatcctggagggcatcggcgtgaagaccgccctgagcttccagaagtaccagatcgacgagctgggcaaggagatccgcccctgccgcctgaagaagcgccctcctgtgcgctaa

SEQ ID NO: 14 - protein, 859 aa, synthetic construct
  REGION 1..859: note = Synthetic Cas9 consensus sequence derived from Sm, Sp, St, and Li Cas9 sequences
  REGION 8..18: note = misc_feature - N-terminal RuvC-like domain
  VARIANT 26: note = Xaa can be any naturally occurring amino acid
  VARIANT 29..31: note = Each Xaa can independently be any naturally occurring amino acid
  VARIANT 33: note = Xaa can be any naturally occurring amino acid
  VARIANT 75: note = Xaa can be any naturally occurring amino acid
  VARIANT 76: note = Xaa can be any naturally occurring amino acid
  REGION 78..80: note = misc_feature - Each Xaa can independently be any naturally occurring amino acid
  VARIANT 85: note = Xaa can be any naturally occurring amino acid
  VARIANT 89: note = Xaa can be any naturally occurring amino acid
  VARIANT 98: note = Xaa can be any naturally occurring amino acid
  VARIANT 106: note = Xaa can be any naturally occurring amino acid
  VARIANT 126: note = Xaa can be any naturally occurring amino acid
  REGION 128..133: note = misc_feature - Each Xaa can independently be any naturally occurring amino acid
  REGION 139..147: note = misc_feature - Each Xaa can independently be any naturally occurring amino acid
  REGION 153..155: note = misc_feature - Each Xaa can independently be any naturally occurring amino acid
  VARIANT 157: note = Xaa can be any naturally occurring amino acid
  VARIANT 159: note = Xaa can be any naturally occurring amino acid
  REGION 166..168: note = misc_feature - Each Xaa can independently be any naturally occurring amino acid
  REGION 173..175: note = misc_feature - Each Xaa can independently be any naturally occurring amino acid
  VARIANT 177: note = Xaa can be any naturally occurring amino acid
  REGION 185..187: note = misc_feature - Each Xaa can independently be any naturally occurring amino acid
  VARIANT 189: note = Xaa can be any naturally occurring amino acid
  REGION 192..195: note = misc_feature - Each Xaa can independently be any naturally occurring amino acid
  VARIANT 206: note = Xaa can be any naturally occurring amino acid
  VARIANT 212: note = Xaa can be any naturally occurring amino acid
  VARIANT 220: note = Xaa can be any naturally occurring amino acid
  REGION 244..246: note = misc_feature - Each Xaa can independently be any naturally occurring amino acid
  REGION 252..254: note = misc_feature - Each Xaa can independently be any naturally occurring amino acid
  VARIANT 257: note = Xaa can be any naturally occurring amino acid
  VARIANT 271: note = Xaa can be any naturally occurring amino acid
  VARIANT 273: note = Xaa can be any naturally occurring amino acid
  VARIANT 290: note = Xaa can be any naturally occurring amino acid
  REGION 292..294: note = misc_feature - Each Xaa can independently be any naturally occurring amino acid
  REGION 319..322: note = misc_feature - Each Xaa can independently be any naturally occurring amino acid
  REGION 335..337: note = misc_feature - Each Xaa can independently be any naturally occurring amino acid
  REGION 356..361: note = misc_feature - Each Xaa can independently be any naturally occurring amino acid
  VARIANT 363: note = Xaa can be any naturally occurring amino acid
  REGION 370..373: note = misc_feature - Each Xaa can independently be any naturally occurring amino acid
  VARIANT 375: note = Xaa can be any naturally occurring amino acid
  VARIANT 376: note = Xaa can be any naturally occurring amino acid
  VARIANT 379: note = Xaa can be any naturally occurring amino acid
  REGION 386..390: note = misc_feature - Each Xaa can independently be any naturally occurring amino acid
  VARIANT 393: note = Xaa can be any naturally occurring amino acid
  VARIANT 407: note = Xaa can be any naturally occurring amino acid
  VARIANT 411: note = Xaa can be any naturally occurring amino acid
  REGION 413..416: note = misc_feature - Each Xaa can independently be any naturally occurring amino acid
  VARIANT 418: note = Xaa can be any naturally occurring amino acid
  VARIANT 428: note = Xaa can be any naturally occurring amino acid
  VARIANT 431: note = Xaa can be any naturally occurring amino acid
  VARIANT 433: note = Xaa can be any naturally occurring amino acid
  REGION 437..439: note = misc_feature - Each Xaa can independently be any naturally occurring amino acid
  REGION 465..469: note = misc_feature - Each Xaa can independently be any naturally occurring amino acid
  VARIANT 482: note = Xaa can be any naturally occurring amino acid
  REGION 495..502: note = misc_feature - RuvC-like domain
  VARIANT 497: note = Xaa can be any naturally occurring amino acid
  VARIANT 510: note = Xaa can be any naturally occurring amino acid
  VARIANT 513: note = Xaa can be any naturally occurring amino acid
  VARIANT 525: note = Xaa can be any naturally occurring amino acid
  VARIANT 531: note = Xaa can be any naturally occurring amino acid
  VARIANT 532: note = Xaa can be any naturally occurring amino acid
  VARIANT 534: note = Xaa can be any naturally occurring amino acid
  VARIANT 21: note = Xaa can be any naturally occurring amino acid
  VARIANT 547: note = Xaa can be any naturally occurring amino acid
  REGION 555..575: note = misc_feature - HNH-like domain
  VARIANT 563: note = Xaa can be any naturally occurring amino acid
  VARIANT 565: note = Xaa can be any naturally occurring amino acid
  VARIANT 579: note = Xaa can be any naturally occurring amino acid
  VARIANT 588: note = Xaa can be any naturally occurring amino acid
  REGION 594..596: note = misc_feature - Each Xaa can independently be any naturally occurring amino acid
  VARIANT 628: note = Xaa can be any naturally occurring amino acid
  VARIANT 634: note = Xaa can be any naturally occurring amino acid
  REGION 638..641: note = misc_feature - Each Xaa can independently be any naturally occurring amino acid
  REGION 643..645: note = misc_feature - Each Xaa can independently be any naturally occurring amino acid
  VARIANT 657: note = Xaa can be any naturally occurring amino acid
  VARIANT 665: note = Xaa can be any naturally occurring amino acid
  VARIANT 669: note = Xaa can be any naturally occurring amino acid
  REGION 670..677: note = misc_feature - RuvC-like domain
  VARIANT 683: note = Xaa can be any naturally occurring amino acid
  VARIANT 710: note = Xaa can be any naturally occurring amino acid
  REGION 717..720: note = misc_feature - Each Xaa can independently be any naturally occurring amino acid
  VARIANT 727: note = Xaa can be any naturally occurring amino acid
  REGION 733..735: note = misc_feature - Each Xaa can independently be any naturally occurring amino acid
  REGION 749..754: note = misc_feature - Each Xaa can independently be any naturally occurring amino acid
  REGION 758..761: note = misc_feature - Each Xaa can independently be any naturally occurring amino acid
  REGION 763..768: note = misc_feature - Each Xaa can independently be any naturally occurring amino acid
  VARIANT 771: note = Xaa can be any naturally occurring amino acid
  REGION 774..777: note = misc_feature - Each Xaa can independently be any naturally occurring amino acid
  VARIANT 782: note = Xaa can be any naturally occurring amino acid
  REGION 784..786: note = misc_feature - Each Xaa can independently be any naturally occurring amino acid
  VARIANT 802: note = Xaa can be any naturally occurring amino acid
  VARIANT 804: note = Xaa can be any naturally occurring amino acid
  REGION 806..813: note = misc_feature - Each Xaa can independently be any naturally occurring amino acid
  REGION 815..818: note = misc_feature - Each Xaa can independently be any naturally occurring amino acid
  REGION 823..827: note = misc_feature - Each Xaa can independently be any naturally occurring amino acid
  REGION 835..837: note = misc_feature - Each Xaa can independently be any naturally occurring amino acid
  REGION 842..844: note = misc_feature - Each Xaa can independently be any naturally occurring amino acid
  VARIANT 857: note = Xaa can be any naturally occurring amino acid
  VARIANT 4: note = Xaa can be any naturally occurring amino acid
  VARIANT 23: note = Xaa can be any naturally occurring amino acid
  VARIANT 24: note = Xaa can be any naturally occurring amino acid
  VARIANT 36: note = Xaa can be any naturally occurring amino acid
  VARIANT 45: note = Xaa can be any naturally occurring amino acid
  VARIANT 54: note = Xaa can be any naturally occurring amino acid
  VARIANT 63: note = Xaa can be any naturally occurring amino acid
  VARIANT 71: note = Xaa can be any naturally occurring amino acid
  VARIANT 82: note = Xaa can be any naturally occurring amino acid
  VARIANT 84: note = Xaa can be any naturally occurring amino acid
  VARIANT 90: note = Xaa can be any naturally occurring amino acid
  VARIANT 100: note = Xaa can be any naturally occurring amino acid
  VARIANT 113: note = Xaa can be any naturally occurring amino acid
  VARIANT 116: note = Xaa can be any naturally occurring amino acid
  VARIANT 125: note = Xaa can be any naturally occurring amino acid
  VARIANT 135: note = Xaa can be any naturally occurring amino acid
  VARIANT 137: note = Xaa can be any naturally occurring amino acid
  VARIANT 161: note = Xaa can be any naturally occurring amino acid
  VARIANT 163: note = Xaa can be any naturally occurring amino acid
  VARIANT 170: note = Xaa can be any naturally occurring amino acid
  VARIANT 171: note = Xaa can be any naturally occurring amino acid
  VARIANT 183: note = Xaa can be any naturally occurring amino acid
  VARIANT 198: note = Xaa can be any naturally occurring amino acid
  VARIANT 199: note = Xaa can be any naturally occurring amino acid
  VARIANT 202: note = Xaa can be any naturally occurring amino acid
  VARIANT 207: note = Xaa can be any naturally occurring amino acid
  VARIANT 210: note = Xaa can be any naturally occurring amino acid
  VARIANT 213: note = Xaa can be any naturally occurring amino acid
  VARIANT 219: note = Xaa can be any naturally occurring amino acid
  VARIANT 222: note = Xaa can be any naturally occurring amino acid
  VARIANT 224: note = Xaa can be any naturally occurring amino acid
  VARIANT 226: note = Xaa can be any naturally occurring amino acid
  VARIANT 236: note = Xaa can be any naturally occurring amino acid
  VARIANT 240: note = Xaa can be any naturally occurring amino acid
  VARIANT 241: note = Xaa can be any naturally occurring amino acid
  VARIANT 248: note = Xaa can be any naturally occurring amino acid
  VARIANT 249: note = Xaa can be any naturally occurring amino acid
  VARIANT 250: note = Xaa can be any naturally occurring amino acid
  VARIANT 256: note = Xaa can be any naturally occurring amino acid
  VARIANT 268: note = Xaa can be any naturally occurring amino acid
  VARIANT 277: note = Xaa can be any naturally occurring amino acid
  VARIANT 280: note = Xaa can be any naturally occurring amino acid
  VARIANT 281: note = Xaa can be any naturally occurring amino acid
  VARIANT 283: note = Xaa can be any naturally occurring amino acid
  VARIANT 289: note = Xaa can be any naturally occurring amino acid
  VARIANT 301: note = Xaa can be any naturally occurring amino acid
  VARIANT 308: note = Xaa can be any naturally occurring amino acid
  VARIANT 328: note = Xaa can be any naturally occurring amino acid
  VARIANT 329: note = Xaa can be any naturally occurring amino acid
  VARIANT 346: note = Xaa can be any naturally occurring amino acid
  VARIANT 347: note = Xaa can be any naturally occurring amino acid
  VARIANT 395: note = Xaa can be any naturally occurring amino acid
  VARIANT 396: note = Xaa can be any naturally occurring amino acid
  VARIANT 398: note = Xaa can be any naturally occurring amino acid
  VARIANT 400: note = Xaa can be any naturally occurring amino acid
  VARIANT 403: note = Xaa can be any naturally occurring amino acid
  VARIANT 410: note = Xaa can be any naturally occurring amino acid
  VARIANT 422: note = Xaa can be any naturally occurring amino acid
  VARIANT 445: note = Xaa can be any naturally occurring amino acid
  VARIANT 451: note = Xaa can be any naturally occurring amino acid
  VARIANT 456: note = Xaa can be any naturally occurring amino acid
  VARIANT 459: note = Xaa can be any naturally occurring amino acid
  VARIANT 481: note = Xaa can be any naturally occurring amino acid
  VARIANT 484: note = Xaa can be any naturally occurring amino acid
  VARIANT 490: note = Xaa can be any naturally occurring amino acid
  VARIANT 494: note = Xaa can be any naturally occurring amino acid
  VARIANT 506: note = Xaa can be any naturally occurring amino acid
  VARIANT 514: note = Xaa can be any naturally occurring amino acid
  VARIANT 517: note = Xaa can be any naturally occurring amino acid
  VARIANT 520: note = Xaa can be any naturally occurring amino acid
  VARIANT 526: note = Xaa can be any naturally occurring amino acid
  VARIANT 529: note = Xaa can be any naturally occurring amino acid
  VARIANT 542: note = Xaa can be any naturally occurring amino acid
  VARIANT 546: note = Xaa can be any naturally occurring amino acid
  VARIANT 553: note = Xaa can be any naturally occurring amino acid
  VARIANT 556: note = Xaa can be any naturally occurring amino acid
  VARIANT 560: note = Xaa can be any naturally occurring amino acid
  VARIANT 567: note = Xaa can be any naturally occurring amino acid
  VARIANT 582: note = Xaa can be any naturally occurring amino acid
  VARIANT 583: note = Xaa can be any naturally occurring amino acid
  VARIANT 585: note = Xaa can be any naturally occurring amino acid
  VARIANT 590: note = Xaa can be any naturally occurring amino acid
  VARIANT 592: note = Xaa can be any naturally occurring amino acid
  VARIANT 610: note = Xaa can be any naturally occurring amino acid
  VARIANT 616: note = Xaa can be any naturally occurring amino acid
  VARIANT 631: note = Xaa can be any naturally occurring amino acid
  VARIANT 633: note = Xaa can be any naturally occurring amino acid
  VARIANT 636: note = Xaa can be any naturally occurring amino acid
  VARIANT 653: note = Xaa can be any naturally occurring amino acid
  VARIANT 659: note = Xaa can be any naturally occurring amino acid
  VARIANT 660: note = Xaa can be any naturally occurring amino acid
  VARIANT 666: note = Xaa can be any naturally occurring amino acid
  VARIANT 668: note = Xaa can be any naturally occurring amino acid
  VARIANT 680: note = Xaa can be any naturally occurring amino acid
  VARIANT 681: note = Xaa can be any naturally occurring amino acid
  VARIANT 686: note = Xaa can be any naturally occurring amino acid
  VARIANT 696: note = Xaa can be any naturally occurring amino acid
  VARIANT 697: note = Xaa can be any naturally occurring amino acid
  VARIANT 704: note = Xaa can be any naturally occurring amino acid
  VARIANT 708: note = Xaa can be any naturally occurring amino acid
  VARIANT 711: note = Xaa can be any naturally occurring amino acid
  VARIANT 714: note = Xaa can be any naturally occurring amino acid
  VARIANT 722: note = Xaa can be any naturally occurring amino acid
  VARIANT 725: note = Xaa can be any naturally occurring amino acid
  VARIANT 740: note = Xaa can be any naturally occurring amino acid
  VARIANT 742: note = Xaa can be any naturally occurring amino acid
  VARIANT 788: note = Xaa can be any naturally occurring amino acid
  VARIANT 790: note = Xaa can be any naturally occurring amino acid
  VARIANT 795: note = Xaa can be any naturally occurring amino acid
  VARIANT 799: note = Xaa can be any naturally occurring amino acid
  VARIANT 801: note = Xaa can be any naturally occurring amino acid
  VARIANT 820: note = Xaa can be any naturally occurring amino acid
  VARIANT 829: note = Xaa can be any naturally occurring amino acid
  VARIANT 830: note = Xaa can be any naturally occurring amino acid
  VARIANT 832: note = Xaa can be any naturally occurring amino acid
  VARIANT 846: note = Xaa can be any naturally occurring amino acid
  VARIANT 848: note = Xaa can be any naturally occurring amino acid
  VARIANT 851: note = Xaa can be any naturally occurring amino acid
MKYXIGLDIGTNSVGWAVTDXYXXKXKGXXXIXKNXGLFDGTARXRTARRRRRXNRIYLQIFXEMDFFRLXSFVXXKXXXPXFXXEYHXXPTIYHLRXLXKDLRLXYLALAHXIKXRGNFLIEGXXNXXXXXXYXFXIXXXXXXXXXPEKGFXXXLXGXFXFXLEXXXKXXYXXXLXLLIGDXYXXXFXAKXXXXLSXXVTXALSXXMIXRXXHDLLKXXYXEXFXKGYAGYIDGXQFYXXKLXXXGXXXKXXXEXXLRKQRTFDNGXIPXQXHLEXAIXXQXYPFLNXXIXXXTFRIPYXVGPLAGXSFAWRKIPWNXXXXDSAFIXXMTDLPXXXVLPKHSLYXXVYNELTKVXXXXXXKXIFKRKVXXXXGXXFNXSTYHDLXXXXXLDXNXXEXIXLTXFEDXMIXXLXXXXKXLRRXYTGWGXLSXLXGIRXXXSTILDXLDNRNXMQLIXDLXFKIKQXXXXXGSPAIKKGILQXXKXVDELVXMGPXIVXEMARENQTXGNSXRKXXKEXGSXILKEXXNLXNXXLXLYYLQNGXDMYXXLDILSXYDXDHIXPQXFXDXSIDNVLSNRKDXVPXXVXKKXWXLXLXXXRKFDLTKAERGGLXDKAFIXRQLVETRQITKXVAXLXXNXDXXXXVXXXTLKSLVSXFRKXFXXLYKVXXNXXHHAHDAYLNVXXLXYPXLEEFVYGDYXXKATKFYXNIMXFXXGEXWKXXXXVXMQXNXVKKEQXXXPKNSXLXKDKYGGXXXXXXKGKXXXXIXXXXXXFLXGYXXXXLPKYXLXXXGXRXLASEXKGNXLXXLXAXXXXXXXXEXXXXFXANXXXXXLXXGXAFXXXIRRYXXXTXIXQSXTGLYEXRL

SEQ ID NO: 15 - protein, 8 aa, synthetic construct
  REGION 1..8: note = RuvC-like domain
  VARIANT 2: note = Xaa is Val or His
  VARIANT 3: note = Xaa is Ile, Leu, or Val
  VARIANT 5: note = Xaa is Met or Thr
IXXEXARE

SEQ ID NO: 16 - protein, 8 aa, synthetic construct
  REGION 1..8: note = RuvC-like domain
  VARIANT 3: note = Xaa is Ile, Leu, or Val
IVXEMARE

SEQ ID NO: 17 - protein, 8 aa, synthetic construct
  REGION 1..8: note = RuvC-like domain
  VARIANT 4: note = Xaa is His or Leu
  VARIANT 7: note = Xaa is Arg or Val
  VARIANT 8: note = Xaa is Glu or Val
HHAXDAXX

SEQ ID NO: 18 - protein, 8 aa, synthetic construct
  REGION 1..8: note = RuvC-like domain
HHAHDAYL

SEQ ID NO: 19 - protein, 30 aa, synthetic construct
  REGION 1..30: note = N-terminal RuvC-like domain
  VARIANT 2: note = Xaa is Lys or Pro
  VARIANT 4: note = Xaa is Val, Leu, Ile, or Phe
  VARIANT 5: note = Xaa is Gly, Ala, or Ser
  VARIANT 6: note = Xaa is Leu, Ile, Val, or Phe
  MOD_RES 7..26: note = N-terminal RuvC-like domain, each Xaa can be any amino acid or absent, region may encompass 5-20 residues
  VARIANT 29: note = Xaa is Asp, Glu, Asn, or Gln
KXYXXXXXXXXXXXXXXXXXXXXXXXTDXY

SEQ ID NO: 20 - skipped

SEQ ID NO: 21 - protein, 12 aa, synthetic construct
  REGION 1..12: note = N-terminal RuvC-like domain
  VARIANT 2: note = Xaa is Ile, Val, Met, Leu, or Thr
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 7: note = Xaa is Val, Ile, Leu, Cys, Thr, or Phe
  VARIANT 9: note = Xaa is Trp, Phe, Val, Tyr, Ser, or Leu
  VARIANT 10: note = Xaa is Ala, Ser, Cys, Val, or Gly
  VARIANT 11: note = Xaa is Val, Ile, Leu, Ala, Met, or His
  VARIANT 12: note = Any amino acid or absent
DXGXXSXGXXXX

SEQ ID NO: 22 - protein, 12 aa, synthetic construct
  REGION 1..12: note = N-terminal RuvC-like domain
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 11: note = Xaa is Val, Ile, Leu, Ala, Met, or His
  VARIANT 12: note = Any amino acid or absent
DIGXXSVGWAXX

SEQ ID NO: 23 - protein, 12 aa, synthetic construct
  REGION 1..12: note = N-terminal RuvC-like domain
  MOD_RES 12: note = Any non-polar alkyl amino acid or a hydroxyl amino acid
DIGTNSVGWAVX

SEQ ID NO: 24 - protein, 73 aa, synthetic construct
  REGION 1..73: note = HNH-like domain
  VARIANT 8: note = Xaa is Lys or Arg
  VARIANT 12: note = Xaa is Val or Thr
  VARIANT 13: note = Xaa is Gly or Asp
  VARIANT 14: note = Xaa is Glu, Gln, or Asp
  VARIANT 15: note = Xaa is Glu or Asp
  VARIANT 19: note = Xaa is Asp, Asn, or His
  VARIANT 20: note = Xaa is Tyr, Arg, or Asn
  VARIANT 23: note = Xaa is Gln, Asp, or Asn
  MOD_RES 25..64: note = HNH-like domain, each Xaa can be any amino acid or absent, region may encompass 15-40 residues
  VARIANT 67: note = Xaa is Gly or Glu
  VARIANT 69: note = Xaa is Ser or Gly
  VARIANT 71: note = Xaa is Asp or Asn
LYYLQNGXDMYXXXXLDIXXLSXYXXXXXXXXXXXXXXXXXXXXXXXXXXXXXXXXXXXXXXXXNRXKXDXVP

SEQ ID NO: 25 - protein, 27 aa, synthetic construct
  REGION 1..27: note = HNH-like domain
  VARIANT 1: note = Xaa is Asp, Glu, Gln, or Asn
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 9: note = Xaa is Ser, Ala, Asp, Thr, or Lys
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 13: note = Xaa is Asp, Ser, Asn, Arg, Leu, or Thr
  VARIANT 14: note = Xaa is Asp, Asn, or Ser
  VARIANT 15: note = Xaa is Ser, Ala, Thr, Gly, or Arg
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 19: note = Xaa is Lys, Leu, Arg, Met, Thr, or Phe
  VARIANT 20: note = Xaa is Val, Leu, Ile, Ala, or Thr
  VARIANT 21: note = Xaa is Leu, Ile, Val, or Ala
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XXXHXXPXXXXXXXXXXNXXXXXXXXN

SEQ ID NO: 26 - protein, 27 aa, synthetic construct
  REGION 1..27: note = HNH-like domain
  VARIANT 1: note = Xaa is Asp or Glu
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XXXHXXPXSXXXDDSXXNKVLXXXXXN

SEQ ID NO: 27 - protein, 27 aa, synthetic construct
  REGION 1..27: note = HNH-like domain
  VARIANT 1: note = Xaa is Asp or Glu
  VARIANT 3: note = Xaa is Asp or Glu
  VARIANT 8: note = Xaa is Gln, His, Arg, Lys, Tyr, Ile, Leu, or Trp
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XVXHIVPXSXXXDDSXXNKVLTXXXXN

SEQ ID NO: 28 - protein, 27 aa, synthetic construct
  REGION 1..27: note = HNH-like domain
  VARIANT 2: note = Xaa is Ile or Val
  VARIANT 6: note = Xaa is Ile or Val
  VARIANT 9: note = Xaa is Ala or Ser
  VARIANT 11: note = Xaa is Ile or Leu
  VARIANT 12: note = Xaa is Lys or Thr
  VARIANT 14: note = Xaa is Asp or Asn
  VARIANT 19: note = Xaa is Arg, Lys, or Leu
  VARIANT 22: note = Xaa is Thr or Val
  VARIANT 23: note = Xaa is Ser or Arg
  VARIANT 25: note = Xaa is Lys, Asp, or Ala
  VARIANT 26: note = Xaa is Glu, Lys, Gly, or Asn
DXDHIXPQXFXXDXSIDNXVLXXSXXN

SEQ ID NO: 29 - RNA, 116 nt, synthetic construct
  misc_feature 1..116: note = gRNA
  misc_difference 1..20: note = a, c, u, g, Unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngttttagagctatgctgttttggaaacaaaacagcatagcaagttaaaataaggctagtccgttatcaacttgaaaaagtggcaccgagtcggtgc

SEQ ID NO: 30 - RNA, 116 nt, synthetic construct
  misc_feature 1..116: note = gRNA
  misc_difference 1..20: note = a, c, u, g, Unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngtattagagctatgctgtattggaaacaatacagcatagcaagttaatataaggctagtccgttatcaacttgaaaaagtggcaccgagtcggtgc

SEQ ID NO: 31 - RNA, 96 nt, synthetic construct
  misc_feature 1..96: note = gRNA
  misc_difference 1..20: note = a, c, u, g, Unknown or other
  misc_feature 1..20: note = targeting domain
  misc_feature 21..32: note = first complementarity domain
  misc_feature 33..36: note = linking domain
  misc_feature 37..50: note = second complementarity domain
  misc_feature 51..62: note = proximal domain
  source 1..96: mol_type = other RNA
nnnnnnnnnnnnnnnnnnnngtttaagagctagaaatagcaagtttaaataaggctagtccgttatcaacttgaaaaagtggcaccgagtcggtgc

SEQ ID NO: 32 - RNA, 47 nt, synthetic construct
  misc_feature 1..47: note = gRNA proximal and tail domains derived from S. pyogenes
  source 1..47: mol_type = other RNA
aaggctagtccgttatcaacttgaaaaagtggcaccgagtcggtgct

SEQ ID NO: 33 - RNA, 49 nt, synthetic construct
  misc_feature 1..49: note = gRNA proximal and tail domains
  source 1..49: mol_type = other RNA
aaggctagtccgttatcaacttgaaaaagtggcaccgagtcggtggtgc

SEQ ID NO: 34 - RNA, 51 nt, synthetic construct
  misc_feature 1..51: note = gRNA proximal and tail domains
  source 1..51: mol_type = other RNA
aaggctagtccgttatcaacttgaaaaagtggcaccgagtcggtgcggatc

SEQ ID NO: 35 - RNA, 31 nt, synthetic construct
  misc_feature 1..31: note = gRNA proximal and tail domains
  source 1..31: mol_type = other RNA
aaggctagtccgttatcaacttgaaaaagtg

SEQ ID NO: 36 - RNA, 18 nt, synthetic construct
  misc_feature 1..18: note = gRNA proximal and tail domains
  source 1..18: mol_type = other RNA
aaggctagtccgttatca

SEQ ID NO: 37 - RNA, 12 nt, synthetic construct
  misc_feature 1..12: note = gRNA proximal and tail domains
  source 1..12: mol_type = other RNA
aaggctagtccg

SEQ ID NO: 38 - RNA, 102 nt, synthetic construct
  misc_feature 1..102: note = Unimolecular gRNA derived from S. aureus
  misc_difference 1..20: note = n is a, c, g, or u
  misc_feature 1..20: note = Targeting domain
  source 1..102: mol_type = other RNA
nnnnnnnnnnnnnnnnnnnngttttagtactctggaaacagaatctactaaaacaaggcaaaatgccgtgtttatctcgtcaacttgttggcgagatttttt

SEQ ID NO: 39 - RNA, 42 nt, synthetic construct
  misc_feature 1..42: note = Modular gRNA derived from S. pyogenes
  misc_difference 1..20: note = a, c, u, g, Unknown or other
  misc_feature 1..20: note = Targeting domain
  misc_feature 21..42: note = First complementarity domain
  source 1..42: mol_type = other RNA
nnnnnnnnnnnnnnnnnnnngttttagagctatgctgttttg

SEQ ID NO: 40 - RNA, 85 nt, synthetic construct
  misc_feature 1..85: note = Modular gRNA derived from S. pyogenes
  misc_feature 1..9: note = 5' extension domain
  misc_feature 10..33: note = Second complementarity domain
  misc_feature 34..45: note = Proximal domain
  misc_feature 46..85: note = Tail domain
  source 1..85: mol_type = other RNA
ggaaccattcaaaacagcatagcaagttaaaataaggctagtccgttatcaacttgaaaaagtggcaccgagtcggtgctttttt

SEQ ID NO: 41 - RNA, 62 nt, synthetic construct
  misc_feature 1..62: note = Unimolecular gRNA derived from S. pyogenes
  misc_difference 1..20: note = a, c, u, g, Unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..62: mol_type = other RNA
nnnnnnnnnnnnnnnnnnnngttttagagctagaaatagcaagttaaaataaggctagtccg

SEQ ID NO: 42 - RNA, 102 nt, synthetic construct
  misc_feature 1..102: note = Unimolecular gRNA derived from S. pyogenes
  misc_difference 1..20: note = a, c, u, g, Unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  misc_feature 63..102: note = Tail domain
  source 1..102: mol_type = other RNA
nnnnnnnnnnnnnnnnnnnngttttagagctagaaatagcaagttaaaataaggctagtccgttatcaacttgaaaaagtggcaccgagtcggtgctttttt

SEQ ID NO: 43 - RNA, 75 nt, synthetic construct
  misc_feature 1..75: note = Unimolecular gRNA derived from S. pyogenes
  misc_feature 1..20: note = Targeting domain
  misc_difference 1..20: note = a, c, u, g, Unknown or other
  misc_feature 21..36: note = First complementarity domain
  misc_feature 37..40: note = Linking domain
  misc_feature 41..58: note = Second complementarity domain
  misc_feature 59..70: note = Proximal domain
  misc_feature 71..75: note = Tail domain
  source 1..75: mol_type = other RNA
nnnnnnnnnnnnnnnnnnnngttttagagctatgctgaaaagcatagcaagttaaaataaggctagtccgttatc

SEQ ID NO: 44 - RNA, 87 nt, synthetic construct
  misc_feature 1..87: note = Unimolecular gRNA derived from S. pyogenes
  misc_difference 1..20: note = a, c, u, g, Unknown or other
  misc_feature 1..20: note = Targeting domain
  misc_feature 21..32: note = First complementarity domain
  misc_feature 43..46: note = Linking domain
  misc_feature 57..70: note = Second complementarity domain
  misc_feature 71..82: note = Proximal domain
  misc_feature 83..87: note = Tail domain
  source 1..87: mol_type = other RNA
nnnnnnnnnnnnnnnnnnnngttttagagctatgctgttttggaaacaaaacagcatagcaagttaaaataaggctagtccgttatc

SEQ ID NO: 45 - RNA, 42 nt, synthetic construct
  misc_feature 1..42: note = Modular gRNA derived from S. thermophilus
  misc_difference 1..20: note = a, c, u, g, Unknown or other
  misc_feature 1..20: note = Targeting domain
  misc_feature 21..42: note = First complementarity domain
  source 1..42: mol_type = other RNA
nnnnnnnnnnnnnnnnnnnngttttagagctgtgttgtttcg

SEQ ID NO: 46 - RNA, 78 nt, synthetic construct
  misc_feature 1..78: note = Modular gRNA derived from S. thermophilus
  misc_feature 1..3: note = 5' extension domain
  misc_feature 4..27: note = Second complementarity domain
  misc_feature 28..40: note = Proximal domain
  misc_feature 41..78: note = Tail domain
  source 1..78: mol_type = other RNA
gggcgaaacaacacagcgagttaaaataaggcttagtccgtactcaacttgaaaaggtggcaccgattcggtgttttt

SEQ ID NO: 47 - RNA, 85 nt, synthetic construct
  misc_feature 1..85: note = Modular gRNA derived from S. pyogenes
  source 1..85: mol_type = other RNA
gaaccattcaaaacagcatagcaagttaaaataaggctagtccgttatcaacttgaaaaagtggcaccgagtcggtgcttttttt

SEQ ID NO: 48 - RNA, 96 nt, synthetic construct
  misc_feature 1..96: note = gRNA from S. pyogenes
  misc_difference 1..20: note = n is a, c, g, or u
  misc_feature 1..20: note = Targeting domain
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 49 - RNA, 96 nt, synthetic construct
  misc_feature 1..96: note = gRNA
  misc_difference 1..20: note = n is a, c, g, or u
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..96: mol_type = other RNA
nnnnnnnnnnnnnnnnnnnngtattagagctagaaatagcaagttaatataaggctagtccgttatcaacttgaaaaagtggcaccgagtcggtgc

SEQ ID NO: 50 - RNA, 104 nt, synthetic construct
  misc_feature 1..104: note = gRNA
  misc_difference 1..20: note = n is a, c, g, or u
  misc_feature 1..20: note = Targeting domain
  misc_feature 21..36: note = First complementarity domain
  misc_feature 37..40: note = Linking domain
  misc_feature 41..58: note = Second complementarity domain
  source 1..104: mol_type = other RNA
nnnnnnnnnnnnnnnnnnnngttttagagctatgctgaaaagcatagcaagttaaaataaggctagtccgttatcaacttgaaaaagtggcaccgagtcggtgc

SEQ ID NO: 51 - RNA, 106 nt, synthetic construct
  misc_feature 1..106: note = gRNA
  misc_difference 1..20: note = n is a, c, g, or u
  misc_feature 1..20: note = Targeting domain
  misc_feature 21..37: note = First complementarity domain
  misc_feature 38..41: note = Linking domain
  misc_feature 42..60: note = Second complementarity domain
  source 1..106: mol_type = other RNA
nnnnnnnnnnnnnnnnnnnngttttagagctatgctggaaacagcatagcaagttaaaataaggctagtccgttatcaacttgaaaaagtggcaccgagtcggtgc

SEQ ID NO: 52 - protein, 12 aa, Peptoniphilus duerdenii
DIGTASVGWAVT

SEQ ID NO: 53 - protein, 12 aa, Treponema denticola
DVGTGSVGWAVT

SEQ ID NO: 54 - protein, 12 aa, Streptococcus mutans
DIGTNSVGWAVV

SEQ ID NO: 55 - protein, 12 aa, Streptococcus mutans
DIGTNSVGWAVI

SEQ ID NO: 56 - protein, 12 aa, Streptococcus mutans
DIGTNSVGWAVL

SEQ ID NO: 57 - protein, 12 aa, Streptococcus mutans
DLGTNSIGWAVV

SEQ ID NO: 58 - protein, 11 aa, Pedobacter glucosidilyticus
DLGTNSIGWAI

SEQ ID NO: 59 - protein, 12 aa, Streptococcus mutans
DLGTNSIGWALV

SEQ ID NO: 60 - protein, 12 aa, Fusobacterium nucleatum
DIGTNSVGWCVT

SEQ ID NO: 61 - protein, 12 aa, Streptococcus mutans
DIGTNSVGYAVT

SEQ ID NO: 62 - protein, 12 aa, Streptococcus mutans
DMGTGSLGWAVT

SEQ ID NO: 63 - protein, 12 aa, Streptococcus mutans
DIGTSSVGWAAI

SEQ ID NO: 64 - protein, 12 aa, Streptococcus mutans
DLGTGSVGWAVV

SEQ ID NO: 65 - protein, 12 aa, Streptococcus mutans
DLGVGSVGWAIV

SEQ ID NO: 66 - protein, 12 aa, Streptococcus mutans
DLGIASIGWAII

SEQ ID NO: 67 - protein, 12 aa, Streptococcus mutans
DLGIASVGWAIV

SEQ ID NO: 68 - protein, 12 aa, Streptococcus mutans
DLGVASVGWSIV

SEQ ID NO: 69 - protein, 12 aa, Streptococcus mutans
DIGIASVGWAIL

SEQ ID NO: 70 - protein, 12 aa, Streptococcus mutans
DLGISSVGWSVI

SEQ ID NO: 71 - protein, 12 aa, Streptococcus mutans
DIGIASVGWSVI

SEQ ID NO: 72 - protein, 12 aa, Streptococcus mutans
DVGIGSIGWAVI

SEQ ID NO: 73 - protein, 12 aa, Streptococcus mutans
DLGVGSIGFAIV

SEQ ID NO: 74 - protein, 12 aa, Akkermansia muciniphila
DIGYASIGWAVI

SEQ ID NO: 75 - protein, 12 aa, Prevotella ruminicola
DTGTNSLGWAIV

SEQ ID NO: 76 - protein, 12 aa, Candidatus Puniceispirillum marinum
DLGTNSIGWCLL

SEQ ID NO: 77 - protein, 12 aa, Rhodospirillum rubrum
DIGTDSLGWAVF

SEQ ID NO: 78 - protein, 12 aa, Streptococcus mutans
DIGSNSIGFAVV

SEQ ID NO: 79 - protein, 12 aa, Streptococcus mutans
DLGVGSIGVAVA

SEQ ID NO: 80 - protein, 12 aa, Rhodopseudomonas palustris
DLGIASCGWGVV

SEQ ID NO: 81 - protein, 12 aa, Streptococcus mutans
DLGIASVGWCLT

SEQ ID NO: 82 - protein, 12 aa, Streptococcus mutans
DIGIGSVGVGIL

SEQ ID NO: 83 - protein, 12 aa, Streptococcus mutans
DIGITSVGYGLI

SEQ ID NO: 84 - protein, 12 aa, Streptococcus mutans
DIGITSVGFGII

SEQ ID NO: 85 - protein, 12 aa, Streptococcus mutans
DVGITSTGYAVL

SEQ ID NO: 86 - protein, 12 aa, Streptococcus mutans
DLGITSFGYAIL

SEQ ID NO: 87 - protein, 12 aa, Streptococcus mutans
DIGNASVGWSAF

SEQ ID NO: 88 - protein, 12 aa, Lactobacillus gasseri
DVGTNSCGWVAM

SEQ ID NO: 89 - protein, 12 aa, Streptococcus mutans
DVGERSIGLAAV

SEQ ID NO: 90 - protein, 12 aa, Streptococcus mutans
DVGLNSVGLAAV

SEQ ID NO: 91 - protein, 12 aa, Bifidobacterium dentium
DVGLMSVGLAAI

SEQ ID NO: 92 - protein, 12 aa, Corynebacterium diphtheriae
DVGTFSVGLAAI

SEQ ID NO: 93 - protein, 12 aa, Streptococcus mutans
DIGTGSVGYACM

SEQ ID NO: 94 - protein, 12 aa, Capnocytophaga ochracea
DLGTTSIGFAHI

SEQ ID NO: 95 - protein, 12 aa, Prevotella denticola
DLGTNSIGSSVR

SEQ ID NO: 96 - protein, 12 aa, Ralstonia solanacearum
DIGTNSIGWAVI

SEQ ID NO: 97 - protein, 12 aa, Streptococcus mutans
DLGIASVGWAVV

SEQ ID NO: 98 - protein, 12 aa, Comamonas granuli
DIGIASVGWAVL

SEQ ID NO: 99 - protein, 12 aa, Streptococcus mutans
DIGIASIGWAVI

SEQ ID NO: 100 - protein, 12 aa, Agathobacter rectalis
DIGIASVGWAII

SEQ ID NO: 101 - protein, 12 aa, Streptococcus mutans
DVGIASVGWAVI

SEQ ID NO: 102 - protein, 11 aa, Streptococcus mutans
DIGIASVGWAL

SEQ ID NO: 103 - protein, 12 aa, Neisseria meningitidis
DIGIASVGWAMV

SEQ ID NO: 104 - protein, 12 aa, Clostridium perfringens
DIGITSVGWAVI

SEQ ID NO: 105 - protein, 12 aa, Streptococcus mutans
DLGISSLGWAIV

SEQ ID NO: 106 - protein, 12 aa, Streptococcus mutans
DLGTNSIGWGLL

SEQ ID NO: 107 - protein, 12 aa, Verminephrobacter eiseniae
DLGSTSLGWAIF

SEQ ID NO: 108 - protein, 12 aa, Streptococcus mutans
DIGISSIGWAFS

SEQ ID NO: 109 - protein, 12 aa, Streptococcus mutans
DIGTTSIGFSVI

SEQ ID NO: 110 - protein, 12 aa, Streptococcus mutans
DIGTSSIGWWLY

SEQ ID NO: 111 - protein, 12 aa, Streptococcus mutans
DLGSNSLGWFVT

SEQ ID NO: 112 - protein, 12 aa, Streptococcus mutans
DLGANSLGWFVV

SEQ ID NO: 113 - protein, 15 aa, Bacillus cereus
DIGLRIGITSCGWSI

SEQ ID NO: 114 - protein, 12 aa, Sutterella wadsworthensis
DMGAKYTGVFYA

SEQ ID NO: 115 - protein, 12 aa, Streptococcus mutans
DLGGKNTGFFSF

SEQ ID NO: 116 - protein, 12 aa, Francisella tularensis
DLGVKNTGVFSA

SEQ ID NO: 117 - protein, 12 aa, Streptococcus mutans
DLGAKFTGVALY

SEQ ID NO: 118 - protein, 12 aa, Streptococcus mutans
DLGGKFTGVCLS

SEQ ID NO: 119 - protein, 12 aa, Parasutterella excrementihominis
DLGGTYTGTFIT

SEQ ID NO: 120 - protein, 12 aa, Streptococcus mutans
DIGTNSVGWAVT

SEQ ID NO: 121 - protein, 12 aa, Eubacterium yurii
DVGTNSVGWAVT

SEQ ID NO: 122 - protein, 12 aa, Butyrivibrio hungatei
DMGTNSVGWAVT

SEQ ID NO: 123 - protein, 12 aa, Streptococcus mutans
DVGTSSVGWAVT

SEQ ID NO: 124 - protein, 27 aa, Treponema denticola
DIDHIYPQSKIKDDSISNRVLVCSSCN

SEQ ID NO: 125 - protein, 27 aa, Streptococcus mutans
DIDHIYPQSKTMDDSLNNRVLVKKNYN

SEQ ID NO: 126 - protein, 27 aa, Peptoniphilus duerdenii
DQDHIYPKSKIYDDSLENRVLVKKNLN

SEQ ID NO: 127 - protein, 27 aa, Streptococcus mutans
QIDHIVPQSLVKDDSFDNRVLVVPSEN

SEQ ID NO: 128 - protein, 27 aa, Streptococcus mutans
DIDHIIPQAFIKDNSIDNRVLTSSKEN

SEQ ID NO: 129 - protein, 27 aa, Streptococcus mutans
DIDHIIPQAFLKDNSIDNKVLVSSASN

SEQ ID NO: 130 - protein, 27 aa, Streptococcus mutans
DIDHIIPQAYTKDNSLDNRVLVSNITN

SEQ ID NO: 131 - protein, 27 aa, Streptococcus mutans
DIDHIVPQSFITDNSIDNLVLTSSAGN

SEQ ID NO: 132 - protein, 27 aa, Streptococcus mutans
DVDHIVPQSFLKDDSIDNKVLTRSDKN

SEQ ID NO: 133 - protein, 27 aa, Streptococcus mutans
NIDHIYPQSMVKDDSLDNKVLVQSEIN

SEQ ID NO: 134 - protein, 27 aa, Streptococcus mutans
DIDHILPQSLIKDDSLDNRVLVNATIN

SEQ ID NO: 135 - protein, 27 aa, Lactobacillus gasseri
DIDHILPQSFIKDDSLENRVLVKKAVN

SEQ ID NO: 136 - protein, 27 aa, Streptococcus mutans
EVDHIFPRSFIKDDSIDNKVLVIKKMN

SEQ ID NO: 137 - protein, 27 aa, Olsenella uli
EVDHIIPRSYIKDDSFENKVLVYREEN

SEQ ID NO: 138 - protein, 27 aa, Streptococcus mutans
DIDHIIPQAVTQNDSIDNRVLVARAEN

SEQ ID NO: 139 - protein, 27 aa, Streptococcus mutans
EIDHIIPYSISFDDSSSNKLLVLAESN

SEQ ID NO: 140 - protein, 27 aa, Streptococcus mutans
EIDHIIPYSLCFDDSSANKVLVHKQSN

SEQ ID NO: 141 - protein, 27 aa, Streptococcus mutans
DIDHIIPYSRSMDDSYSNKVLVLSGEN

SEQ ID NO: 142 - protein, 27 aa, Streptococcus mutans
DIDHIIPYSKSMDDSFNNKVLCLAEEN

SEQ ID NO: 143 - protein, 27 aa, Campylobacter jejuni
EIDHIYPYSRSFDDSYMNKVLVFTKQN

SEQ ID NO: 144 - protein, 27 aa, Streptococcus mutans
QIDHIYPYSRSMDDSYMNKVLVLTDEN

SEQ ID NO: 145 - protein, 27 aa, Clostridium perfringens
EIDHIIPFSRSFDDSLSNKILVLGSEN

SEQ ID NO: 146 - protein, 27 aa, Streptococcus mutans
EIDHALPFSRTWDDSFNNKVLVLGSEN

SEQ ID NO: 147 - protein, 27 aa, Streptococcus mutans
EIDHALPFSRTWDDSFNNKVLVLASEN

SEQ ID NO: 148 - protein, 27 aa, Streptococcus mutans
EIDHIIPISISLDDSINNKVLVLSKAN

SEQ ID NO: 149 - protein, 27 aa, Streptococcus mutans
EVDHIIPISISLDDSITNKVLVTHREN

SEQ ID NO: 150 - protein, 27 aa, Acidovorax ebreus
QVDHALPYSRSYDDSKNNKVLVLTHEN

SEQ ID NO: 151 - protein, 27 aa, Streptococcus mutans
EVDHILPLSITFDDSLANKVLVYATAN

SEQ ID NO: 152 - protein, 27 aa, Eubacterium rectale
EIDHIIPRSISFDDARSNKVLVYRSEN

SEQ ID NO: 153 - protein, 27 aa, Streptococcus mutans
EVDHIIPRSVSFDNSYHNKVLVKQSEN

SEQ ID NO: 154 - protein, 27 aa, Roseburia intestinalis
DIDHILPYSITFDDSFRNKVLVTSQEN

SEQ ID NO: 155 - protein, 27 aa, Streptococcus mutans
EIDHILPRSRSADDSFANKVLCLARAN

SEQ ID NO: 156 - protein, 27 aa, Candidatus Puniceispirillum marinum
EIEHLLPFSLTLDDSMANKTVCFRQAN

SEQ ID NO: 157 - protein, 27 aa, Streptococcus mutans
DIDHILPFSVSLDDSAANKVVCLREAN

SEQ ID NO: 158 - protein, 27 aa, Streptococcus mutans
DIDHLIPFSISWDDSAANKVVCMRYAN

SEQ ID NO: 159 - protein, 27 aa, Streptococcus mutans
DIDHILPVAMTLDDSPANKIICMRYAN

SEQ ID NO: 160 - protein, 27 aa, Dinoroseobacter shibae
DVDHILPYSRTLDDSFPNRTLCLREAN

SEQ ID NO: 161 - protein, 27 aa, Verminephrobacter eiseniae
EIEHILPFSRTLDDSLNNRTVAMRRAN

SEQ ID NO: 162 - protein, 27 aa, Streptococcus mutans
EVDHIIPYSISWDDSYTNKVLTSAKCN

SEQ ID NO: 163 - protein, 27 aa, Rhodopseudomonas palustris
QVDHILPWSRFGDDSYLNKTLCTARSN

SEQ ID NO: 164 - protein, 27 aa, Streptococcus mutans
QVDHILPFSKTLDDSFANKVLAQHDAN

SEQ ID NO: 165 - protein, 27 aa, Streptococcus mutans
QIDHAFPLSRSLDDSQSNKVLCLTSSN

SEQ ID NO: 166 - protein, 27 aa, Streptococcus mutans
DIDHIVPRSISFDDSFSNLVIVNKLDN

SEQ ID NO: 167 - protein, 27 aa, Streptococcus mutans
EIEHIIPYSMSYDNSQANKILTEKAEN

SEQ ID NO: 168 - protein, 27 aa, Streptococcus mutans
EIDHVIPYSKSADDSWFNKLLVKKSTN

SEQ ID NO: 169 - protein, 27 aa, Streptococcus mutans
EMDHILPYSRSLDNGWHNRVLVHGKDN

SEQ ID NO: 170 - protein, 27 aa, Streptococcus mutans
EVDHIVPYSLILDNTINNKALVYAEEN

SEQ ID NO: 171 - protein, 27 aa, Fibrobacter succinogenes
EIEHVIPQSLYFDDSFSNKVICEAEVN

SEQ ID NO: 172 - protein, 27 aa, Streptococcus mutans
DIEHIIPQARLFDDSFSNKTLEARSVN

SEQ ID NO: 173 - protein, 27 aa, Capnocytophaga sputigena
EIEHIVPKARVFDDSFSNKTLTFHRIN

SEQ ID NO: 174 - protein, 28 aa, Finegoldia magna
DKDHIIPQSMKKDDSIINNLVLVNKNAN

SEQ ID NO: 175 - protein, 27 aa, Streptococcus mutans
EVEHIWPRSRSFDNSPRNKTLCRKDVN

SEQ ID NO: 176 - protein, 27 aa, Bacillus cereus
IVNHIIPYNRSFDDTYHNRVLTLTETK

SEQ ID NO: 177 - protein, 27 aa, Prevotella micans
DMEHTIPKSISFDNSDQNLTLCESYYN

SEQ ID NO: 178 - protein, 27 aa, Prevotella ruminicola
DIEHTIPRSAGGDSTKMNLTLCSSRFN

SEQ ID NO: 179 - protein, 27 aa, Flavobacterium columnare
DIEHTIPRSISQDNSQMNKTLCSLKFN

SEQ ID NO: 180 - protein, 27 aa, Rhodospirillum rubrum
DIDHVIPLARGGRDSLDNMVLCQSDAN

SEQ ID NO: 181 - protein, 27 aa, Streptococcus mutans
DIEHLFPIAESEDNGRNNLVISHSACN

SEQ ID NO: 182 - protein, 27 aa, Streptococcus mutans
DVDHIFPRDDTADNSYGNKVVAHRQCN

SEQ ID NO: 183 - protein, 27 aa, Streptococcus mutans
DIEHIVPQSLGGLSTDYNTIVTLKSVN

SEQ ID NO: 184 - protein, 27 aa, Streptococcus mutans
ELDHIVPRTDGGSNRHENLAITCGACN

SEQ ID NO: 185 - protein, 28 aa, Streptococcus mutans
EMDHIVPRKGVGSTNTRTNFAAVCAECN

SEQ ID NO: 186 - protein, 28 aa, Bifidobacterium dentium
EMDHIVPRKGVGSTNTRVNLAAACAACN

SEQ ID NO: 187 - protein, 28 aa, Corynebacterium diphtheriae
EMDHIVPRAGQGSTNTRENLVAVCHRCN

SEQ ID NO: 188 - protein, 33 aa, Sutterella wadsworthensis
EIDHILPRSLIKDARGIVFNAEPNLIYASSRGN

SEQ ID NO: 189 - protein, 33 aa, Streptococcus mutans
EIDHIIPRSLTGRTKKTVFNSEANLIYCSSKGN

SEQ ID NO: 190 - protein, 33 aa, Parasutterella excrementihominis
EIDHIIPRSLTLKKSESIYNSEVNLIFVSAQGN

SEQ ID NO: 191 - protein, 33 aa, Streptococcus mutans
EIDHIYPRSLSKKHFGVIFNSEVNLIYCSSQGN

SEQ ID NO: 192 - protein, 33 aa, Streptococcus mutans
EIDHILPRSHTLKIYGTVFNPEGNLIYVHQKCN

SEQ ID NO: 193 - protein, 30 aa, Francisella tularensis
ELDHIIPRSHKKYGTLNDEANLICVTRGDN

SEQ ID NO: 194 - protein, 27 aa, Akkermansia muciniphila
ELEHIVPHSFRQSNALSSLVLTWPGVN

SEQ ID NO: 195 - protein, 27 aa, Streptococcus mutans
DIDHIYPRSKIKDDSITNRVLVEKDIN

SEQ ID NO: 196 - protein, 28 aa, Streptococcus mutans
YDIDHIYPRSLTKDDSFDNLVLCERTAN

SEQ ID NO: 197 - protein, 28 aa, Fusobacterium nucleatum
DIDHIYPRSKVIKDDSFDNLVLVLKNEN

SEQ ID NO: 198 - protein, 27 aa, Filifactor alocis
DRDHIYPQSKIKDDSIDNLVLVNKTYN

SEQ ID NO: 199-250 - skipped (52 entries)

SEQ ID NO: 251 - RNA, 17 nt, synthetic construct
  misc_feature 1..17: note = Targeting domain
  source 1..17: mol_type = other RNA
gaaggaaactagctaaa

SEQ ID NO: 252 - RNA, 20 nt, synthetic construct
  misc_feature 1..20: note = Targeting domain
  source 1..20: mol_type = other RNA
ggagaaggaaactagctaaa

SEQ ID NO: 253 - RNA, 20 nt, synthetic construct
  misc_feature 1..20: note = Targeting domain
  source 1..20: mol_type = other RNA
gggagaaggaaactagctaa

SEQ ID NO: 254 - RNA, 20 nt, synthetic construct
  misc_feature 1..20: note = Targeting domain
  source 1..20: mol_type = other RNA
gtatcctctatgatgggaga

SEQ ID NO: 255 - RNA, 20 nt, synthetic construct
  misc_feature 1..20: note = Targeting domain
  source 1..20: mol_type = other RNA
gtttccttctcccatcatag

SEQ ID NO: 256 - RNA, 20 nt, synthetic construct
  misc_feature 1..20: note = Targeting domain
  source 1..20: mol_type = other RNA
gtcctggtatcctctatgat

SEQ ID NO: 257 - RNA, 17 nt, synthetic construct
  misc_feature 1..17: note = Targeting domain
  source 1..17: mol_type = other RNA
agaaggaaactagctaa

SEQ ID NO: 258 - RNA, 17 nt, synthetic construct
  misc_feature 1..17: note = Targeting domain
  source 1..17: mol_type = other RNA
tcctctatgatgggaga

SEQ ID NO: 259 - RNA, 17 nt, synthetic construct
  misc_feature 1..17: note = Targeting domain
  source 1..17: mol_type = other RNA
cctggtatcctctatga

SEQ ID NO: 260 - RNA, 17 nt, synthetic construct
  misc_feature 1..17: note = Targeting domain
  source 1..17: mol_type = other RNA
ccatcatagaggatacc

SEQ ID NO: 261 - RNA, 17 nt, synthetic construct
  misc_feature 1..17: note = Targeting domain
  source 1..17: mol_type = other RNA
tccttctcccatcatag

SEQ ID NO: 262 - RNA, 17 nt, synthetic construct
  misc_feature 1..17: note = Targeting domain
  source 1..17: mol_type = other RNA
tagcagtatcctcttgg

SEQ ID NO: 263 - RNA, 17 nt, synthetic construct
  misc_feature 1..17: note = Targeting domain
  source 1..17: mol_type = other RNA
ttagcagtatcctcttg

SEQ ID NO: 264 - RNA, 17 nt, synthetic construct
  misc_feature 1..17: note = Targeting domain
  source 1..17: mol_type = other RNA
aactggaatgactgaat

SEQ ID NO: 265 - RNA, 17 nt, synthetic construct
  misc_feature 1..17: note = Targeting domain
  source 1..17: mol_type = other RNA
ctggtatcctctatgat

SEQ ID NO: 266 - RNA, 17 nt, synthetic construct
  misc_feature 1..17: note = Targeting domain
  source 1..17: mol_type = other RNA
aattagcagtatcctct

SEQ ID NO: 267 - RNA, 17 nt, synthetic construct
  misc_feature 1..17: note = Targeting domain
  source 1..17: mol_type = other RNA
attagcagtatcctctt

SEQ ID NO: 268 - RNA, 20 nt, synthetic construct
  misc_feature 1..20: note = Targeting domain
  source 1..20: mol_type = other RNA
agtcctggtatcctctatga

SEQ ID NO: 269 - RNA, 20 nt, synthetic construct
  misc_feature 1..20: note = Targeting domain
  source 1..20: mol_type = other RNA
ctcccatcatagaggatacc

SEQ ID NO: 270 - RNA, 20 nt, synthetic construct
  misc_feature 1..20: note = Targeting domain
  source 1..20: mol_type = other RNA
aattagcagtatcctcttgg

SEQ ID NO: 271 - RNA, 20 nt, synthetic construct
  misc_feature 1..20: note = Targeting domain
  source 1..20: mol_type = other RNA
aaattagcagtatcctcttg

SEQ ID NO: 272 - RNA, 20 nt, synthetic construct
  misc_feature 1..20: note = Targeting domain
  source 1..20: mol_type = other RNA
aaaaactggaatgactgaat

SEQ ID NO: 273 - RNA, 20 nt, synthetic construct
  misc_feature 1..20: note = Targeting domain
  source 1..20: mol_type = other RNA
aaaaattagcagtatcctct

SEQ ID NO: 274 - RNA, 20 nt, synthetic construct
  misc_feature 1..20: note = Targeting domain
  source 1..20: mol_type = other RNA
aaaattagcagtatcctctt

SEQ ID NO: 275 - RNA, 17 nt, synthetic construct
  misc_feature 1..17: note = Targeting domain
  source 1..17: mol_type = other RNA
gaatcggaacaaggcaa

SEQ ID NO: 276 - RNA, 17 nt, synthetic construct
  misc_feature 1..17: note = Targeting domain
  source 1..17: mol_type = other RNA
gaccaatagccttgaca

SEQ ID NO: 277 - RNA, 17 nt, synthetic construct
  misc_feature 1..17: note = Targeting domain
  source 1..17: mol_type = other RNA
ggctattggtcaaggca

SEQ ID NO: 278 - RNA, 17 nt, synthetic construct
  misc_feature 1..17: note = Targeting domain
  source 1..17: mol_type = other RNA
gtcaaggctattggtca

SEQ ID NO: 279 - RNA, 17 nt, synthetic construct
  misc_feature 1..17: note = Targeting domain
  source 1..17: mol_type = other RNA
gtgtgtggaactgctga

SEQ ID NO: 280 - RNA, 17 nt, synthetic construct
  misc_feature 1..17: note = Targeting domain
  source 1..17: mol_type = other RNA
gggccggcggctggcta

SEQ ID NO: 281 - RNA, 17 nt, synthetic construct
  misc_feature 1..17: note = Targeting domain
  source 1..17: mol_type = other RNA
gagtatccagtgaggcc

SEQ ID NO: 282 - RNA, 17 nt, synthetic construct
  misc_feature 1..17: note = Targeting domain
  source 1..17: mol_type = other RNA
gctgacaaaagaagtcc

SEQ ID NO: 283 - RNA, 17 nt, synthetic construct
  misc_feature 1..17: note = Targeting domain
  source 1..17: mol_type = other RNA
ggccaggggccggcggc

SEQ ID NO: 284 - RNA, 17 nt, synthetic construct
  misc_feature 1..17: note = Targeting domain
  source 1..17: mol_type = other RNA
gggaaggggcccccaag

SEQ ID NO: 285 - RNA, 17 nt, synthetic construct
  misc_feature 1..17: note = Targeting domain
  source 1..17: mol_type = other RNA
gagatagtgtggggaag

SEQ ID NO: 286 - RNA, 17 nt, synthetic construct
  misc_feature 1..17: note = Targeting domain
  source 1..17: mol_type = other RNA
gtatccagtgaggccag

SEQ ID NO: 287 - RNA, 17 nt, synthetic construct
  misc_feature 1..17: note = Targeting domain
  source 1..17: mol_type = other RNA
gtgaggccaggggccgg

SEQ ID NO: 288 - RNA, 17 nt, synthetic construct
  misc_feature 1..17: note = Targeting domain
  source 1..17: mol_type = other RNA
gctggccaacccatggg

SEQ ID NO: 289 - RNA, 17 nt, synthetic construct
  misc_feature 1..17: note = Targeting domain
  source 1..17: mol_type = other RNA
ggctaaactccacccat

SEQ ID NO: 290 - RNA, 17 nt, synthetic construct
  misc_feature 1..17: note = Targeting domain
  source 1..17: mol_type = other RNA
ggatactctaagactat

SEQ ID NO: 291 - RNA, 17 nt, synthetic construct
  misc_feature 1..17: note = Targeting domain
  source 1..17: mol_type = other RNA
ggggccggcggctggct

SEQ ID NO: 292 - RNA, 20 nt, synthetic construct
  misc_feature 1..20: note = Targeting domain
  source 1..20: mol_type = other RNA
ggctagggatgaagaataaa

SEQ ID NO: 293 - RNA, 20 nt, synthetic construct
  misc_feature 1..20: note = Targeting domain
  source 1..20: mol_type = other RNA
gagtgtgtggaactgctgaa

SEQ ID NO: 294 - RNA, 20 nt, synthetic construct
  misc_feature 1..20: note = Targeting domain
  source 1..20: mol_type = other RNA
ggaatgactgaatcggaaca

SEQ ID NO: 295 - RNA, 20 nt, synthetic construct
  misc_feature 1..20: note = Targeting domain
  source 1..20: mol_type = other RNA
gcattgagatagtgtgggga

SEQ ID NO: 296 - RNA, 20 nt, synthetic construct
  misc_feature 1..20: note = Targeting domain
  source 1..20: mol_type = other RNA
gctattggtcaaggcaaggc

SEQ ID NO: 297 - RNA, 20 nt, synthetic construct
  misc_feature 1..20: note = Targeting domain
  source 1..20: mol_type = other RNA
gtggggaaggggcccccaag

SEQ ID NO: 298 - RNA, 20 nt, synthetic construct
  misc_feature 1..20: note = Targeting domain
  source 1..20: mol_type = other RNA
ggcaaggctggccaacccat

SEQ ID NO: 299 - RNA, 20 nt, synthetic construct
  misc_feature 1..20: note = Targeting domain
  source 1..20: mol_type = other RNA
gtttgccttgtcaaggctat

SEQ ID NO: 300 - RNA, 20 nt, synthetic construct
  misc_feature 1..20: note = Targeting domain
  source 1..20: mol_type = other RNA
gctaaactccacccatgggt

SEQ ID NO: 301 - RNA, 17 nt, synthetic construct
  misc_feature 1..17: note = Targeting domain
  source 1..17: mol_type = other RNA
caaatatctgtctgaaa

SEQ ID NO: 302 - RNA, 17 nt, synthetic construct
  misc_feature 1..17: note = Targeting domain
  source 1..17: mol_type = other RNA
tagggatgaagaataaa

SEQ ID NO: 303 - RNA, 17 nt, synthetic construct
  misc_feature 1..17: note = Targeting domain
  source 1..17: mol_type = other RNA
tgagatagtgtggggaa

SEQ ID NO: 304 - RNA, 17 nt, synthetic construct
  misc_feature 1..17: note = Targeting domain
  source 1..17: mol_type = other RNA
tgtgtggaactgctgaa

SEQ ID NO: 305 - RNA, 17 nt, synthetic construct
  misc_feature 1..17: note = Targeting domain
  source 1..17: mol_type = other RNA
atgactgaatcggaaca

SEQ ID NO: 306 - RNA, 17 nt, synthetic construct
  misc_feature 1..17: note = Targeting domain
  source 1..17: mol_type = other RNA
caaggctggccaaccca

SEQ ID NO: 307 - RNA, 17 nt, synthetic construct
  misc_feature 1..17: note = Targeting domain
  source 1..17: mol_type = other RNA
tggctaaactccaccca

SEQ ID NO: 308 - RNA, 17 nt, synthetic construct
  misc_feature 1..17: note = Targeting domain
  source 1..17: mol_type = other RNA
tgggtggagtttagcca

SEQ ID NO: 309 - RNA, 17 nt, synthetic construct
  misc_feature 1..17: note = Targeting domain
  source 1..17: mol_type = other RNA
agtatccagtgaggcca

SEQ ID NO: 310 - RNA, 17 nt, synthetic construct
  misc_feature 1..17: note = Targeting domain
  source 1..17: mol_type = other RNA
tcaagtttgccttgtca

SEQ ID NO: 311 - RNA, 17 nt, synthetic construct
  misc_feature 1..17: note = Targeting domain
  source 1..17: mol_type = other RNA
ttgagatagtgtgggga

SEQ ID NO: 312 - RNA, 17 nt, synthetic construct
  misc_feature 1..17: note = Targeting domain
  source 1..17: mol_type = other RNA
ataaattagagaaaaac

SEQ ID NO: 313 - RNA, 17 nt, synthetic construct
  misc_feature 1..17: note = Targeting domain
  source 1..17: mol_type = other RNA
ccggcccctggcctcac

SEQ ID NO: 314 - RNA, 17 nt, synthetic construct
  misc_feature 1..17: note = Targeting domain
  source 1..17: mol_type = other RNA
agccagccgccggcccc

SEQ ID NO: 315 - RNA, 17 nt, synthetic construct
  misc_feature 1..17: note = Targeting domain
  source 1..17: mol_type = other RNA
ctgtctgaaacggtccc

SEQ ID NO: 316 - RNA, 17 nt, synthetic construct
  misc_feature 1..17: note = Targeting domain
  source 1..17: mol_type = other RNA
atgggtggagtttagcc

SEQ ID NO: 317 - RNA, 17 nt, synthetic construct
  misc_feature 1..17: note = Targeting domain
  source 1..17: mol_type = other RNA
catccctagccagccgc

SEQ ID NO: 318 - RNA, 17 nt, synthetic construct
  misc_feature 1..17: note = Targeting domain
  source 1..17: mol_type = other RNA
attggtcaaggcaaggc

SEQ ID NO: 319 - RNA, 17 nt, synthetic construct
  misc_feature 1..17: note = Targeting domain
  source 1..17: mol_type = other RNA
ccagtgaggccaggggc

SEQ ID NO: 320 - RNA, 17 nt, synthetic construct
  misc_feature 1..17: note = Targeting domain
  source 1..17: mol_type = other RNA
ttccacacactcgcttc

SEQ ID NO: 321 - RNA, 17 nt, synthetic construct
  misc_feature 1..17: note = Targeting domain
  source 1..17: mol_type = other RNA
cgcttctggaacgtctg

SEQ ID NO: 322 - RNA, 17 nt, synthetic construct
  misc_feature 1..17: note = Targeting domain
  source 1..17: mol_type = other RNA
tcttagagtatccagtg

SEQ ID NO: 323 - RNA, 17 nt, synthetic construct
  misc_feature 1..17: note = Targeting domain
  source 1..17: mol_type = other RNA
tttgcattgagatagtg

SEQ ID NO: 324 - RNA, 17 nt, synthetic construct
  misc_feature 1..17: note = Targeting domain
  source 1..17: mol_type = other RNA
ttccagaagcgagtgtg

SEQ ID NO: 325 - RNA, 17 nt, synthetic construct
  misc_feature 1..17: note = Targeting domain
  source 1..17: mol_type = other RNA
tgcattgagatagtgtg

SEQ ID NO: 326 - RNA, 17 nt, synthetic construct
  misc_feature 1..17: note = Targeting domain
  source 1..17: mol_type = other RNA
aaggctggccaacccat

SEQ ID NO: 327 - RNA, 17 nt, synthetic construct
  misc_feature 1..17: note = Targeting domain
  source 1..17: mol_type = other RNA
tgccttgtcaaggctat

SEQ ID NO: 328 - RNA, 17 nt, synthetic construct
  misc_feature 1..17: note = Targeting domain
  source 1..17: mol_type = other RNA
aaactccacccatgggt

SEQ ID NO: 329 - RNA, 17 nt, synthetic construct
  misc_feature 1..17: note = Targeting domain
  source 1..17: mol_type = other RNA
ttgcattgagatagtgt

SEQ ID NO: 330 - RNA, 20 nt, synthetic construct
  misc_feature 1..20: note = Targeting domain
  source 1..20: mol_type = other RNA
atgcaaatatctgtctgaaa

SEQ ID NO: 331 - RNA, 20 nt, synthetic construct
  misc_feature 1..20: note = Targeting domain
  source 1..20: mol_type = other RNA
actgaatcggaacaaggcaa

SEQ ID NO: 332 - RNA, 20 nt, synthetic construct
  misc_feature 1..20: note = Targeting domain
  source 1..20: mol_type = other RNA
cattgagatagtgtggggaa

SEQ ID NO: 333 - RNA, 20 nt, synthetic construct
  misc_feature 1..20: note = Targeting domain
  source 1..20: mol_type = other RNA
cttgaccaatagccttgaca

SEQ ID NO: 334 - RNA, 20 nt, synthetic construct
  misc_feature 1..20: note = Targeting domain
  source 1..20: mol_type = other RNA
aggcaaggctggccaaccca

SEQ ID NO: 335 - RNA, 20 nt, synthetic construct
  misc_feature 1..20: note = Targeting domain
  source 1..20: mol_type = other RNA
ccctggctaaactccaccca

SEQ ID NO: 336 - RNA, 20 nt, synthetic construct
  misc_feature 1..20: note = Targeting domain
  source 1..20: mol_type = other RNA
ccatgggtggagtttagcca

SEQ ID NO: 337 - RNA, 20 nt, synthetic construct
  misc_feature 1..20: note = Targeting domain
  source 1..20: mol_type = other RNA
tagagtatccagtgaggcca

SEQ ID NO: 338 - RNA, 20 nt, synthetic construct
  misc_feature 1..20: note = Targeting domain
  source 1..20: mol_type = other RNA
caaggctattggtcaaggca

SEQ ID NO: 339 - RNA, 20 nt, synthetic construct
  misc_feature 1..20: note = Targeting domain
  source 1..20: mol_type = other RNA
cttgtcaaggctattggtca

SEQ ID NO: 340 - RNA, 20 nt, synthetic construct
  misc_feature 1..20: note = Targeting domain
  source 1..20: mol_type = other RNA
tggtcaagtttgccttgtca

SEQ ID NO: 341 - RNA, 20 nt, synthetic construct
  misc_feature 1..20: note = Targeting domain
  source 1..20: mol_type = other RNA
cgagtgtgtggaactgctga

SEQ ID NO: 342 - RNA, 20 nt, synthetic construct
  misc_feature 1..20: note = Targeting domain
  source 1..20: mol_type = other RNA
caggggccggcggctggcta

SEQ ID NO: 343 - RNA, 20 nt, synthetic construct
  misc_feature 1..20: note = Targeting domain
  source 1..20: mol_type = other RNA
agaataaattagagaaaaac

SEQ ID NO: 344 - RNA, 20 nt, synthetic construct
  misc_feature 1..20: note = Targeting domain
  source 1..20: mol_type = other RNA
ccgccggcccctggcctcac

SEQ ID NO: 345 - RNA, 20 nt, synthetic construct
  misc_feature 1..20: note = Targeting domain
  source 1..20: mol_type = other RNA
cctagccagccgccggcccc

SEQ ID NO: 346 - RNA, 20 nt, synthetic construct
  misc_feature 1..20: note = Targeting domain
  source 1..20: mol_type = other RNA
tatctgtctgaaacggtccc

SEQ ID NO: 347 - RNA, 20 nt, synthetic construct
  misc_feature 1..20: note = Targeting domain
  source 1..20: mol_type = other RNA
cccatgggtggagtttagcc

SEQ ID NO: 348 - RNA, 20 nt, synthetic construct
  misc_feature 1..20: note = Targeting domain
  source 1..20: mol_type = other RNA
ttagagtatccagtgaggcc

SEQ ID NO: 349 - RNA, 20 nt, synthetic construct
  misc_feature 1..20: note = Targeting domain
  source 1..20: mol_type = other RNA
acggctgacaaaagaagtcc

SEQ ID NO: 350 - RNA, 20 nt, synthetic construct
  misc_feature 1..20: note = Targeting domain
  source 1..20: mol_type = other RNA
cttcatccctagccagccgc

SEQ ID NO: 351 - RNA, 20 nt, synthetic construct
  misc_feature 1..20: note = Targeting domain
  source 1..20: mol_type = other RNA
tgaggccaggggccggcggc

SEQ ID NO: 352 - RNA, 20 nt, synthetic construct
  misc_feature 1..20: note = Targeting domain
  source 1..20: mol_type = other RNA
tatccagtgaggccaggggc

SEQ ID NO: 353 - RNA, 20 nt, synthetic construct
  misc_feature 1..20: note = Targeting domain
  source 1..20: mol_type = other RNA
cagttccacacactcgcttc

SEQ ID NO: 354 - RNA, 20 nt, synthetic construct
  misc_feature 1..20: note = Targeting domain
  source 1..20: mol_type = other RNA
attgagatagtgtggggaag

SEQ ID NO: 355 - RNA, 20 nt, synthetic construct
  misc_feature 1..20: note = Targeting domain
  source 1..20: mol_type = other RNA
agagtatccagtgaggccag

SEQ ID NO: 356 - RNA, 20 nt, synthetic construct
  misc_feature 1..20: note = Targeting domain
  source 1..20: mol_type = other RNA
ccagtgaggccaggggccgg

SEQ ID NO: 357 - RNA, 20 nt, synthetic construct
  misc_feature 1..20: note = Targeting domain
  source 1..20: mol_type = other RNA
aaggctggccaacccatggg

SEQ ID NO: 358 - RNA, 20 nt, synthetic construct
  misc_feature 1..20: note = Targeting domain
  source 1..20: mol_type = other RNA
actcgcttctggaacgtctg

SEQ ID NO: 359 - RNA, 20 nt, synthetic construct
  misc_feature 1..20: note = Targeting domain
  source 1..20: mol_type = other RNA
tagtcttagagtatccagtg

SEQ ID NO: 360 - RNA, 20 nt, synthetic construct
  misc_feature 1..20: note = Targeting domain
  source 1..20: mol_type = other RNA
atatttgcattgagatagtg

SEQ ID NO: 361 - RNA, 20 nt, synthetic construct
  misc_feature 1..20: note = Targeting domain
  source 1..20: mol_type = other RNA
acgttccagaagcgagtgtg

SEQ ID NO: 362 - RNA, 20 nt, synthetic construct
  misc_feature 1..20: note = Targeting domain
  source 1..20: mol_type = other RNA
atttgcattgagatagtgtg

SEQ ID NO: 363 - RNA, 20 nt, synthetic construct
  misc_feature 1..20: note = Targeting domain
  source 1..20: mol_type = other RNA
cctggctaaactccacccat

SEQ ID NO: 364 - RNA, 20 nt, synthetic construct
  misc_feature 1..20: note = Targeting domain
  source 1..20: mol_type = other RNA
actggatactctaagactat

SEQ ID NO: 365 - RNA, 20 nt, synthetic construct
  misc_feature 1..20: note = Targeting domain
  source 1..20: mol_type = other RNA
ccaggggccggcggctggct

SEQ ID NO: 366 - RNA, 20 nt, synthetic construct
  misc_feature 1..20: note = Targeting domain
  source 1..20: mol_type = other RNA
tatttgcattgagatagtgt

SEQ ID NO: 367 - RNA, 19 nt, synthetic construct
  misc_feature 1..19: note = Targeting domain
  source 1..19: mol_type = other RNA
gtttccttctcccatcata

SEQ ID NO: 368 - RNA, 23 nt, synthetic construct
  misc_feature 1..23: note = Targeting domain
  source 1..23: mol_type = other RNA
gctagtttccttctcccatcata

SEQ ID NO: 369 - RNA, 19 nt, synthetic construct
  misc_feature 1..19: note = Targeting domain
  source 1..19: mol_type = other RNA
gaataaattagagaaaaac

SEQ ID NO: 370 - RNA, 22 nt, synthetic construct
  misc_feature 1..22: note = Targeting domain
  source 1..22: mol_type = other RNA
gaagaataaattagagaaaaac

SEQ ID NO: 371 - RNA, 23 nt, synthetic construct
  misc_feature 1..23: note = Targeting domain
  source 1..23: mol_type = other RNA
ggaagaataaattagagaaaaac

SEQ ID NO: 372 - RNA, 24 nt, synthetic construct
  misc_feature 1..24: note = Targeting domain
  source 1..24: mol_type = other RNA
gggaagaataaattagagaaaaac

SEQ ID NO: 373 - RNA, 20 nt, synthetic construct
  misc_feature 1..20: note = Targeting domain
  source 1..20: mol_type = other RNA
gaaggaaactagctaaaggg

SEQ ID NO: 374 - RNA, 22 nt, synthetic construct
  misc_feature 1..22: note = Targeting domain
  source 1..22: mol_type = other RNA
gagaaggaaactagctaaaggg

SEQ ID NO: 375 - RNA, 23 nt, synthetic construct
  misc_feature 1..23: note = Targeting domain
  source 1..23: mol_type = other RNA
ggagaaggaaactagctaaaggg

SEQ ID NO: 376 - RNA, 24 nt, synthetic construct
  misc_feature 1..24: note = Targeting domain
  source 1..24: mol_type = other RNA
gggagaaggaaactagctaaaggg

SEQ ID NO: 377 - RNA, 18 nt, synthetic construct
  misc_feature 1..18: note = Targeting domain
  source 1..18: mol_type = other RNA
tttccttctcccatcata

SEQ ID NO: 378 - RNA, 20 nt, synthetic construct
  misc_feature 1..20: note = Targeting domain
  source 1..20: mol_type = other RNA
agtttccttctcccatcata

SEQ ID NO: 379 - RNA, 21 nt, synthetic construct
  misc_feature 1..21: note = Targeting domain
  source 1..21: mol_type = other RNA
tagtttccttctcccatcata

SEQ ID NO: 380 - RNA, 22 nt, synthetic construct
  misc_feature 1..22: note = Targeting domain
  source 1..22: mol_type = other RNA
ctagtttccttctcccatcata

SEQ ID NO: 381 - RNA, 24 nt, synthetic construct
  misc_feature 1..24: note = Targeting domain
  source 1..24: mol_type = other RNA
agctagtttccttctcccatcata

SEQ ID NO: 382 - RNA, 18 nt, synthetic construct
  misc_feature 1..18: note = Targeting domain
  source 1..18: mol_type = other RNA
agagaaaaactggaatga

SEQ ID NO: 383 - RNA, 19 nt, synthetic construct
  misc_feature 1..19: note = Targeting domain
  source 1..19: mol_type = other RNA
tagagaaaaactggaatga

SEQ ID NO: 384 - RNA, 20 nt, synthetic construct
  misc_feature 1..20: note = Targeting domain
  source 1..20: mol_type = other RNA
ttagagaaaaactggaatga

SEQ ID NO: 385 - RNA, 21 nt, synthetic construct
  misc_feature 1..21: note = Targeting domain
  source 1..21: mol_type = other RNA
attagagaaaaactggaatga

SEQ ID NO: 386 - RNA, 22 nt, synthetic construct
  misc_feature 1..22: note = Targeting domain
  source 1..22: mol_type = other RNA
aattagagaaaaactggaatga

SEQ ID NO: 387 - RNA, 23 nt, synthetic construct
  misc_feature 1..23: note = Targeting domain
  source 1..23: mol_type = other RNA
aaattagagaaaaactggaatga

SEQ ID NO: 388 - RNA, 24 nt, synthetic construct
  misc_feature 1..24: note = Targeting domain
  source 1..24: mol_type = other RNA
taaattagagaaaaactggaatga

SEQ ID NO: 389 - RNA, 18 nt, synthetic construct
  misc_feature 1..18: note = Targeting domain
  source 1..18: mol_type = other RNA
aataaattagagaaaaac

SEQ ID NO: 390 - RNA, 21 nt, synthetic construct
  misc_feature 1..21: note = Targeting domain
  source 1..21: mol_type = other RNA
aagaataaattagagaaaaac

SEQ ID NO: 391 - RNA, 18 nt, synthetic construct
  misc_feature 1..18: note = Targeting domain
  source 1..18: mol_type = other RNA
aggaaactagctaaaggg

SEQ ID NO: 392 - RNA, 19 nt, synthetic construct
  misc_feature 1..19: note = Targeting domain
  source 1..19: mol_type = other RNA
aaggaaactagctaaaggg

SEQ ID NO: 393 - RNA, 21 nt, synthetic construct
  misc_feature 1..21: note = Targeting domain
  source 1..21: mol_type = other RNA
agaaggaaactagctaaaggg

SEQ ID NO: 394 - RNA, 18 nt, synthetic construct
  misc_feature 1..18: note = Targeting domain
  source 1..18: mol_type = other RNA
tggggaaggggcccccaa

SEQ ID NO: 395 - RNA, 19 nt, synthetic construct
  misc_feature 1..19: note = Targeting domain
  source 1..19: mol_type = other RNA
gtggggaaggggcccccaa

SEQ ID NO: 396 - RNA, 20 nt, synthetic construct
  misc_feature 1..20: note = Targeting domain
  source 1..20: mol_type = other RNA
tgtggggaaggggcccccaa

SEQ ID NO: 397 - RNA, 21 nt, synthetic construct
  misc_feature 1..21: note = Targeting domain
  source 1..21: mol_type = other RNA
gtgtggggaaggggcccccaa

SEQ ID NO: 398 - RNA, 22 nt, synthetic construct
  misc_feature 1..22: note = Targeting domain
  source 1..22: mol_type = other RNA
agtgtggggaaggggcccccaa

SEQ ID NO: 399 - RNA, 23 nt, synthetic construct
  misc_feature 1..23: note = Targeting domain
  source 1..23: mol_type = other RNA
tagtgtggggaaggggcccccaa

SEQ ID NO: 400 - RNA, 24 nt, synthetic construct
  misc_feature 1..24: note = Targeting domain
  source 1..24: mol_type = other RNA
atagtgtggggaaggggcccccaa

SEQ ID NO: 401 - RNA, 18 nt, synthetic construct
  misc_feature 1..18: note = Targeting domain
  source 1..18: mol_type = other RNA
acctcagacgttccagaa

SEQ ID NO: 402 - RNA, 19 nt, synthetic construct
  misc_feature 1..19: note = Targeting domain
  source 1..19: mol_type = other RNA
aacctcagacgttccagaa

SEQ ID NO: 403 - RNA, 20 nt, synthetic construct
  misc_feature 1..20: note = Targeting domain
  source 1..20: mol_type = other RNA
taacctcagacgttccagaa

SEQ ID NO: 404 - RNA, 21 nt, synthetic construct
  misc_feature 1..21: note = Targeting domain
  source 1..21: mol_type = other RNA
ataacctcagacgttccagaa

SEQ ID NO: 405 - RNA, 22 nt, synthetic construct
  misc_feature 1..22: note = Targeting domain
  source 1..22: mol_type = other RNA
gataacctcagacgttccagaa

SEQ ID NO: 406 - RNA, 23 nt, synthetic construct
  misc_feature 1..23: note = Targeting domain
  source 1..23: mol_type = other RNA
tgataacctcagacgttccagaa

SEQ ID NO: 407 - RNA, 24 nt, synthetic construct
  misc_feature 1..24: note = Targeting domain
  source 1..24: mol_type = other RNA
ttgataacctcagacgttccagaa

SEQ ID NO: 408 - RNA, 18 nt, synthetic construct
  misc_feature 1..18: note = Targeting domain
  source 1..18: mol_type = other RNA
cgccggcccctggcctca

SEQ ID NO: 409 - RNA, 19 nt, synthetic construct
  misc_feature 1..19: note = Targeting domain
  source 1..19: mol_type = other RNA
ccgccggcccctggcctca

SEQ ID NO: 410 - RNA, 20 nt, synthetic construct
  misc_feature 1..20: note = Targeting domain
  source 1..20: mol_type = other RNA
gccgccggcccctggcctca

SEQ ID NO: 411 - RNA, 21 nt, synthetic construct
  misc_feature 1..21: note = Targeting domain
  source 1..21: mol_type = other RNA
agccgccggcccctggcctca

SEQ ID NO: 412 - RNA, 22 nt, synthetic construct
  misc_feature 1..22: note = Targeting domain
  source 1..22: mol_type = other RNA
cagccgccggcccctggcctca

SEQ ID NO: 413 - RNA, 23 nt, synthetic construct
  misc_feature 1..23: note = Targeting domain
  source 1..23: mol_type = other RNA
ccagccgccggcccctggcctca

SEQ ID NO: 414 - RNA, 24 nt, synthetic construct
  misc_feature 1..24: note = Targeting domain
  source 1..24: mol_type = other RNA
gccagccgccggcccctggcctca

SEQ ID NO: 415 - RNA, 18 nt, synthetic construct
  misc_feature 1..18: note = Targeting domain
  source 1..18: mol_type = other RNA
ggcaaggctggccaaccc

SEQ ID NO: 416 - RNA, 19 nt, synthetic construct
  misc_feature 1..19: note = Targeting domain
  source 1..19: mol_type = other RNA
aggcaaggctggccaaccc

SEQ ID NO: 417 - RNA, 20 nt, synthetic construct
  misc_feature 1..20: note = Targeting domain
  source 1..20: mol_type = other RNA
aaggcaaggctggccaaccc

SEQ ID NO: 418 - RNA, 21 nt, synthetic construct
  misc_feature 1..21: note = Targeting domain
  source 1..21: mol_type = other RNA
caaggcaaggctggccaaccc

SEQ ID NO: 419 - RNA, 22 nt, synthetic construct
  misc_feature 1..22: note = Targeting domain
  source 1..22: mol_type = other RNA
tcaaggcaaggctggccaaccc

SEQ ID NO: 420 - RNA, 23 nt, synthetic construct
  misc_feature 1..23: note = Targeting domain
  source 1..23: mol_type = other RNA
gtcaaggcaaggctggccaaccc

SEQ ID NO: 421 - RNA, 24 nt, synthetic construct
  misc_feature 1..24: note = Targeting domain
  source 1..24: mol_type = other RNA
ggtcaaggcaaggctggccaaccc

SEQ ID NO: 422 - RNA, 18 nt, synthetic construct
  misc_feature 1..18: note = Targeting domain
  source 1..18: mol_type = other RNA
ggctggccaacccatggg

SEQ ID NO: 423 - RNA, 19 nt, synthetic construct
  misc_feature 1..19: note = Targeting domain
  source 1..19: mol_type = other RNA
aggctggccaacccatggg

SEQ ID NO: 424 - RNA, 21 nt, synthetic construct
  misc_feature 1..21: note = Targeting domain
  source 1..21: mol_type = other RNA
caaggctggccaacccatggg

SEQ ID NO: 425 - RNA, 22 nt, synthetic construct
  misc_feature 1..22: note = Targeting domain
  source 1..22: mol_type = other RNA
gcaaggctggccaacccatggg

SEQ ID NO: 426 - RNA, 23 nt, synthetic construct
  misc_feature 1..23: note = Targeting domain
  source 1..23: mol_type = other RNA
ggcaaggctggccaacccatggg

SEQ ID NO: 427 - RNA, 24 nt, synthetic construct
  misc_feature 1..24: note = Targeting domain
  source 1..24: mol_type = other RNA
aggcaaggctggccaacccatggg

SEQ ID NO: 428 - RNA, 18 nt, synthetic construct
  misc_feature 1..18: note = Targeting domain
  source 1..18: mol_type = other RNA
gagtgtgtggaactgctg

SEQ ID NO: 429 - RNA, 19 nt, synthetic construct
  misc_feature 1..19: note = Targeting domain
  source 1..19: mol_type = other RNA
cgagtgtgtggaactgctg

SEQ ID NO: 430 - RNA, 20 nt, synthetic construct
  misc_feature 1..20: note = Targeting domain
  source 1..20: mol_type = other RNA
gcgagtgtgtggaactgctg

SEQ ID NO: 431 - RNA, 21 nt, synthetic construct
  misc_feature 1..21: note = Targeting domain
  source 1..21: mol_type = other RNA
agcgagtgtgtggaactgctg

SEQ ID NO: 432 - RNA, 22 nt, synthetic construct
  misc_feature 1..22: note = Targeting domain
  source 1..22: mol_type = other RNA
aagcgagtgtgtggaactgctg

SEQ ID NO: 433 - RNA, 23 nt, synthetic construct
  misc_feature 1..23: note = Targeting domain
  source 1..23: mol_type = other RNA
gaagcgagtgtgtggaactgctg

SEQ ID NO: 434 - RNA, 24 nt, synthetic construct
  misc_feature 1..24: note = Targeting domain
  source 1..24: mol_type = other RNA
agaagcgagtgtgtggaactgctg

SEQ ID NO: 435 - RNA, 18 nt, synthetic construct
  misc_feature 1..18: note = Targeting domain
  source 1..18: mol_type = other RNA
cctggctaaactccaccc

SEQ ID NO: 436 - RNA, 19 nt, synthetic construct
  misc_feature 1..19: note = Targeting domain
  source 1..19: mol_type = other RNA
ccctggctaaactccaccc

SEQ ID NO: 437 - RNA, 20 nt, synthetic construct
  misc_feature 1..20: note = Targeting domain
  source 1..20: mol_type = other RNA
tccctggctaaactccaccc

SEQ ID NO: 438 - RNA, 21 nt, synthetic construct
  misc_feature 1..21: note = Targeting domain
  source 1..21: mol_type = other RNA
gtccctggctaaactccaccc

SEQ ID NO: 439 - RNA, 22 nt, synthetic construct
  misc_feature 1..22: note = Targeting domain
  source 1..22: mol_type = other RNA
ggtccctggctaaactccaccc

SEQ ID NO: 440 - RNA, 23 nt, synthetic construct
  misc_feature 1..23: note = Targeting domain
  source 1..23: mol_type = other RNA
cggtccctggctaaactccaccc

SEQ ID NO: 441 - RNA, 24 nt, synthetic construct
  misc_feature 1..24: note = Targeting domain
  source 1..24: mol_type = other RNA
acggtccctggctaaactccaccc

SEQ ID NO: 442 - RNA, 18 nt, synthetic construct
  misc_feature 1..18: note = Targeting domain
  source 1..18: mol_type = other RNA
ggcggctggctagggatg

SEQ ID NO: 443 - RNA, 19 nt, synthetic construct
  misc_feature 1..19: note = Targeting domain
  source 1..19: mol_type = other RNA
cggcggctggctagggatg

SEQ ID NO: 444 - RNA, 20 nt, synthetic construct
  misc_feature 1..20: note = Targeting domain
  source 1..20: mol_type = other RNA
ccggcggctggctagggatg

SEQ ID NO: 445 - RNA, 21 nt, synthetic construct
  misc_feature 1..21: note = Targeting domain
  source 1..21: mol_type = other RNA
gccggcggctggctagggatg

SEQ ID NO: 446 - RNA, 22 nt, synthetic construct
  misc_feature 1..22: note = Targeting domain
  source 1..22: mol_type = other RNA
ggccggcggctggctagggatg

SEQ ID NO: 447 - RNA, 23 nt, synthetic construct
  misc_feature 1..23: note = Targeting domain
  source 1..23: mol_type = other RNA
gggccggcggctggctagggatg

SEQ ID NO: 448 - RNA, 24 nt, synthetic construct
  misc_feature 1..24: note = Targeting domain
  source 1..24: mol_type = other RNA
ggggccggcggctggctagggatg

SEQ ID NO: 449 - RNA, 18 nt, synthetic construct
  misc_feature 1..18: note = Targeting domain
  source 1..18: mol_type = other RNA
aggggccggcggctggct

SEQ ID NO: 450 - RNA, 19 nt, synthetic construct
  misc_feature 1..19: note = Targeting domain
  source 1..19: mol_type = other RNA
caggggccggcggctggct

SEQ ID NO: 451 - RNA, 21 nt, synthetic construct
  misc_feature 1..21: note = Targeting domain
  source 1..21: mol_type = other RNA
gccaggggccggcggctggct

SEQ ID NO: 452 - RNA, 22 nt, synthetic construct
  misc_feature 1..22: note = Targeting domain
  source 1..22: mol_type = other RNA
ggccaggggccggcggctggct

SEQ ID NO: 453 - RNA, 23 nt, synthetic construct
  misc_feature 1..23: note = Targeting domain
  source 1..23: mol_type = other RNA
aggccaggggccggcggctggct

SEQ ID NO: 454 - RNA, 24 nt, synthetic construct
  misc_feature 1..24: note = Targeting domain
  source 1..24: mol_type = other RNA
gaggccaggggccggcggctggct

SEQ ID NO: 455 - RNA, 18 nt, synthetic construct
  misc_feature 1..18: note = Targeting domain
  source 1..18: mol_type = other RNA
aaacttgaccaatagtct

SEQ ID NO: 456 - RNA, 19 nt, synthetic construct
  misc_feature 1..19: note = Targeting domain
  source 1..19: mol_type = other RNA
caaacttgaccaatagtct

SEQ ID NO: 457 - RNA, 20 nt, synthetic construct
  misc_feature 1..20: note = Targeting domain
  source 1..20: mol_type = other RNA
gcaaacttgaccaatagtct

SEQ ID NO: 458 - RNA, 21 nt, synthetic construct
  misc_feature 1..21: note = Targeting domain
  source 1..21: mol_type = other RNA
ggcaaacttgaccaatagtct

SEQ ID NO: 459 - RNA, 22 nt, synthetic construct
  misc_feature 1..22: note = Targeting domain
  source 1..22: mol_type = other RNA
aggcaaacttgaccaatagtct

SEQ ID NO: 460 - RNA, 23 nt, synthetic construct
  misc_feature 1..23: note = Targeting domain
  source 1..23: mol_type = other RNA
aaggcaaacttgaccaatagtct

SEQ ID NO: 461 - RNA, 24 nt, synthetic construct
  misc_feature 1..24: note = Targeting domain
  source 1..24: mol_type = other RNA
caaggcaaacttgaccaatagtct

SEQ ID NO: 462 - RNA, 18 nt, synthetic construct
  misc_feature 1..18: note = Targeting domain
  source 1..18: mol_type = other RNA
ttcagacagatatttgca

SEQ ID NO: 463 - RNA, 19 nt, synthetic construct
  misc_feature 1..19: note = Targeting domain
  source 1..19: mol_type = other RNA
tttcagacagatatttgca

SEQ ID NO: 464 - RNA, 20 nt, synthetic construct
  misc_feature 1..20: note = Targeting domain
  source 1..20: mol_type = other RNA
gtttcagacagatatttgca

SEQ ID NO: 465 - RNA, 21 nt, synthetic construct
  misc_feature 1..21: note = Targeting domain
  source 1..21: mol_type = other RNA
cgtttcagacagatatttgca

SEQ ID NO: 466 - RNA, 22 nt, synthetic construct
  misc_feature 1..22: note = Targeting domain
  source 1..22: mol_type = other RNA
ccgtttcagacagatatttgca

SEQ ID NO: 467 - RNA, 23 nt, synthetic construct
  misc_feature 1..23: note = Targeting domain
  source 1..23: mol_type = other RNA
accgtttcagacagatatttgca

SEQ ID NO: 468 - RNA, 24 nt, synthetic construct
  misc_feature 1..24: note = Targeting domain
  source 1..24: mol_type = other RNA
gaccgtttcagacagatatttgca

SEQ ID NO: 469 - RNA, 18 nt, synthetic construct
  misc_feature 1..18: note = Targeting domain
  source 1..18: mol_type = other RNA
agtttccttctcccatca

SEQ ID NO: 470 - RNA, 19 nt, synthetic construct
  misc_feature 1..19: note = Targeting domain
  source 1..19: mol_type = other RNA
tagtttccttctcccatca

SEQ ID NO: 471 - RNA, 20 nt, synthetic construct
  misc_feature 1..20: note = Targeting domain
  source 1..20: mol_type = other RNA
ctagtttccttctcccatca

SEQ ID NO: 472 - RNA, 21 nt, synthetic construct
  misc_feature 1..21: note = Targeting domain
  source 1..21: mol_type = other RNA
gctagtttccttctcccatca

SEQ ID NO: 473 - RNA, 22 nt, synthetic construct
  misc_feature 1..22: note = Targeting domain
  source 1..22: mol_type = other RNA
agctagtttccttctcccatca

SEQ ID NO: 474 - RNA, 23 nt, synthetic construct
  misc_feature 1..23: note = Targeting domain
  source 1..23: mol_type = other RNA
tagctagtttccttctcccatca

SEQ ID NO: 475 - RNA, 24 nt, synthetic construct
  misc_feature 1..24: note = Targeting domain
  source 1..24: mol_type = other RNA
ttagctagtttccttctcccatca

SEQ ID NO: 476 - RNA, 18 nt, synthetic construct
  misc_feature 1..18: note = Targeting domain
  source 1..18: mol_type = other RNA
attgagatagtgtgggga

SEQ ID NO: 477 - RNA, 19 nt, synthetic construct
  misc_feature 1..19: note = Targeting domain
  source 1..19: mol_type = other RNA
cattgagatagtgtgggga

SEQ ID NO: 478 - RNA, 21 nt, synthetic construct
  misc_feature 1..21: note = Targeting domain
  source 1..21: mol_type = other RNA
tgcattgagatagtgtgggga

SEQ ID NO: 479 - RNA, 22 nt, synthetic construct
  misc_feature 1..22: note = Targeting domain
  source 1..22: mol_type = other RNA
ttgcattgagatagtgtgggga

SEQ ID NO: 480 - RNA, 23 nt, synthetic construct
  misc_feature 1..23: note = Targeting domain
  source 1..23: mol_type = other RNA
tttgcattgagatagtgtgggga

SEQ ID NO: 481 - RNA, 24 nt, synthetic construct
  misc_feature 1..24: note = Targeting domain
  source 1..24: mol_type = other RNA
atttgcattgagatagtgtgggga

SEQ ID NO: 482 - RNA, 18 nt, synthetic construct
  misc_feature 1..18: note = Targeting domain
  source 1..18: mol_type = other RNA
tcccatcatagaggatac

SEQ ID NO: 483 - RNA, 19 nt, synthetic construct
  misc_feature 1..19: note = Targeting domain
  source 1..19: mol_type = other RNA
ctcccatcatagaggatac

SEQ ID NO: 484 - RNA, 20 nt, synthetic construct
  misc_feature 1..20: note = Targeting domain
  source 1..20: mol_type = other RNA
tctcccatcatagaggatac

SEQ ID NO: 485 - RNA, 21 nt, synthetic construct
  misc_feature 1..21: note = Targeting domain
  source 1..21: mol_type = other RNA
ttctcccatcatagaggatac

SEQ ID NO: 486 - RNA, 22 nt, synthetic construct
  misc_feature 1..22: note = Targeting domain
  source 1..22: mol_type = other RNA
cttctcccatcatagaggatac

SEQ ID NO: 487 - RNA, 23 nt, synthetic construct
  misc_feature 1..23: note = Targeting domain
  source 1..23: mol_type = other RNA
ccttctcccatcatagaggatac

SEQ ID NO: 488 - RNA, 24 nt, synthetic construct
  misc_feature 1..24: note = Targeting domain
  source 1..24: mol_type = other RNA
tccttctcccatcatagaggatac

SEQ ID NO: 489 - RNA, 18 nt, synthetic construct
  misc_feature 1..18: note = Targeting domain
  source 1..18: mol_type = other RNA
tgtggggaaggggccccc

SEQ ID NO: 490 - RNA, 19 nt, synthetic construct
  misc_feature 1..19: note = Targeting domain
  source 1..19: mol_type = other RNA
gtgtggggaaggggccccc

SEQ ID NO: 491 - RNA, 20 nt, synthetic construct
  misc_feature 1..20: note = Targeting domain
  source 1..20: mol_type = other RNA
agtgtggggaaggggccccc

SEQ ID NO: 492 - RNA, 21 nt, synthetic construct
  misc_feature 1..21: note = Targeting domain
  source 1..21: mol_type = other RNA
tagtgtggggaaggggccccc

SEQ ID NO: 493 - RNA, 22 nt, synthetic construct
  misc_feature 1..22: note = Targeting domain
  source 1..22: mol_type = other RNA
atagtgtggggaaggggccccc

SEQ ID NO: 494 - RNA, 23 nt, synthetic construct
  misc_feature 1..23: note = Targeting domain
  source 1..23: mol_type = other RNA
gatagtgtggggaaggggccccc

SEQ ID NO: 495 - RNA, 24 nt, synthetic construct
  misc_feature 1..24: note = Targeting domain
  source 1..24: mol_type = other RNA
agatagtgtggggaaggggccccc

SEQ ID NO: 496 - RNA, 18 nt, synthetic construct
  misc_feature 1..18: note = Targeting domain
  source 1..18: mol_type = other RNA
catgggtggagtttagcc

SEQ ID NO: 497 - RNA, 19 nt, synthetic construct
  misc_feature 1..19: note = Targeting domain
  source 1..19: mol_type = other RNA
ccatgggtggagtttagcc

SEQ ID NO: 498 - RNA, 21 nt, synthetic construct
  misc_feature 1..21: note = Targeting domain
  source 1..21: mol_type = other RNA
acccatgggtggagtttagcc

SEQ ID NO: 499 - RNA, 22 nt, synthetic construct
  misc_feature 1..22: note = Targeting domain
  source 1..22: mol_type = other RNA
aacccatgggtggagtttagcc

SEQ ID NO: 500 - RNA, 23 nt, synthetic construct
  misc_feature 1..23: note = Targeting domain
  source 1..23: mol_type = other RNA
caacccatgggtggagtttagcc

SEQ ID NO: 501 - RNA, 24 nt, synthetic construct
  misc_feature 1..24: note = Targeting domain
  source 1..24: mol_type = other RNA
ccaacccatgggtggagtttagcc

SEQ ID NO: 502 - RNA, 18 nt, synthetic construct
  misc_feature 1..18: note = Targeting domain
  source 1..18: mol_type = other RNA
ccatgggtggagtttagc

SEQ ID NO: 503 - RNA, 19 nt, synthetic construct
  misc_feature 1..19: note = Targeting domain
  source 1..19: mol_type = other RNA
cccatgggtggagtttagc

SEQ ID NO: 504 - RNA, 20 nt, synthetic construct
  misc_feature 1..20: note = Targeting domain
  source 1..20: mol_type = other RNA
acccatgggtggagtttagc

SEQ ID NO: 505 - RNA, 21 nt, synthetic construct
  misc_feature 1..21: note = Targeting domain
  source 1..21: mol_type = other RNA
aacccatgggtggagtttagc

SEQ ID NO: 506 - RNA, 22 nt, synthetic construct
  misc_feature 1..22: note = Targeting domain
  source 1..22: mol_type = other RNA
caacccatgggtggagtttagc

SEQ ID NO: 507 - RNA, 23 nt, synthetic construct
  misc_feature 1..23: note = Targeting domain
  source 1..23: mol_type = other RNA
ccaacccatgggtggagtttagc

SEQ ID NO: 508 - RNA, 24 nt, synthetic construct
  misc_feature 1..24: note = Targeting domain
  source 1..24: mol_type = other RNA
gccaacccatgggtggagtttagc

SEQ ID NO: 509 - RNA, 18 nt, synthetic construct
  misc_feature 1..18: note = Targeting domain
  source 1..18: mol_type = other RNA
tgataacctcagacgttc

SEQ ID NO: 510 - RNA, 19 nt, synthetic construct
  misc_feature 1..19: note = Targeting domain
  source 1..19: mol_type = other RNA
ttgataacctcagacgttc

SEQ ID NO: 511 - RNA, 20 nt, synthetic construct
  misc_feature 1..20: note = Targeting domain
  source 1..20: mol_type = other RNA
attgataacctcagacgttc

SEQ ID NO: 512 - RNA, 21 nt, synthetic construct
  misc_feature 1..21: note = Targeting domain
  source 1..21: mol_type = other RNA
tattgataacctcagacgttc

SEQ ID NO: 513 - RNA, 22 nt, synthetic construct
  misc_feature 1..22: note = Targeting domain
  source 1..22: mol_type = other RNA
ttattgataacctcagacgttc

SEQ ID NO: 514 - RNA, 23 nt, synthetic construct
  misc_feature 1..23: note = Targeting domain
  source 1..23: mol_type = other RNA
cttattgataacctcagacgttc

SEQ ID NO: 515 - RNA, 24 nt, synthetic construct
  misc_feature 1..24: note = Targeting domain
  source 1..24: mol_type = other RNA
gcttattgataacctcagacgttc

SEQ ID NO: 516 - RNA, 18 nt, synthetic construct
  misc_feature 1..18: note = Targeting domain
  source 1..18: mol_type = other RNA
cattgagatagtgtgggg

SEQ ID NO: 517 - RNA, 19 nt, synthetic construct
  misc_feature 1..19: note = Targeting domain
  source 1..19: mol_type = other RNA
gcattgagatagtgtgggg

SEQ ID NO: 518 - RNA, 20 nt, synthetic construct
  misc_feature 1..20: note = Targeting domain
  source 1..20: mol_type = other RNA
tgcattgagatagtgtgggg

SEQ ID NO: 519 - RNA, 21 nt, synthetic construct
  misc_feature 1..21: note = Targeting domain
  source 1..21: mol_type = other RNA
ttgcattgagatagtgtgggg

SEQ ID NO: 520 - RNA, 22 nt, synthetic construct
  misc_feature 1..22: note = Targeting domain
  source 1..22: mol_type = other RNA
tttgcattgagatagtgtgggg

SEQ ID NO: 521 - RNA, 23 nt, synthetic construct
  misc_feature 1..23: note = Targeting domain
  source 1..23: mol_type = other RNA
atttgcattgagatagtgtgggg

SEQ ID NO: 522 - RNA, 24 nt, synthetic construct
  misc_feature 1..24: note = Targeting domain
  source 1..24: mol_type = other RNA
tatttgcattgagatagtgtgggg

SEQ ID NO: 523 - RNA, 18 nt, synthetic construct
  misc_feature 1..18: note = Targeting domain
  source 1..18: mol_type = other RNA
aggctggccaacccatgg

SEQ ID NO: 524 - RNA, 19 nt, synthetic construct
  misc_feature 1..19: note = Targeting domain
  source 1..19: mol_type = other RNA
aaggctggccaacccatgg

SEQ ID NO: 525 - RNA, 20 nt, synthetic construct
  misc_feature 1..20: note = Targeting domain
  source 1..20: mol_type = other RNA
caaggctggccaacccatgg

SEQ ID NO: 526 - RNA, 21 nt, synthetic construct
  misc_feature 1..21: note = Targeting domain
  source 1..21: mol_type = other RNA
gcaaggctggccaacccatgg

SEQ ID NO: 527 - RNA, 22 nt, synthetic construct
  misc_feature 1..22: note = Targeting domain
  source 1..22: mol_type = other RNA
ggcaaggctggccaacccatgg

SEQ ID NO: 528 - RNA, 23 nt, synthetic construct
  misc_feature 1..23: note = Targeting domain
  source 1..23: mol_type = other RNA
aggcaaggctggccaacccatgg

SEQ ID NO: 529 - RNA, 24 nt, synthetic construct
  misc_feature 1..24: note = Targeting domain
  source 1..24: mol_type = other RNA
aaggcaaggctggccaacccatgg

SEQ ID NO: 530 - RNA, 18 nt, synthetic construct
  misc_feature 1..18: note = Targeting domain
  source 1..18: mol_type = other RNA
agcgagtgtgtggaactg

SEQ ID NO: 531 - RNA, 19 nt, synthetic construct
  misc_feature 1..19: note = Targeting domain
  source 1..19: mol_type = other RNA
aagcgagtgtgtggaactg

SEQ ID NO: 532 - RNA, 20 nt, synthetic construct
  misc_feature 1..20: note = Targeting domain
  source 1..20: mol_type = other RNA
gaagcgagtgtgtggaactg

SEQ ID NO: 533 - RNA, 21 nt, synthetic construct
  misc_feature 1..21: note = Targeting domain
  source 1..21: mol_type = other RNA
agaagcgagtgtgtggaactg

SEQ ID NO: 534 - RNA, 22 nt, synthetic construct
  misc_feature 1..22: note = Targeting domain
  source 1..22: mol_type = other RNA
cagaagcgagtgtgtggaactg

SEQ ID NO: 535 - RNA, 23 nt, synthetic construct
  misc_feature 1..23: note = Targeting domain
  source 1..23: mol_type = other RNA
ccagaagcgagtgtgtggaactg

SEQ ID NO: 536 - RNA, 24 nt, synthetic construct
  misc_feature 1..24: note = Targeting domain
  source 1..24: mol_type = other RNA
tccagaagcgagtgtgtggaactg

SEQ ID NO: 537 - RNA, 18 nt, synthetic construct
  misc_feature 1..18: note = Targeting domain
  source 1..18: mol_type = other RNA
atttgcattgagatagtg

SEQ ID NO: 538 - RNA, 19 nt, synthetic construct
  misc_feature 1..19: note = Targeting domain
  source 1..19: mol_type = other RNA
tatttgcattgagatagtg

SEQ ID NO: 539 - RNA, 21 nt, synthetic construct
  misc_feature 1..21: note = Targeting domain
  source 1..21: mol_type = other RNA
gatatttgcattgagatagtg

SEQ ID NO: 540 - RNA, 22 nt, synthetic construct
  misc_feature 1..22: note = Targeting domain
  source 1..22: mol_type = other RNA
agatatttgcattgagatagtg

SEQ ID NO: 541 - RNA, 23 nt, synthetic construct
  misc_feature 1..23: note = Targeting domain
  source 1..23: mol_type = other RNA
cagatatttgcattgagatagtg

SEQ ID NO: 542 - RNA, 24 nt, synthetic construct
  misc_feature 1..24: note = Targeting domain
  source 1..24: mol_type = other RNA
acagatatttgcattgagatagtg

SEQ ID NO: 543 - RNA, 18 nt, synthetic construct
  misc_feature 1..18: note = Targeting domain
  source 1..18: mol_type = other RNA
gttccagaagcgagtgtg

SEQ ID NO: 544 - RNA, 19 nt, synthetic construct
  misc_feature 1..19: note = Targeting domain
  source 1..19: mol_type = other RNA
cgttccagaagcgagtgtg

SEQ ID NO: 545 - RNA, 21 nt, synthetic construct
  misc_feature 1..21: note = Targeting domain
  source 1..21: mol_type = other RNA
gacgttccagaagcgagtgtg

SEQ ID NO: 546 - RNA, 22 nt, synthetic construct
  misc_feature 1..22: note = Targeting domain
  source 1..22: mol_type = other RNA
agacgttccagaagcgagtgtg

SEQ ID NO: 547 - RNA, 23 nt, synthetic construct
  misc_feature 1..23: note = Targeting domain
  source 1..23: mol_type = other RNA
cagacgttccagaagcgagtgtg

SEQ ID NO: 548 - RNA, 24 nt, synthetic construct
  misc_feature 1..24: note = Targeting domain
  source 1..24: mol_type = other RNA
tcagacgttccagaagcgagtgtg

SEQ ID NO: 549 - RNA, 18 nt, synthetic construct
  misc_feature 1..18: note = Targeting domain
  source 1..18: mol_type = other RNA
ttgcattgagatagtgtg

SEQ ID NO: 550 - RNA, 19 nt, synthetic construct
  misc_feature 1..19: note = Targeting domain
  source 1..19: mol_type = other RNA
tttgcattgagatagtgtg

SEQ ID NO: 551 - RNA, 21 nt, synthetic construct
  misc_feature 1..21: note = Targeting domain
  source 1..21: mol_type = other RNA
tatttgcattgagatagtgtg

SEQ ID NO: 552 - RNA, 22 nt, synthetic construct
  misc_feature 1..22: note = Targeting domain
  source 1..22: mol_type = other RNA
atatttgcattgagatagtgtg

SEQ ID NO: 553 - RNA, 23 nt, synthetic construct
  misc_feature 1..23: note = Targeting domain
  source 1..23: mol_type = other RNA
gatatttgcattgagatagtgtg

SEQ ID NO: 554 - RNA, 24 nt, synthetic construct
  misc_feature 1..24: note = Targeting domain
  source 1..24: mol_type = other RNA
agatatttgcattgagatagtgtg

SEQ ID NO: 555 - RNA, 18 nt, synthetic construct
  misc_feature 1..18: note = Targeting domain
  source 1..18: mol_type = other RNA
tatttgcattgagatagt

SEQ ID NO: 556 - RNA, 19 nt, synthetic construct
  misc_feature 1..19: note = Targeting domain
  source 1..19: mol_type = other RNA
atatttgcattgagatagt

SEQ ID NO: 557 - RNA, 20 nt, synthetic construct
  misc_feature 1..20: note = Targeting domain
  source 1..20: mol_type = other RNA
gatatttgcattgagatagt

SEQ ID NO: 558 - RNA, 21 nt, synthetic construct
  misc_feature 1..21: note = Targeting domain
  source 1..21: mol_type = other RNA
agatatttgcattgagatagt

SEQ ID NO: 559 - RNA, 22 nt, synthetic construct
  misc_feature 1..22: note = Targeting domain
  source 1..22: mol_type = other RNA
cagatatttgcattgagatagt

SEQ ID NO: 560 - RNA, 23 nt, synthetic construct
  misc_feature 1..23: note = Targeting domain
  source 1..23: mol_type = other RNA
acagatatttgcattgagatagt

SEQ ID NO: 561 - RNA, 24 nt, synthetic construct
  misc_feature 1..24: note = Targeting domain
  source 1..24: mol_type = other RNA
gacagatatttgcattgagatagt

SEQ ID NO: 562 - RNA, 18 nt, synthetic construct
  misc_feature 1..18: note = Targeting domain
  source 1..18: mol_type = other RNA
cgttccagaagcgagtgt

SEQ ID NO: 563 - RNA, 19 nt, synthetic construct
  misc_feature 1..19: note = Targeting domain
  source 1..19: mol_type = other RNA
acgttccagaagcgagtgt

SEQ ID NO: 564 - RNA, 20 nt, synthetic construct
  misc_feature 1..20: note = Targeting domain
  source 1..20: mol_type = other RNA
gacgttccagaagcgagtgt

SEQ ID NO: 565 - RNA, 21 nt, synthetic construct
  misc_feature 1..21: note = Targeting domain
  source 1..21: mol_type = other RNA
agacgttccagaagcgagtgt

SEQ ID NO: 566 - RNA, 22 nt, synthetic construct
  misc_feature 1..22: note = Targeting domain
  source 1..22: mol_type = other RNA
cagacgttccagaagcgagtgt

SEQ ID NO: 567 - RNA, 23 nt, synthetic construct
  misc_feature 1..23: note = Targeting domain
  source 1..23: mol_type = other RNA
tcagacgttccagaagcgagtgt

SEQ ID NO: 568 - RNA, 24 nt, synthetic construct
  misc_feature 1..24: note = Targeting domain
  source 1..24: mol_type = other RNA
ctcagacgttccagaagcgagtgt

SEQ ID NO: 569 - RNA, 18 nt, synthetic construct
  misc_feature 1..18: note = Targeting domain
  source 1..18: mol_type = other RNA
tttgcattgagatagtgt

SEQ ID NO: 570 - RNA, 19 nt, synthetic construct
  misc_feature 1..19: note = Targeting domain
  source 1..19: mol_type = other RNA
atttgcattgagatagtgt

SEQ ID NO: 571 - RNA, 21 nt, synthetic construct
  misc_feature 1..21: note = Targeting domain
  source 1..21: mol_type = other RNA
atatttgcattgagatagtgt

SEQ ID NO: 572 - RNA, 22 nt, synthetic construct
  misc_feature 1..22: note = Targeting domain
  source 1..22: mol_type = other RNA
gatatttgcattgagatagtgt

SEQ ID NO: 573 - RNA, 23 nt, synthetic construct
  misc_feature 1..23: note = Targeting domain
  source 1..23: mol_type = other RNA
agatatttgcattgagatagtgt

SEQ ID NO: 574 - RNA, 24 nt, synthetic construct
  misc_feature 1..24: note = Targeting domain
  source 1..24: mol_type = other RNA
cagatatttgcattgagatagtgt

SEQ ID NO: 575 - RNA, 18 nt, synthetic construct
  misc_feature 1..18: note = Targeting domain
  source 1..18: mol_type = other RNA
gaataaattagagaaaaa

SEQ ID NO: 576 - RNA, 19 nt, synthetic construct
  misc_feature 1..19: note = Targeting domain
  source 1..19: mol_type = other RNA
agaataaattagagaaaaa

SEQ ID NO: 577 - RNA, 20 nt, synthetic construct
  misc_feature 1..20: note = Targeting domain
  source 1..20: mol_type = other RNA
aagaataaattagagaaaaa

SEQ ID NO: 578 - RNA, 21 nt, synthetic construct
  misc_feature 1..21: note = Targeting domain
  source 1..21: mol_type = other RNA
gaagaataaattagagaaaaa

SEQ ID NO: 579 - RNA, 22 nt, synthetic construct
  misc_feature 1..22: note = Targeting domain
  source 1..22: mol_type = other RNA
ggaagaataaattagagaaaaa

SEQ ID NO: 580 - RNA, 23 nt, synthetic construct
  misc_feature 1..23: note = Targeting domain
  source 1..23: mol_type = other RNA
gggaagaataaattagagaaaaa

SEQ ID NO: 581 - RNA, 24 nt, synthetic construct
  misc_feature 1..24: note = Targeting domain
  source 1..24: mol_type = other RNA
agggaagaataaattagagaaaaa

SEQ ID NO: 582 - RNA, 18 nt, synthetic construct
  misc_feature 1..18: note = Targeting domain
  source 1..18: mol_type = other RNA
ctagggatgaagaataaa

SEQ ID NO: 583 - RNA, 19 nt, synthetic construct
  misc_feature 1..19: note = Targeting domain
  source 1..19: mol_type = other RNA
gctagggatgaagaataaa

SEQ ID NO: 584 - RNA, 21 nt, synthetic construct
  misc_feature 1..21: note = Targeting domain
  source 1..21: mol_type = other RNA
tggctagggatgaagaataaa

SEQ ID NO: 585 - RNA, 22 nt, synthetic construct
  misc_feature 1..22: note = Targeting domain
  source 1..22: mol_type = other RNA
ctggctagggatgaagaataaa

SEQ ID NO: 586 - RNA, 23 nt, synthetic construct
  misc_feature 1..23: note = Targeting domain
  source 1..23: mol_type = other RNA
gctggctagggatgaagaataaa

SEQ ID NO: 587 - RNA, 24 nt, synthetic construct
  misc_feature 1..24: note = Targeting domain
  source 1..24: mol_type = other RNA
ggctggctagggatgaagaataaa

SEQ ID NO: 588 - RNA, 18 nt, synthetic construct
  misc_feature 1..18: note = Targeting domain
  source 1..18: mol_type = other RNA
agaaggaaactagctaaa

SEQ ID NO: 589 - RNA, 19 nt, synthetic construct
  misc_feature 1..19: note = Targeting domain
  source 1..19: mol_type = other RNA
gagaaggaaactagctaaa

SEQ ID NO: 590 - RNA, 21 nt, synthetic construct
  misc_feature 1..21: note = Targeting domain
  source 1..21: mol_type = other RNA
gggagaaggaaactagctaaa

SEQ ID NO: 591 - RNA, 22 nt, synthetic construct
  misc_feature 1..22: note = Targeting domain
  source 1..22: mol_type = other RNA
tgggagaaggaaactagctaaa

SEQ ID NO: 592 - RNA, 23 nt, synthetic construct
  misc_feature 1..23: note = Targeting domain
  source 1..23: mol_type = other RNA
atgggagaaggaaactagctaaa

SEQ ID NO: 593 - RNA, 24 nt, synthetic construct
  misc_feature 1..24: note = Targeting domain
  source 1..24: mol_type = other RNA
gatgggagaaggaaactagctaaa

SEQ ID NO: 594 - RNA, 18 nt, synthetic construct
  misc_feature 1..18: note = Targeting domain
  source 1..18: mol_type = other RNA
aaaactggaatgactgaa

SEQ ID NO: 595 - RNA, 19 nt, synthetic construct
  misc_feature 1..19: note = Targeting domain
  source 1..19: mol_type = other RNA
aaaaactggaatgactgaa

SEQ ID NO: 596 - RNA, 20 nt, synthetic construct
  misc_feature 1..20: note = Targeting domain
  source 1..20: mol_type = other RNA
gaaaaactggaatgactgaa

SEQ ID NO: 597 - RNA, 21 nt, synthetic construct
  misc_feature 1..21: note = Targeting domain
  source 1..21: mol_type = other RNA
agaaaaactggaatgactgaa

SEQ ID NO: 598 - RNA, 22 nt, synthetic construct
  misc_feature 1..22: note = Targeting domain
  source 1..22: mol_type = other RNA
gagaaaaactggaatgactgaa

SEQ ID NO: 599 - RNA, 23 nt, synthetic construct
  misc_feature 1..23: note = Targeting domain
  source 1..23: mol_type = other RNA
agagaaaaactggaatgactgaa

SEQ ID NO: 600 - RNA, 24 nt, synthetic construct
  misc_feature 1..24: note = Targeting domain
  source 1..24: mol_type = other RNA
tagagaaaaactggaatgactgaa

SEQ ID NO: 601 - RNA, 18 nt, synthetic construct
  misc_feature 1..18: note = Targeting domain
  source 1..18: mol_type = other RNA
gctagggatgaagaataa

SEQ ID NO: 602 - RNA, 19 nt, synthetic construct
  misc_feature 1..19: note = Targeting domain
  source 1..19: mol_type = other RNA
ggctagggatgaagaataa

SEQ ID NO: 603 - RNA, 20 nt, synthetic construct
  misc_feature 1..20: note = Targeting domain
  source 1..20: mol_type = other RNA
tggctagggatgaagaataa

SEQ ID NO: 604 - RNA, 21 nt, synthetic construct
  misc_feature 1..21: note = Targeting domain
  source 1..21: mol_type = other RNA
ctggctagggatgaagaataa

SEQ ID NO: 605 - RNA, 22 nt, synthetic construct
  misc_feature 1..22: note = Targeting domain
  source 1..22: mol_type = other RNA
gctggctagggatgaagaataa

SEQ ID NO: 606 - RNA, 23 nt, synthetic construct
  misc_feature 1..23: note = Targeting domain
  source 1..23: mol_type = other RNA
ggctggctagggatgaagaataa

SEQ ID NO: 607 - RNA, 24 nt, synthetic construct
  misc_feature 1..24: note = Targeting domain
  source 1..24: mol_type = other RNA
cggctggctagggatgaagaataa

SEQ ID NO: 608 - RNA, 18 nt, synthetic construct
  misc_feature 1..18: note = Targeting domain
  source 1..18: mol_type = other RNA
gagaaggaaactagctaa

SEQ ID NO: 609 - RNA, 19 nt, synthetic construct
  misc_feature 1..19: note = Targeting domain
  source 1..19: mol_type = other RNA
ggagaaggaaactagctaa

SEQ ID NO: 610 - RNA, 21 nt, synthetic construct
  misc_feature 1..21: note = Targeting domain
  source 1..21: mol_type = other RNA
tgggagaaggaaactagctaa

SEQ ID NO: 611 - RNA, 22 nt, synthetic construct
  misc_feature 1..22: note = Targeting domain
  source 1..22: mol_type = other RNA
atgggagaaggaaactagctaa

SEQ ID NO: 612 - RNA, 23 nt, synthetic construct
  misc_feature 1..23: note = Targeting domain
  source 1..23: mol_type = other RNA
gatgggagaaggaaactagctaa

SEQ ID NO: 613 - RNA, 24 nt, synthetic construct
  misc_feature 1..24: note = Targeting domain
  source 1..24: mol_type = other RNA
tgatgggagaaggaaactagctaa

SEQ ID NO: 614 - RNA, 18 nt, synthetic construct
  misc_feature 1..18: note = Targeting domain
  source 1..18: mol_type = other RNA
atagtcttagagtatcca

SEQ ID NO: 615 - RNA, 19 nt, synthetic construct
  misc_feature 1..19: note = Targeting domain
  source 1..19: mol_type = other RNA
aatagtcttagagtatcca

SEQ ID NO: 616 - RNA, 20 nt, synthetic construct
  misc_feature 1..20: note = Targeting domain
  source 1..20: mol_type = other RNA
caatagtcttagagtatcca

SEQ ID NO: 617 - RNA, 21 nt, synthetic construct
  misc_feature 1..21: note = Targeting domain
  source 1..21: mol_type = other RNA
ccaatagtcttagagtatcca

SEQ ID NO: 618 - RNA, 22 nt, synthetic construct
  misc_feature 1..22: note = Targeting domain
  source 1..22: mol_type = other RNA
accaatagtcttagagtatcca

SEQ ID NO: 619 - RNA, 23 nt, synthetic construct
  misc_feature 1..23: note = Targeting domain
  source 1..23: mol_type = other RNA
gaccaatagtcttagagtatcca

SEQ ID NO: 620 - RNA, 24 nt, synthetic construct
  misc_feature 1..24: note = Targeting domain
  source 1..24: mol_type = other RNA
tgaccaatagtcttagagtatcca

SEQ ID NO: 621 - RNA, 18 nt, synthetic construct
  misc_feature 1..18: note = Targeting domain
  source 1..18: mol_type = other RNA
atcctctatgatgggaga

SEQ ID NO: 622 - RNA, 19 nt, synthetic construct
  misc_feature 1..19: note = Targeting domain
  source 1..19: mol_type = other RNA
tatcctctatgatgggaga

SEQ ID NO: 623 - RNA, 21 nt, synthetic construct
  misc_feature 1..21: note = Targeting domain
  source 1..21: mol_type = other RNA
ggtatcctctatgatgggaga

SEQ ID NO: 624 - RNA, 22 nt, synthetic construct
  misc_feature 1..22: note = Targeting domain
  source 1..22: mol_type = other RNA
tggtatcctctatgatgggaga

SEQ ID NO: 625 - RNA, 23 nt, synthetic construct
  misc_feature 1..23: note = Targeting domain
  source 1..23: mol_type = other RNA
ctggtatcctctatgatgggaga

SEQ ID NO: 626 - RNA, 24 nt, synthetic construct
  misc_feature 1..24: note = Targeting domain
  source 1..24: mol_type = other RNA
cctggtatcctctatgatgggaga

SEQ ID NO: 627 - RNA, 18 nt, synthetic construct
  misc_feature 1..18: note = Targeting domain
  source 1..18: mol_type = other RNA
tcctggtatcctctatga

SEQ ID NO: 628 - RNA, 19 nt, synthetic construct
  misc_feature 1..19: note = Targeting domain
  source 1..19: mol_type = other RNA
gtcctggtatcctctatga

SEQ ID NO: 629 - RNA, 21 nt, synthetic construct
  misc_feature 1..21: note = Targeting domain
  source 1..21: mol_type = other RNA
aagtcctggtatcctctatga

SEQ ID NO: 630 - RNA, 22 nt, synthetic construct
  misc_feature 1..22: note = Targeting domain
  source 1..22: mol_type = other RNA
gaagtcctggtatcctctatga

SEQ ID NO: 631 - RNA, 23 nt, synthetic construct
  misc_feature 1..23: note = Targeting domain
  source 1..23: mol_type = other RNA
agaagtcctggtatcctctatga

SEQ ID NO: 632 - RNA, 24 nt, synthetic construct
  misc_feature 1..24: note = Targeting domain
  source 1..24: mol_type = other RNA
aagaagtcctggtatcctctatga

SEQ ID NO: 633 - RNA, 18 nt, synthetic construct
  misc_feature 1..18: note = Targeting domain
  source 1..18: mol_type = other RNA
ggagaaggaaactagcta

SEQ ID NO: 634 - RNA, 19 nt, synthetic construct
  misc_feature 1..19: note = Targeting domain
  source 1..19: mol_type = other RNA
gggagaaggaaactagcta

SEQ ID NO: 635 - RNA, 20 nt, synthetic construct
  misc_feature 1..20: note = Targeting domain
  source 1..20: mol_type = other RNA
tgggagaaggaaactagcta

SEQ ID NO: 636 - RNA, 21 nt, synthetic construct
  misc_feature 1..21: note = Targeting domain
  source 1..21: mol_type = other RNA
atgggagaaggaaactagcta

SEQ ID NO: 637 - RNA, 22 nt, synthetic construct
  misc_feature 1..22: note = Targeting domain
  source 1..22: mol_type = other RNA
gatgggagaaggaaactagcta

SEQ ID NO: 638 - RNA, 23 nt, synthetic construct
  misc_feature 1..23: note = Targeting domain
  source 1..23: mol_type = other RNA
tgatgggagaaggaaactagcta

SEQ ID NO: 639 - RNA, 24 nt, synthetic construct
  misc_feature 1..24: note = Targeting domain
  source 1..24: mol_type = other RNA
atgatgggagaaggaaactagcta

SEQ ID NO: 640 - RNA, 18 nt, synthetic construct
  misc_feature 1..18: note = Targeting domain
  source 1..18: mol_type = other RNA
aaagggaagaataaatta

SEQ ID NO: 641 - RNA, 19 nt, synthetic construct
  misc_feature 1..19: note = Targeting domain
  source 1..19: mol_type = other RNA
taaagggaagaataaatta

SEQ ID NO: 642 - RNA, 20 nt, synthetic construct
  misc_feature 1..20: note = Targeting domain
  source 1..20: mol_type = other RNA
ctaaagggaagaataaatta

SEQ ID NO: 643 - RNA, 21 nt, synthetic construct
  misc_feature 1..21: note = Targeting domain
  source 1..21: mol_type = other RNA
gctaaagggaagaataaatta

SEQ ID NO: 644 - RNA, 22 nt, synthetic construct
  misc_feature 1..22: note = Targeting domain
  source 1..22: mol_type = other RNA
agctaaagggaagaataaatta

SEQ ID NO: 645 - RNA, 23 nt, synthetic construct
  misc_feature 1..23: note = Targeting domain
  source 1..23: mol_type = other RNA
tagctaaagggaagaataaatta

SEQ ID NO: 646 - RNA, 24 nt, synthetic construct
  misc_feature 1..24: note = Targeting domain
  source 1..24: mol_type = other RNA
ctagctaaagggaagaataaatta

SEQ ID NO: 647 - RNA, 18 nt, synthetic construct
  misc_feature 1..18: note = Targeting domain
  source 1..18: mol_type = other RNA
agagtatccagtgaggcc

SEQ ID NO: 648 - RNA, 19 nt, synthetic construct
  misc_feature 1..19: note = Targeting domain
  source 1..19: mol_type = other RNA
tagagtatccagtgaggcc

SEQ ID NO: 649 - RNA, 21 nt, synthetic construct
  misc_feature 1..21: note = Targeting domain
  source 1..21: mol_type = other RNA
cttagagtatccagtgaggcc

SEQ ID NO: 650 - RNA, 22 nt, synthetic construct
  misc_feature 1..22: note = Targeting domain
  source 1..22: mol_type = other RNA
tcttagagtatccagtgaggcc

SEQ ID NO: 651 - RNA, 23 nt, synthetic construct
  misc_feature 1..23: note = Targeting domain
  source 1..23: mol_type = other RNA
gtcttagagtatccagtgaggcc

SEQ ID NO: 652 - RNA, 24 nt, synthetic construct
  misc_feature 1..24: note = Targeting domain
  source 1..24: mol_type = other RNA
agtcttagagtatccagtgaggcc

SEQ ID NO: 653 - RNA, 18 nt, synthetic construct
  misc_feature 1..18: note = Targeting domain
  source 1..18: mol_type = other RNA
tagagtatccagtgaggc

SEQ ID NO: 654 - RNA, 19 nt, synthetic construct
  misc_feature 1..19: note = Targeting domain
  source 1..19: mol_type = other RNA
ttagagtatccagtgaggc

SEQ ID NO: 655 - RNA, 20 nt, synthetic construct
  misc_feature 1..20: note = Targeting domain
  source 1..20: mol_type = other RNA
cttagagtatccagtgaggc

SEQ ID NO: 656 - RNA, 21 nt, synthetic construct
  misc_feature 1..21: note = Targeting domain
  source 1..21: mol_type = other RNA
tcttagagtatccagtgaggc

SEQ ID NO: 657 - RNA, 22 nt, synthetic construct
  misc_feature 1..22: note = Targeting domain
  source 1..22: mol_type = other RNA
gtcttagagtatccagtgaggc

SEQ ID NO: 658 - RNA, 23 nt, synthetic construct
  misc_feature 1..23: note = Targeting domain
  source 1..23: mol_type = other RNA
agtcttagagtatccagtgaggc

SEQ ID NO: 659 - RNA, 24 nt, synthetic construct
  misc_feature 1..24: note = Targeting domain
  source 1..24: mol_type = other RNA
tagtcttagagtatccagtgaggc

SEQ ID NO: 660 - RNA, 18 nt, synthetic construct
  misc_feature 1..18: note = Targeting domain
  source 1..18: mol_type = other RNA
caggggccggcggctggc

SEQ ID NO: 661 - RNA, 19 nt, synthetic construct
  misc_feature 1..19: note = Targeting domain
  source 1..19: mol_type = other RNA
ccaggggccggcggctggc

SEQ ID NO: 662 - RNA, 20 nt, synthetic construct
  misc_feature 1..20: note = Targeting domain
  source 1..20: mol_type = other RNA
gccaggggccggcggctggc

SEQ ID NO: 663 - RNA, 21 nt, synthetic construct
  misc_feature 1..21: note = Targeting domain
  source 1..21: mol_type = other RNA
ggccaggggccggcggctggc

SEQ ID NO: 664 - RNA, 22 nt, synthetic construct
  misc_feature 1..22: note = Targeting domain
  source 1..22: mol_type = other RNA
aggccaggggccggcggctggc

SEQ ID NO: 665 - RNA, 23 nt, synthetic construct
  misc_feature 1..23: note = Targeting domain
  source 1..23: mol_type = other RNA
gaggccaggggccggcggctggc

SEQ ID NO: 666 - RNA, 24 nt, synthetic construct
  misc_feature 1..24: note = Targeting domain
  source 1..24: mol_type = other RNA
tgaggccaggggccggcggctggc

SEQ ID NO: 667 - RNA, 18 nt, synthetic construct
  misc_feature 1..18: note = Targeting domain
  source 1..18: mol_type = other RNA
aaaattagcagtatcctc

SEQ ID NO: 668 - RNA, 19 nt, synthetic construct
  misc_feature 1..19: note = Targeting domain
  source 1..19: mol_type = other RNA
aaaaattagcagtatcctc

SEQ ID NO: 669 - RNA, 20 nt, synthetic construct
  misc_feature 1..20: note = Targeting domain
  source 1..20: mol_type = other RNA
aaaaaattagcagtatcctc

SEQ ID NO: 670 - RNA, 21 nt, synthetic construct
  misc_feature 1..21: note = Targeting domain
  source 1..21: mol_type = other RNA
aaaaaaattagcagtatcctc

SEQ ID NO: 671 - RNA, 22 nt, synthetic construct
  misc_feature 1..22: note = Targeting domain
  source 1..22: mol_type = other RNA
aaaaaaaattagcagtatcctc

SEQ ID NO: 672 - RNA, 23 nt, synthetic construct
  misc_feature 1..23: note = Targeting domain
  source 1..23: mol_type = other RNA
taaaaaaaattagcagtatcctc

SEQ ID NO: 673 - RNA, 24 nt, synthetic construct
  misc_feature 1..24: note = Targeting domain
  source 1..24: mol_type = other RNA
ataaaaaaaattagcagtatcctc

SEQ ID NO: 674 - RNA, 18 nt, synthetic construct
  misc_feature 1..18: note = Targeting domain
  source 1..18: mol_type = other RNA
gttccacacactcgcttc

SEQ ID NO: 675 - RNA, 19 nt, synthetic construct
  misc_feature 1..19: note = Targeting domain
  source 1..19: mol_type = other RNA
agttccacacactcgcttc

SEQ ID NO: 676 - RNA, 21 nt, synthetic construct
  misc_feature 1..21: note = Targeting domain
  source 1..21: mol_type = other RNA
gcagttccacacactcgcttc

SEQ ID NO: 677 - RNA, 22 nt, synthetic construct
  misc_feature 1..22: note = Targeting domain
  source 1..22: mol_type = other RNA
agcagttccacacactcgcttc

SEQ ID NO: 678 - RNA, 23 nt, synthetic construct
  misc_feature 1..23: note = Targeting domain
  source 1..23: mol_type = other RNA
cagcagttccacacactcgcttc

SEQ ID NO: 679 - RNA, 24 nt, synthetic construct
  misc_feature 1..24: note = Targeting domain
  source 1..24: mol_type = other RNA
tcagcagttccacacactcgcttc

SEQ ID NO: 680 - RNA, 18 nt, synthetic construct
  misc_feature 1..18: note = Targeting domain
  source 1..18: mol_type = other RNA
tatcctctatgatgggag

SEQ ID NO: 681 - RNA, 19 nt, synthetic construct
  misc_feature 1..19: note = Targeting domain
  source 1..19: mol_type = other RNA
gtatcctctatgatgggag

SEQ ID NO: 682 - RNA, 20 nt, synthetic construct
  misc_feature 1..20: note = Targeting domain
  source 1..20: mol_type = other RNA
ggtatcctctatgatgggag

SEQ ID NO: 683 - RNA, 21 nt, synthetic construct
  misc_feature 1..21: note = Targeting domain
  source 1..21: mol_type = other RNA
tggtatcctctatgatgggag

SEQ ID NO: 684 - RNA, 22 nt, synthetic construct
  misc_feature 1..22: note = Targeting domain
  source 1..22: mol_type = other RNA
ctggtatcctctatgatgggag

SEQ ID NO: 685 - RNA, 23 nt, synthetic construct
  misc_feature 1..23: note = Targeting domain
  source 1..23: mol_type = other RNA
cctggtatcctctatgatgggag

SEQ ID NO: 686 - RNA, 24 nt, synthetic construct
  misc_feature 1..24: note = Targeting domain
  source 1..24: mol_type = other RNA
tcctggtatcctctatgatgggag

SEQ ID NO: 687 - RNA, 18 nt, synthetic construct
  misc_feature 1..18: note = Targeting domain
  source 1..18: mol_type = other RNA
gccggcggctggctaggg

SEQ ID NO: 688 - RNA, 19 nt, synthetic construct
  misc_feature 1..19: note = Targeting domain
  source 1..19: mol_type = other RNA
ggccggcggctggctaggg

SEQ ID NO: 689 - RNA, 20 nt, synthetic construct
  misc_feature 1..20: note = Targeting domain
  source 1..20: mol_type = other RNA
gggccggcggctggctaggg

SEQ ID NO: 690 - RNA, 21 nt, synthetic construct
  misc_feature 1..21: note = Targeting domain
  source 1..21: mol_type = other RNA
ggggccggcggctggctaggg

SEQ ID NO: 691 - RNA, 22 nt, synthetic construct
  misc_feature 1..22: note = Targeting domain
  source 1..22: mol_type = other RNA
aggggccggcggctggctaggg

SEQ ID NO: 692 - RNA, 23 nt, synthetic construct
  misc_feature 1..23: note = Targeting domain
  source 1..23: mol_type = other RNA
caggggccggcggctggctaggg

SEQ ID NO: 693 - RNA, 24 nt, synthetic construct
  misc_feature 1..24: note = Targeting domain
  source 1..24: mol_type = other RNA
ccaggggccggcggctggctaggg

SEQ ID NO: 694 - RNA, 18 nt, synthetic construct
  misc_feature 1..18: note = Targeting domain
  source 1..18: mol_type = other RNA
tggtatcctctatgatgg

SEQ ID NO: 695 - RNA, 19 nt, synthetic construct
  misc_feature 1..19: note = Targeting domain
  source 1..19: mol_type = other RNA
ctggtatcctctatgatgg

SEQ ID NO: 696 - RNA, 20 nt, synthetic construct
  misc_feature 1..20: note = Targeting domain
  source 1..20: mol_type = other RNA
cctggtatcctctatgatgg

SEQ ID NO: 697 - RNA, 21 nt, synthetic construct
  misc_feature 1..21: note = Targeting domain
  source 1..21: mol_type = other RNA
tcctggtatcctctatgatgg

SEQ ID NO: 698 - RNA, 22 nt, synthetic construct
  misc_feature 1..22: note = Targeting domain
  source 1..22: mol_type = other RNA
gtcctggtatcctctatgatgg

SEQ ID NO: 699 - RNA, 23 nt, synthetic construct
  misc_feature 1..23: note = Targeting domain
  source 1..23: mol_type = other RNA
agtcctggtatcctctatgatgg

SEQ ID NO: 700 - RNA, 24 nt, synthetic construct
  misc_feature 1..24: note = Targeting domain
  source 1..24: mol_type = other RNA
aagtcctggtatcctctatgatgg

SEQ ID NO: 701 - RNA, 18 nt, synthetic construct
  misc_feature 1..18: note = Targeting domain
  source 1..18: mol_type = other RNA
gtcctggtatcctctatg

SEQ ID NO: 702 - RNA, 19 nt, synthetic construct
  misc_feature 1..19: note = Targeting domain
  source 1..19: mol_type = other RNA
agtcctggtatcctctatg

SEQ ID NO: 703 - RNA, 20 nt, synthetic construct
  misc_feature 1..20: note = Targeting domain
  source 1..20: mol_type = other RNA
aagtcctggtatcctctatg

SEQ ID NO: 704 - RNA, 21 nt, synthetic construct
  misc_feature 1..21: note = Targeting domain
  source 1..21: mol_type = other RNA
gaagtcctggtatcctctatg

SEQ ID NO: 705 - RNA, 22 nt, synthetic construct
  misc_feature 1..22: note = Targeting domain
  source 1..22: mol_type = other RNA
agaagtcctggtatcctctatg

SEQ ID NO: 706 - RNA, 23 nt, synthetic construct
  misc_feature 1..23: note = Targeting domain
  source 1..23: mol_type = other RNA
aagaagtcctggtatcctctatg

SEQ ID NO: 707 - RNA, 24 nt, synthetic construct
  misc_feature 1..24: note = Targeting domain
  source 1..24: mol_type = other RNA
aaagaagtcctggtatcctctatg

SEQ ID NO: 708 - RNA, 18 nt, synthetic construct
  misc_feature 1..18: note = Targeting domain
  source 1..18: mol_type = other RNA
ctaaagggaagaataaat

SEQ ID NO: 709 - RNA, 19 nt, synthetic construct
  misc_feature 1..19: note = Targeting domain
  source 1..19: mol_type = other RNA
gctaaagggaagaataaat

SEQ ID NO: 710 - RNA, 20 nt, synthetic construct
  misc_feature 1..20: note = Targeting domain
  source 1..20: mol_type = other RNA
agctaaagggaagaataaat

SEQ ID NO: 711 - RNA, 21 nt, synthetic construct
  misc_feature 1..21: note = Targeting domain
  source 1..21: mol_type = other RNA
tagctaaagggaagaataaat

SEQ ID NO: 712 - RNA, 22 nt, synthetic construct
  misc_feature 1..22: note = Targeting domain
  source 1..22: mol_type = other RNA
ctagctaaagggaagaataaat

SEQ ID NO: 713 - RNA, 23 nt, synthetic construct
  misc_feature 1..23: note = Targeting domain
  source 1..23: mol_type = other RNA
actagctaaagggaagaataaat

SEQ ID NO: 714 - RNA, 24 nt, synthetic construct
  misc_feature 1..24: note = Targeting domain
  source 1..24: mol_type = other RNA
aactagctaaagggaagaataaat

SEQ ID NO: 715 - RNA, 18 nt, synthetic construct
  misc_feature 1..18: note = Targeting domain
  source 1..18: mol_type = other RNA
aaactggaatgactgaat

SEQ ID NO: 716 - RNA, 19 nt, synthetic construct
  misc_feature 1..19: note = Targeting domain
  source 1..19: mol_type = other RNA
aaaactggaatgactgaat

SEQ ID NO: 717 - RNA, 21 nt, synthetic construct
  misc_feature 1..21: note = Targeting domain
  source 1..21: mol_type = other RNA
gaaaaactggaatgactgaat

SEQ ID NO: 718 - RNA, 22 nt, synthetic construct
  misc_feature 1..22: note = Targeting domain
  source 1..22: mol_type = other RNA
agaaaaactggaatgactgaat

SEQ ID NO: 719 - RNA, 23 nt, synthetic construct
  misc_feature 1..23: note = Targeting domain
  source 1..23: mol_type = other RNA
gagaaaaactggaatgactgaat

SEQ ID NO: 720 - RNA, 24 nt, synthetic construct
  misc_feature 1..24: note = Targeting domain
  source 1..24: mol_type = other RNA
agagaaaaactggaatgactgaat

SEQ ID NO: 721 - RNA, 18 nt, synthetic construct
  misc_feature 1..18: note = Targeting domain
  source 1..18: mol_type = other RNA
cctggtatcctctatgat

SEQ ID NO: 722 - RNA, 19 nt, synthetic construct
  misc_feature 1..19: note = Targeting domain
  source 1..19: mol_type = other RNA
tcctggtatcctctatgat

SEQ ID NO: 723 - RNA, 21 nt, synthetic construct
  misc_feature 1..21: note = Targeting domain
  source 1..21: mol_type = other RNA
agtcctggtatcctctatgat

SEQ ID NO: 724 - RNA, 22 nt, synthetic construct
  misc_feature 1..22: note = Targeting domain
  source 1..22: mol_type = other RNA
aagtcctggtatcctctatgat

SEQ ID NO: 725 - RNA, 23 nt, synthetic construct
  misc_feature 1..23: note = Targeting domain
  source 1..23: mol_type = other RNA
gaagtcctggtatcctctatgat

SEQ ID NO: 726 - RNA, 24 nt, synthetic construct
  misc_feature 1..24: note = Targeting domain
  source 1..24: mol_type = other RNA
agaagtcctggtatcctctatgat

SEQ ID NO: 727 - RNA, 18 nt, synthetic construct
  misc_feature 1..18: note = Targeting domain
  source 1..18: mol_type = other RNA
aaattagcagtatcctct

SEQ ID NO: 728 - RNA, 19 nt, synthetic construct
  misc_feature 1..19: note = Targeting domain
  source 1..19: mol_type = other RNA
aaaattagcagtatcctct

SEQ ID NO: 729 - RNA, 21 nt, synthetic construct
  misc_feature 1..21: note = Targeting domain
  source 1..21: mol_type = other RNA
aaaaaattagcagtatcctct

SEQ ID NO: 730 - RNA, 22 nt, synthetic construct
  misc_feature 1..22: note = Targeting domain
  source 1..22: mol_type = other RNA
aaaaaaattagcagtatcctct

SEQ ID NO: 731 - RNA, 23 nt, synthetic construct
  misc_feature 1..23: note = Targeting domain
  source 1..23: mol_type = other RNA
aaaaaaaattagcagtatcctct

SEQ ID NO: 732 - RNA, 24 nt, synthetic construct
  misc_feature 1..24: note = Targeting domain
  source 1..24: mol_type = other RNA
taaaaaaaattagcagtatcctct

SEQ ID NO: 733 - RNA, 18 nt, synthetic construct
  misc_feature 1..18: note = Targeting domain
  source 1..18: mol_type = other RNA
cactcgcttctggaacgt

SEQ ID NO: 734 - RNA, 19 nt, synthetic construct
  misc_feature 1..19: note = Targeting domain
  source 1..19: mol_type = other RNA
acactcgcttctggaacgt

SEQ ID NO: 735 - RNA, 20 nt, synthetic construct
  misc_feature 1..20: note = Targeting domain
  source 1..20: mol_type = other RNA
cacactcgcttctggaacgt

SEQ ID NO: 736 - RNA, 21 nt, synthetic construct
  misc_feature 1..21: note = Targeting domain
  source 1..21: mol_type = other RNA
acacactcgcttctggaacgt

SEQ ID NO: 737 - RNA, 22 nt, synthetic construct
  misc_feature 1..22: note = Targeting domain
  source 1..22: mol_type = other RNA
cacacactcgcttctggaacgt

SEQ ID NO: 738 - RNA, 23 nt, synthetic construct
  misc_feature 1..23: note = Targeting domain
  source 1..23: mol_type = other RNA
ccacacactcgcttctggaacgt

SEQ ID NO: 739 - RNA, 24 nt, synthetic construct
  misc_feature 1..24: note = Targeting domain
  source 1..24: mol_type = other RNA
tccacacactcgcttctggaacgt

SEQ ID NO: 740 - RNA, 18 nt, synthetic construct
  misc_feature 1..18: note = Targeting domain
  source 1..18: mol_type = other RNA
ctcaatgcaaatatctgt

SEQ ID NO: 741 - RNA, 19 nt, synthetic construct
  misc_feature 1..19: note = Targeting domain
  source 1..19: mol_type = other RNA
tctcaatgcaaatatctgt

SEQ ID NO: 742 - RNA, 20 nt, synthetic construct
  misc_feature 1..20: note = Targeting domain
  source 1..20: mol_type = other RNA
atctcaatgcaaatatctgt

SEQ ID NO: 743 - RNA, 21 nt, synthetic construct
  misc_feature 1..21: note = Targeting domain
  source 1..21: mol_type = other RNA
tatctcaatgcaaatatctgt

SEQ ID NO: 744 - RNA, 22 nt, synthetic construct
  misc_feature 1..22: note = Targeting domain
  source 1..22: mol_type = other RNA
ctatctcaatgcaaatatctgt

SEQ ID NO: 745 - RNA, 23 nt, synthetic construct
  misc_feature 1..23: note = Targeting domain
  source 1..23: mol_type = other RNA
actatctcaatgcaaatatctgt

SEQ ID NO: 746 - RNA, 24 nt, synthetic construct
  misc_feature 1..24: note = Targeting domain
  source 1..24: mol_type = other RNA
cactatctcaatgcaaatatctgt

SEQ ID NO: 747 - RNA, 18 nt, synthetic construct
  misc_feature 1..18: note = Targeting domain
  source 1..18: mol_type = other RNA
agttccacacactcgctt

SEQ ID NO: 748 - RNA, 19 nt, synthetic construct
  misc_feature 1..19: note = Targeting domain
  source 1..19: mol_type = other RNA
cagttccacacactcgctt

SEQ ID NO: 749 - RNA, 20 nt, synthetic construct
  misc_feature 1..20: note = Targeting domain
  source 1..20: mol_type = other RNA
gcagttccacacactcgctt

SEQ ID NO: 750 - RNA, 21 nt, synthetic construct
  misc_feature 1..21: note = Targeting domain
  source 1..21: mol_type = other RNA
agcagttccacacactcgctt

SEQ ID NO: 751 - RNA, 22 nt, synthetic construct
  misc_feature 1..22: note = Targeting domain
  source 1..22: mol_type = other RNA
cagcagttccacacactcgctt

SEQ ID NO: 752 - RNA, 23 nt, synthetic construct
  misc_feature 1..23: note = Targeting domain
  source 1..23: mol_type = other RNA
tcagcagttccacacactcgctt

SEQ ID NO: 753 - RNA, 24 nt, synthetic construct
  misc_feature 1..24: note = Targeting domain
  source 1..24: mol_type = other RNA
ttcagcagttccacacactcgctt

SEQ ID NO: 754 - RNA, 18 nt, synthetic construct
  misc_feature 1..18: note = Targeting domain
  source 1..18: mol_type = other RNA
aattagcagtatcctctt

SEQ ID NO: 755 - RNA, 19 nt, synthetic construct
  misc_feature 1..19: note = Targeting domain
  source 1..19: mol_type = other RNA
aaattagcagtatcctctt

SEQ ID NO: 756 - RNA, 21 nt, synthetic construct
  misc_feature 1..21: note = Targeting domain
  source 1..21: mol_type = other RNA
aaaaattagcagtatcctctt

SEQ ID NO: 757 - RNA, 22 nt, synthetic construct
  misc_feature 1..22: note = Targeting domain
  source 1..22: mol_type = other RNA
aaaaaattagcagtatcctctt

SEQ ID NO: 758 - RNA, 23 nt, synthetic construct
  misc_feature 1..23: note = Targeting domain
  source 1..23: mol_type = other RNA
aaaaaaattagcagtatcctctt

SEQ ID NO: 759 - RNA, 24 nt, synthetic construct
  misc_feature 1..24: note = Targeting domain
  source 1..24: mol_type = other RNA
aaaaaaaattagcagtatcctctt

SEQ ID NO: 760 - RNA, 17 nt, synthetic construct
  misc_feature 1..17: note = Targeting domain
  source 1..17: mol_type = other RNA
gaagaaaactagctaaa

SEQ ID NO: 761 - RNA, 17 nt, synthetic construct
  misc_feature 1..17: note = Targeting domain
  source 1..17: mol_type = other RNA
gcagcagtatcctcttg

SEQ ID NO: 762 - RNA, 20 nt, synthetic construct
  misc_feature 1..20: note = Targeting domain
  source 1..20: mol_type = other RNA
ggagaagaaaactagctaaa

SEQ ID NO: 763 - RNA, 20 nt, synthetic construct
  misc_feature 1..20: note = Targeting domain
  source 1..20: mol_type = other RNA
gggagaagaaaactagctaa

SEQ ID NO: 764 - RNA, 20 nt, synthetic construct
  misc_feature 1..20: note = Targeting domain
  source 1..20: mol_type = other RNA
gtcctggtatcttctatggt

SEQ ID NO: 765 - RNA, 17 nt, synthetic construct
  misc_feature 1..17: note = Targeting domain
  source 1..17: mol_type = other RNA
agaagaaaactagctaa

SEQ ID NO: 766 - RNA, 17 nt, synthetic construct
  misc_feature 1..17: note = Targeting domain
  source 1..17: mol_type = other RNA
agtcctggtatcttcta

SEQ ID NO: 767 - RNA, 17 nt, synthetic construct
  misc_feature 1..17: note = Targeting domain
  source 1..17: mol_type = other RNA
ccaccatagaagatacc

SEQ ID NO: 768 - RNA, 17 nt, synthetic construct
  misc_feature 1..17: note = Targeting domain
  source 1..17: mol_type = other RNA
cctggtatcttctatgg

SEQ ID NO: 769 - RNA, 17 nt, synthetic construct
  misc_feature 1..17: note = Targeting domain
  source 1..17: mol_type = other RNA
cagcagtatcctcttgg

SEQ ID NO: 770 - RNA, 17 nt, synthetic construct
  misc_feature 1..17: note = Targeting domain
  source 1..17: mol_type = other RNA
aattggaatgactgaat

SEQ ID NO: 771 - RNA, 17 nt, synthetic construct
  misc_feature 1..17: note = Targeting domain
  source 1..17: mol_type = other RNA
ctggtatcttctatggt

SEQ ID NO: 772 - RNA, 17 nt, synthetic construct
  misc_feature 1..17: note = Targeting domain
  source 1..17: mol_type = other RNA
agcagcagtatcctctt

SEQ ID NO: 773 - RNA, 20 nt, synthetic construct
  misc_feature 1..20: note = Targeting domain
  source 1..20: mol_type = other RNA
agaagtcctggtatcttcta

SEQ ID NO: 774 - RNA, 20 nt, synthetic construct
  misc_feature 1..20: note = Targeting domain
  source 1..20: mol_type = other RNA
ctcccaccatagaagatacc

SEQ ID NO: 775 - RNA, 20 nt, synthetic construct
  misc_feature 1..20: note = Targeting domain
  source 1..20: mol_type = other RNA
agtcctggtatcttctatgg

SEQ ID NO: 776 - RNA, 20 nt, synthetic construct
  misc_feature 1..20: note = Targeting domain
  source 1..20: mol_type = other RNA
aagcagcagtatcctcttgg

SEQ ID NO: 777 - RNA, 20 nt, synthetic construct
  misc_feature 1..20: note = Targeting domain
  source 1..20: mol_type = other RNA
taagcagcagtatcctcttg

SEQ ID NO: 778 - RNA, 20 nt, synthetic construct
  misc_feature 1..20: note = Targeting domain
  source 1..20: mol_type = other RNA
agaataaattagagaaaaat

SEQ ID NO: 779 - RNA, 20 nt, synthetic construct
  misc_feature 1..20: note = Targeting domain
  source 1..20: mol_type = other RNA
aaaaattggaatgactgaat

SEQ ID NO: 780 - RNA, 20 nt, synthetic construct
  misc_feature 1..20: note = Targeting domain
  source 1..20: mol_type = other RNA
attaagcagcagtatcctct

SEQ ID NO: 781 - RNA, 20 nt, synthetic construct
  misc_feature 1..20: note = Targeting domain
  source 1..20: mol_type = other RNA
ttaagcagcagtatcctctt

SEQ ID NO: 782 - RNA, 17 nt, synthetic construct
  misc_feature 1..17: note = Targeting domain
  source 1..17: mol_type = other RNA
ataaattagagaaaaat

SEQ ID NO: 783 - RNA, 17 nt, synthetic construct
  misc_feature 1..17: note = Targeting domain
  source 1..17: mol_type = other RNA
aagcagcagtatcctct

SEQ ID NO: 784 - RNA, 20 nt, synthetic construct
  misc_feature 1..20: note = Targeting domain
  source 1..20: mol_type = other RNA
gaagaaaactagctaaaggg

SEQ ID NO: 785 - RNA, 22 nt, synthetic construct
  misc_feature 1..22: note = Targeting domain
  source 1..22: mol_type = other RNA
gagaagaaaactagctaaaggg

SEQ ID NO: 786 - RNA, 23 nt, synthetic construct
  misc_feature 1..23: note = Targeting domain
  source 1..23: mol_type = other RNA
ggagaagaaaactagctaaaggg

SEQ ID NO: 787 - RNA, 24 nt, synthetic construct
  misc_feature 1..24: note = Targeting domain
  source 1..24: mol_type = other RNA
gggagaagaaaactagctaaaggg

SEQ ID NO: 788 - RNA, 19 nt, synthetic construct
  misc_feature 1..19: note = Targeting domain
  source 1..19: mol_type = other RNA
gaataaattagagaaaaat

SEQ ID NO: 789 - RNA, 22 nt, synthetic construct
  misc_feature 1..22: note = Targeting domain
  source 1..22: mol_type = other RNA
gaagaataaattagagaaaaat

SEQ ID NO: 790 - RNA, 23 nt, synthetic construct
  misc_feature 1..23: note = Targeting domain
  source 1..23: mol_type = other RNA
ggaagaataaattagagaaaaat

SEQ ID NO: 791 - RNA, 24 nt, synthetic construct
  misc_feature 1..24: note = Targeting domain
  source 1..24: mol_type = other RNA
gggaagaataaattagagaaaaat

SEQ ID NO: 792 - RNA, 18 nt, synthetic construct
  misc_feature 1..18: note = Targeting domain
  source 1..18: mol_type = other RNA
agagaaaaattggaatga

SEQ ID NO: 793 - RNA, 19 nt, synthetic construct
  misc_feature 1..19: note = Targeting domain
  source 1..19: mol_type = other RNA
tagagaaaaattggaatga

SEQ ID NO: 794 - RNA, 20 nt, synthetic construct
  misc_feature 1..20: note = Targeting domain
  source 1..20: mol_type = other RNA
ttagagaaaaattggaatga

SEQ ID NO: 795 - RNA, 21 nt, synthetic construct
  misc_feature 1..21: note = Targeting domain
  source 1..21: mol_type = other RNA
attagagaaaaattggaatga

SEQ ID NO: 796 - RNA, 22 nt, synthetic construct
  misc_feature 1..22: note = Targeting domain
  source 1..22: mol_type = other RNA
aattagagaaaaattggaatga

SEQ ID NO: 797 - RNA, 23 nt, synthetic construct
  misc_feature 1..23: note = Targeting domain
  source 1..23: mol_type = other RNA
aaattagagaaaaattggaatga

SEQ ID NO: 798 - RNA, 24 nt, synthetic construct
  misc_feature 1..24: note = Targeting domain
  source 1..24: mol_type = other RNA
taaattagagaaaaattggaatga

SEQ ID NO: 799 - RNA, 18 nt, synthetic construct
  misc_feature 1..18: note = Targeting domain
  source 1..18: mol_type = other RNA
agaaaactagctaaaggg

SEQ ID NO: 800 - RNA, 19 nt, synthetic construct
  misc_feature 1..19: note = Targeting domain
  source 1..19: mol_type = other RNA
aagaaaactagctaaaggg

SEQ ID NO: 801 - RNA, 21 nt, synthetic construct
  misc_feature 1..21: note = Targeting domain
  source 1..21: mol_type = other RNA
agaagaaaactagctaaaggg

SEQ ID NO: 802 - RNA, 18 nt, synthetic construct
  misc_feature 1..18: note = Targeting domain
  source 1..18: mol_type = other RNA
aataaattagagaaaaat

SEQ ID NO: 803 - RNA, 21 nt, synthetic construct
  misc_feature 1..21: note = Targeting domain
  source 1..21: mol_type = other RNA
aagaataaattagagaaaaat

SEQ ID NO: 804 - RNA, 18 nt, synthetic construct
  misc_feature 1..18: note = Targeting domain
  source 1..18: mol_type = other RNA
agttttcttctcccacca

SEQ ID NO: 805 - RNA, 19 nt, synthetic construct
  misc_feature 1..19: note = Targeting domain
  source 1..19: mol_type = other RNA
tagttttcttctcccacca

SEQ ID NO: 806 - RNA, 20 nt, synthetic construct
  misc_feature 1..20: note = Targeting domain
  source 1..20: mol_type = other RNA
ctagttttcttctcccacca

SEQ ID NO: 807 - RNA, 21 nt, synthetic construct
  misc_feature 1..21: note = Targeting domain
  source 1..21: mol_type = other RNA
gctagttttcttctcccacca

SEQ ID NO: 808 - RNA, 22 nt, synthetic construct
  misc_feature 1..22: note = Targeting domain
  source 1..22: mol_type = other RNA
agctagttttcttctcccacca

SEQ ID NO: 809 - RNA, 23 nt, synthetic construct
  misc_feature 1..23: note = Targeting domain
  source 1..23: mol_type = other RNA
tagctagttttcttctcccacca

SEQ ID NO: 810 - RNA, 24 nt, synthetic construct
  misc_feature 1..24: note = Targeting domain
  source 1..24: mol_type = other RNA
ttagctagttttcttctcccacca

SEQ ID NO: 811 - RNA, 18 nt, synthetic construct
  misc_feature 1..18: note = Targeting domain
  source 1..18: mol_type = other RNA
tcccaccatagaagatac

SEQ ID NO: 812 - RNA, 19 nt, synthetic construct
  misc_feature 1..19: note = Targeting domain
  source 1..19: mol_type = other RNA
ctcccaccatagaagatac

SEQ ID NO: 813 - RNA, 20 nt, synthetic construct
  misc_feature 1..20: note = Targeting domain
  source 1..20: mol_type = other RNA
tctcccaccatagaagatac

SEQ ID NO: 814 - RNA, 21 nt, synthetic construct
  misc_feature 1..21: note = Targeting domain
  source 1..21: mol_type = other RNA
ttctcccaccatagaagatac

SEQ ID NO: 815 - RNA, 22 nt, synthetic construct
  misc_feature 1..22: note = Targeting domain
  source 1..22: mol_type = other RNA
cttctcccaccatagaagatac

SEQ ID NO: 816 - RNA, 23 nt, synthetic construct
  misc_feature 1..23: note = Targeting domain
  source 1..23: mol_type = other RNA
tcttctcccaccatagaagatac

SEQ ID NO: 817 - RNA, 24 nt, synthetic construct
  misc_feature 1..24: note = Targeting domain
  source 1..24: mol_type = other RNA
ttcttctcccaccatagaagatac

SEQ ID NO: 818 - RNA, 18 nt, synthetic construct
  misc_feature 1..18: note = Targeting domain
  source 1..18: mol_type = other RNA
agaagaaaactagctaaa

SEQ ID NO: 819 - RNA, 19 nt, synthetic construct
  misc_feature 1..19: note = Targeting domain
  source 1..19: mol_type = other RNA
gagaagaaaactagctaaa

SEQ ID NO: 820 - RNA, 21 nt, synthetic construct
  misc_feature 1..21: note = Targeting domain
  source 1..21: mol_type = other RNA
gggagaagaaaactagctaaa

SEQ ID NO: 821 - RNA, 22 nt, synthetic construct
  misc_feature 1..22: note = Targeting domain
  source 1..22: mol_type = other RNA
tgggagaagaaaactagctaaa

SEQ ID NO: 822 - RNA, 23 nt, synthetic construct
  misc_feature 1..23: note = Targeting domain
  source 1..23: mol_type = other RNA
gtgggagaagaaaactagctaaa

SEQ ID NO: 823 - RNA, 24 nt, synthetic construct
  misc_feature 1..24: note = Targeting domain
  source 1..24: mol_type = other RNA
ggtgggagaagaaaactagctaaa

SEQ ID NO: 824 - RNA, 18 nt, synthetic construct
  misc_feature 1..18: note = Targeting domain
  source 1..18: mol_type = other RNA
aaaattggaatgactgaa

SEQ ID NO: 825 - RNA, 19 nt, synthetic construct
  misc_feature 1..19: note = Targeting domain
  source 1..19: mol_type = other RNA
aaaaattggaatgactgaa

SEQ ID NO: 826 - RNA, 20 nt, synthetic construct
  misc_feature 1..20: note = Targeting domain
  source 1..20: mol_type = other RNA
gaaaaattggaatgactgaa

SEQ ID NO: 827 - RNA, 21 nt, synthetic construct
  misc_feature 1..21: note = Targeting domain
  source 1..21: mol_type = other RNA
agaaaaattggaatgactgaa

SEQ ID NO: 828 - RNA, 22 nt, synthetic construct
  misc_feature 1..22: note = Targeting domain
  source 1..22: mol_type = other RNA
gagaaaaattggaatgactgaa

SEQ ID NO: 829 - RNA, 23 nt, synthetic construct
  misc_feature 1..23: note = Targeting domain
  source 1..23: mol_type = other RNA
agagaaaaattggaatgactgaa

SEQ ID NO: 830 - RNA, 24 nt, synthetic construct
  misc_feature 1..24: note = Targeting domain
  source 1..24: mol_type = other RNA
tagagaaaaattggaatgactgaa

SEQ ID NO: 831 - RNA, 18 nt, synthetic construct
  misc_feature 1..18: note = Targeting domain
  source 1..18: mol_type = other RNA
gagaagaaaactagctaa

SEQ ID NO: 832 - RNA, 19 nt, synthetic construct
  misc_feature 1..19: note = Targeting domain
  source 1..19: mol_type = other RNA
ggagaagaaaactagctaa

SEQ ID NO: 833 - RNA, 21 nt, synthetic construct
  misc_feature 1..21: note = Targeting domain
  source 1..21: mol_type = other RNA
tgggagaagaaaactagctaa

SEQ ID NO: 834 - RNA, 22 nt, synthetic construct
  misc_feature 1..22: note = Targeting domain
  source 1..22: mol_type = other RNA
gtgggagaagaaaactagctaa

SEQ ID NO: 835 - RNA, 23 nt, synthetic construct
  misc_feature 1..23: note = Targeting domain
  source 1..23: mol_type = other RNA
ggtgggagaagaaaactagctaa

SEQ ID NO: 836 - RNA, 24 nt, synthetic construct
  misc_feature 1..24: note = Targeting domain
  source 1..24: mol_type = other RNA
tggtgggagaagaaaactagctaa

SEQ ID NO: 837 - RNA, 18 nt, synthetic construct
  misc_feature 1..18: note = Targeting domain
  source 1..18: mol_type = other RNA
ggagaagaaaactagcta

SEQ ID NO: 838 - RNA, 19 nt, synthetic construct
  misc_feature 1..19: note = Targeting domain
  source 1..19: mol_type = other RNA
gggagaagaaaactagcta

SEQ ID NO: 839 - RNA, 20 nt, synthetic construct
  misc_feature 1..20: note = Targeting domain
  source 1..20: mol_type = other RNA
tgggagaagaaaactagcta

SEQ ID NO: 840 - RNA, 21 nt, synthetic construct
  misc_feature 1..21: note = Targeting domain
  source 1..21: mol_type = other RNA
gtgggagaagaaaactagcta

SEQ ID NO: 841 - RNA, 22 nt, synthetic construct
  misc_feature 1..22: note = Targeting domain
  source 1..22: mol_type = other RNA
ggtgggagaagaaaactagcta

SEQ ID NO: 842 - RNA, 23 nt, synthetic construct
  misc_feature 1..23: note = Targeting domain
  source 1..23: mol_type = other RNA
tggtgggagaagaaaactagcta

SEQ ID NO: 843 - RNA, 24 nt, synthetic construct
  misc_feature 1..24: note = Targeting domain
  source 1..24: mol_type = other RNA
atggtgggagaagaaaactagcta

SEQ ID NO: 844 - RNA, 18 nt, synthetic construct
  misc_feature 1..18: note = Targeting domain
  source 1..18: mol_type = other RNA
ttaagcagcagtatcctc

SEQ ID NO: 845 - RNA, 19 nt, synthetic construct
  misc_feature 1..19: note = Targeting domain
  source 1..19: mol_type = other RNA
attaagcagcagtatcctc

SEQ ID NO: 846 - RNA, 20 nt, synthetic construct
  misc_feature 1..20: note = Targeting domain
  source 1..20: mol_type = other RNA
aattaagcagcagtatcctc

SEQ ID NO: 847 - RNA, 21 nt, synthetic construct
  misc_feature 1..21: note = Targeting domain
  source 1..21: mol_type = other RNA
aaattaagcagcagtatcctc

SEQ ID NO: 848 - RNA, 22 nt, synthetic construct
  misc_feature 1..22: note = Targeting domain
  source 1..22: mol_type = other RNA
aaaattaagcagcagtatcctc

SEQ ID NO: 849 - RNA, 23 nt, synthetic construct
  misc_feature 1..23: note = Targeting domain
  source 1..23: mol_type = other RNA
aaaaattaagcagcagtatcctc

SEQ ID NO: 850 - RNA, 24 nt, synthetic construct
  misc_feature 1..24: note = Targeting domain
  source 1..24: mol_type = other RNA
aaaaaattaagcagcagtatcctc

SEQ ID NO: 851 - RNA, 18 nt, synthetic construct
  misc_feature 1..18: note = Targeting domain
  source 1..18: mol_type = other RNA
tatcttctatggtgggag

SEQ ID NO: 852 - RNA, 19 nt, synthetic construct
  misc_feature 1..19: note = Targeting domain
  source 1..19: mol_type = other RNA
gtatcttctatggtgggag

SEQ ID NO: 853 - RNA, 20 nt, synthetic construct
  misc_feature 1..20: note = Targeting domain
  source 1..20: mol_type = other RNA
ggtatcttctatggtgggag

SEQ ID NO: 854 - RNA, 21 nt, synthetic construct
  misc_feature 1..21: note = Targeting domain
  source 1..21: mol_type = other RNA
tggtatcttctatggtgggag

SEQ ID NO: 855 - RNA, 22 nt, synthetic construct
  misc_feature 1..22: note = Targeting domain
  source 1..22: mol_type = other RNA
ctggtatcttctatggtgggag

SEQ ID NO: 856 - RNA, 23 nt, synthetic construct
  misc_feature 1..23: note = Targeting domain
  source 1..23: mol_type = other RNA
cctggtatcttctatggtgggag

SEQ ID NO: 857 - RNA, 24 nt, synthetic construct
  misc_feature 1..24: note = Targeting domain
  source 1..24: mol_type = other RNA
tcctggtatcttctatggtgggag

SEQ ID NO: 858 - RNA, 18 nt, synthetic construct
  misc_feature 1..18: note = Targeting domain
  source 1..18: mol_type = other RNA
tcctggtatcttctatgg

SEQ ID NO: 859 - RNA, 19 nt, synthetic construct
  misc_feature 1..19: note = Targeting domain
  source 1..19: mol_type = other RNA
gtcctggtatcttctatgg

SEQ ID NO: 860 - RNA, 21 nt, synthetic construct
  misc_feature 1..21: note = Targeting domain
  source 1..21: mol_type = other RNA
aagtcctggtatcttctatgg

SEQ ID NO: 861 - RNA, 22 nt, synthetic construct
  misc_feature 1..22: note = Targeting domain
  source 1..22: mol_type = other RNA
gaagtcctggtatcttctatgg

SEQ ID NO: 862 - RNA, 23 nt, synthetic construct
  misc_feature 1..23: note = Targeting domain
  source 1..23: mol_type = other RNA
agaagtcctggtatcttctatgg

SEQ ID NO: 863 - RNA, 24 nt, synthetic construct
  misc_feature 1..24: note = Targeting domain
  source 1..24: mol_type = other RNA
aagaagtcctggtatcttctatgg

SEQ ID NO: 864 - RNA, 18 nt, synthetic construct
  misc_feature 1..18: note = Targeting domain
  source 1..18: mol_type = other RNA
tggtatcttctatggtgg

SEQ ID NO: 865 - RNA, 19 nt, synthetic construct
  misc_feature 1..19: note = Targeting domain
  source 1..19: mol_type = other RNA
ctggtatcttctatggtgg

SEQ ID NO: 866 - RNA, 20 nt, synthetic construct
  misc_feature 1..20: note = Targeting domain
  source 1..20: mol_type = other RNA
cctggtatcttctatggtgg

SEQ ID NO: 867 - RNA, 21 nt, synthetic construct
  misc_feature 1..21: note = Targeting domain
  source 1..21: mol_type = other RNA
tcctggtatcttctatggtgg

SEQ ID NO: 868 - RNA, 22 nt, synthetic construct
  misc_feature 1..22: note = Targeting domain
  source 1..22: mol_type = other RNA
gtcctggtatcttctatggtgg

SEQ ID NO: 869 - RNA, 23 nt, synthetic construct
  misc_feature 1..23: note = Targeting domain
  source 1..23: mol_type = other RNA
agtcctggtatcttctatggtgg

SEQ ID NO: 870 - RNA, 24 nt, synthetic construct
  misc_feature 1..24: note = Targeting domain
  source 1..24: mol_type = other RNA
aagtcctggtatcttctatggtgg

SEQ ID NO: 871 - RNA, 18 nt, synthetic construct
  misc_feature 1..18: note = Targeting domain
  source 1..18: mol_type = other RNA
gtcctggtatcttctatg

SEQ ID NO: 872 - RNA, 19 nt, synthetic construct
  misc_feature 1..19: note = Targeting domain
  source 1..19: mol_type = other RNA
agtcctggtatcttctatg

SEQ ID NO: 873 - RNA, 20 nt, synthetic construct
  misc_feature 1..20: note = Targeting domain
  source 1..20: mol_type = other RNA
aagtcctggtatcttctatg

SEQ ID NO: 874 - RNA, 21 nt, synthetic construct
  misc_feature 1..21: note = Targeting domain
  source 1..21: mol_type = other RNA
gaagtcctggtatcttctatg

SEQ ID NO: 875 - RNA, 22 nt, synthetic construct
  misc_feature 1..22: note = Targeting domain
  source 1..22: mol_type = other RNA
agaagtcctggtatcttctatg

SEQ ID NO: 876 - RNA, 23 nt, synthetic construct
  misc_feature 1..23: note = Targeting domain
  source 1..23: mol_type = other RNA
aagaagtcctggtatcttctatg

SEQ ID NO: 877 - RNA, 24 nt, synthetic construct
  misc_feature 1..24: note = Targeting domain
  source 1..24: mol_type = other RNA
aaagaagtcctggtatcttctatg

SEQ ID NO: 878 - RNA, 18 nt, synthetic construct
  misc_feature 1..18: note = Targeting domain
  source 1..18: mol_type = other RNA
aaattggaatgactgaat

SEQ ID NO: 879 - RNA, 19 nt, synthetic construct
  misc_feature 1..19: note = Targeting domain
  source 1..19: mol_type = other RNA
aaaattggaatgactgaat

SEQ ID NO: 880 - RNA, 21 nt, synthetic construct
  misc_feature 1..21: note = Targeting domain
  source 1..21: mol_type = other RNA
gaaaaattggaatgactgaat

SEQ ID NO: 881 - RNA, 22 nt, synthetic construct
  misc_feature 1..22: note = Targeting domain
  source 1..22: mol_type = other RNA
agaaaaattggaatgactgaat

SEQ ID NO: 882 - RNA, 23 nt, synthetic construct
  misc_feature 1..23: note = Targeting domain
  source 1..23: mol_type = other RNA
gagaaaaattggaatgactgaat

SEQ ID NO: 883 - RNA, 24 nt, synthetic construct
  misc_feature 1..24: note = Targeting domain
  source 1..24: mol_type = other RNA
agagaaaaattggaatgactgaat

SEQ ID NO: 884 - RNA, 18 nt, synthetic construct
  misc_feature 1..18: note = Targeting domain
  source 1..18: mol_type = other RNA
taagcagcagtatcctct

SEQ ID NO: 885 - RNA, 19 nt, synthetic construct
  misc_feature 1..19: note = Targeting domain
  source 1..19: mol_type = other RNA
ttaagcagcagtatcctct

SEQ ID NO: 886 - RNA, 21 nt, synthetic construct
  misc_feature 1..21: note = Targeting domain
  source 1..21: mol_type = other RNA
aattaagcagcagtatcctct

SEQ ID NO: 887 - RNA, 22 nt, synthetic construct
  misc_feature 1..22: note = Targeting domain
  source 1..22: mol_type = other RNA
aaattaagcagcagtatcctct

SEQ ID NO: 888 - RNA, 23 nt, synthetic construct
  misc_feature 1..23: note = Targeting domain
  source 1..23: mol_type = other RNA
aaaattaagcagcagtatcctct

SEQ ID NO: 889 - RNA, 24 nt, synthetic construct
  misc_feature 1..24: note = Targeting domain
  source 1..24: mol_type = other RNA
aaaaattaagcagcagtatcctct

SEQ ID NO: 890 - RNA, 18 nt, synthetic construct
  misc_feature 1..18: note = Targeting domain
  source 1..18: mol_type = other RNA
cctggtatcttctatggt

SEQ ID NO: 891 - RNA, 19 nt, synthetic construct
  misc_feature 1..19: note = Targeting domain
  source 1..19: mol_type = other RNA
tcctggtatcttctatggt

SEQ ID NO: 892 - RNA, 21 nt, synthetic construct
  misc_feature 1..21: note = Targeting domain
  source 1..21: mol_type = other RNA
agtcctggtatcttctatggt

SEQ ID NO: 893 - RNA, 22 nt, synthetic construct
  misc_feature 1..22: note = Targeting domain
  source 1..22: mol_type = other RNA
aagtcctggtatcttctatggt

SEQ ID NO: 894 - RNA, 23 nt, synthetic construct
  misc_feature 1..23: note = Targeting domain
  source 1..23: mol_type = other RNA
gaagtcctggtatcttctatggt

SEQ ID NO: 895 - RNA, 24 nt, synthetic construct
  misc_feature 1..24: note = Targeting domain
  source 1..24: mol_type = other RNA
agaagtcctggtatcttctatggt

SEQ ID NO: 896 - RNA, 18 nt, synthetic construct
  misc_feature 1..18: note = Targeting domain
  source 1..18: mol_type = other RNA
aagcagcagtatcctctt

SEQ ID NO: 897 - RNA, 19 nt, synthetic construct
  misc_feature 1..19: note = Targeting domain
  source 1..19: mol_type = other RNA
taagcagcagtatcctctt

SEQ ID NO: 898 - RNA, 21 nt, synthetic construct
  misc_feature 1..21: note = Targeting domain
  source 1..21: mol_type = other RNA
attaagcagcagtatcctctt

SEQ ID NO: 899 - RNA, 22 nt, synthetic construct
  misc_feature 1..22: note = Targeting domain
  source 1..22: mol_type = other RNA
aattaagcagcagtatcctctt

SEQ ID NO: 900 - RNA, 23 nt, synthetic construct
  misc_feature 1..23: note = Targeting domain
  source 1..23: mol_type = other RNA
aaattaagcagcagtatcctctt

SEQ ID NO: 901 - RNA, 24 nt, synthetic construct
  misc_feature 1..24: note = Targeting domain
  source 1..24: mol_type = other RNA
aaaattaagcagcagtatcctctt

SEQ ID NO: 902 - DNA, 4758 bp, Homo sapiens
  misc_feature 2716..2719: note = HPFH deletion site (4 bp del -225 to -222)
  misc_feature 2748..2753: note = GATA1 binding motif
  misc_feature 2762..2767: note = GATA1 binding motif
  misc_feature 2791..2799: note = FKLF transcription factor binding motif
  misc_feature 2823..2830: note = CP1/Coup TFII binding motif
  misc_feature 2824..2836: note = HPFH deletion site (13 bp del -114 to -102)
  misc_feature 2991..2993: note = Start codon
  source 1..4758: mol_type = other DNA
tttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccg

SEQ ID NO: 903 - DNA, 4773 bp, Homo sapiens
  misc_feature 1233..1238: note = GATA1 binding site
  misc_feature 2672..2677: note = GATA1 binding site
  misc_feature 2686..2691: note = GATA1 binding site
  misc_feature 2715..2723: note = FKLF transcription factor binding motif
  misc_feature 2747..2754: note = CP1/Coup TFII binding motif
  misc_feature 2748..2760: note = HPFH deletion site (13 bp del -114 to -102)
  misc_feature 2915..2917: note = Start codon
  source 1..4773: mol_type = other DNA
ttatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaa

SEQ ID NO: 904 - DNA, 87 bp, synthetic construct
  misc_feature 1..87: note = ssODN1 5' homology arm
  source 1..87: mol_type = other DNA
gggtgcttccttttattcttcatccctagccagccgccggcccctggcctcactggatactctaagactattggtcaagtttgcctt

SEQ ID NO: 905 - DNA, 89 bp, synthetic construct
  misc_feature 1..89: note = ssODN1 3' homology arm
  source 1..89: mol_type = other DNA
gtcaaggcaaggctggccaacccatgggtggagtttagccagggaccgtttcagacagatatttgcattgagatagtgtggggaagggg

SEQ ID NO: 906 - DNA, 176 bp, synthetic construct
  misc_feature 1..176: note = ssODN1
  source 1..176: mol_type = other DNA
gggtgcttccttttattcttcatccctagccagccgccggcccctggcctcactggatactctaagactattggtcaagtttgccttgtcaaggcaaggctggccaacccatgggtggagtttagccagggaccgtttcagacagatatttgcattgagatagtgtggggaagggg

SEQ ID NO: 907 - DNA, 87 bp, synthetic construct; identical to SEQ ID NO: 904
  misc_feature 1..87: note = PhTx ssODN1 5' homology arm
  misc_feature 1: note = Modified to contain phosphorothioate
  source 1..87: mol_type = other DNA

SEQ ID NO: 908 - DNA, 89 bp, synthetic construct; identical to SEQ ID NO: 905
  misc_feature 1..89: note = PhTx ssODN1 3' homology arm
  misc_feature 89: note = Modified to contain phosphorothioate
  source 1..89: mol_type = other DNA

SEQ ID NO: 909 - DNA, 176 bp, synthetic construct; identical to SEQ ID NO: 906
  misc_feature 1..176: note = PhTx ssODN1
  misc_feature 1: note = Modified to contain phosphorothioate
  misc_feature 176: note = Modified to contain phosphorothioate
  source 1..176: mol_type = other DNA

SEQ ID NO: 910 - DNA, 17 bp, synthetic construct
  misc_feature 1..17: note = targeting domain
  source 1..17: mol_type = other DNA
ggctattggtcaaggca

SEQ ID NO: 911 - DNA, 20 bp, synthetic construct
  misc_feature 1..20: note = targeting domain
  source 1..20: mol_type = other DNA
caaggctattggtcaaggca

SEQ ID NO: 912 - DNA, 17 bp, synthetic construct
  misc_feature 1..17: note = targeting domain
  source 1..17: mol_type = other DNA
tgccttgtcaaggctat

SEQ ID NO: 913 - DNA, 20 bp, synthetic construct
  misc_feature 1..20: note = targeting domain
  source 1..20: mol_type = other DNA
gtttgccttgtcaaggctat

SEQ ID NO: 914 - DNA, 17 bp, synthetic construct
  misc_feature 1..17: note = targeting domain
  source 1..17: mol_type = other DNA
gaccaatagccttgaca

SEQ ID NO: 915 - DNA, 20 bp, synthetic construct
  misc_feature 1..20: note = targeting domain
  source 1..20: mol_type = other DNA
cttgaccaatagccttgaca

SEQ ID NO: 916 - DNA, 17 bp, synthetic construct
  misc_feature 1..17: note = targeting domain
  source 1..17: mol_type = other DNA
gtcaaggctattggtca

SEQ ID NO: 917 - DNA, 20 bp, synthetic construct
  misc_feature 1..20: note = targeting domain
  source 1..20: mol_type = other DNA
cttgtcaaggctattggtca

SEQ ID NO: 918 - DNA, 17 bp, synthetic construct
  misc_feature 1..17: note = targeting domain
  source 1..17: mol_type = other DNA
tcaagtttgccttgtca

SEQ ID NO: 919 - DNA, 20 bp, synthetic construct
  misc_feature 1..20: note = targeting domain
  source 1..20: mol_type = other DNA
tggtcaagtttgccttgtca

SEQ ID NO: 920 - RNA, 20 nt, synthetic construct
  misc_feature 1..20: note = targeting domain plus PAM (NGG)
  source 1..20: mol_type = other RNA
ggctattggtcaaggcaagg

SEQ ID NO: 921 - RNA, 23 nt, synthetic construct
  misc_feature 1..23: note = targeting domain plus PAM (NGG)
  source 1..23: mol_type = other RNA
caaggctattggtcaaggcaagg

SEQ ID NO: 922 - RNA, 20 nt, synthetic construct
  misc_feature 1..20: note = targeting domain plus PAM (NGG)
  source 1..20: mol_type = other RNA
tgccttgtcaaggctattgg

SEQ ID NO: 923 - RNA, 23 nt, synthetic construct
  misc_feature 1..23: note = targeting domain plus PAM (NGG)
  source 1..23: mol_type = other RNA
gtttgccttgtcaaggctattgg

SEQ ID NO: 924 - RNA, 20 nt, synthetic construct
  misc_feature 1..20: note = targeting domain plus PAM (NGG)
  source 1..20: mol_type = other RNA
gaccaatagccttgacaagg

SEQ ID NO: 925 - RNA, 23 nt, synthetic construct
  misc_feature 1..23: note = targeting domain plus PAM (NGG)
  source 1..23: mol_type = other RNA
cttgaccaatagccttgacaagg

SEQ ID NO: 926 - RNA, 20 nt, synthetic construct
  misc_feature 1..20: note = targeting domain plus PAM (NGG)
  source 1..20: mol_type = other RNA
gtcaaggctattggtcaagg

SEQ ID NO: 927 - RNA, 23 nt, synthetic construct
  misc_feature 1..23: note = targeting domain plus PAM (NGG)
  source 1..23: mol_type = other RNA
cttgtcaaggctattggtcaagg

SEQ ID NO: 928 - RNA, 20 nt, synthetic construct
  misc_feature 1..20: note = targeting domain plus PAM (NGG)
  source 1..20: mol_type = other RNA
tcaagtttgccttgtcaagg

SEQ ID NO: 929 - RNA, 23 nt, synthetic construct
  misc_feature 1..23: note = targeting domain plus PAM (NGG)
  source 1..23: mol_type = other RNA
tggtcaagtttgccttgtcaagg

SEQ ID NO: 930 - DNA, 20 bp, synthetic construct
  misc_feature 1..20: note = targeting domain plus PAM (NGG)
  source 1..20: mol_type = other DNA
ggctattggtcaaggcaagg

SEQ ID NO: 931 - DNA, 23 bp, synthetic construct
  misc_feature 1..23: note = targeting domain plus PAM (NGG)
  source 1..23: mol_type = other DNA
caaggctattggtcaaggcaagg

SEQ ID NO: 932 - DNA, 20 bp, synthetic construct
  misc_feature 1..20: note = targeting domain plus PAM (NGG)
  source 1..20: mol_type = other DNA
tgccttgtcaaggctattgg

SEQ ID NO: 933 - DNA, 23 bp, synthetic construct
  misc_feature 1..23: note = targeting domain plus PAM (NGG)
  source 1..23: mol_type = other DNA
gtttgccttgtcaaggctattgg

SEQ ID NO: 934 - DNA, 20 bp, synthetic construct
  misc_feature 1..20: note = targeting domain plus PAM (NGG)
  source 1..20: mol_type = other DNA
gaccaatagccttgacaagg

SEQ ID NO: 935 - DNA, 23 bp, synthetic construct
  misc_feature 1..23: note = targeting domain plus PAM (NGG)
  source 1..23: mol_type = other DNA
cttgaccaatagccttgacaagg

SEQ ID NO: 936 - DNA, 20 bp, synthetic construct
  misc_feature 1..20: note = targeting domain plus PAM (NGG)
  source 1..20: mol_type = other DNA
gtcaaggctattggtcaagg

SEQ ID NO: 937 - DNA, 23 bp, synthetic construct
  misc_feature 1..23: note = targeting domain plus PAM (NGG)
  source 1..23: mol_type = other DNA
cttgtcaaggctattggtcaagg

SEQ ID NO: 938 - DNA, 20 bp, synthetic construct
  misc_feature 1..20: note = targeting domain plus PAM (NGG)
  source 1..20: mol_type = other DNA
tcaagtttgccttgtcaagg

SEQ ID NO: 939 - DNA, 23 bp, synthetic construct
  misc_feature 1..23: note = targeting domain plus PAM (NGG)
  source 1..23: mol_type = other DNA
tggtcaagtttgccttgtcaagg

(19) United States
(12) Patent Application Publication
(10) Pub. No.: US 2024/0360471 A1
(43) Pub. Date: Oct. 31, 2024

(54) CRISPR/CAS-RELATED METHODS AND COMPOSITIONS FOR TREATING BETA HEMOGLOBINOPATHIES

(71) Applicant: EDITAS MEDICINE, INC., Cambridge, MA (US)

(72) Inventors: Jennifer Leah GORI, Jamaica Plain, MA (US); Luis A. BARRERA, Somerville, MA (US)

(73) Assignee: EDITAS MEDICINE, INC., Cambridge, MA (US)

(21) Appl. No.: 18/457,258

(22) Filed: Aug. 28, 2023

Related U.S. Application Data
(63) Continuation of application No. 16/085,480, filed on Sep. 14, 2018, now abandoned.
(60) Provisional application No. 62/456,615, filed on Feb. 8, 2017.
(60) Provisional application No. 62/308,190, filed on Mar. 14, 2016.

(51) Int. Cl. C12N 15/85; C12N 5/071; C12N 9/22; C12N 15/113

(52) U.S. Cl. CPC C12N 15/85; C12N 5/0602; C12N 9/22; C12N 15/113; C12N 2310/20; C12N 2310/315; C12N 2310/322; C12N 2510/00; C12N 2800/80

(57) ABSTRACT

Provided herein are CRISPR/Cas-related methods and components for editing a target nucleic acid sequence in a HBG1 and/or HBG2 gene regulatory region, and applications thereof in connection with methods of increasing expression of fetal hemoglobin and treating β-hemoglobinopathies including sickle cell disease and β-thalassemia.

### REFERENCE TO RELATED APPLICATIONS

The present application is a continuation of U.S. patent application Ser. No. 16/085,480, filed Sep. 14, 2018, which is a national phase application of International Patent Application No. PCT/US2017/02237, filed Mar. 14, 2017, which claims the benefit of U.S. Provisional Application No. 62/308,190, filed Mar. 14, 2016, and U.S. Provisional Application No. 62/456,615, filed Feb. 8, 2017, the contents of which are hereby incorporated by reference in their entirety.

### SEQUENCE LISTING

This application contains a ST.26 compliant Substitute Sequence Listing, which was submitted in XML format via Patent Center, and is hereby incorporated by reference in its entirety. The XML copy, created on Jul. 10, 2024, is named SecondSubstituteSequenceListing.xml and is 1,256,000 bytes in size.

### FIELD OF THE INVENTION

The invention relates to CRISPR/Cas-related methods and components for editing a target nucleic acid sequence, or modulating expression of a target nucleic acid sequence, and applications thereof in connection with β-hemoglobinopathies including sickle cell disease and β-thalassemia.

### BACKGROUND

Hemoglobin (Hb) carries oxygen from the lungs to tissues in erythrocytes or red blood cells (RBCs). During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. HbF is more efficient than HbA at carrying oxygen. The average adult makes less than 1% HbF out of total hemoglobin (Thein 2009). The α-hemoglobin gene is located on chromosome 16, while the β-hemoglobin gene (HBB), A gamma (γ^{A})-globin chain (HBG1, also known as gamma globin A), and G gamma (γ^{G})-globin chain (HBG2, also known as gamma globin G) are located on chromosome 11 within the globin gene cluster (i.e., globin locus).

Mutations in HBB can cause hemoglobin disorders (i.e., hemoglobinopathies) including sickle cell disease (SCD) and beta-thalassemia (β-Thal). Approximately 93,000 people in the United States are diagnosed with a hemoglobinopathy. Worldwide, 300,000 children are born with hemoglobinopathies every year (Angastiniotis 1998). Because these conditions are associated with HBB mutations, their symptoms typically do not manifest until after globin switching from HbF to HbA.

SCD is the most common inherited hematologic disease in the United States, affecting approximately 80,000 people (Brousseau 2010). SCD is most common in people of African ancestry, for whom the prevalence of SCD is 1 in 500. In Africa, the prevalence of SCD is 15 million (Aliyu 2008). SCD is also more common in people of Indian, Saudi Arabian and Mediterranean descent. In those of Hispanic-American descent, the prevalence of sickle cell disease is 1 in 1,000 (Lewis 2014).

SCD is caused by a single homozygous mutation in the HBB gene, c.17A>T (HbS mutation). The sickle mutation is a point mutation (GAG→GTG) on HBB that results in substitution of valine for glutamic acid at amino acid position 6 in exon 1. The valine at position 6 of the β-hemoglobin chain is hydrophobic and causes a change in conformation of the β-globin protein when it is not bound to oxygen. This change of conformation causes HbS proteins to polymerize in the absence of oxygen, leading to deformation (i.e., sickling) of RBCs. SCD is inherited in an autosomal recessive manner, so that only patients with two HbS alleles have the disease. Heterozygous subjects have sickle cell trait, and may suffer from anemia and/or painful crises if they are severely dehydrated or oxygen deprived.

Sickle shaped RBCs cause multiple symptoms, including anemia, sickle cell crises, vaso-occlusive crises, aplastic crises, and acute chest syndrome. Sickle shaped RBCs are less elastic than wild-type RBCs and therefore cannot pass as easily through capillary beds and cause occlusion and ischemia (i.e., vaso-occlusion). Vaso-occlusive crisis occurs when sickle cells obstruct blood flow in the capillary bed of an organ leading to pain, ischemia, and necrosis. These episodes typically last 5-7 days. The spleen plays a role in clearing dysfunctional RBCs, and is therefore typically enlarged during early childhood and subject to frequent vaso-occlusive crises. By the end of childhood the spleen in SCD patients is often infarcted, which leads to autosplenectomy. Hemolysis is a constant feature of SCD and causes anemia. Sickle cells survive for 10-20 days in circulation, while healthy RBCs survive for 90-120 days. SCD subjects are transfused as necessary to maintain adequate hemoglobin levels. Frequent transfusions place subjects at risk for infection with HIV, hepatitis B, and hepatitis C. Subjects may also suffer from acute chest crisis and infarcts of extremities, end organs, and the central nervous system.

Subjects with SCD have decreased life expectancies. The prognosis for patients with SCD is steadily improving with careful, life-long management of crises and anemia. As of 2001, the average life expectancy of subjects with sickle cell disease was the mid-to-late 50's. Current treatments for SCD involve hydration and pain management during crises, and transfusions as needed to correct anemia.

Thalassemias (e.g., β-Thal, δ-Thal, and β/δ-Thal) cause chronic anemia. β-Thal is estimated to affect approximately 1 in 100,000 people worldwide. Its prevalence is higher in certain populations, including those of European descent, where its prevalence is approximately 1 in 10,000. β-Thal major, the more severe form of the disease, is life-threatening unless treated with lifelong blood transfusions and chelation therapy. In the United States, there are approximately 3,000 subjects with β-Thal major. β-Thal intermedia does not require blood transfusions, but it may cause growth delay and significant systemic abnormalities, and it frequently requires lifelong chelation therapy. Although HbA makes up the majority of hemoglobin in adult RBCs, approximately 3% of adult hemoglobin is in the form of HbA₂, an HbA variant in which the two γ-globin chains are replaced with two delta (Δ)-globin chains. δ-Thal is associated with mutations in the A hemoglobin gene (HBD) that cause a loss of HBD expression. Co-inheritance of the HBD mutation can mask a diagnosis of β-Thal (i.e., β/δ-Thal) by decreasing the level of HbA₂ to the normal range (Bouva 2006). β/δ-Thal is usually caused by deletion of the HBB and HBD sequences in both alleles. In homozygous (δ°/δ° β°/β°) patients, HBG is expressed, leading to production of HbF alone.

Like SCD, β-Thal is caused by mutations in the HBB gene. The most common HBB mutations leading to β-Thal are: c.−136C>G, c.92+1G>A, c.92+6T>C, c.93-21G>A, c.118C>T, c.316-106C>G, c.25_26delAA, c.27_28insG, c.92+5G>C, c.118C>T, c.135delC, c.315+1G>A, c.−78A>G, c.52A>T, c.59A>G, c.92+5G>C, c.124_127delTTCT, c.316-197C>T, c.−78A>G, c.52A>T, c.124_127delTTCT, c.316-197C>T, c.−138C>T, c.−79A>G, c.92+5G>C, c.75T>A, c.316-2A>G, and c.316-2A>C. These and other mutations associated with β-Thal cause mutated or absent β-globin chains, which causes a disruption of the normal Hb α-hemoglobin to 0-hemoglobin ratio. Excess α-globin chains precipitate in erythroid precursors in the bone marrow.

In β-Thal major, both alleles of HBB contain nonsense, frameshift, or splicing mutations that leads to complete absence of β-globin production (denoted β°/β°). β-Thal major results in severe reduction in β-globin chains, leading to significant precipitation of α-globin chains in erythroid cells and more severe anemia. β-Thal intermedia results from mutations in the 5′ or 3′ untranslated region of HBB, mutations in the promoter region or polyadenylation signal of HBB, or splicing mutations within the HBB gene. Patient genotypes are denoted β°/β⁺ or β⁺/β⁺. β° represents absent expression of a β-globin chain; β⁺ represents a dysfunctional but present β-globin chain. Phenotypic expression varies among patients. Since there is some production of β-globin, β-Thal intermedia results in less precipitation of α-globin chains in the erythroid precursors and less severe anemia than β-Thal major. However, there are more significant consequences of erythroid lineage expansion secondary to chronic anemia.

Subjects with β-Thal major present between the ages of 6 months and 2 years, and suffer from failure to thrive, fevers, hepatosplenomegaly, and diarrhea. Adequate treatment includes regular transfusions. Therapy for β-Thal major also includes splenectomy and treatment with hydroxyurea. If patients are regularly transfused, they will develop normally until the beginning of the second decade. At that time, they require chelation therapy (in addition to continued transfusions) to prevent complications of iron overload. Iron overload may manifest as growth delay or delay of sexual maturation. In adulthood, inadequate chelation therapy may lead to cardiomyopathy, cardiac arrhythmias, hepatic fibrosis and/or cirrhosis, diabetes, thyroid and parathyroid abnormalities, thrombosis, and osteoporosis. Frequent transfusions also put subjects at risk for infection with HIV, hepatitis B and hepatitis C.

β-Thal intermedia subjects generally present between the ages of 2-6 years. They do not generally require blood transfusions. However, bone abnormalities occur due to chronic hypertrophy of the erythroid lineage to compensate for chronic anemia. Subjects may have fractures of the long bones due to osteoporosis. Extramedullary erythropoiesis is common and leads to enlargement of the spleen, liver, and lymph nodes. It may also cause spinal cord compression and neurologic problems. Subjects also suffer from lower extremity ulcers and are at increased risk for thrombotic events, including stroke, pulmonary embolism, and deep vein thrombosis. Treatment of β-Thal intermedia includes splenectomy, folic acid supplementation, hydroxyurea therapy, and radiotherapy for extramedullary masses. Chelation therapy is used in subjects who develop iron overload.

Life expectancy is often diminished in β-Thal patients. Subjects with β-Thal major who do not receive transfusion therapy generally die in their second or third decade. Subjects with β-Thal major who receive regular transfusions and adequate chelation therapy can live into their fifth decade and beyond. Cardiac failure secondary to iron toxicity is the leading cause of death in β-Thal major subjects due to iron toxicity.

A variety of new treatments are currently in development for SCD and β-Thal. Delivery of a corrected HBB gene via gene therapy is currently being investigated in clinical trials. However, the long-term efficacy and safety of this approach is unknown. Transplantation with hematopoietic stem cells from an HLA-matched allogeneic stem cell donor has been demonstrated to cure SCD and β-Thal, but this procedure involves risks including those associated with ablation therapy to prepare the subject for transplant and risk of graft vs. host disease after transplantation. In addition, matched allogeneic donors often cannot be identified. Thus, there is a need for improved methods of managing these and other hemoglobinopathies.

### SUMMARY OF THE INVENTION

Provided herein in certain embodiments are methods for increasing expression (i.e., transcriptional activity) of one or more γ-globin genes (e.g., HBG1, HBG2, or HBG1 and HBG2) in a subject or cell using a genome editing system (e.g., CRISPR/Cas-mediated genome editing system). In certain embodiments, these methods may utilize any repair mechanism to alter (e.g., delete, disrupt, or modify) all or a portion of one or more γ-globin gene regulatory elements. In certain embodiments, these methods may utilize a DNA repair mechanism, e.g., NHEJ or HDR to delete or disrupt one or more γ-globin gene regulatory elements (e.g., silencer, enhancer, promoter, or insulator). In certain embodiments, these methods utilize a DNA repair mechanism, e.g., HDR, to alter, including mutate, insert, delete or disrupt, the sequence of one or more nucleotides in γ-globin gene regulatory element (e.g., silencer, enhancer, promoter, or insulator). In certain embodiments, these methods utilize a combination of one or more DNA repair mechanisms, e.g., NHEJ and HDR. In certain embodiments, these methods result in a mutation or variation in an γ-globin regulatory element that is associated with a naturally occurring HPFH variant, including, for example, HBG1 13 bp del c.−114 to −102; 4 bp del c.−225 to −222; c.−114 C>T; c.−117 G>A; c.−158 C>T; c.−167 C>T; c.−170 G>A; c.−175 T>G; c.−175 T>C; c.−195 C>G; c.−196 C>T; c.−198 T>C; c.−201 C>T; c.−251 T>C; or c.−499 T>A; or HBG2 13 bp del c.−114 to −102; c.−109 G>T; c.−114 C>A; c.−114 C>T; c.−157 C>T; c.−158 C>T; c.−167 C>T; c.−167 C>A; c.−175 T>C; c.−202 C>G; c.−211 C>T; c.−228 T>C; c.−255 C>G; c.−309 A>G; c.−369 C>G; or c.−567 T>G.

Provided herein in certain embodiments are methods for treating a β-hemoglobinopathy in a subject in need thereof using CRISPR/Cas-mediated genome editing to increase expression (i.e., transcriptional activity) of one or more γ-globin genes (e.g., HBG1, HBG2, or HBG1 and HBG2). In certain embodiments, these methods utilize a DNA repair mechanism, e.g., NHEJ or HDR to delete or disrupt one or more γ-globin gene regulatory elements (e.g., silencer, enhancer, promoter, or insulator). In certain embodiments, these methods utilize a DNA repair mechanism, e.g., HDR to alter, including mutate, insert, delete or disrupt, the sequence of one or more nucleotides in γ-globin gene regulatory element (e.g., silencer, enhancer, promoter, or insulator). In certain embodiments, these methods utilize a combination of one or more DNA repair mechanisms, e.g., NHEJ and HDR. In certain embodiments, these methods result in a mutation or variation in an γ-globin regulatory element that is associated with a naturally occurring HPFH variant, including for example HBG1 13 bp del c.−114 to −102; 4 bp del c.−225 to −222; c.−114 C>T; c.−117 G>A; c.−158 C>T; c.−167 C>T; c.−170 G>A; c.−175 T>G; c.−175 T>C; c.−195 C>G; c.−196 C>T; c.−198 T>C; c.−201 C>T; c.−251 T>C; or c.−499 T>A; or HBG2 13 bp del c.−114 to −102; c.−109 G>T; c.−114 C>A; c.−114 C>T; c.−157 C>T; c.−158 C>T; c.−167 C>T; c.−167 C>A; c.−175 T>C; c.−202 C>G; c.−211 C>T; c.−228 T>C; c.−255 C>G; c.−309 A>G; c.−369 C>G; or c.−567 T>G. In certain embodiments, the β-hemoglobinopathy is SCD or β-Thal.

Provided herein in certain embodiments are gRNAs for use in CRISPR/Cas-mediated methods of increasing expression (i.e., transcriptional activity) of one or more γ-globin genes (e.g., HBG1, HBG2, or HBG1 and HBG2). In certain embodiments, these gRNAs comprise a targeting domain comprising a nucleotide sequence set forth in SEQ ID NOs:251-901. In certain embodiments, these gRNAs further comprise one or more of a first complementarity domain, second complementarity domain, linking domain, 5′ extension domain, proximal domain, or tail domain. In certain embodiments, the gRNA is modular. In other embodiments, the gRNA is unimolecular (or chimeric).

### BRIEF DESCRIPTION OF THE DRAWINGS

FIGS. 1A-1I are representations of several exemplary gRNAs.

FIG. 1A depicts a modular gRNA molecule derived in part (or modeled on a sequence in part) from Streptococcus pyogenes (S. pyogenes) as a duplexed structure (SEQ ID NOs:39 and 40, respectively, in order of appearance);

FIG. 1B depicts a unimolecular gRNA molecule derived in part from S. pyogenes as a duplexed structure (SEQ ID NO:41);

FIG. 1C depicts a unimolecular gRNA molecule derived in part from S. pyogenes as a duplexed structure (SEQ ID NO:42);

FIG. 1D depicts a unimolecular gRNA molecule derived in part from S. pyogenes as a duplexed structure (SEQ ID NO:43);

FIG. 1E depicts a unimolecular gRNA molecule derived in part from S. pyogenes as a duplexed structure (SEQ ID NO:44);

FIG. 1F depicts a modular gRNA molecule derived in part from Streptococcus thermophilus (S. thermophilus) as a duplexed structure (SEQ ID NOs:45 and 46, respectively, in order of appearance);

FIG. 1G depicts an alignment of modular gRNA molecules of S. pyogenes and S. thermophilus (SEQ ID NOs:39, 45, 47, and 46, respectively, in order of appearance).

FIGS. 1H-1I depicts additional exemplary structures of unimolecular gRNA molecules.

FIG. 1H shows an exemplary structure of a unimolecular gRNA molecule derived in part from S. pyogenes as a duplexed structure (SEQ ID NO:42).

FIG. 1I shows an exemplary structure of a unimolecular gRNA molecule derived in part from S. aureus as a duplexed structure (SEQ ID NO:38).

FIGS. 2A-2G depict an alignment of Cas9 sequences (Chylinski 2013). The N-terminal RuvC-like domain is boxed and indicated with a “Y.” The other two RuvC-like domains are boxed and indicated with a “B.” The HNH-like domain is boxed and indicated by a “G.” Sm: S. mutans (SEQ ID NO:1); Sp: S. pyogenes (SEQ ID NO:2); St: S. thermophilus (SEQ ID NO:4); and Li: L. innocua (SEQ ID NO:5). “Motif” (SEQ ID NO:14) is a consensus sequence based on the four sequences. Residues conserved in all four sequences are indicated by single letter amino acid abbreviation; “*” indicates any amino acid found in the corresponding position of any of the four sequences; and “-” indicates absent.

FIGS. 3A-3B show an alignment of the N-terminal RuvC-like domain from the Cas9 molecules disclosed in Chylinski 2013 (SEQ ID NOs:52-95, 120-123). The last line of FIG. 3B identifies 4 highly conserved residues.

FIGS. 4A-4B show an alignment of the N-terminal RuvC-like domain from the Cas9 molecules disclosed in Chylinski 2013 with sequence outliers removed (SEQ ID NOs:52-123). The last line of FIG. 4B identifies 3 highly conserved residues.

FIGS. 5A-5C show an alignment of the HNH-like domain from the Cas9 molecules disclosed in Chylinski 2013 (SEQ ID NOs:124-198). The last line of FIG. 5C identifies conserved residues.

FIGS. 6A-6B show an alignment of the HNH-like domain from the Cas9 molecules disclosed in Chylinski 2013 with sequence outliers removed (SEQ ID NOs:124-141, 148, 149, 151-153, 162, 163, 166-174, 177-187, 194-198). The last line of FIG. 6B identifies 3 highly conserved residues.

FIG. 7 illustrates gRNA domain nomenclature using an exemplary gRNA sequence (SEQ ID NO:42).

FIGS. 8A and 8B provide schematic representations of the domain organization of S. pyogenes Cas9. FIG. 8A shows the organization of the Cas9 domains, including amino acid positions, in reference to the two lobes of Cas9 (recognition (REC) and nuclease (NUC) lobes). FIG. 8B shows the percent homology of each domain across 83 Cas9 orthologs.

FIG. 9 provides schematics of the HBG1 and HBG2 gene(s) in the context of the globin locus. The coding sequences (CDS), mRNA regions, and genes are indicated. The letter A indicates regions that were targeted for gRNA design (dashed lines and brackets indicating the genetic regions proximal to the HBG1 and HBG2 genes) are shown. The letter B indicates where core promoter elements are indicated. The letter C indicates motifs in the gene regulatory regions to which transcriptional activators and transcriptional repressors may bind to regulate gene expression are indicated. Note the overlap between the motifs and the genomic region targeted for gRNA design. Examples of deletions in the HBG1 and HBG2 gene regulatory regions that cause HPFH are indicated, as well as the % HbF associated with each.

FIGS. 10A-F shows data from gRNA screening for incorporation of the 13 bp del c.−114 to −102 HPFH mutation in human K562 erythroleukemia cells. FIG. 10A shows gene editing as determined by T7E1 endonuclease assay analysis of HBG1 and HBG2 locus-specific PCR products amplified from genomic DNA extracted from K562 cells after electroporation with DNA encoding S. pyogenes-specific gRNAs and plasmid DNA encoding S. pyogenes Cas9. FIG. 10B shows gene editing as determined by DNA sequence analysis of PCR products amplified from the HBG1 locus in genomic DNA extracted from K562 cells after electroporation with DNA encoding the indicated gRNA and Cas9 plasmid. FIG. 10C shows gene editing as determined by DNA sequence analysis of PCR products amplified from the HBG2 locus in genomic DNA extracted from K562 cells after electroporation with DNA encoding the indicated gRNA and Cas9 plasmid. For FIGS. 10B-C, the types of editing events (insertions, deletions) and subtypes of deletions (13 nt target partially [12 nt HPFH] or fully [13-26 nt HPFH] deleted, other sequences deleted [other deletions]) are indicated by the differently shaded/patterned bars. FIGS. 10D-F show examples of HBG1 gene regulatory region deletions.

FIGS. 11A-C depict results of gene editing in human cord blood (CB) and human adult CD34⁺ cells after electroporation with RNPs complexed to in vitro transcribed S. pyogenes gRNAs that target a specific 13 nt sequence for deletion (HBG gRNAs Sp35 (comprising SEQ ID NO:339) and Sp37 (comprising SEQ ID NO:333)). FIG. 11A depicts the percentage of indels detected by T7E1 analysis of HBG1 and HBG2 specific PCR products amplified from gDNA extracted from CB CD34⁺ cells treated with the indicated RNPs or donor matched untreated control cells (n=3 CB CD34⁺ cells, 3 separate experiments). Data shown represent the mean and error bars correspond to standard deviation across the three separate donors/experiments. FIG. 11B depicts the percentage of indels detected by T7E1 analysis of HBG2 specific PCR product amplified from gDNA extracted from CB CD34⁺ cells or adult CD34⁺ cells treated with the indicated RNPs or donor matched untreated control cells (n=3 CB CD34⁺ cells, n=3 mPB CD34⁺ cells, three separate experiments). Data shown represent the mean and error bars correspond to standard deviation across the three separate donors/experiments. FIG. 11C (top panel) depicts edits as detected by T7E1 analysis of HBG2 PCR products amplified from gDNA extracted from human CB CD34⁺ cells electroporated with HBG Sp35 RNP or HBG Sp37 RNP+/−ssODN1 (SEQ ID NO:906) or PhTx ssODN1 (SEQ ID NO:909). FIG. 11C (lower left panel) shows the level of gene editing as determined by Sanger DNA sequence analysis of gDNA from cells edited with HBG Sp37 RNP and ssODN1 and PhTx ssODN1. FIG. 11C (lower right panel) shows the specific types of deletions detected within total deletions from the data presented in the lower left panel.

FIGS. 12A-C depict gene editing of HBG1 and HBG2 in K562 erythroleukemia cells. FIG. 12A depicts NHEJ (indels) detected by T7E1 analysis of HBG1 and HBG2 PCR products amplified from gDNA extracted from K562 cells three days after nucleofection with RNPs complexed to the indicated gRNAs. FIG. 12B depicts Sanger DNA sequence analysis of PCR products amplified from the HBG1 locus for cells nucleofected with Cas9 protein complexed to gRNAs targeting the 13 nt HPFH sequence (Sp35 (comprising SEQ ID NO:339), Sp36 (comprising SEQ ID NO:338), Sp37 (comprising SEQ ID NO:333)). FIG. 12C depicts Sanger DNA sequence analysis of PCR products amplified from the HBG2 locus for cells nucleofected with Cas9 protein complexed to gRNAs targeting the 13 bp HPFH sequence (Sp35, Sp36, Sp37). For FIG. 12B and FIG. 12C the deletions were subdivided into deletions that contained the 13 bp targeted deletion (HPFH deletion, 18-26 nt deletion, >26 nt deletion) and deletions that did not contain the 13 bp deletion (<12 nt deletion, other deletion, insertion).

FIGS. 13A-B depict gene editing of HBG in adult human mobilized peripheral blood (mPB) CD34⁺ cells and induction of fetal hemoglobin in erythroid progeny of RNP treated cells after electroporation of mPB CD34⁺ cells with HBG Sp37 RNP+/−ssODN encoding the 13 bp deletion. FIG. 13A depicts the percentage of edits detected by T7E1 analysis of HBG2 PCR product amplified from gDNA extracted from mPB CD34⁺ cells treated with the RNP or donor matched untreated control cells. FIG. 13B depicts the fold change in HBG mRNA expression in day seven erythroblasts that were differentiated from RNP treated and untreated donor matched control mPB CD34⁺ cells. mRNA levels are normalized to GAPDH and calibrated to the levels detected in untreated controls on the corresponding days of differentiation.

FIGS. 14 A-B depict the ex vivo differentiation potential of RNP treated and untreated mPB CD34⁺ cells from the same donor. FIG. 14A shows hematopoietic myeloid/erythroid colony forming cell (CFC) potential, where the number and subtype of colonies are indicated (GEMM: granulocyte-erythroid-monocyte-macrophage colony, E: erythroid colony, GM: granulocyte-macrophage colony, M: macrophage colony, G: granulocyte colony). FIG. 14B depicts the percentage of Glycophorin A expressed over the time course of erythroid differentiation as determined by flow cytometry analysis at the indicated time points and for the indicated samples.

### DETAILED DESCRIPTION

### Definitions

“Domain” as used herein is used to describe segments of a protein or nucleic acid. Unless otherwise indicated, a domain is not required to have any specific functional property.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Polypeptide” as used herein refers to a polymer of amino acids having less than 100 amino acid residues. In an embodiment, it has less than 50, 20, or 10 amino acid residues.

“Alt-HDR,” “alternative homology-directed repair,” or “alternative HDR” as used herein refers to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Also, alt-HDR uses a single-stranded or nicked homologous nucleic acid for repair of the break.

“Canonical HDR” or “canonical homology-directed repair” as used herein refers to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” as used herein refers to ligation mediated repair and/or non-template mediated repair including canonical NHEJ (cNHEJ), alternative NHEJ (altNHEJ), microhomology-mediated end joining (MMEJ), single-strand annealing (SSA), and synthesis-dependent microhomology-mediated end joining (SD-MMEJ).

A “reference molecule” as used herein refers to a molecule to which a modified or candidate molecule is compared. For example, a reference Cas9 molecule refers to a Cas9 molecule to which a modified or candidate Cas9 molecule is compared. Likewise, a reference gRNA refers to a gRNA molecule to which a modified or candidate gRNA molecule is compared. The modified or candidate molecule may be compared to the reference molecule on the basis of sequence (e.g., the modified or candidate molecule may have X % sequence identity or homology with the reference molecule) or activity (e.g., the modified or candidate molecule may have X % of the activity of the reference molecule). For example, where the reference molecule is a Cas9 molecule, a modified or candidate molecule may be characterized as having no more than 10% of the nuclease activity of the reference Cas9 molecule. Examples of reference Cas9 molecules include naturally occurring unmodified Cas9 molecules, e.g., a naturally occurring Cas9 molecule from S. pyogenes, S. aureus, S. thermophilus, or N. meningitidis. In certain embodiments, the reference Cas9 molecule is the naturally occurring Cas9 molecule having the closest sequence identity or homology with the modified or candidate Cas9 molecule to which it is being compared. In certain embodiments, the reference Cas9 molecule is a parental molecule having a naturally occurring or known sequence on which a mutation has been made to arrive at the modified or candidate Cas9 molecule.

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

“Replacement” or “replaced” as used herein with reference to a modification of a molecule does not require a process limitation but merely indicates that the replacement entity is present.

“Subject” as used herein may mean a human, mouse, or non-human primate.

“Treat,” “treating,” and “treatment” as used herein mean the treatment of a disease in a subject, e.g., in a human, including (a) inhibiting the disease, i.e., arresting or preventing its development or progression; (b) relieving the disease, i.e., causing regression of the disease state; (c) relieving one or more symptoms of the disease; and (d) curing the disease. For example, “treating” SCD or β-Thal may refer to, among other possibilities, preventing development or progression of SCD or β-Thal, relieving one or more symptoms of SCD or β-Thal (e.g., anemia, sickle cell crises, vaso-occlusive crises), or curing SCD or β-Thal.

“Prevent,” “preventing,” and “prevention” as used herein means the prevention of a disease in a subject, e.g., in a human, including (a) avoiding or precluding the disease; (b) affecting the predisposition toward the disease; and (c) preventing or delaying the onset of at least one symptom of the disease.

“X” as used herein in the context of an amino acid sequence refers to any amino acid (e.g., any of the twenty natural amino acids) unless otherwise specified.

“Regulatory region” as used herein refers to a DNA sequence comprising one or more regulatory elements (e.g., silencer, enhancer, promoter, or insulator) controlling or regulating expression of a gene. For example, a γ-globin gene regulatory region comprises one or more regulatory elements controlling or regulating expression of a γ-globin gene. In certain embodiments, a regulatory region is adjacent to the gene being controlled or regulated. For example, a γ-globin gene regulatory region may be adjacent to or associated with the γ-globin gene. In other embodiments, the regulatory region may be adjacent to or associated with another gene, the expression of which can lead to up- or down-regulation of the gene being controlled or regulated. For example, a γ-globin gene regulatory region may be adjacent to a gene expressing a repressor of γ-globin gene expression. For HBG1, the regulatory region comprises at least nucleotides 1-2990 in SEQ ID NO:902. For HBG2, the regulatory region comprises at least nucleotides 1-2914 in SEQ ID NO:903.

“HBG target position” as used herein refers to a position in an HBG1 or HBG2 regulatory region (“HBG1 target position” and “HBG2 target position,” respectively) containing a target site (e.g., target sequence to be deleted or mutated) which, when altered (e.g., disrupted or deleted by introduction of a DNA repair mechanism-mediated (e.g., an NHEJ- or HDR-mediated) insertion or deletion, modified by a DNA repair mechanism-mediated (e.g., HDR-mediated) sequence alteration)) results in increased expression (e.g., de-repression) of HBG1 or HBG2 gene product (i.e., γ-globin). In certain embodiments, the HBG target position is in an HBG1 or HBG2 regulatory element (e.g., silencer, enhancer, promoter, or insulator) in a regulatory region adjacent to HBG1 or HBG2. In certain of these embodiments, alteration of the HBG target position results in decreased repressor binding, i.e., de-repression, leading to increased expression of HBG1 or HBG2. In other embodiments, the HBG target position is in a regulatory element of a gene other than HBG1 or HBG2 that encodes a gene product involved in controlling HBG1 or HBG2 gene expression (e.g., a repressor of HBG1 or HBG2 gene expression). In certain embodiments, the HBG target position is that region of an HBG1 or HBG2 regulatory region with the greatest density of binding motifs involved in the regulation of HBG1 or HBG2 expression. In certain embodiments, the methods provided herein target multiple HBG target positions simultaneously or sequentially.

“Target sequence” as used herein refers to a nucleic acid sequence comprising an HBG target position.

A “Cas9 molecule” or “Cas9 polypeptide” as used herein refers to a molecule or polypeptide, respectively, that can interact with a gRNA molecule and, in concert with the gRNA molecule, localize to a site comprising a target domain and, in certain embodiments, a PAM sequence. Cas9 molecules and Cas9 polypeptides include both naturally occurring Cas9 molecules and Cas9 polypeptides and engineered, altered, or modified Cas9 molecules or Cas9 polypeptides that differ, e.g., by at least one amino acid residue, from a reference sequence, e.g., the most similar naturally occurring Cas9 molecule.

### Overview

Provided herein are methods for increasing expression (i.e., transcriptional activity) of one or more γ-globin genes (e.g., HBG1, HBG2, or HBG1 and HBG2) using a genome editing system (e.g., CRISPR/Cas-mediated genome editing). These methods utilize a genome editing system (e.g., CRISPR/Cas-mediated genome editing) to alter (e.g., delete, disrupt, or modify) one or more γ-globin gene regulatory regions to increase (e.g., de-repress, enhance) γ-globin gene expression. In certain of these embodiments, the methods alter one or more regulatory elements (e.g., silencer, enhancer, promoter, or insulator) associated with the γ-globin gene being targeted. In other embodiments, the methods alter one or more regulatory elements in genes other than the γ-globin gene being targeted (e.g., genes encoding γ-globin gene repressors). In certain embodiments, a genome editing system (e.g., CRISPR/Cas-mediated genome editing) is used to alter a regulatory element (e.g., silencer, enhancer, promoter, or insulator) of HBG1, HBG2, or both HBG1 and HBG2. In certain embodiments, a genome editing system (e.g., CRISPR/Cas-mediated genome editing) results in a mutation or variation in an γ-globin regulatory element that is associated with a naturally occurring HPFH variant, including, for example, HBG1 13 bp del c.−114 to −102; 4 bp del c.−225 to −222; c.−114 C>T; c.−117 G>A; c.−158 C>T; c.−167 C>T; c.−170 G>A; c.−175 T>G; c.−175 T>C; c.−195 C>G; c.−196 C>T; c.−198 T>C; c.−201 C>T; c.−251 T>C; or c.−499 T>A; or HBG2 13 bp del c.−114 to −102; c.−109 G>T; c.−114 C>A; c.−114 C>T; c.−157 C>T; c.−158 C>T; c.−167 C>T; c.−167 C>A; c.−175 T>C; c.−202 C>G; c.−211 C>T; c.−228 T>C; c.−255 C>G; c.−309 A>G; c.−369 C>G; or c.−567 T>G.

In some embodiments, the methods using a genome editing system (e.g., CRISPR/Cas-mediated genome editing) described herein may utilize any repair mechanism to alter (e.g., delete, disrupt, or modify) all or a portion of one or more γ-globin gene regulatory elements. In certain embodiments, the methods utilize DNA repair mechanism-mediated (e.g., NHEJ or HDR-mediated) insertions or deletions to disrupt all or a portion of one or more γ-globin gene regulatory elements. For example, the methods may utilize a DNA repair mechanism (e.g., NHEJ or HDR) to delete all or a portion of a γ-globin gene negative regulatory element (e.g., silencer), resulting in inactivation of the negative regulatory element (e.g., loss of binding between a silencer and repressor) and increased expression of the γ-globin gene. In other embodiments, the methods utilize DNA repair mechanism-mediated (e.g., NHEJ or HDR-mediated) insertions or deletions to disrupt all or a portion of one or more regulatory elements associated with a gene encoding a γ-globin gene repressor. For example, the methods may utilize a DNA repair mechanism (e.g., NHEJ or HDR) to delete all or a portion of a positive regulatory element (e.g., promoter) of a γ-globin repressor gene, resulting in decreased expression of the repressor, decreased binding of the repressor to a γ-globin gene silencer, and increased expression of the γ-globin gene. In other embodiments, the methods utilize a DNA repair mechanism (e.g., HDR) to modify the sequence of one or more γ-globin gene regulatory elements (e.g., inserting a mutation in an HBG1 and/or HBG2 regulatory element corresponding to a naturally occurring HPFH mutation or deleting all or a portion of an HBG1 and/or HBG2 regulatory element). In some embodiments, the methods may use a combination of one or more DNA repair mechanisms (e.g., NHEJ and HDR). In certain embodiments, the methods create persistence of HbF in a subject. Also provided herein are compositions (e.g., gRNAs, Cas9 polypeptides and molecules, template nucleic acids, vectors) and kits for use in these methods.

The transition from expression of γ-globin genes (i.e., HBG1, HBG2) to expression of HBB (i.e., globin switching) is associated with the onset of symptoms of β-hemoglobinopathies including SCD and β-Thal. Therefore, in certain embodiments, methods, compositions, and kits are provided herein for treating or preventing β-hemoglobinopathies including SCD and β-Thal using CRISPR/Cas-mediated genome editing to increase expression of one or more γ-globin genes (e.g., HBG1, HBG2, or HBG1 and HBG2). In certain of these embodiments, the methods alter one or more regulatory elements (e.g., silencer, enhancer, promoter, or insulator) associated with the γ-globin gene being targeted. In other embodiments, the methods alter one or more regulatory elements in genes other than the γ-globin gene being targeted (e.g., genes encoding γ-globin gene repressors). In certain embodiments, CRISPR/Cas-mediated genome editing is used to alter a regulatory element (e.g., silencer, enhancer, promoter, or insulator) of HBG1, HBG2, or both HBG1 and HBG2. In some embodiments, the methods utilize DNA repair mechanism-mediated (e.g., NHEJ or HDR-mediated) insertions or deletions to disrupt all or a portion of one or more γ-globin gene regulatory elements. For example, the methods may utilize a DNA repair mechanism (e.g., NHEJ or HDR) to delete all or a portion of a γ-globin gene negative regulatory element (e.g., silencer), resulting in inactivation of the negative regulatory element (e.g., loss of binding between a silencer and repressor) and increased expression of the γ-globin gene. In other embodiments, the methods utilize DNA repair mechanism-mediated (e.g., NHEJ or HDR-mediated) insertions or deletions to disrupt all or a portion of one or more regulatory elements associated with a gene encoding a γ-globin gene repressor. For example, the methods may utilize a DNA repair mechanism (e.g., NHEJ or HDR) to delete all or a portion of a positive regulatory element (e.g., promoter) of a γ-globin repressor gene, resulting in decreased expression of the repressor, decreased binding of the repressor to a γ-globin gene silencer, and increased expression of the γ-globin gene. In other embodiments, the methods utilize a DNA repair mechanism (e.g., HDR) to modify the sequence of one or more γ-globin gene regulatory elements (e.g., inserting a mutation in an HBG1 and/or HBG2 regulatory element corresponding to a naturally occurring HPFH mutation or deleting all or a portion of an HBG1 and/or HBG2 regulatory element). In some embodiments, the methods may use a combination of one or more DNA repair mechanisms (e.g., NHEJ and HDR). In certain embodiments, the methods create persistence of HbF in a subject.

In certain embodiments, increased expression of one or more γ-globin genes (e.g., HBG1, HBG2) using the methods provided herein results in preferential formation of HbF over HbA and/or increased HbF levels as a percentage of total hemoglobin. Accordingly, further provided herein are methods of using CRISPR/Cas-mediated genome editing to increase total HbF levels, increase HbF levels as a percentage of total hemoglobin levels, or increase the ratio of HbF to HbA in a subject by increasing the expression of one or more γ-globin genes (e.g., HBG1, HBG2, or HBG1 and HBG2). Similarly, in certain embodiments increased expression of one or more γ-globin genes results in preferential formation of HbF versus HbS and/or decreased percentage of HbS as a percentage of total hemoglobin. Accordingly, further provided herein are methods of using CRISPR/Cas-mediated genome editing to decrease total HbS levels, decrease HbS levels as a percentage of total hemoglobin levels, or increase the ratio of HbF to HbS in a subject by increasing the expression of one or more γ-globin genes (e.g., HBG1, HBG2, or HBG1 and HBG2).

Provided herein in certain embodiments are gRNAs for use in the methods disclosed herein. In certain embodiments, these gRNAs comprise a targeting domain complementary or partially complementary to a target domain in or adjacent to an HBG target position. In certain embodiments, the targeting domain comprises, consists of, or consists essentially of a nucleotide sequence set forth in one of SEQ ID NOs:251-901.

Genomic studies have led to the identification of several genes that regulate globin switching, including BCL11A, Kruppel-like factor 1 (KLF1), MYB, and genes within the β globin locus. Mutations in certain of these genes may result in inhibited or incomplete globin switching, also known as hereditary persistence of fetal hemoglobin (HPFH). HPFH mutations may be deletional or non-deletional (e.g., point mutations). Subjects with HPFH exhibit lifelong expression of HbF, i.e., they do not undergo or undergo only partial globin switching, with no symptoms of anemia. Heterozygous subjects exhibit 20-40% pancellular HbF, and co-inheritance results in alleviation of β-hemoglobinopathies (Thein 2009; Akinbami 2016). Compound heterozygotes for hemoglobinopathies and HPFH, e.g., subjects who are compound heterozygotes for SCD and HPFH, β-Thal and HPFH, sickle cell trait and HPFH, or delta-β-Thal and HPFH, have milder disease and symptoms relative to subjects without HPFH mutations. Patients homozygous for HbS who also co-inherit an HPFH mutation, e.g., a mutation that induces expression of HbF through de-repression of HBG1 or HBG2, do not develop SCD symptoms or β-Thal symptoms (Steinberg et al., Disorders of Hemoglobin, Cambridge Univ. Press, 2009, p. 570). HPFH is clinically benign (Chassanidis 2009).

While the occurrence of HPFH is rare in the global population, it is more common in populations with greater prevalence of hemoglobinopathies, including those of Southern European, South American, and African descent. In these populations, the prevalence of HPFH can reach 1-2 in 1,000 individuals (Costa 2002: Ahern 1973). Theoretically, HPFH mutations persist in these populations because they ameliorate disease in subjects with hemoglobinopathies.

The most common naturally occurring HPFH mutations are deletions within the R globin locus. Common examples of deletional HPFH mutations include French HPFH (23 kb deletion), Caucasian HPFH (19 kb deletion), HPFH-1 (84 kb deletion), HPFH-2 (84 kb deletion), and HPFH-3 (50 kb deletion). In subjects with these mutations, β-globin synthesis is reduced, and 7-globin synthesis is secondarily increased.

Other HPFH mutations are located in γ-globin gene regulatory regions. One such mutation is a 13 nucleotide deletion (13 base pair (bp) del c.−114 to −102; CAATAGCCTTGAC del, sequence based on reverse complement of HBG1/HBG2) located upstream of both the HBG1 and HBG2 genes. This deletion disrupts a silencer element that normally prevents HBG1/HBG2 expression, and adult subjects heterozygous for this deletion exhibit approximately 30% HbF. Another HPFH mutation is a 4 nucleotide deletion (4 base pair (bp) del c.−225 to −222 (AGCA del)). Other HPFH mutations found in both HBG1 and HBG2 regulatory elements include, for example, non-deletional point mutations (non-del HPFH) such as c.−114 C>T; c.−158 C>T; c.−167 C>T; and c.−175 T>C.

Non-del HPFH mutations associated with HBG1 regulatory elements include, for example, c.−117 G>A; c.−170 G>A; c.−175 T>G; c.−195 C>G; c.−196 C>T; c.−198 T>C; c.−201 C>T; c.−251 T>C; and c.−499 T>A.

Non-del HPFH mutations associated with HBG2 regulatory elements include, for example, c.−109 G>T; c.−114 C>A; c.−157 C>T; c.−167 C>A; c.−202 C>G; c.−211 C>T; c.−228 T>C; c.−255 C>G; and c.−567 T>G.

Additional polymorphisms in HBG1 and HBG2 promoter regions have been identified in a cohort of Brazilian SCD patients that corrected HbF levels >5% (Barbosa 2010). These include c.−309 A>G and c.−369 C>G in the HBG2 promoter.

HBG1 and HBG2 promoter elements that may be altered to recreate HPFH mutations include, for example, erythroid Kruppel-like factor (EKLF-2) and fetal Kruppel-like factor (FKLF) transcription factor binding motifs (CTCCACCCA), CP1/Coup TFII binding motifs (CCAATAGC), GATA1 binding motifs (CTATCT, ATATCT), or stage selector element (SSE) binding motifs. HBG1 and HBG2 enhancer elements that may be altered to recreate HPFH mutations include, for example, SOX binding motifs, e.g., SOX14, SOX2, or SOX1 (CCAATAGCCTTGA).

In certain embodiments of the methods provided herein, CRISPR/Cas-mediated alteration is used to alter one regulatory element or motif in a γ-globin gene regulatory region, e.g., a silencer sequence in an HBG1 or HBG2 regulatory region, or a promoter or enhancer sequence associated with a gene encoding an HBG1 or HBG2 repressor. In other embodiments, CRISPR/Cas-mediated alteration is used to alter two or more (e.g., three, four, or five or more) regulatory elements or motifs in a γ-globin gene regulatory region, e.g., an HBG1 or HBG2 silencer sequence and an HBG1 or HBG2 enhancer sequence; an HBG1 or HBG2 silencer sequence and a promoter or enhancer sequence associated with a gene encoding an HBG1 or HBG2 repressor; or an HBG1 or HBG2 silencer sequence and a promoter or enhancer sequence associated with a gene encoding an HBG1 or HBG2 repressor. The introduction of multiple variants into the regulatory region of a single gene or the introduction of one variant into the regulatory regions of two or more genes is referred to herein as “multiplexing.” Thus, multiplexing constitutes either (a) the modification of more than one location in one gene regulatory region in the same cell or cells or (b) the modification of one location in more than one gene regulatory region.

In certain embodiments of the methods provided herein, CRISPR/Cas-mediated alteration of one or more γ-globin gene regulatory elements produces a phenotype the same as or similar to a phenotype associated with a naturally occurring HPFH mutation. In certain embodiments, CRISPR/Cas-mediated alteration results in a γ-globin gene regulatory element comprising an alteration corresponding to a naturally occurring HPFH mutation. In other embodiments, alterations of one or more γ-globin gene regulatory elements results in an alteration that is not observed in a naturally occurring HPFH mutation (i.e., a non-naturally occurring variant).

In certain embodiments of the methods provided herein, CRISPR/Cas-mediated alteration of one or more γ-globin gene regulatory elements produces a mutation or variation in an 7-globin regulatory element that is associated with a naturally occurring HPFH variant, including, for example, HBG1 13 bp del c.−114 to −102; 4 bp del c.−225 to −222; c.−114 C>T; c.−117 G>A; c.−158 C>T; c.−167 C>T; c.−170 G>A; c.−175 T>G; c.−175 T>C; c.−195 C>G; c.−196 C>T; c.−198 T>C; c.−201 C>T; c.−251 T>C; or c.−499 T>A; or HBG2 13 bp del c.−114 to −102; c.−109 G>T; c.−114 C>A; c.−114 C>T; c.−157 C>T; c.−158 C>T; c.−167 C>T; c.−167 C>A; c.−175 T>C; c.−202 C>G; c.−211 C>T; c.−228 T>C; c.−255 C>G; c.−309 A>G; c.−369 C>G; or c.−567 T>G.

In certain embodiments, the methods provided herein comprise altering one or more transcription factor binding motifs (e.g., gene regulatory motif) in a γ-globin gene regulatory element. These transcription factor binding motifs include, for example, binding motifs that are occupied by transcription factors (TFs), TF complexes, and transcriptional repressors within the promoter regions of HBG1 and/or HBG2. In certain embodiments of the methods provided herein, introduction of a CRISPR/Cas-mediated alteration in one or more γ-globin gene regulatory elements alters binding of a transcription factor, e.g., a repressor, at one, two, three, or more than three motifs. In certain embodiments, introduction of a CRISPR/Cas-mediated alteration in one or more γ-globin gene regulatory elements results in increased RNA polymerase II initiation of transcription proximal to or at a γ-globin gene promoter region, e.g., by increasing transcription factor binding to an enhancer region, e.g., by decreased repressor binding at a silencer region.

In certain embodiments, the methods provided herein utilize a DNA repair mechanism-mediated (e.g., NHEJ- or HDR-mediated) deletion to delete all or a portion of nucleotides −114 to −102 in one or both alleles of HBG1, HBG2, or both HBG1 and HBG2, resulting in an HPFH phenotype the same as or similar to that associated with the naturally occurring 13 bp del c.−114 to −102 mutation. In other embodiments, a DNA repair mechanism-mediated (e.g., NHEJ- or HDR-mediated) deletion is utilized to delete all or a portion of nucleotides −225 to −222 of one or both alleles of HBG1, resulting in an HPFH phenotype the same as or similar to that associated with the naturally occurring HBG1 4 bp del −225 to −222 mutation. In other embodiments, a DNA repair mechanism-mediated (e.g., NHEJ- or HDR-mediated) deletion is utilized to delete all or a portion of nucleotides −225 to −222 of one or both alleles of HBG2.

In certain embodiments, the methods provided herein utilize a DNA repair mechanism-mediated (e.g., NHEJ- or HDR-mediated) deletion to delete all or a portion of nucleotides −114 to −102 in one or both alleles of HBG1 and one or both alleles of HBG2.

In certain embodiments, the methods provided herein utilize a DNA repair mechanism-mediated (e.g., NHEJ or HDR-mediated) deletion to delete all or a portion of nucleotides −225 to −222 in one or both alleles of HBG1 and all or a portion of nucleotides −114 to −102 in one or both HBG2 alleles. In other embodiments, a DNA repair mechanism (e.g., NHEJ- or HDR-mediated deletion) is utilized to delete all or a portion of nucleotides −225 to −222 in one or both alleles of HBG1 and all or a portion of nucleotides −114 to −102 in one or both HBG1 alleles.

In those embodiments wherein a DNA repair mechanism-mediated (e.g., NHEJ- or HDR-mediated) deletion is used to delete one or more nucleotides from HBG1, HBG2, or HBG1 and HBG2 regulatory elements, the deletions may be identical to those observed in naturally occurring HPFH mutations, i.e., the deletion may consist of nucleotides −114 to −102 of HBG1 or HBG2, or nucleotides −225 to −222 of HBG1. In other embodiments, the DNA repair mechanism-mediated (e.g., the NHEJ- or HDR-mediated) deletion results in removal of only a portion of these nucleotides, e.g., deletion of 12 or fewer nucleotides falling within −114 to −102 of HBG1 or HBG2 or three of fewer nucleotides falling within −225 to −222 of HBG1. In certain embodiments, one more nucleotides may be knocked out on either side of the naturally occurring HPFH mutation deletion boundaries (i.e., outside of −114 to −102 or −225 to −222) in addition to all or a portion of the nucleotides within the naturally occurring deletion boundaries.

In certain embodiments, the methods provided herein utilize a DNA repair mechanism-mediated (e.g., NHEJ- or HDR-mediated) insertion to insert one or more nucleotides into the region spanning nucleotides −114 to −102 of an HBG1 regulatory region, HBG2 regulatory region, or both HBG1 and HBG2 regulatory regions, or the region spanning nucleotides −225 to −222 of an HBG1 regulatory region, in order to disrupt a repressor binding site.

In certain embodiments, the methods provided herein utilize a DNA repair mechanism (e.g., HDR) to generate single nucleotide alterations (i.e., non-deletion mutants) corresponding to naturally occurring mutations associated with HPFH. For example, in certain embodiments the methods utilize a DNA repair mechanism (e.g., HDR) to generate a single nucleotide alteration in an HBG1 regulatory region that corresponds to a naturally occurring mutation associated with HPFH, including for example c.−114 C>T; c.−117 G>A; c.−158 C>T; c.−167 C>T; c.−170 G>A; c.−175 T>G; c.−175 T>C; c.−195 C>G; c.−196 C>T; c.−198 T>C; c.−201 C>T; c.−251 T>C; or c.−499 T>A. In other embodiments, a DNA repair mechanism (e.g., HDR) is utilized to generate a single nucleotide alteration in an HBG2 regulatory region that corresponds to a naturally occurring mutation associated with HPFH, including for example c.−109 G>T; c.−114 C>A; c.−114 C>T; c.−157 C>T; c.−158 C>T; c.−167 C>T; c.−167 C>A; c.−175 T>C; c.−202 C>G; c.−211 C>T; c.−228 T>C; c.−255 C>G; c.−309 A>G; c.−369 C>G; c.−567 T>G.

In certain embodiments, a DNA repair mechanism (e.g., HDR) is utilized to generate a single nucleotide alteration in an HBG1 regulatory region corresponding to a naturally occurring HPFH mutation found in an HBG2 regulatory region but not an HBG1 regulatory region. Such alterations include, for example, c.−109 G>T; c.−114 C>A; c.−157 C>T; c.−167 C>A; c.−202 C>G; c.−211 C>T; c.−228 T>C; c.−255 C>G; c.−309 A>G; c.−369 C>G; or c.−567 T>G.

Likewise, in certain embodiments a DNA repair mechanism (e.g., HDR) is utilized to generate a single nucleotide alteration in an HBG2 regulatory region corresponding to a naturally occurring HPFH mutation found in an HBG1 regulatory region but not an HBG2 regulatory region. Such alterations include, for example, c.−117 G>A; c.−170 G>A; c.−175 T>G; c.−195 C>G; c.−196 C>T; c.−198 T>C; c.−201 C>T; c.−251 T>C; or c.−499 T>A.

In certain embodiments, the methods provided herein comprise inserting the non-deletion HPFH variant c.−114 C>T into an HBG1 and/or HBG2 regulatory region by a DNA repair mechanism (e.g., HDR).

In certain embodiments, the methods provided herein comprise inserting the non-deletion HPFH variant c.−158 C>T (i.e., rs7482144 or XmnI-HBG2 variant) into an HBG1 and/or HBG2 regulatory region by a DNA repair mechanism (e.g., HDR).

In certain embodiments, the methods provided herein comprise inserting the non-deletion HPFH variant c.−167 C>T into an HBG1 and/or HBG2 regulatory region by a DNA repair mechanism (e.g., HDR).

In certain embodiments, the methods provided herein comprise inserting the non-deletion HPFH variant c.−175 T>C (i.e., T→C substitution at position c.−175 in a conserved octamer [ATGCAAAT] sequence) into an HBG1 regulatory region by a DNA repair mechanism (e.g., HDR). This variant, which is associated with 40% HbF, has been shown to abolish the ability of a ubiquitous octamer binding nuclear protein to bind the HBG promoter fragment, while simultaneously increased the ability of two erythroid specific proteins to bind the same fragment by 3-5 fold (Mantovani 1988).

In certain embodiments, the methods provided herein comprise inserting the non-deletion HPFH variant c.−175 T>C into an HBG2 regulatory region by a DNA repair mechanism (e.g., HDR). This variant is associated with 20-30% HbF expression.

In certain embodiments, the methods provided herein comprise inserting the non-deletion HPFH variant c.−117 G>A into an HBG1 regulatory region by a DNA repair mechanism (e.g., HDR). This variant, referred to as “Greek type,” is the most common nondeletion HPFH mutant and maps two nucleotides upstream from the distal CCAAT box (Waber 1986). HBG1 c.−117 G>A greatly decreases binding of erythroid-specific factors, but not of the ubiquitous protein, to the CCAAT box region fragment, and is associated with 10-20% HbF (Mantovani 1988). The mutation is thought to interfere with binding of nuclear factor E (NF-E), which is likely to play a role in repression of γ-globin transcription in adult erythroid cells (Superti-Furga 1988). In other embodiments, the methods provided herein comprise inserting the non-deletion HPFH variant c.−117 G>A into an HBG2 regulatory region, creating a non-naturally occurring HPFH variant.

In certain embodiments, the methods provided herein comprise inserting the non-deletion HPFH variant c.−170 G>A into an HBG1 regulatory region by a DNA repair mechanism (e.g., HDR).

In certain embodiments, the methods provided herein comprise inserting the non-deletion HPFH variant c.−175 T>G into an HBG1 regulatory region by a DNA repair mechanism (e.g., HDR).

In certain embodiments, the methods provided herein comprise inserting the non-deletion HPFH variant c.−195 C>G into an HBG1 regulatory region.

In certain embodiments, the methods provided herein comprise inserting the non-deletion HPFH variant c.−196 C>T into an HBG1 regulatory region by a DNA repair mechanism (e.g., HDR). This variant is associated with 10-20% HbF.

In certain embodiments, the methods provided herein comprise inserting the non-deletion HPFH variant c.−198 T>C into an HBG1 regulatory region by a DNA repair mechanism (e.g., HDR). This variant is associated with 18-21% HbF.

In certain embodiments, the methods provided herein comprise inserting the non-deletion HPFH variant c.−201 C>T into an HBG1 regulatory region.

In certain embodiments, the methods provided herein comprise inserting the non-deletion HPFH variant c.−251 T>C into an HBG1 regulatory region by a DNA repair mechanism (e.g., HDR).

In certain embodiments, the methods provided herein comprise inserting the non-deletion HPFH variant c.−499 T>A into an HBG1 regulatory region by a DNA repair mechanism (e.g., HDR).

In certain embodiments, the methods provided herein comprise inserting the non-deletion HPFH variant c.−109 G>T (“Hellenic mutation”) into an HBG2 regulatory region by a DNA repair mechanism (e.g., HDR). This mutation is located at the 3′ end of the HBG2 CCAAT box in the promoter region (Chassanidis 2009).

In certain embodiments, the methods provided herein comprise inserting the non-deletion HPFH variant c.−114 C>A into an HBG2 regulatory region by a DNA repair mechanism (e.g., HDR).

In certain embodiments, the methods provided herein comprise inserting the non-deletion HPFH variant c.−157 C>T into an HBG2 regulatory region by a DNA repair mechanism (e.g., HDR).

In certain embodiments, the methods provided herein comprise inserting the non-deletion HPFH variant c.−167 C>A into an HBG2 regulatory region by a DNA repair mechanism (e.g., HDR).

In certain embodiments, the methods provided herein comprise inserting the non-deletion HPFH variant c.−202 C>G into an HBG2 regulatory region by a DNA repair mechanism (e.g., HDR). This variant is associated with 15-25% HbF expression.

In certain embodiments, the methods provided herein comprise inserting the non-deletion HPFH variant c.−211 C>T into an HBG2 regulatory region by a DNA repair mechanism (e.g., HDR).

In certain embodiments, the methods provided herein comprise inserting the non-deletion HPFH variant c.−228 T>C into an HBG2 regulatory region by a DNA repair mechanism (e.g., HDR).

In certain embodiments, the methods provided herein comprise inserting the non-deletion HPFH variant c.−255 C>G into an HBG2 regulatory region by a DNA repair mechanism (e.g., HDR).

In certain embodiments, the methods provided herein comprise inserting the non-deletion HPFH variant c.−309 A>G into an HBG2 regulatory region by a DNA repair mechanism (e.g., HDR).

In certain embodiments, the methods provided herein comprise inserting the non-deletion HPFH variant c.−369 C>G into an HBG2 regulatory region by a DNA repair mechanism (e.g., HDR).

In certain embodiments, the methods provided herein comprise inserting the non-deletion HPFH variant c.−567 T>G into an HBG2 regulatory region by a DNA repair mechanism (e.g., HDR).

In certain embodiments, the methods provided herein comprise deletion, disruption, or mutation of a BCL11a core binding motif (i.e., GGCCGG) located at position c.−56 relative to HBG1 and/or HBG2 and/or at another location in a γ-globin gene regulatory region.

In certain embodiments, the methods provided herein comprise altering one or more nucleotides in a GATA (e.g., GATA1) motif. In certain of these embodiments, a DNA repair mechanism (e.g., HDR) is used to insert a T>C mutation into the HBG1 GATA binding motif within the sequence AAATATCTGT, resulting in the altered sequence AAACATCTGT. This naturally occurring T>C HPFH mutation is associated with 40% HbF.

In certain embodiments, the methods provided herein utilize one or more DNA repair mechanism (e.g., both NHEJ and HDR) approaches. For example, in certain embodiments, the methods utilize NHEJ-mediated deletion, e.g., introduction of 13 bp del c.−114 to −102 into one or both alleles of HBG1 and/or HBG2 and/or 4 bp del c.−225 to −222 into one or both alleles of HBG1, in combination with HDR-mediated single nucleotide alteration, e.g., introduction of one or more of c.−109 G>T; c.−114 C>A; c.−114 C>T; c.−117 G>A; c.−157 C>T; c.−158 C>T; c.−167 C>T; c.−167 C>A; c.−170 G>A; c.−175 T>C; c.−175 T>G; c.−195 C>G; c.−196 C>T; c.−198 T>C; c.−201 C>T; c.−202 C>G; c.−211 C>T; c.−228 T>C; c.−251 T>C; c.−255 C>G; c.−309 A>G; c.−369 C>G; c.−499 T>A; or c.−567 T>G into one or both alleles of HBG1 and/or HBG2.

In certain embodiments, the methods utilize HDR-mediated deletion, e.g., introduction of 13 bp del c.−114 to −102 into one or both alleles of HBG1 and/or HBG2 and/or 4 bp del c.−225 to −222 into one or both alleles of HBG1, in combination with HDR-mediated single nucleotide alteration, e.g., introduction of one or more of c.−109 G>T; c.−114 C>A; c.−114 C>T; c.−117 G>A; c.−157 C>T; c.−158 C>T; c.−167 C>T; c.−167 C>A; c.−170 G>A; c.−175 T>C; c.−175 T>G; c.−195 C>G; c.−196 C>T; c.−198 T>C; c.−201 C>T; c.−202 C>G; c.−211 C>T; c.−228 T>C; c.−251 T>C; c.−255 C>G; c.−309 A>G; c.−369 C>G; c.−499 T>A; or c.−567 T>G into one or both alleles of HBG1 and/or HBG2.

While not wishing to be bound by theory, introduction of 4 bp del c.−225 to −222 into the HBG1 gene regulatory region reverses the normal ratio of 70% γ^{A}-globin (γ-globin product of the HBG1 gene) to 30% γ^{G}-globin (γ-globin product of the HBG2 gene), so that γ-globin is produced as approximately 30% γ^{A}-globin and 70% γ^{G}-globin. While not wishing to be bound by theory, reversal of γ^{G}-globin and γ^{A}-globin ratio results in increased production of γ^{G}-globin in a subject. While not wishing to be bound by theory, concomitant introduction of 4 bp del c.−225 to −222 into the HBG1 gene regulatory region and 13 bp del c.−114 to −102 into the HBG2 gene regulatory region leads to increased transcriptional activity of HBG2, increased production of γ^{G}-globin, and increased HbF in a subject. While not wishing to be bound by theory, concomitant introduction of (a) 4 bp del c.−225 to −222 into the HBG1 gene regulatory region, e.g., by NHEJ- or HDR-mediated deletion, and (b) a non-deletion HPFH variant, e.g., by HDR, e.g., c.−109 G>T; c.−114 C>T; c.−114 C>A; c.−157 C>T; c.−158 C>T; c.−167 C>T; c.−167 C>A; c.−175 T>C; c.−202 C>G; c.−211 C>T; c.−228 T>C; c.−255 C>G; c.−309 A>G; c.−369 C>G; c.−567 T>G, into the HBG2 gene regulatory region leads to increased transcriptional activity of HBG2, increased production of γ^{G}-globin and increased HbF in a subject.

While not wishing to be bound by theory, introduction of 4 bp del c.−225 to −222 into the HBG2 gene regulatory region may decrease the production of γ^{G}-globin (γ-globin product of the HBG2 gene) relative to production of γ^{A}-globin (γ-globin product of the HBG1 gene), so that more γ^{A}-globin is produced than by γ^{G}-globin. While not wishing to be bound by theory, concomitant introduction of 4 bp del c.−225 to −222 into the HBG2 gene regulatory region and 13 bp del c.−114 to −102 into the HBG1 gene regulatory region may lead to increased transcriptional activity of HBG1, increased production of γ^{A}-globin and increased HbF in a subject. While not wishing to be bound by theory, concomitant introduction of (a) 4 bp del c.−225 to −222 into the HBG2 gene regulatory region, e.g., by NHEJ- or HDR-mediated deletion, and (b) a non-deletion HPFH variant, e.g., by HDR, e.g., c.−114 C>T; c.−117 G>A; c.−158 C>T; c.−167 C>T; c.−170 G>A; c.−175 T>G; c.−175 T>C; c.−195 C>G; c.−196 C>T; c.−198 T>C; c.−201 C>T; c.−251 T>C; or c.−499 T>A, into the HBG1 gene regulatory region may lead to increased transcriptional activity of HBG1, increased production of γ^{A}-globin, and increased HbF in a subject.

While not wishing to be bound by theory, concomitant introduction of (a) 13 bp del c.−114 to −102 into the HBG1 gene regulatory region, e.g., by NHEJ- or HDR-mediated deletion, and (b) a non-deletion HPFH variant, e.g., by HDR, e.g., c.−109 G>T; c.−114 C>T; c.−114 C>A; c.−157 C>T; c.−158 C>T; c.−167 C>A; c.−167 C>T; c.−175 T>C; c.−202 C>G; c.−211 C>T; c.−228 T>C; c.−255 C>G; c.−309 A>G; c.−369 C>G; or c.−567 T>G, into the HBG2 gene regulatory region leads to increased transcriptional activity of HBG2, increased production of γ^{G}-globin, and increased HbF in a subject.

While not wishing to be bound by theory, concomitant introduction of (a) 13 bp del c.−114 to −102 into the HBG2 gene regulatory region, e.g., by NHEJ- or HDR-mediated deletion, and (b) a non-deletion HPFH variant, e.g., by HDR, e.g., c.−114 C>T; c.−117 G>A; c.−158 C>T; c.−167 C>T; c.−170 G>A; c.−175 T>C; c.−175 T>G; c.−195 C>G; c.−196 C>T; c.−198 T>C; c.−201 C>T; c.−251 T>C; or c.−499 T>A, into the HBG1 gene regulatory region leads to increased transcriptional activity of HBG1, increased production of γ^{A}-globin and increased HbF in a subject.

Concomitant (a) BCL11A knockdown by siRNA and (b) SOX6 knockdown by siRNA leads to increased expression of HBG1 and HBG2 (Xu 2010). In certain embodiments, the methods provided herein comprise disrupting the action of BCL11A, SOX6, or BCL11A and SOX6 on the expression of HBG1 and HBG2 using a DNA repair mechanism (e.g., HDR, NHEJ, or NHEJ and HDR) modification of the HBG1 and HBG2 promoter regions and the erythroid-specific enhancer of BCL11A, alone or in parallel. In certain embodiments, the methods provided herein comprise decreasing BCL11A expression by disrupting the function of its intronic erythroid-specific enhancer by NHEJ and HDR and simultaneously inducing HPFH mutations for a synergistic effect on the production of HbF.

The embodiments described herein may be used in all classes of vertebrate including, but not limited to, primates, mice, rats, rabbits, pigs, dogs, and cats.

### Timing and Subject Selection

Initiation of treatment using the methods disclosed herein may occur prior to disease onset, for example in a subject who has been deemed at risk of developing a β-hemoglobinopathy (e.g., SCD, β-Thal) based on genetic testing, familial history, or other factors, but who has not yet displayed any manifestations or symptoms of the disease. In certain of these embodiments, treatment may be initiated prior to naturally occurring globin switching, i.e., prior to the transition from predominantly HbF to predominantly HbA. In other embodiments, treatment may be initiated after naturally occurring globin switching has occurred.

In certain embodiments, treatment is initiated after disease onset, e.g., 1, 2, 3, 4, 5, 6, 7, 8, 9, 10, 12, 16, 24, 36, or 48 or more months after onset of SCD or β-Thal or one or more symptoms associated therewith. In certain of these embodiments, treatment is initiated at an early stage of disease progression, e.g., when a subject has displayed only minor symptoms or only a subset of symptoms. Exemplary symptoms include, but are not limited to, anemia, diarrhea, fever, failure to thrive, sickle cell crises, vaso-occlusive crises, aplastic crises, and acute chest syndrome anemia, vaso-occlusion, hepatomegaly, thrombosis, pulmonary embolus, stroke, leg ulcer, cardiomyopathy, cardia arrhythmia, splenomegaly, delayed bone growth and/or puberty, and evidence of extramedullary erythropoiesis. In other embodiments, treatment is initiated well after disease onset or at a more advanced stage of disease progression, e.g., 1, 2, 3, 4, 5, 6, 7, 8, 9, 10, 12, 16, 24, 36, or 48 or more months after onset of SCD or β-Thal. While not wishing to be bound by theory, it is believed that this treatment will be effective if subjects present well into the course of illness.

In certain embodiments, the methods provided herein prevent or slow the development of one or more symptoms associated with the disease being treated. In certain embodiments, the methods provided herein result in prevention or delay of disease progression as compared to a subject who has not received the therapy. In certain embodiments, the methods provided herein result in the disease being cured entirely.

In certain embodiments, the methods provided herein are performed on a one-time basis. In other embodiments, the methods provided herein utilize multi-dose therapy.

In certain embodiments, a subject being treated using the methods provided herein is transfusion-dependent.

In certain embodiments, the methods provided herein comprise altering expression of one or more γ-globin genes (e.g., HBG1, HBG2) using CRISPR/Cas-mediated genome editing in a cell in vivo. In other embodiments, the methods provided herein comprise altering expression of one or more γ-globin genes using CRISPR/Cas-mediated genome editing in a cell ex vivo, then transplanting the cell into a subject. In certain of these embodiments, the cell is originally from the subject. In certain embodiments, the cell undergoing alteration is an adult erythroid cell. In other embodiments, the cell is a hematopoietic stem cell (HSC).

In certain embodiments, the methods provided herein comprise delivery to a cell of one or more gRNA molecules and one or more Cas9 polypeptides or nucleic acid sequences encoding a Cas9 polypeptide. In certain embodiments, the methods further comprise delivery of one or more nucleic acids, e.g., HDR donor templates.

In certain embodiments, one or more of these components (i.e., one or more gRNA molecules, one or more Cas9 polypeptides or nucleic acid sequences encoding a Cas9 polypeptide, and one or more nucleic acids, e.g., HDR donor templates) are delivered using one or more AAV vectors, lentiviral vectors, nanoparticles, or a combination thereof.

In certain embodiments, the methods provided herein are performed on a subject who has one or more mutations in an HBB gene, including one or more mutations associated with a 0-hemoglobinopathy such as SCD or β-Thal. Examples of such mutations include, but are not limited to, c.17A>T, c.−136C>G, c.92+1G>A, c.92+6T>C, c.93-21G>A, c.118C>T, c.316-106C>G, c.25_26delAA, c.27_28insG, c.92+5G>C, c.118C>T, c.135delC, c.315+1G>A, c.−78A>G, c.52A>T, c.59A>G, c.92+5G>C, c.124_127delTTCT, c.316-197C>T, c.−78A>G, c.52A>T, c.124_127delTTCT, c.316-197C>T, c.−138C>T, c.−79A>G, c.92+5G>C, c.75T>A, c.316-2A>G, and c.316-2A>C.

NHEJ-Mediated Introduction of an Indel within a γ-Globin Gene Regulatory Element

In certain embodiments, the methods provided herein utilize NHEJ-mediated insertions or deletions to disrupt all or a portion of a γ-globin gene regulatory element in order to increase expression of the γ-globin gene (e.g., HBG1, HBG2, or HBG1 and HBG2).

In certain embodiments, methods provided herein that utilize NHEJ comprise deletion or disruption of all or a portion of an HBG1 or HBG2 silencer element via NHEJ, resulting in inactivation of the silencer and a subsequent increase in HBG1 and/or HBG2 expression. In certain embodiments, NHEJ-mediated deletion results in removal of all or a part of c.−114 to −102 or −225 to −222 in one or both alleles of HBG1 and/or removal of all or a part of c.−114 to −102 in one or both alleles of HBG2. In certain of these embodiments, one or more nucleotides 5′ or 3′ to these regions are also deleted.

In certain embodiments, methods provided herein that utilize NHEJ comprise introduction of one or more breaks (e.g., single strand breaks or double strand breaks) within a γ-globin gene regulatory region, and in certain of these embodiments the one or more breaks are located sufficiently close to an HBG target position that a break-induced indel could be reasonably expected to span all or part of the HBG target position.

In certain embodiments, the targeting domain of a first gRNA molecule is configured to provide a cleavage event, e.g., a double strand break or a single strand break, sufficiently close to an HBG target position to allow NHEJ-mediated insertion or deletion at the HBG target position. In certain embodiments, the gRNA targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 250, 300, 350, 400, 450, or 500 nucleotides of an HBG target position. The break, e.g., a double strand or single strand break, can be positioned upstream or downstream of an HBG target position.

In certain embodiments, a second gRNA molecule comprising a second targeting domain is configured to provide a cleavage event, e.g., a double strand break or a single strand break, sufficiently close to an HBG target position to allow NHEJ-mediated insertion or deletion at the HBG target position, either alone or in combination with the break positioned by said first gRNA molecule. In certain embodiments, the targeting domains of the first and second gRNA molecules are configured such that a cleavage event, e.g., a double strand or single strand break, is positioned, independently for each of the gRNA molecules, within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position. In certain embodiments, the breaks, e.g., double strand or single strand breaks, are positioned on either side of a nucleotide of an HBG target position. In other embodiments, the breaks, e.g., double strand or single strand breaks, are both positioned on one side, e.g., upstream or downstream, of a nucleotide of an HBG target position.

In certain embodiments, a single strand break is accompanied by an additional single strand break, positioned by a second gRNA molecule, as discussed below. For example, the gRNA targeting domains may be configured such that a cleavage event, e.g., two single strand breaks, is positioned within 1, 2, 3, 4, 5, 10, 15, 20, 25, 30, 35, 40, 45, 50, 60, 70, 80, 90, 100, 150, 200, 250, 300, 350, 400, 450, or 500 nucleotides of an HBG target position. In certain embodiments, the first and second gRNA molecules are configured such that, when guiding a Cas9 nickase, a single strand break will be accompanied by an additional single strand break, positioned by a second gRNA, sufficiently close to one another to result in alteration of the HBG target position. In certain embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In certain embodiments, the two gRNA molecules are configured to position cuts at the same position, or within a few nucleotides of one another, on different strands, e.g., essentially mimicking a double strand break.

In certain embodiments, a double strand break can be accompanied by an additional double strand break, positioned by a second gRNA molecule, as is discussed below. For example, the targeting domain of a first gRNA molecule is configured such that a double strand break is positioned upstream of HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position; and the targeting domain of a second gRNA molecule is configured such that a double strand break is positioned downstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position.

In certain embodiments, a double strand break can be accompanied by two additional single strand breaks, positioned by a second gRNA molecule and a third gRNA molecule. For example, the targeting domain of a first gRNA molecule is configured such that a double strand break is positioned upstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position; and the targeting domains of a second and third gRNA molecule are configured such that two single strand breaks are positioned downstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position. In certain embodiments, the targeting domains of the first, second and third gRNA molecules are configured such that a cleavage event, e.g., a double strand or single strand break, is positioned, independently for each of the gRNA molecules.

In certain embodiments, a first and second single strand break can be accompanied by two additional single strand breaks positioned by a third and fourth gRNA molecule. For example, the targeting domains of a first and second gRNA molecule are configured such that two single strand breaks are positioned upstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position; and the targeting domains of a third and fourth gRNA molecule are configured such that two single strand breaks are positioned downstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position.

In certain embodiments, the methods provided herein comprise introducing an NHEJ-mediated deletion of a genomic sequence including an HBG target position. In certain embodiments, the methods comprise introduction of two double strand breaks, one 5′ to and the other 3′ to (i.e., flanking) the HBG target position. Two gRNAs, e.g., unimolecular (or chimeric) or modular gRNA molecules, are configured to position the two double strand breaks on opposite sides of the HBG target position. In certain embodiments, the first double strand break is positioned upstream of the mutation, and the second double strand break is positioned downstream of the mutation. In certain embodiments, the two double strand breaks are positioned to remove all or a portion of HBG1 c.−114 to −102, HBG1 4 bp del −225 to −222. In an embodiment, the breaks (i.e., the two double strand breaks) are positioned to avoid unwanted target chromosome elements, such as repeat elements, e.g., an Alu repeat, or the endogenous splice sites.

In other embodiments, the methods comprise the introduction of two sets of breaks, one double strand break and a pair of single strand breaks. The two sets flank the HBG target position, i.e., one set is 5′ to and the other is 3′ to the HBG target position. Two gRNAs, e.g., unimolecular (or chimeric) or modular gRNA molecules, are configured to position the two sets of breaks (either the double strand break or the pair of single strand breaks) on opposite sides of the HBG target position. In certain embodiments, the breaks (i.e., the two sets of breaks (either the double strand break or the pair of single strand breaks)) are positioned to avoid unwanted target chromosome elements, such as repeat elements, e.g., an Alu repeat, or the endogenous splice sites.

In other embodiments, the methods comprise the introduction of two pairs of single strand breaks, one 5′ to and the other 3′ to (i.e., flanking) the HBG target position. Two gRNAs, e.g., unimolecular (or chimeric) or modular gRNA molecules, are configured to position the two sets of breaks on opposite sides of the HBG target position. In certain embodiments, the breaks (i.e., the two pairs of single strand breaks) are positioned to avoid unwanted target chromosome elements, such as repeat elements, e.g., an Alu repeat, or the endogenous splice sites.

### HDR-Mediated Introduction of a Sequence Alteration in a γ-Globin Gene Regulatory Element

In certain embodiments, the methods provided herein utilize HDR to modify one or more nucleotides in a γ-globin gene regulatory element in order to increase expression of the γ-globin gene (e.g., HBG1, HBG2, or HBG1 and HBG2). In certain of these embodiments, HDR is utilized to incorporate one or more nucleotide modifications corresponding to naturally occurring mutations associated with HPFH. For example, in certain embodiments HDR is used to incorporate one or more of the following single nucleotide alterations into an HBG1 regulatory region: c.−114 C>T; c.−117 G>A; c.−158 C>T; c.−167 C>T; c.−170 G>A; c.−175 T>C; c.−175 T>G; c.−195 C>G; c.−196 C>T; c.−198 T>C; c.−201 C>T; c.−251 T>C; or c.−499 T>A. In other embodiments, HDR is used to incorporate one or more of the following single nucleotide alterations into an HBG2 regulatory region: c.−109 G>T; c.−114 C>A; c.−114 C>T; c.−157 C>T; c.−158 C>T; c.−167 C>T; c.−167 C>A; c.−175 T>C; c.−202 C>G; c.−211 C>T; c.−228 T>C; c.−255 C>G; c.−309 A>G; c.−369 C>G; c.−567 T>G.

In certain embodiments, the methods provided herein utilize HDR-mediated alteration (e.g., insertions or deletions) to disrupt all or a portion of a γ-globin gene regulatory element in order to increase expression of the γ-globin gene (e.g., HBG1, HBG2, or HBG1 and HBG2).

In certain embodiments, methods provided herein that utilize HDR comprise deletion or disruption of all or a portion of an HBG1 or HBG2 silencer element via HDR, resulting in inactivation of the silencer and a subsequent increase in HBG1 and/or HBG2 expression. In certain embodiments, HDR-mediated deletion results in removal of all or a part of c.−114 to −102 or −225 to −222 in one or both alleles of HBG1 and/or removal of all or a part of c.−114 to −102 in one or both alleles of HBG2. In certain of these embodiments, one or more nucleotides 5′ or 3′ to these regions are also deleted.

In certain embodiments, methods provided herein that utilize HDR comprise introduction of one or more breaks (e.g., single strand breaks or double strand breaks) within a γ-globin gene regulatory region, and in certain of these embodiments the one or more breaks are located sufficiently close to an HBG target position that a break-induced alteration could be reasonably expected to span all or part of the HBG target position.

In certain embodiments, HDR-mediated alteration may include the use of a template nucleic acid.

In certain embodiments, an HDR-mediated genetic alteration is incorporated into one 7-globin gene allele (e.g., one allele of HBG1 and/or HBG2). In another embodiment, the genetic alteration is incorporated into both alleles (e.g., both alleles of HBG1 and/or HBG2). In either situation, the treated subject exhibits increased γ-globin gene expression (e.g., HBG1, HBG2, or HBG1 and HBG2 expression).

In certain embodiments, methods provided herein that utilize HDR comprise introduction of one or more breaks (e.g., single strand breaks or double strand breaks) sufficiently close to (e.g., either 5′ or 3′ to) an HBG target position to allow for an alteration associated with HDR at the target position.

In certain embodiments, the targeting domain of a first gRNA molecule is configured to provide a cleavage event, e.g., a double strand break or a single strand break, sufficiently close to an HBG target position to allow for an alteration associated with HDR at the target position. In certain embodiments, the gRNA targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 250, 300, 350, 400, 450, or 500 nucleotides of an HBG target position. The break, e.g., a double strand or single strand break, can be positioned upstream or downstream of an HBG target position.

In certain embodiments, a second, third, and/or fourth gRNA molecule is configured to provide a cleavage event, e.g., a double strand break or a single strand break, sufficiently close to (e.g., either 5′ or 3′ to) an HBG target position to allow for an alteration associated with HDR at the target position. In certain embodiments, the gRNA targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 250, 300, 350, 400, 450 or 500 nucleotides of an HBG target position. The break, e.g., a double strand or single strand break, can be positioned upstream or downstream of the target position.

In certain embodiments, a single strand break is accompanied by an additional single strand break, positioned by a second, third and/or fourth gRNA molecule. For example, the gRNA targeting domains may be configured such that a cleavage event, e.g., the two single strand breaks, is positioned within 1, 2, 3, 4, 5, 10, 15, 20, 25, 30, 35, 40, 45, 50, 60, 70, 80, 90, 100, 150, 200, 250, 300, 350, 400, 450, or 500 nucleotides of an HBG target position. In certain embodiments, the first and second gRNA molecules are configured such that when guiding a Cas9 nickase, a single strand break will be accompanied by an additional single strand break positioned by a second gRNA sufficiently close to the first strand break to result in alteration of the HBG target position. In certain embodiments, the first and second gRNA molecules are configured such that a single strand break positioned by said second gRNA is within 10, 20, 30, 40, 50, 100, 200, 300, 400, 500, 600, 700, 800, 900, or 1000 nucleotides of the break positioned by said first gRNA molecule, e.g., when the Cas9 is a nickase. In certain embodiments, the two gRNA molecules are configured to position cuts at the same position, or within a few nucleotides of one another, on different strands, e.g., essentially mimicking a double strand break.

In certain embodiments, a double strand break can be accompanied by an additional double strand break, positioned by a second, third and/or fourth gRNA molecule. For example, the targeting domain of a first gRNA molecule may be configured such that a double strand break is positioned upstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position; and the targeting domain of a second gRNA molecule may be configured such that a double strand break is positioned downstream from the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position.

In certain embodiments, a double strand break can be accompanied by two additional single strand breaks, positioned by a second and third gRNA molecule. For example, the targeting domain of a first gRNA molecule may be configured such that a double strand break is positioned upstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position; and the targeting domains of a second and third gRNA molecule may be configured such that two single strand breaks are positioned downstream of the target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position. In certain embodiments, the targeting domains of the first, second and third gRNA molecules are configured such that a cleavage event, e.g., a double strand or single strand break, is positioned independently for each of the gRNA molecules.

In certain embodiments, first and second single strand breaks can be accompanied by two additional single strand breaks positioned by a third gRNA molecule and a fourth gRNA molecule. For example, the targeting domains of a first and second gRNA molecule may be configured such that two single strand breaks are positioned upstream of an HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position; and the targeting domains of a third and fourth gRNA molecule may be configured such that two single strand breaks are positioned downstream of the HBG target position, e.g., within 1, 2, 3, 4, 5, 10, 15, 20, 25, 30, 35, 40, 45, 50, 60, 70, 80, 90, 100, 150, 200, 250, 300, 350, 400, 450, or 500 nucleotides of the target position.

Guide RNA (gRNA) Molecules

A gRNA molecule, as that term is used herein, refers to a nucleic acid that promotes the specific targeting or homing of a gRNA molecule/Cas9 molecule complex to a target nucleic acid. gRNA molecules can be unimolecular (having a single RNA molecule) (e.g., chimeric), or modular (comprising more than one, and typically two, separate RNA molecules). The gRNA molecules provided herein comprise a targeting domain comprising, consisting of, or consisting essentially of a nucleic acid sequence fully or partially complementary to a target domain. In certain embodiments, the gRNA molecule further comprises one or more additional domains, including for example a first complementarity domain, a linking domain, a second complementarity domain, a proximal domain, a tail domain, and a 5′ extension domain. Each of these domains is discussed in detail below. In certain embodiments, one or more of the domains in the gRNA molecule comprises a nucleotide sequence identical to or sharing sequence homology with a naturally occurring sequence, e.g., from S. pyogenes, S. aureus, or S. thermophilus.

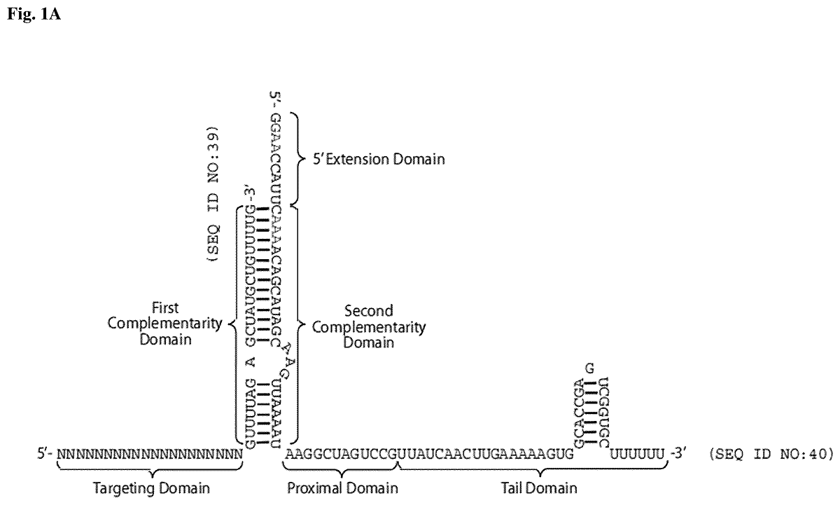

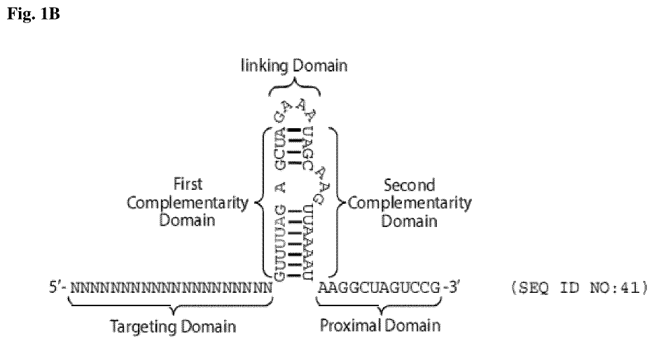

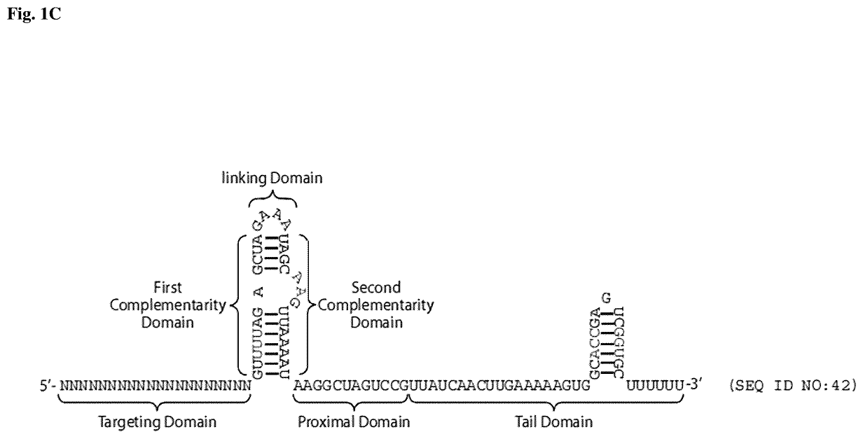

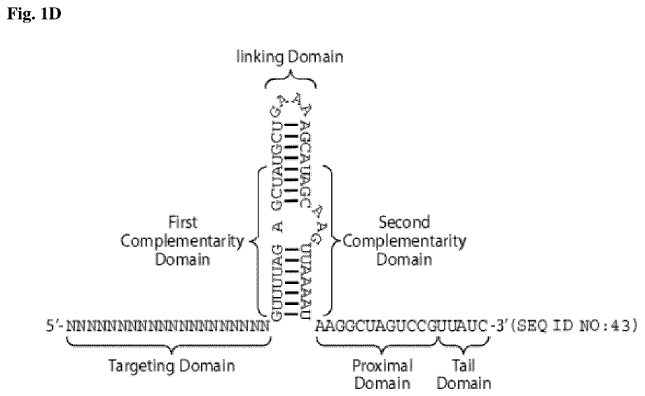

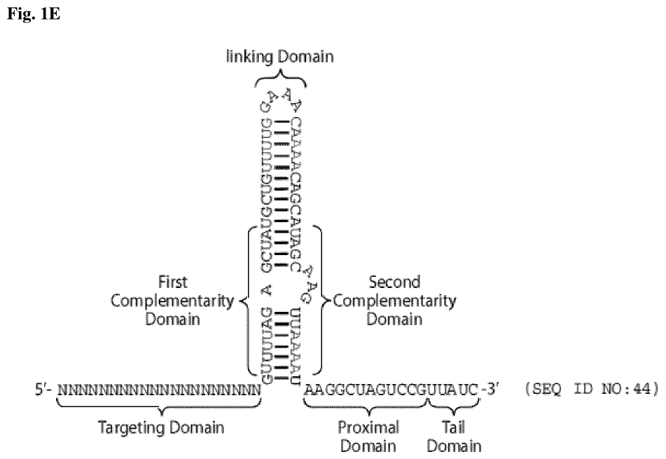

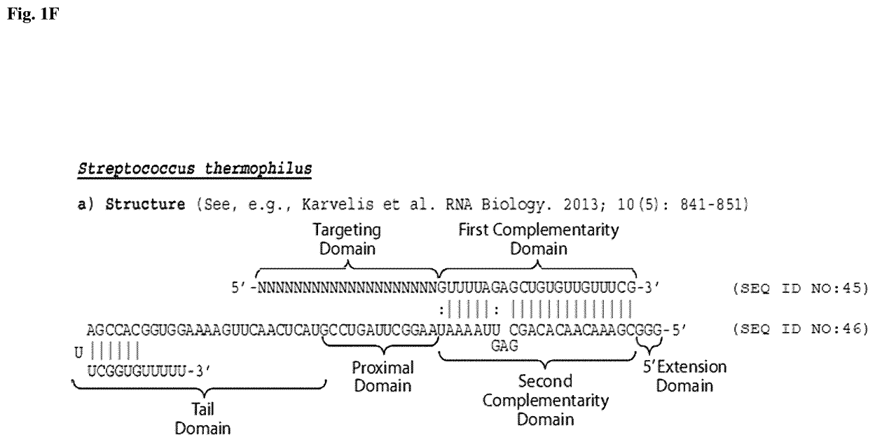

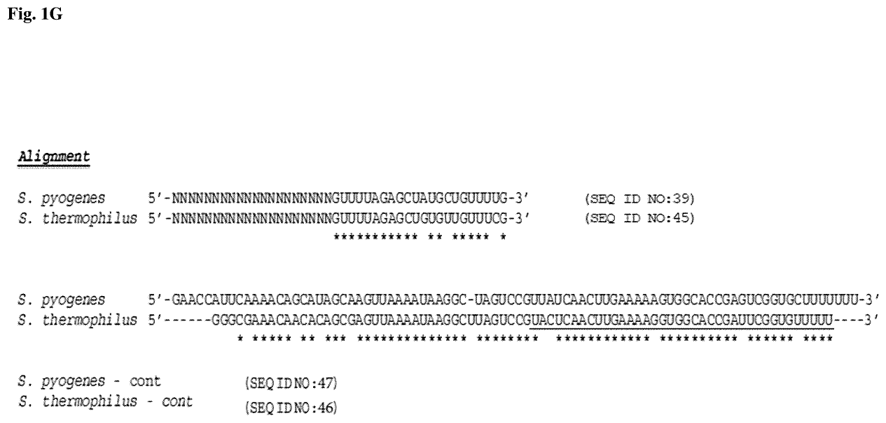

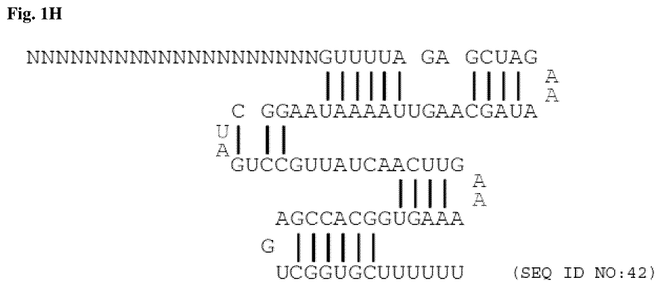

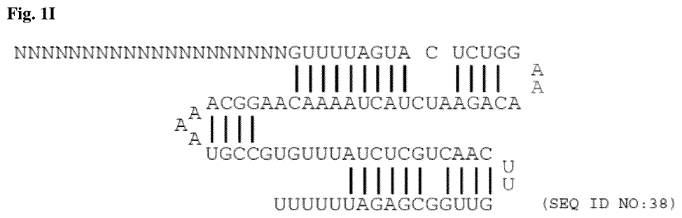

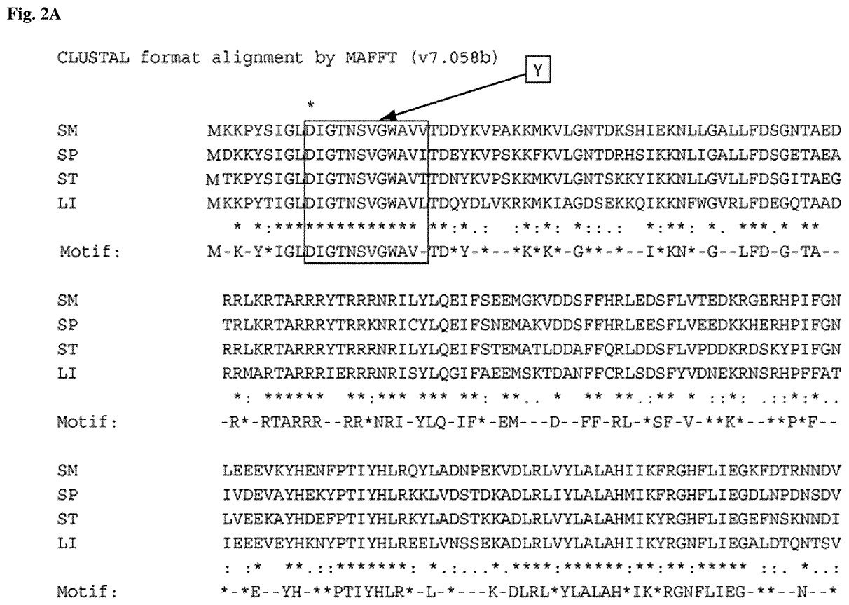

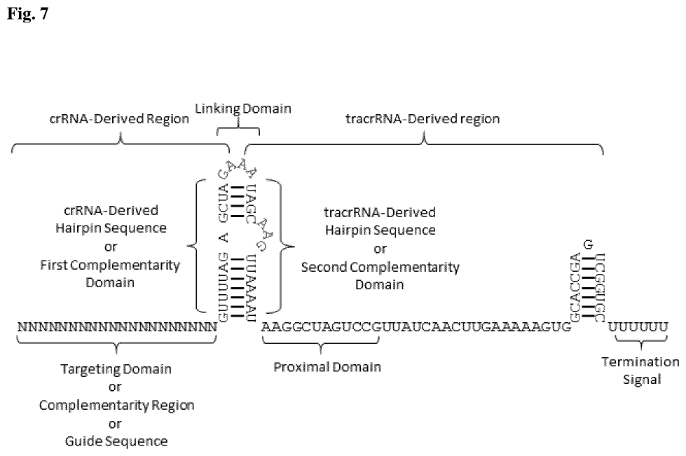

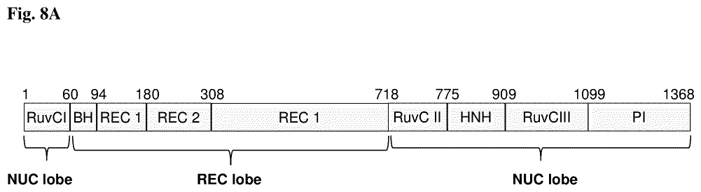

Several exemplary gRNA structures are provided in FIGS. 1A-1I. With regard to the three-dimensional form, or intra- or inter-strand interactions of an active form of a gRNA, regions of high complementarity are sometimes shown as duplexes in FIGS. 1A-1I and other depictions provided herein. FIG. 7 illustrates gRNA domain nomenclature using the gRNA sequence of SEQ ID NO:42, which contains one hairpin loop in the tracrRNA-derived region. In certain embodiments, a gRNA may contain more than one (e.g., two, three, or more) hairpin loops in this region (see, e.g., FIGS. 1H-1I).

In certain embodiments, a unimolecular, or chimeric, gRNA comprises, preferably from 5′ to 3′:
a targeting domain complementary to a target domain in a γ-globin gene regulatory region, e.g., a targeting domain from any of SEQ ID NOs:251-901;
a first complementarity domain;
a linking domain;
a second complementarity domain (which is complementary to the first complementarity domain);
a proximal domain; and
optionally, a tail domain.

In certain embodiments, a modular gRNA comprises:
a first strand comprising, preferably from 5′ to 3′:
   a targeting domain complementary to a target domain in in a γ-globin gene regulatory region, e.g., a targeting domain from any of SEQ ID NOs:251-901; and
   a first complementarity domain; and
a second strand, comprising, preferably from 5′ to 3′:
   optionally, a 5′ extension domain;
   a second complementarity domain;
   a proximal domain; and
   optionally, a tail domain.

### Targeting Domain

The targeting domain (sometimes referred to alternatively as the guide sequence or complementarity region) comprises, consists of, or consists essentially of a nucleic acid sequence that is complementary or partially complementary to a target nucleic acid sequence in a γ-globin gene regulatory region. The nucleic acid sequence in a γ-globin gene regulatory region to which all or a portion of the targeting domain is complementary or partially complementary is referred to herein as the target domain. In certain embodiments, the target domain comprises an HBG target position. In other embodiments, an HBG target position lies outside (i.e., upstream or downstream of) the target domain. In certain embodiments, the target domain is located entirely within a γ-globin gene regulatory region, e.g., in a regulatory element associated with a γ-globin gene or a regulatory element associated with a gene encoding a repressor of γ-globin gene expression. In other embodiments, all or part of the target domain is located outside of γ-globin gene regulatory region, e.g., in an HBG1 or HBG2 coding region, exon, or intron.

Methods for selecting targeting domains are known in the art (see, e.g., Fu 2014; Sternberg 2014). Examples of suitable targeting domains for use in the methods, compositions, and kits described herein include those set forth in SEQ ID NOs:251-901.

The strand of the target nucleic acid comprising the target domain is referred to herein as the complementary strand because it is complementary to the targeting domain sequence. Since the targeting domain is part of a gRNA molecule, it comprises the base uracil (U) rather than thymine (T); conversely, any DNA molecule encoding the gRNA molecule will comprise thymine rather than uracil. In a targeting domain/target domain pair, the uracil bases in the targeting domain will pair with the adenine bases in the target domain. In certain embodiments, the degree of complementarity between the targeting domain and target domain is sufficient to allow targeting of a Cas9 molecule to the target nucleic acid.

In certain embodiments, the targeting domain comprises a core domain and an optional secondary domain. In certain of these embodiments, the core domain is located 3′ to the secondary domain, and in certain of these embodiments the core domain is located at or near the 3′ end of the targeting domain. In certain of these embodiments, the core domain consists of or consists essentially of about 8 to about 13 nucleotides at the 3′ end of the targeting domain. In certain embodiments, only the core domain is complementary or partially complementary to the corresponding portion of the target domain, and in certain of these embodiments the core domain is fully complementary to the corresponding portion of the target domain. In other embodiments, the secondary domain is also complementary or partially complementary to a portion of the target domain. In certain embodiments, the core domain is complementary or partially complementary to a core domain target in the target domain, while the secondary domain is complementary or partially complementary to a secondary domain target in the target domain. In certain embodiments, the core domain and secondary domain have the same degree of complementarity with their respective corresponding portions of the target domain. In other embodiments, the degree of complementarity between the core domain and its target and the degree of complementarity between the secondary domain and its target may differ. In certain of these embodiments, the core domain may have a higher degree of complementarity for its target than the secondary domain, whereas in other embodiments the secondary domain may have a higher degree of complementarity than the core domain.

In certain embodiments, the targeting domain and/or the core domain within the targeting domain is 3 to 100, 5 to 100, 10 to 100, or 20 to 100 nucleotides in length, and in certain of these embodiments the targeting domain or core domain is 3 to 15, 3 to 20, 5 to 20, 10 to 20, 15 to 20, 5 to 50, 10 to 50, or 20 to 50 nucleotides in length. In certain embodiments, the targeting domain and/or the core domain within the targeting domain is 6, 7, 8, 9, 10, 11, 12, 13, 14, 15, 16, 17, 18, 19, 20, 21, 22, 23, 24, 25, or 26 nucleotides in length. In certain embodiments, the targeting domain and/or the core domain within the targeting domain is 6+/−2, 7+/−2, 8+/−2, 9+/−2, 10+/−2, 10+/−4, 10+/−5, 11+/−2, 12+/−2, 13+/−2, 14+/−2, 15+/−2, or 16+−2, 20+/−5, 30+/−5, 40+/−5, 50+/−5, 60+/−5, 70+/−5, 80+/−5, 90+/−5, or 100+/−5 nucleotides in length.

In certain embodiments wherein the targeting domain includes a core domain, the core domain is 3 to 20 nucleotides in length, and in certain of these embodiments the core domain 5 to 15 or 8 to 13 nucleotides in length. In certain embodiments wherein the targeting domain includes a secondary domain, the secondary domain is 0, 1, 2, 3, 4, 5, 6, 7, 8, 9, 10, 11, 12, 13, 14 or 15 nucleotides in length. In certain embodiments wherein the targeting domain comprises a core domain that is 8 to 13 nucleotides in length, the targeting domain is 26, 25, 24, 23, 22, 21, 20, 19, 18, 17, or 16 nucleotides in length, and the secondary domain is 13 to 18, 12 to 17, 11 to 16, 10 to 15, 9 to 14, 8 to 13, 7 to 12, 6 to 11, 5 to 10, 4 to 9, or 3 to 8 nucleotides in length, respectively.

In certain embodiments, the targeting domain is fully complementary to the target domain. Likewise, where the targeting domain comprises a core domain and/or a secondary domain, in certain embodiments one or both of the core domain and the secondary domain are fully complementary to the corresponding portions of the target domain. In other embodiments, the targeting domain is partially complementary to the target domain, and in certain of these embodiments where the targeting domain comprises a core domain and/or a secondary domain, one or both of the core domain and the secondary domain are partially complementary to the corresponding portions of the target domain. In certain of these embodiments, the nucleic acid sequence of the targeting domain, or the core domain or targeting domain within the targeting domain, is at least 80, 85, 90, or 95% complementary to the target domain or to the corresponding portion of the target domain. In certain embodiments, the targeting domain and/or the core or secondary domains within the targeting domain include one or more nucleotides that are not complementary with the target domain or a portion thereof, and in certain of these embodiments the targeting domain and/or the core or secondary domains within the targeting domain include 1, 2, 3, 4, 5, 6, 7, or 8 nucleotides that are not complementary with the target domain. In certain embodiments, the core domain includes 1, 2, 3, 4, or 5 nucleotides that are not complementary with the corresponding portion of the target domain. In certain embodiments wherein the targeting domain includes one or more nucleotides that are not complementary with the target domain, one or more of said non-complementary nucleotides are located within five nucleotides of the 5′ or 3′ end of the targeting domain. In certain of these embodiments, the targeting domain includes 1, 2, 3, 4, or 5 nucleotides within five nucleotides of its 5′ end, 3′ end, or both its 5′ and 3′ ends that are not complementary to the target domain. In certain embodiments wherein the targeting domain includes two or more nucleotides that are not complementary to the target domain, two or more of said non-complementary nucleotides are adjacent to one another, and in certain of these embodiments the two or more consecutive non-complementary nucleotides are located within five nucleotides of the 5′ or 3′ end of the targeting domain. In other embodiments, the two or more consecutive non-complementary nucleotides are both located more than five nucleotides from the 5′ and 3′ ends of the targeting domain.

In certain embodiments, the targeting domain, core domain, and/or secondary domain do not comprise any modifications. In other embodiments, the targeting domain, core domain, and/or secondary domain, or one or more nucleotides therein, have a modification, including but not limited to the modifications set forth below. In certain embodiments, one or more nucleotides of the targeting domain, core domain, and/or secondary domain may comprise a 2′ modification (e.g., a modification at the 2′ position on ribose), e.g., a 2-acetylation, e.g., a 2′ methylation. In certain embodiments, the backbone of the targeting domain can be modified with a phosphorothioate. In certain embodiments, modifications to one or more nucleotides of the targeting domain, core domain, and/or secondary domain render the targeting domain and/or the gRNA comprising the targeting domain less susceptible to degradation or more bio-compatible, e.g., less immunogenic. In certain embodiments, the targeting domain and/or the core or secondary domains include 1, 2, 3, 4, 5, 6, 7, or 8 or more modifications, and in certain of these embodiments the targeting domain and/or core or secondary domains include 1, 2, 3, or 4 modifications within five nucleotides of their respective 5′ ends and/or 1, 2, 3, or 4 modifications within five nucleotides of their respective 3′ ends. In certain embodiments, the targeting domain and/or the core or secondary domains comprise modifications at two or more consecutive nucleotides.

In certain embodiments wherein the targeting domain includes core and secondary domains, the core and secondary domains contain the same number of modifications. In certain of these embodiments, both domains are free of modifications. In other embodiments, the core domain includes more modifications than the secondary domain, or vice versa.

In certain embodiments, modifications to one or more nucleotides in the targeting domain, including in the core or secondary domains, are selected to not interfere with targeting efficacy, which can be evaluated by testing a candidate modification using a system as set forth below. gRNAs having a candidate targeting domain having a selected length, sequence, degree of complementarity, or degree of modification can be evaluated using a system as set forth below. The candidate targeting domain can be placed, either alone or with one or more other candidate changes in a gRNA molecule/Cas9 molecule system known to be functional with a selected target, and evaluated.

In certain embodiments, all of the modified nucleotides are complementary to and capable of hybridizing to corresponding nucleotides present in the target domain. In another embodiment, 1, 2, 3, 4, 5, 6, 7, or 8 or more modified nucleotides are not complementary to or capable of hybridizing to corresponding nucleotides present in the target domain.

FIGS. 1A-1I provide examples of the placement of the targeting domain within a gRNA molecule.

### First and Second Complementarity Domains

The first and second complementarity (sometimes referred to alternatively as the crRNA-derived hairpin sequence and tracrRNA-derived hairpin sequences, respectively) domains are fully or partially complementary to one another. In certain embodiments, the degree of complementarity is sufficient for the two domains to form a duplexed region under at least some physiological conditions. In certain embodiments, the degree of complementarity between the first and second complementarity domains, together with other properties of the gRNA, is sufficient to allow targeting of a Cas9 molecule to a target nucleic acid. Examples of first and second complementarity domains are set forth in FIGS. 1A-1G.

In certain embodiments (see, e.g., FIGS. 1A-1B) the first and/or second complementarity domain includes one or more nucleotides that lack complementarity with the corresponding complementarity domain. In certain embodiments, the first and/or second complementarity domain includes 1, 2, 3, 4, 5, or 6 nucleotides that do not complement with the corresponding complementarity domain. For example, the second complementarity domain may contain 1, 2, 3, 4, 5, or 6 nucleotides that do not pair with corresponding nucleotides in the first complementarity domain. In certain embodiments, the nucleotides on the first or second complementarity domain that do not complement with the corresponding complementarity domain loop out from the duplex formed between the first and second complementarity domains. In certain of these embodiments, the unpaired loop-out is located on the second complementarity domain, and in certain of these embodiments the unpaired region begins 1, 2, 3, 4, 5, or 6 nucleotides from the 5′ end of the second complementarity domain.

In certain embodiments, the first complementarity domain is 5 to 30, 5 to 25, 7 to 25, 5 to 24, 5 to 23, 7 to 22, 5 to 22, 5 to 21, 5 to 20, 7 to 18, 7 to 15, 9 to 16, or 10 to 14 nucleotides in length, and in certain of these embodiments the first complementarity domain is 5, 6, 7, 8, 9, 10, 11, 12, 13, 14, 15, 16, 17, 18, 19, 20, 21, 22, 23, 24, or 25 nucleotides in length. In certain embodiments, the second complementarity domain is 5 to 27, 7 to 27, 7 to 25, 5 to 24, 5 to 23, 5 to 22, 5 to 21, 7 to 20, 5 to 20, 7 to 18, 7 to 17, 9 to 16, or 10 to 14 nucleotides in length, and in certain of these embodiments the second complementarity domain is 5, 6, 7, 8, 9, 10, 11, 12, 13, 14, 15, 16, 17, 18, 19, 20, 21, 22, 23, 24, 25, or 26 nucleotides in length. In certain embodiments, the first and second complementarity domains are each independently 6+/−2, 7+/−2, 8+/−2, 9+/−2, 10+/−2, 11+/−2, 12+/−2, 13+/−2, 14+/−2, 15+/−2, 16+/−2, 17+/−2, 18+/−2, 19+/−2, or 20+/−2, 21+/−2, 22+/−2, 23+/−2, or 24+/−2 nucleotides in length. In certain embodiments, the second complementarity domain is longer than the first complementarity domain, e.g., 2, 3, 4, 5, or 6 nucleotides longer.

In certain embodiments, the first and/or second complementarity domains each independently comprise three subdomains, which, in the 5′ to 3′ direction are: a 5′ subdomain, a central subdomain, and a 3′ subdomain. In certain embodiments, the 5′ subdomain and 3′ subdomain of the first complementarity domain are fully or partially complementary to the 3′ subdomain and 5′ subdomain, respectively, of the second complementarity domain.

In certain embodiments, the 5′ subdomain of the first complementarity domain is 4 to 9 nucleotides in length, and in certain of these embodiments the 5′ domain is 4, 5, 6, 7, 8, or 9 nucleotides in length. In certain embodiments, the 5′ subdomain of the second complementarity domain is 3 to 25, 4 to 22, 4 to 18, or 4 to 10 nucleotides in length, and in certain of these embodiments the 5′ domain is 3, 4, 5, 6, 7, 8, 9, 10, 11, 12, 13, 14, 15, 16, 17, 18, 19, 20, 21, 22, 23, 24, or 25 nucleotides in length. In certain embodiments, the central subdomain of the first complementarity domain is 1, 2, or 3 nucleotides in length. In certain embodiments, the central subdomain of the second complementarity domain is 1, 2, 3, 4, or 5 nucleotides in length. In certain embodiments, the 3′ subdomain of the first complementarity domain is 3 to 25, 4 to 22, 4 to 18, or 4 to 10 nucleotides in length, and in certain of these embodiments the 3′ subdomain is 3, 4, 5, 6, 7, 8, 9, 10, 11, 12, 13, 14, 15, 16, 17, 18, 19, 20, 21, 22, 23, 24, or 25 nucleotides in length. In certain embodiments, the 3′ subdomain of the second complementarity domain is 4 to 9, e.g., 4, 5, 6, 7, 8, or 9 nucleotides in length.

The first and/or second complementarity domains can share homology with, or be derived from, naturally occurring or reference first and/or second complementarity domains. In certain of these embodiments, the first and/or second complementarity domains have at least 50%, 60%, 70%, 80%, 85%, 90%, or 95% homology with, or differ by no more than 1, 2, 3, 4, 5, or 6 nucleotides from, the naturally occurring or reference first and/or second complementarity domain. In certain of these embodiments, the first and/or second complementarity domains may have at least 50%, 60%, 70%, 80%, 85%, 90%, or 95% homology with homology with a first and/or second complementarity domain from S. pyogenes or S. aureus.

In certain embodiments, the first and/or second complementarity domains do not comprise any modifications. In other embodiments, the first and/or second complementarity domains or one or more nucleotides therein have a modification, including but not limited to a modification set forth below. In certain embodiments, one or more nucleotides of the first and/or second complementarity domain may comprise a 2′ modification (e.g., a modification at the 2′ position on ribose), e.g., a 2-acetylation, e.g., a 2′ methylation. In certain embodiments, the backbone of the targeting domain can be modified with a phosphorothioate. In certain embodiments, modifications to one or more nucleotides of the first and/or second complementarity domain render the first and/or second complementarity domain and/or the gRNA comprising the first and/or second complementarity less susceptible to degradation or more bio-compatible, e.g., less immunogenic. In certain embodiments, the first and/or second complementarity domains each independently include 1, 2, 3, 4, 5, 6, 7, or 8 or more modifications, and in certain of these embodiments the first and/or second complementarity domains each independently include 1, 2, 3, or 4 modifications within five nucleotides of their respective 5′ ends, 3′ ends, or both their 5′ and 3′ ends. In other embodiments, the first and/or second complementarity domains each independently contain no modifications within five nucleotides of their respective 5′ ends, 3′ ends, or both their 5′ and 3′ ends. In certain embodiments, one or both of the first and second complementarity domains comprise modifications at two or more consecutive nucleotides.

In certain embodiments, modifications to one or more nucleotides in the first and/or second complementarity domains are selected to not interfere with targeting efficacy, which can be evaluated by testing a candidate modification in a system as set forth below. gRNAs having a candidate first or second complementarity domain having a selected length, sequence, degree of complementarity, or degree of modification can be evaluated in a system as set forth below. The candidate complementarity domain can be placed, either alone or with one or more other candidate changes in a gRNA molecule/Cas9 molecule system known to be functional with a selected target, and evaluated.

In certain embodiments, the duplexed region formed by the first and second complementarity domains is, for example, 6, 7, 8, 9, 10, 11, 12, 13, 14, 15, 16, 17, 18, 19, 20, 21, or 22 bp in length, excluding any looped out or unpaired nucleotides.

In certain embodiments, the first and second complementarity domains, when duplexed, comprise 11 paired nucleotides (see, for e.g., gRNA of SEQ ID NO:48). In certain embodiments, the first and second complementarity domains, when duplexed, comprise 15 paired nucleotides (see, e.g., gRNA of SEQ ID NO:50). In certain embodiments, the first and second complementarity domains, when duplexed, comprise 16 paired nucleotides (see, e.g., gRNA of SEQ ID NO:51). In certain embodiments, the first and second complementarity domains, when duplexed, comprise 21 paired nucleotides (see, e.g., gRNA of SEQ ID NO:29).

In certain embodiments, one or more nucleotides are exchanged between the first and second complementarity domains to remove poly-U tracts. For example, nucleotides 23 and 48 or nucleotides 26 and 45 of the gRNA of SEQ ID NO:48 may be exchanged to generate the gRNA of SEQ ID NOs:49 or 31, respectively. Similarly, nucleotides 23 and 39 of the gRNA of SEQ ID NO:29 may be exchanged with nucleotides 50 and 68 to generate the gRNA of SEQ ID NO:30.

### Linking Domain

The linking domain is disposed between and serves to link the first and second complementarity domains in a unimolecular or chimeric gRNA. FIGS. 1B-1E provide examples of linking domains. In certain embodiments, part of the linking domain is from a crRNA-derived region, and another part is from a tracrRNA-derived region.

In certain embodiments, the linking domain links the first and second complementarity domains covalently. In certain of these embodiments, the linking domain consists of or comprises a covalent bond. In other embodiments, the linking domain links the first and second complementarity domains non-covalently. In certain embodiments, the linking domain is ten or fewer nucleotides in length, e.g., 1, 2, 3, 4, 5, 6, 7, 8, 9, or 10 nucleotides. In other embodiments, the linking domain is greater than 10 nucleotides in length, e.g., 11, 12, 13, 14, 15, 16, 17, 18, 19, 20, 21, 22, 23, 24, or 25 or more nucleotides. In certain embodiments, the linking domain is 2 to 50, 2 to 40, 2 to 30, 2 to 20, 2 to 10, 2 to 5, 10 to 100, 10 to 90, 10 to 80, 10 to 70, 10 to 60, 10 to 50, 10 to 40, 10 to 30, 10 to 20, 10 to 15, 20 to 100, 20 to 90, 20 to 80, 20 to 70, 20 to 60, 20 to 50, 20 to 40, 20 to 30, or 20 to 25 nucleotides in length. In certain embodiments, the linking domain is 10+/−5, 20+/−5, 20+/−10, 30+/−5, 30+/−10, 40+/−5, 40+/−10, 50+/−5, 50+/−10, 60+/−5, 60+/−10, 70+/−5, 70+/−10, 80+/−5, 80+/−10, 90+/−5, 90+/−10, 100+/−5, or 100+/−10 nucleotides in length.

In certain embodiments, the linking domain shares homology with, or is derived from, a naturally occurring sequence, e.g., the sequence of a tracrRNA that is 5′ to the second complementarity domain. In certain embodiments, the linking domain has at least 50%, 60%, 70%, 80%, 90%, or 95% homology with or differs by no more than 1, 2, 3, 4, 5, or 6 nucleotides from a linking domain disclosed herein, e.g., the linking domains of FIGS. 1B-1E.

In certain embodiments, the linking domain does not comprise any modifications. In other embodiments, the linking domain or one or more nucleotides therein have a modification, including but not limited to the modifications set forth below. In certain embodiments, one or more nucleotides of the linking domain may comprise a 2′ modification (e.g., a modification at the 2′ position on ribose), e.g., a 2-acetylation, e.g., a 2′ methylation. In certain embodiments, the backbone of the linking domain can be modified with a phosphorothioate. In certain embodiments, modifications to one or more nucleotides of the linking domain render the linking domain and/or the gRNA comprising the linking domain less susceptible to degradation or more bio-compatible, e.g., less immunogenic. In certain embodiments, the linking domain includes 1, 2, 3, 4, 5, 6, 7, or 8 or more modifications, and in certain of these embodiments the linking domain includes 1, 2, 3, or 4 modifications within five nucleotides of its 5′ and/or 3′ end. In certain embodiments, the linking domain comprises modifications at two or more consecutive nucleotides.

In certain embodiments, modifications to one or more nucleotides in the linking domain are selected to not interfere with targeting efficacy, which can be evaluated by testing a candidate modification in a system as set forth below. gRNAs having a candidate linking domain having a selected length, sequence, degree of complementarity, or degree of modification can be evaluated in a system as set forth below. The candidate linking domain can be placed, either alone or with one or more other candidate changes in a gRNA molecule/Cas9 molecule system known to be functional with a selected target, and evaluated.

In certain embodiments, the linking domain comprises a duplexed region, typically adjacent to or within 1, 2, or 3 nucleotides of the 3′ end of the first complementarity domain and/or the 5′ end of the second complementarity domain. In certain of these embodiments, the duplexed region of the linking region is 10+/−5, 15+/−5, 20+/−5, 20+/−10, or 30+/−5 bp in length. In certain embodiments, the duplexed region of the linking domain is 1, 2, 3, 4, 5, 6, 7, 8, 9, 10, 11, 12, 13, 14, or 15 bp in length. In certain embodiments, the sequences forming the duplexed region of the linking domain are fully complementarity. In other embodiments, one or both of the sequences forming the duplexed region contain one or more nucleotides (e.g., 1, 2, 3, 4, 5, 6, 7, or 8 nucleotides) that are not complementary with the other duplex sequence.

### 5′ Extension Domain

In certain embodiments, a modular gRNA as disclosed herein comprises a 5′ extension domain, i.e., one or more additional nucleotides 5′ to the second complementarity domain (see, e.g., FIG. 1A). In certain embodiments, the 5′ extension domain is 2 to 10 or more, 2 to 9, 2 to 8, 2 to 7, 2 to 6, 2 to 5, or 2 to 4 nucleotides in length, and in certain of these embodiments the 5′ extension domain is 2, 3, 4, 5, 6, 7, 8, 9, or 10 or more nucleotides in length.

In certain embodiments, the 5′ extension domain nucleotides do not comprise modifications, e.g., modifications of the type provided below. However, in certain embodiments, the 5′ extension domain comprises one or more modifications, e.g., modifications that it render it less susceptible to degradation or more bio-compatible, e.g., less immunogenic. By way of example, the backbone of the 5′ extension domain can be modified with a phosphorothioate, or other modification(s) as set forth below. In certain embodiments, a nucleotide of the 5′ extension domain can comprise a 2′ modification (e.g., a modification at the 2′ position on ribose), e.g., a 2-acetylation, e.g., a 2′ methylation, or other modification(s) as set forth below.

In certain embodiments, the 5′ extension domain can comprise as many as 1, 2, 3, 4, 5, 6, 7, or 8 modifications. In certain embodiments, the 5′ extension domain comprises as many as 1, 2, 3, or 4 modifications within 5 nucleotides of its 5′ end, e.g., in a modular gRNA molecule. In certain embodiments, the 5′ extension domain comprises as many as 1, 2, 3, or 4 modifications within 5 nucleotides of its 3′ end, e.g., in a modular gRNA molecule.

In certain embodiments, the 5′ extension domain comprises modifications at two consecutive nucleotides, e.g., two consecutive nucleotides that are within 5 nucleotides of the 5′ end of the 5′ extension domain, within 5 nucleotides of the 3′ end of the 5′ extension domain, or more than 5 nucleotides away from one or both ends of the 5′ extension domain. In certain embodiments, no two consecutive nucleotides are modified within 5 nucleotides of the 5′ end of the 5′ extension domain, within 5 nucleotides of the 3′ end of the 5′ extension domain, or within a region that is more than 5 nucleotides away from one or both ends of the 5′ extension domain. In certain embodiments, no nucleotide is modified within 5 nucleotides of the 5′ end of the 5′ extension domain, within 5 nucleotides of the 3′ end of the 5′ extension domain, or within a region that is more than 5 nucleotides away from one or both ends of the 5′ extension domain.

Modifications in the 5′ extension domain can be selected so as to not interfere with gRNA molecule efficacy, which can be evaluated by testing a candidate modification in a system as set forth below. gRNAs having a candidate 5′ extension domain having a selected length, sequence, degree of complementarity, or degree of modification, can be evaluated in a system as set forth below. The candidate 5′ extension domain can be placed, either alone, or with one or more other candidate changes in a gRNA molecule/Cas9 molecule system known to be functional with a selected target and evaluated.

In certain embodiments, the 5′ extension domain has at least 60, 70, 80, 85, 90, or 95% homology with, or differs by no more than 1, 2, 3, 4, 5, or 6 nucleotides from, a reference 5′ extension domain, e.g., a naturally occurring, e.g., an S. pyogenes, S. aureus, or S. thermophilus, 5′ extension domain, or a 5′ extension domain described herein, e.g., from FIGS. 1A-1G.

### Proximal Domain

FIGS. 1A-1G provide examples of proximal domains.

In certain embodiments, the proximal domain is 5 to 20 or more nucleotides in length, e.g., 5, 6, 7, 8, 9, 10, 11, 12, 13, 14, 15, 16, 17, 18, 19, 20, 21, 22, 23, 24, 25, or 26 nucleotides in length. In certain of these embodiments, the proximal domain is 6+/−2, 7+/−2, 8+/−2, 9+/−2, 10+/−2, 11+/−2, 12+/−2, 13+/−2, 14+/−2, 14+/−2, 16+/−2, 17+/−2, 18+/−2, 19+/−2, or 20+/−2 nucleotides in length. In certain embodiments, the proximal domain is 5 to 20, 7, to 18, 9 to 16, or 10 to 14 nucleotides in length.

In certain embodiments, the proximal domain can share homology with or be derived from a naturally occurring proximal domain. In certain of these embodiments, the proximal domain has at least 50%, 60%, 70%, 80%, 85%, 90%, or 95% homology with or differs by no more than 1, 2, 3, 4, 5, or 6 nucleotides from a proximal domain disclosed herein, e.g., an S. pyogenes, S. aureus, or S. thermophilus proximal domain, including those set forth in FIGS. 1A-1G.

In certain embodiments, the proximal domain does not comprise any modifications. In other embodiments, the proximal domain or one or more nucleotides therein have a modification, including but not limited to the modifications set forth in herein. In certain embodiments, one or more nucleotides of the proximal domain may comprise a 2′ modification (e.g., a modification at the 2′ position on ribose), e.g., a 2-acetylation, e.g., a 2′ methylation. In certain embodiments, the backbone of the proximal domain can be modified with a phosphorothioate. In certain embodiments, modifications to one or more nucleotides of the proximal domain render the proximal domain and/or the gRNA comprising the proximal domain less susceptible to degradation or more bio-compatible, e.g., less immunogenic. In certain embodiments, the proximal domain includes 1, 2, 3, 4, 5, 6, 7, or 8 or more modifications, and in certain of these embodiments the proximal domain includes 1, 2, 3, or 4 modifications within five nucleotides of its 5′ and/or 3′ end. In certain embodiments, the proximal domain comprises modifications at two or more consecutive nucleotides.

In certain embodiments, modifications to one or more nucleotides in the proximal domain are selected to not interfere with targeting efficacy, which can be evaluated by testing a candidate modification in a system as set forth below. gRNAs having a candidate proximal domain having a selected length, sequence, degree of complementarity, or degree of modification can be evaluated in a system as set forth below. The candidate proximal domain can be placed, either alone or with one or more other candidate changes in a gRNA molecule/Cas9 molecule system known to be functional with a selected target, and evaluated.

### Tail Domain

A broad spectrum of tail domains are suitable for use in the gRNA molecules disclosed herein. FIGS. 1A and 1C-1G provide examples of such tail domains.

In certain embodiments, the tail domain is absent. In other embodiments, the tail domain is 1 to 100 or more nucleotides in length, e.g., 1, 2, 3, 4, 5, 6, 7, 8, 9, 10, 20, 30, 40, 50, 60, 70, 80, 90, or 100 nucleotides in length. In certain embodiments, the tail domain is 1 to 5, 1 to 10, 1 to 15, 1 to 20, 1 to 50, 10 to 100, 20 to 100, 10 to 90, 20 to 90, 10 to 80, 20 to 80, 10 to 70, 20 to 70, 10 to 60, 20 to 60, 10 to 50, 20 to 50, 10 to 40, 20 to 40, 10 to 30, 20 to 30, 20 to 25, 10 to 20, or 10 to 15 nucleotides in length. In certain embodiments, the tail domain is 5+/−5, 10+/−5, 20+/−10, 20+/−5, 25+/−10, 30+/−10, 30+/−5, 40+/−10, 40+/−5, 50+/−10, 50+/−5, 60+/−10, 60+/−5, 70+/−10, 70+/−5, 80+/−10, 80+/−5, 90+/−10, 90+/−5, 100+/−10, or 100+/−5 nucleotides in length.

In certain embodiments, the tail domain can share homology with or be derived from a naturally occurring tail domain or the 5′ end of a naturally occurring tail domain. In certain of these embodiments, the proximal domain has at least 50%, 60%, 70%, 80%, 85%, 90%, or 95% homology with or differs by no more than 1, 2, 3, 4, 5, or 6 nucleotides from a naturally occurring tail domain disclosed herein, e.g., an S. pyogenes, S. aureus, or S. thermophilus tail domain, including those set forth in FIGS. 1A and 1C-1G.

In certain embodiments, the tail domain includes sequences that are complementary to each other and which, under at least some physiological conditions, form a duplexed region. In certain of these embodiments, the tail domain comprises a tail duplex domain which can form a tail duplexed region. In certain embodiments, the tail duplexed region is 3, 4, 5, 6, 7, 8, 9, 10, 11, or 12 bp in length. In certain embodiments, the tail domain comprises a single stranded domain 3′ to the tail duplex domain that does not form a duplex. In certain of these embodiments, the single stranded domain is 3 to 10 nucleotides in length, e.g., 3, 4, 5, 6, 7, 8, 9, 10, or 4 to 6 nucleotides in length.

In certain embodiments, the tail domain does not comprise any modifications. In other embodiments, the tail domain or one or more nucleotides therein have a modification, including but not limited to the modifications set forth herein. In certain embodiments, one or more nucleotides of the tail domain may comprise a 2′ modification (e.g., a modification at the 2′ position on ribose), e.g., a 2-acetylation, e.g., a 2′ methylation. In certain embodiments, the backbone of the tail domain can be modified with a phosphorothioate. In certain embodiments, modifications to one or more nucleotides of the tail domain render the tail domain and/or the gRNA comprising the tail domain less susceptible to degradation or more bio-compatible, e.g., less immunogenic. In certain embodiments, the tail domain includes 1, 2, 3, 4, 5, 6, 7, or 8 or more modifications, and in certain of these embodiments the tail domain includes 1, 2, 3, or 4 modifications within five nucleotides of its 5′ and/or 3′ end. In certain embodiments, the tail domain comprises modifications at two or more consecutive nucleotides.

In certain embodiments, modifications to one or more nucleotides in the tail domain are selected to not interfere with targeting efficacy, which can be evaluated by testing a candidate modification as set forth below. gRNAs having a candidate tail domain having a selected length, sequence, degree of complementarity, or degree of modification can be evaluated using a system as set forth below. The candidate tail domain can be placed, either alone or with one or more other candidate changes in a gRNA molecule/Cas9 molecule system known to be functional with a selected target, and evaluated.

In certain embodiments, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When an H1 promoter is used for transcription, these nucleotides may be the sequence UUUU. When alternate pol-III promoters are used, these nucleotides may be various numbers of uracil bases depending on, e.g., the termination signal of the pol-III promoter, or they may include alternate bases.

In certain embodiments, the proximal and tail domain taken together comprise, consist of, or consist essentially of the sequence set forth in SEQ ID NOs:32, 33, 34, 35, 36, or 37.

Exemplary Unimolecular/Chimeric gRNAs

In certain embodiments, a unimolecular or chimeric gRNA as disclosed herein has the structure: 5′ [targeting domain]-[first complementarity domain]-[linking domain]-[second complementarity domain]-[proximal domain]-[tail domain]-3′, wherein:
the targeting domain comprises a core domain and optionally a secondary domain, and is 10 to 50 nucleotides in length;
the first complementarity domain is 5 to 25 nucleotides in length and, in certain embodiments has at least 50, 60, 70, 80, 85, 90, or 95% homology with a reference first complementarity domain disclosed herein;
the linking domain is 1 to 5 nucleotides in length;
the second complementarity domain is 5 to 27 nucleotides in length and, in certain embodiments has at least 50, 60, 70, 80, 85, 90, or 95% homology with a reference second complementarity domain disclosed herein;
the proximal domain is 5 to 20 nucleotides in length and, in certain embodiments has at least 50, 60, 70, 80, 85, 90, or 95% homology with a reference proximal domain disclosed herein; and
the tail domain is absent or a nucleotide sequence is 1 to 50 nucleotides in length and, in certain embodiments has at least 50, 60, 70, 80, 85, 90, or 95% homology with a reference tail domain disclosed herein.

In certain embodiments, a unimolecular gRNA as disclosed herein comprises, preferably from 5′ to 3′:
a targeting domain, e.g., comprising 10-50 nucleotides;
a first complementarity domain, e.g., comprising 15, 16, 17, 18, 19, 20, 21, 22, 23, 24, 25, or 26 nucleotides;
a linking domain;
a second complementarity domain;
a proximal domain; and
a tail domain,

wherein,
(a) the proximal and tail domain, when taken together, comprise at least 15, 18, 20, 25, 30, 31, 35, 40, 45, 49, 50, or 53 nucleotides;
(b) there are at least 15, 18, 20, 25, 30, 31, 35, 40, 45, 49, 50, or 53 nucleotides 3′ to the last nucleotide of the second complementarity domain; or
(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the sequence from (a), (b), and/or (c) has at least 50%, 60%, 70%, 75%, 80%, 85%, 90%, 95%, or 99% homology with the corresponding sequence of a naturally occurring gRNA, or with a gRNA described herein.

In certain embodiments, the proximal and tail domain, when taken together, comprise at least 15, 18, 20, 25, 30, 31, 35, 40, 45, 49, 50, or 53 nucleotides.

In certain embodiments, there are at least 15, 18, 20, 25, 30, 31, 35, 40, 45, 49, 50, or 53 nucleotides 3′ to the last nucleotide of the second complementarity domain.

In certain embodiments, there are at least 16, 19, 21, 26, 31, 32, 36, 41, 46, 50, 51, or 54 nucleotides 3′ to the last nucleotide of the second complementarity domain that are complementary to the corresponding nucleotides of the first complementarity domain.

In certain embodiments, the targeting domain consists of, consists essentially of, or comprises 16, 17, 18, 19, 20, 21, 22, 23, 24, 25, or 26 nucleotides (e.g., 16, 17, 18, 19, 20, 21, 22, 23, 24, 25, or 26 consecutive nucleotides) complementary or partially complementary to the target domain or a portion thereof, e.g., the targeting domain is 16, 17, 18, 19, 20, 21, 22, 23, 24, 25, or 26 nucleotides in length. In certain of these embodiments, the targeting domain is complementary to the target domain over the entire length of the targeting domain, the entire length of the target domain, or both.

In certain embodiments, a unimolecular or chimeric gRNA molecule disclosed herein (comprising a targeting domain, a first complementarity domain, a linking domain, a second complementarity domain, a proximal domain and, optionally, a tail domain) comprises the nucleotide sequence set forth in SEQ ID NO:42, wherein the targeting domain is listed as 20 N's (residues 1-20) but may range in length from 16 to 26 nucleotides, and wherein the final six residues (residues 97-102) represent a termination signal for the U6 promoter but may be absent or fewer in number. In certain embodiments, the unimolecular, or chimeric, gRNA molecule is a S. pyogenes gRNA molecule.

In certain embodiments, a unimolecular or chimeric gRNA molecule disclosed herein (comprising a targeting domain, a first complementarity domain, a linking domain, a second complementarity domain, a proximal domain and, optionally, a tail domain) comprises the nucleotide sequence set forth in SEQ ID NO:38, wherein the targeting domain is listed as 20 Ns (residues 1-20) but may range in length from 16 to 26 nucleotides, and wherein the final six residues (residues 97-102) represent a termination signal for the U6 promoter but may be absent or fewer in number. In certain embodiments, the unimolecular or chimeric gRNA molecule is an S. aureus gRNA molecule.

The sequences and structures of exemplary chimeric gRNAs are also shown in FIGS. 1H-1I.

Exemplary Modular gRNAs

In certain embodiments, a modular gRNA disclosed herein comprises:
a first strand comprising, preferably from 5′ to 3′;
   a targeting domain, e.g., comprising 15, 16, 17, 18, 19, 20, 21, 22, 23, 24, 25, or 26 nucleotides;
   a first complementarity domain; and
a second strand, comprising, preferably from 5′ to 3′:
   optionally a 5′ extension domain;
   a second complementarity domain;
   a proximal domain; and
   a tail domain,

wherein:
(a) the proximal and tail domain, when taken together, comprise at least 15, 18, 20, 25, 30, 31, 35, 40, 45, 49, 50, or 53 nucleotides;
(b) there are at least 15, 18, 20, 25, 30, 31, 35, 40, 45, 49, 50, or 53 nucleotides 3′ to the last nucleotide of the second complementarity domain; or
(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the sequence from (a), (b), or (c), has at least 60, 75, 80, 85, 90, 95, or 99% homology with the corresponding sequence of a naturally occurring gRNA, or with a gRNA described herein.

In certain embodiments, the proximal and tail domain, when taken together, comprise at least 15, 18, 20, 25, 30, 31, 35, 40, 45, 49, 50, or 53 nucleotides.

In certain embodiments, there are at least 15, 18, 20, 25, 30, 31, 35, 40, 45, 49, 50, or 53 nucleotides 3′ to the last nucleotide of the second complementarity domain.

In certain embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain. In certain embodiments, the targeting domain comprises, has, or consists of, 16, 17, 18, 19, 20, 21, 22, 23, 24, 25, or 26 nucleotides (e.g., 16, 17, 18, 19, 20, 21, 22, 23, 24, 25, or 26 consecutive nucleotides) having complementarity with the target domain, e.g., the targeting domain is 16, 17, 18, 19, 20, 21, 22, 23, 24, 25, or 26 nucleotides in length.

In certain embodiments, the targeting domain consists of, consists essentially of, or comprises 16, 17, 18, 19, 20, 21, 22, 23, 24, 25, or 26 nucleotides (e.g., 16, 17, 18, 19, 20, 21, 22, 23, 24, 25, or 26 consecutive nucleotides) complementary to the target domain or a portion thereof. In certain of these embodiments, the targeting domain is complementary to the target domain over the entire length of the targeting domain, the entire length of the target domain, or both.

In certain embodiments, the targeting domain comprises, consists of, or consists essentially of 16 nucleotides (e.g., 16 consecutive nucleotides) having complementarity with the target domain, e.g., the targeting domain is 16 nucleotides in length. In certain embodiments of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 17 nucleotides (e.g., 17 consecutive nucleotides) having complementarity with the target domain, e.g., the targeting domain is 17 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 18 nucleotides (e.g., 18 consecutive nucleotides) having complementarity with the target domain, e.g., the targeting domain is 18 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 19 nucleotides (e.g., 19 consecutive nucleotides) having complementarity with the target domain, e.g., the targeting domain is 19 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 20 nucleotides (e.g., 20 consecutive nucleotides) having complementarity with the target domain, e.g., the targeting domain is 20 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 21 nucleotides (e.g., 21 consecutive nucleotides) having complementarity with the target domain, e.g., the targeting domain is 21 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 22 nucleotides (e.g., 22 consecutive nucleotides) having complementarity with the target domain, e.g., the targeting domain is 22 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 23 nucleotides (e.g., 23 consecutive nucleotides) having complementarity with the target domain, e.g., the targeting domain is 23 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 24 nucleotides (e.g., 24 consecutive nucleotides) having complementarity with the target domain, e.g., the targeting domain is 24 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 25 nucleotides (e.g., 25 consecutive nucleotides) having complementarity with the target domain, e.g., the targeting domain is 25 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, consists of, or consists essentially of 26 nucleotides (e.g., 26 consecutive nucleotides) having complementarity with the target domain, e.g., the targeting domain is 26 nucleotides in length. In certain of these embodiments, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; and/or there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

gRNA Delivery

In certain embodiments of the methods provided herein, the methods comprise delivery of one or more (e.g., two, three, or four) gRNA molecules as described herein. In certain of these embodiments, the gRNA molecules are delivered by intravenous injection, intramuscular injection, subcutaneous injection, or inhalation.

Methods for Designing gRNAs

Methods for selecting, designing, and validating targeting domains for use in the gRNAs described herein are provided. Exemplary targeting domains for incorporation into gRNAs are also provided herein.

Methods for selection and validation of target sequences as well as off-target analyses have been described previously (see, e.g., Mali 2013; Hsu 2013; Fu 2014; Heigwer 2014; Bae 2014; Xiao 2014). For example, a software tool can be used to optimize the choice of potential targeting domains corresponding to a user's target sequence, e.g., to minimize total off-target activity across the genome. Off-target activity may be other than cleavage. For each possible targeting domain choice using S. pyogenes Cas9, the tool can identify all off-target sequences (preceding either NAG or NGG PAMs) across the genome that contain up to certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible targeting domain is then ranked according to its total predicted off-target cleavage; the top-ranked targeting domains represent those that are likely to have the greatest on-target cleavage and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate targeting domains and gRNAs comprising those targeting domains can be functionally evaluated using methods known in the art and/or as set forth herein.

As a non-limiting example, targeting domains for use in gRNAs for use with S. pyogenes and S. aureus Cas9s were identified using a DNA sequence searching algorithm. 17-mer and 20-mer targeting domains were designed for S. pyogenes targets, while 18-mer, 19-mer, 20-mer, 21-mer, 22-mer, 23-mer, and 24-mer targeting domains were designed for S. aureus targets. gRNA design was carried out using custom gRNA design software based on the public tool cas-offinder (Bae 2014). This software scores guides after calculating their genome-wide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential target sites adjacent to PAM sequences, the software also identifies all PAM adjacent sequences that differ by 1, 2, 3, or more than 3 nucleotides from the selected target sites. Genomic DNA sequences for HBG1 and HBG2 regulatory regions were obtained from the UCSC Genome browser and sequences were screened for repeat elements using the publically available RepeatMasker program. RepeatMasker searches input DNA sequences for repeated elements and regions of low complexity. The output is a detailed annotation of the repeats present in a given query sequence.

Following identification, targeting domains were ranked into tiers based on their distance to the target site, their orthogonality, and presence of a 5′ G (based on identification of close matches in the human genome containing a relevant PAM, e.g., an NGG PAM for S. pyogenes, or an NNGRRT (SEQ ID NO:204) or NNGRRV (SEQ ID NO:205) PAM for S. aureus). Orthogonality refers to the number of sequences in the human genome that contain a minimum number of mismatches to the target sequence. A “high level of orthogonality” or “good orthogonality” may, for example, refer to 20-mer targeting domain that have no identical sequences in the human genome besides the intended target, nor any sequences that contain one or two mismatches in the target sequence. Targeting domains with good orthogonality are selected to minimize off-target DNA cleavage.

Targeting domains were identified for both single-gRNA nuclease cleavage and for a dual-gRNA paired “nickase” strategy. Criteria for selecting targeting domains and the determination of which targeting domains can be used for the dual-gRNA paired “nickase” strategy is based on two considerations:
(1) Targeting domain pairs should be oriented on the DNA such that PAMs are facing out and cutting with the D10A Cas9 nickase will result in 5′ overhangs; and
(2) An assumption that cleaving with dual nickase pairs will result in deletion of the entire intervening sequence at a reasonable frequency. However, cleaving with dual nickase pairs can also result in indel mutations at the site of only one of the gRNAs. Candidate pair members can be tested for how efficiently they remove the entire sequence versus causing indel mutations at the target site of one targeting domain.

### Targeting Domains for Use in Deleting HBG1 c.−114 to −102

Targeting domains for use in gRNAs for deleting c.−114 to −102 of HBG1 in conjunction with the methods disclosed herein were identified and ranked into 4 tiers for S. pyogenes and S. aureus.

For S. pyogenes, tier 1 targeting domains were selected based on (1) distance upstream or downstream from either end of the target site (i.e., HBG1 c.−114 to −102), specifically within 400 bp of either end of the target site, (2) a high level of orthogonality, and (3) the presence of 5′ G. Tier 2 targeting domains were selected based on (1) distance upstream or downstream from either end of the target site (i.e., HBG1 c.−114 to −102), specifically within 400 bp of either end of the target site, and (2) a high level of orthogonality. Tier 3 targeting domains were selected based on (1) distance upstream or downstream from either end of the target site (i.e., HBG1 c.−114 to −102), specifically within 400 bp of either end of the target site and (2) the presence of 5′ G. Tier 4 targeting domains were selected based on distance upstream or downstream from either end of the target site (i.e., HBG1 c.−114 to −102), specifically within 400 bp of either end of the target site.

For S. aureus, tier 1 targeting domains were selected based on (1) distance upstream or downstream from either end of the target site (i.e., HBG1 c.−114 to −102), specifically within 400 bp of either end of the target site, (2) a high level of orthogonality, (3) the presence of 5′ G, and (4) PAM having the sequence NNGRRT (SEQ ID NO:204). Tier 2 targeting domains were selected based on (1) distance upstream or downstream from either end of the target site (i.e., HBG1 c.−114 to −102), specifically within 400 bp of either end of the target site, (2) a high level of orthogonality, and (3) PAM having the sequence NNGRRT (SEQ ID NO:204). Tier 3 targeting domains were selected based on (1) distance upstream or downstream from either end of the target site (i.e., HBG1 c.−114 to −102), specifically within 400 bp of either end of the target site, and (2) PAM having the sequence NNGRRT (SEQ ID NO:204). Tier 4 targeting domains were selected based on (1) distance upstream or downstream from either end of the target site (i.e., HBG1 c.−114 to −102), specifically within 400 bp of either end of the target site, and (2) PAM having the sequence NNGRRV (SEQ ID NO:205).

Note that tiers are non-inclusive (each targeting domain is listed only once for the strategy). In certain instances, no targeting domain was identified based on the criteria of the particular tier. The identified targeting domains are summarized below in Table 6.

TABLE 6
Nucleotide sequences of S. pyogenes and S. aureus targeting domains

| | S. pyogenes | S. aureus |
|---|---|---|
| Tier 1 | SEQ ID NOs: 251-256 | SEQ ID NOs: 367-376 |
| Tier 2 | SEQ ID NOs: 257-274 | SEQ ID NOs: 343, 377- |
| | | 393 |
| Tier 3 | SEQ ID NOs: 275-300 | SEQ ID NOs: 357, 365, |
| | | 394-461 |
| Tier 4 | SEQ ID NOs: 301-366 | SEQ ID NOs: 252-254, |
| | | 256, 268, 272-274, 292, |
| | | 295, 347, 348, 353, |
| | | 360-362, 366, 598-759 |

### Targeting Domains for Use in Deleting HBG2 c.−114 to −102

Targeting domains for use in gRNAs for deleting c.−114 to −102 of HBG2 in conjunction with the methods disclosed herein were identified and ranked into 4 tiers for S. pyogenes and S. aureus.

For S. pyogenes, tier 1 targeting domains were selected based on (1) distance upstream or downstream from either end of the target site (i.e., HBG2 c.−114 to −102), specifically within 400 bp of either end of the target site, (2) a high level of orthogonality, and (3) the presence of 5′ G. Tier 2 targeting domains were selected based on (1) distance upstream or downstream from either end of the target site (i.e., HBG2 c.−114 to −102), specifically within 400 bp of either end of the target site, and (2) a high level of orthogonality. Tier 3 targeting domains were selected based on (1) distance upstream or downstream from either end of the target site (i.e., HBG2 c.−114 to −102), specifically within 400 bp of either end of the target site and (2) the presence of 5′ G. Tier 4 targeting domains were selected based on distance upstream or downstream from either end of the target site (i.e., HBG2 c.−114 to −102), specifically within 400 bp of either end of the target site.

For S. aureus, tier 1 targeting domains were selected based on (1) distance upstream or downstream from either end of the target site (i.e., HBG2 c.−114 to −102), specifically within 400 bp of either end of the target site, (2) a high level of orthogonality, (3) the presence of 5′ G, and (4) PAM having the sequence NNGRRT (SEQ ID NO:204). Tier 2 targeting domains were selected based on (1) distance upstream or downstream from either end of the target site (i.e., HBG2 c.−114 to −102), specifically within 400 bp of either end of the target site, (2) a high level of orthogonality, and (3) PAM having the sequence NNGRRT (SEQ ID NO:204). Tier 3 targeting domains were selected based on (1) distance upstream or downstream from either end of the target site (i.e., HBG2 c.−114 to −102), specifically within 400 bp of either end of the target site, and (2) PAM having the sequence NNGRRT (SEQ ID NO:204). Tier 4 targeting domains were selected based on (1) distance upstream or downstream from either end of the target site (i.e., HBG2 c.−114 to −102), specifically within 400 bp of either end of the target site, and (2) PAM having the sequence NNGRRV (SEQ ID NO:205).

Note that tiers are non-inclusive (each targeting domain is listed only once for the strategy). In certain instances, no targeting domain was identified based on the criteria of the particular tier. The identified targeting domains are summarized below in Table 7.

TABLE 7
Nucleotide sequences of S. pyogenes and S. aureus targeting domains

| | S. pyogenes | S. aureus |
|---|---|---|
| Tier 1 | SEQ ID NOs: 760-764 | SEQ ID NOs: 784-791 |
| Tier 2 | SEQ ID NOs: 765-781 | SEQ ID NOs: 778, 792- |
| | | 803 |
| Tier 3 | SEQ ID NOs: 275- | SEQ ID NOs: 357, 365, |
| | 281, 283-300 | 394-461 |
| Tier 4 | SEQ ID NOs: 301- | SEQ ID NOs: 292, 295, |
| | 311, 313-342, 344- | 347, 348, 353, 360-362, |
| | 348, 350-366, 782, | 366, 462-468, 476-481, |
| | 783 | 489-587, 601-607, 614- |
| | | 620, 640-666, 674-679, |
| | | 687-693, 708-714, 733- |
| | | 753, 762-764, 775, 779- |
| | | 781, 804-901 |

In certain embodiments, two or more (e.g., three or four) gRNA molecules are used with one Cas9 molecule. In another embodiment, when two or more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

Any of the targeting domains in the tables described herein can be used with a Cas9 molecule that generates a single strand break (i.e., S. pyogenes or S. aureus Cas9 nickase) or with a Cas9 molecule that generates a double strand break (i.e., S. pyogenes or S. aureus Cas9 nuclease).

When two gRNAs are designed for use with two Cas9 molecules, the two Cas9 molecules may be different species. Both Cas9 species may be used to generate a single or double-strand break, as desired.

It is contemplated herein that any upstream gRNA described herein may be paired with any downstream gRNA described herein. When an upstream gRNA designed for use with one species of Cas9 is paired with a downstream gRNA designed for use from a different species of Cas9, both Cas9 species are used to generate a single or double-strand break, as desired.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) a PAM. RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer as visualized relative to the top or complementary strand:

5′--------------------[protospacer]----------------------------3′
3′-------------------------------------[PAM]-------------------5′

Cpf1, on the other hand, generally recognizes PAM sequences that are 5′ of the protospacer:

5′-----------------------------[protospacer] -------------------3′
3′--------------------[PAM]------------------------------------5′

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran 2013, incorporated by reference herein), or that that do not cut at all.

### Cas9 Molecules

Cas9 molecules of a variety of species can be used in the methods and compositions described herein. While S. pyogenes and S. aureus Cas9 molecules are the subject of much of the disclosure herein, Cas9 molecules of, derived from, or based on the Cas9 proteins of other species listed herein can be used as well. These include, for example, Cas9 molecules from Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

### Cas9 Domains

Crystal structures have been determined for two different naturally occurring bacterial Cas9 molecules (Jinek 2014) and for S. pyogenes Cas9 with a guide RNA (e.g., a synthetic fusion of crRNA and tracrRNA) (Nishimasu 2014; Anders 2014).

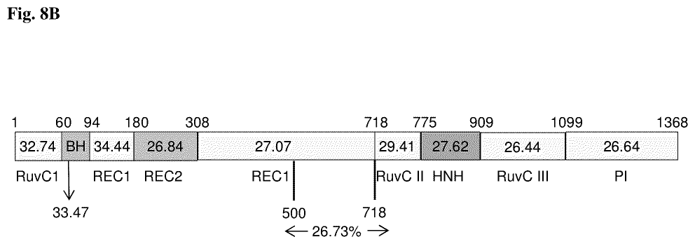

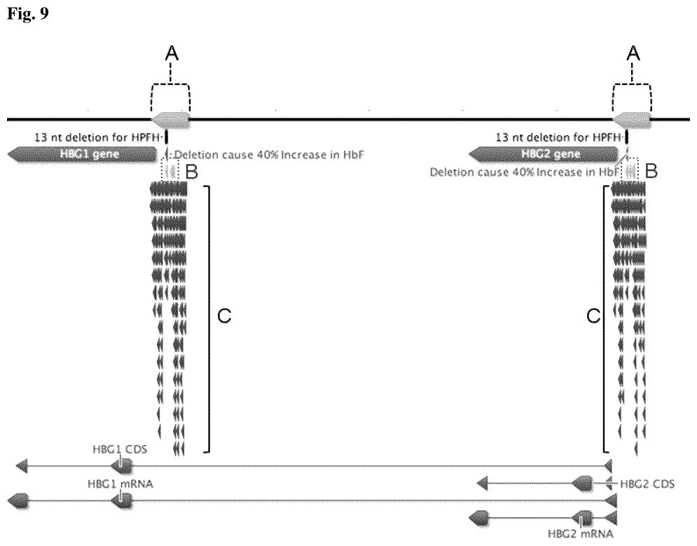

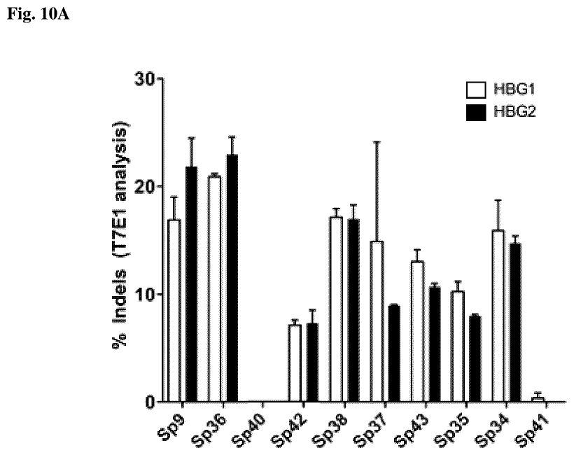

A naturally occurring Cas9 molecule comprises two lobes: a recognition (REC) lobe and a nuclease (NUC) lobe; each of which further comprise domains described herein. FIGS. 8A-8B provide a schematic of the organization of important Cas9 domains in the primary structure. The domain nomenclature and the numbering of the amino acid residues encompassed by each domain used throughout this disclosure is as described previously (Nishimasu 2014). The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long a helix and arginine rich region and comprises amino acids 60-93 of S. pyogenes Cas9 (SEQ ID NO:2). The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of S. pyogenes Cas9 (SEQ ID NO:2). These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of S. pyogenes Cas9 (SEQ ID NO:2).

The NUC lobe comprises the RuvC domain, the HNH domain, and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvCI, RuvCII, and RuvCIII, which are often commonly referred to in the art as RuvCI domain or N-terminal RuvC domain, RuvCII domain, and RuvCIII domain, respectively) at amino acids 1-59, 718-769, and 909-1098, respectively, of S. pyogenes Cas9 (SEQ ID NO:2). Similar to the REC1 domain, the three RuvC motifs are linearly separated by other domains in the primary structure. However, in the tertiary structure, the three RuvC motifs assemble and form the RuvC domain. The HNH domain shares structural similarity with HNH endonucleases and cleaves a single strand, e.g., the complementary strand of the target nucleic acid molecule. The HNH domain lies between the RuvC II-III motifs and comprises amino acids 775-908 of S. pyogenes Cas9 (SEQ ID NO:2). The PI domain interacts with the PAM of the target nucleic acid molecule, and comprises amino acids 1099-1368 of S. pyogenes Cas9 (SEQ ID NO:2).

### RuvC-Like Domain and HNH-Like Domain

In certain embodiments, a Cas9 molecule or Cas9 polypeptide comprises an HNH-like domain and a RuvC-like domain, and in certain of these embodiments cleavage activity is dependent on the RuvC-like domain and the HNH-like domain. A Cas9 molecule or Cas9 polypeptide can comprise one or more of a RuvC-like domain and an HNH-like domain. In certain embodiments, a Cas9 molecule or Cas9 polypeptide comprises a RuvC-like domain, e.g., a RuvC-like domain described below, and/or an HNH-like domain, e.g., an HNH-like domain described below.

### RuvC-Like Domains

In certain embodiments, a RuvC-like domain cleaves a single strand, e.g., the non-complementary strand of the target nucleic acid molecule. The Cas9 molecule or Cas9 polypeptide can include more than one RuvC-like domain (e.g., one, two, three or more RuvC-like domains). In certain embodiments, a RuvC-like domain is at least 5, 6, 7, 8 amino acids in length but not more than 20, 19, 18, 17, 16 or 15 amino acids in length. In certain embodiments, the Cas9 molecule or Cas9 polypeptide comprises an N-terminal RuvC-like domain of about 10 to 20 amino acids, e.g., about 15 amino acids in length.

### N-Terminal RuvC-Like Domains

Some naturally occurring Cas9 molecules comprise more than one RuvC-like domain with cleavage being dependent on the N-terminal RuvC-like domain. Accordingly, a Cas9 molecule or Cas9 polypeptide can comprise an N-terminal RuvC-like domain. Exemplary N-terminal RuvC-like domains are described below.

In certain embodiments, a Cas9 molecule or Cas9 polypeptide comprises an N-terminal RuvC-like domain comprising an amino acid sequence of Formula I:

(SEQ ID NO: 20)
D-X₁-G-X₂-X₃-X₄-X₅-G-X₆-X₇-X₈-X₉,

wherein
X₁ is selected from I, V, M, L, and T (e.g., selected from I, V, and L);
X₂ is selected from T, I, V, S, N, Y, E, and L (e.g., selected from T, V, and I);
X₃ is selected from N, S, G, A, D, T, R, M, and F (e.g., A or N);
X₄ is selected from S, Y, N, and F (e.g., S);
X₅ is selected from V, I, L, C, T, and F (e.g., selected from V, I and L);
X₆ is selected from W, F, V, Y, S, and L (e.g., W);
X₇ is selected from A, S, C, V, and G (e.g., selected from A and S);
X₈ is selected from V, I, L, A, M, and H (e.g., selected from V, I, M and L); and
X₉ is selected from any amino acid or is absent (e.g., selected from T, V, I, L, A, F, S, A, Y, M, and R, or, e.g., selected from T, V, I, L, and A).

In certain embodiments, the N-terminal RuvC-like domain differs from a sequence of SEQ ID NO:20 by as many as 1 but no more than 2, 3, 4, or 5 residues.

In certain embodiments, the N-terminal RuvC-like domain is cleavage competent. In other embodiments, the N-terminal RuvC-like domain is cleavage incompetent.

In certain embodiments, a Cas9 molecule or Cas9 polypeptide comprises an N-terminal RuvC-like domain comprising an amino acid sequence of Formula II:

(SEQ ID NO: 21)
D-X₁-G-X₂-X₃-S-X₅-G-X₆-X₇-X₈-X₉,

wherein
X₁ is selected from I, V, M, L, and T (e.g., selected from I, V, and L);
X₂ is selected from T, I, V, S, N, Y, E, and L (e.g., selected from T, V, and I);
X₃ is selected from N, S, G, A, D, T, R, M and F (e.g., A or N);
X₅ is selected from V, I, L, C, T, and F (e.g., selected from V, I and L);
X₆ is selected from W, F, V, Y, S, and L (e.g., W);
X₇ is selected from A, S, C, V, and G (e.g., selected from A and S);
X₈ is selected from V, I, L, A, M, and H (e.g., selected from V, I, M and L); and
X₉ is selected from any amino acid or is absent (e.g., selected from T, V, I, L, A, F, S, A, Y, M, and R or selected from e.g., T, V, I, L, and A).

In certain embodiments, the N-terminal RuvC-like domain differs from a sequence of SEQ ID NO:21 by as many as 1 but not more than 2, 3, 4, or 5 residues.

In certain embodiments, the N-terminal RuvC-like domain comprises an amino acid sequence of Formula III:

(SEQ ID NO: 22)
D-I-G-X₂-X₃-S-V-G-W-A-X₈-X₉,

wherein
X₂ is selected from T, I, V, S, N, Y, E, and L (e.g., selected from T, V, and I);
X₃ is selected from N, S, G, A, D, T, R, M, and F (e.g., A or N);
X₈ is selected from V, I, L, A, M, and H (e.g., selected from V, I, M and L); and
X₉ is selected from any amino acid or is absent (e.g., selected from T, V, I, L, A, F, S, A, Y, M, and R or selected from e.g., T, V, I, L, and A).

In certain embodiments, the N-terminal RuvC-like domain differs from a sequence of SEQ ID NO:22 by as many as 1 but not more than, 2, 3, 4, or 5 residues.

In certain embodiments, the N-terminal RuvC-like domain comprises an amino acid sequence of Formula IV:

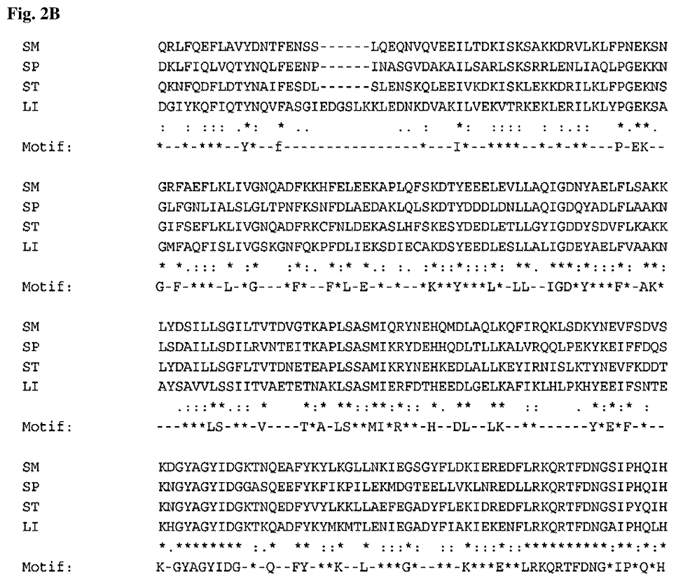

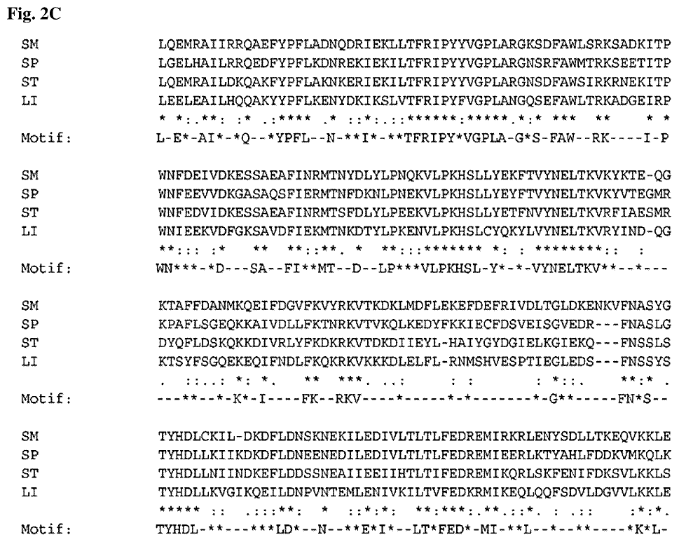

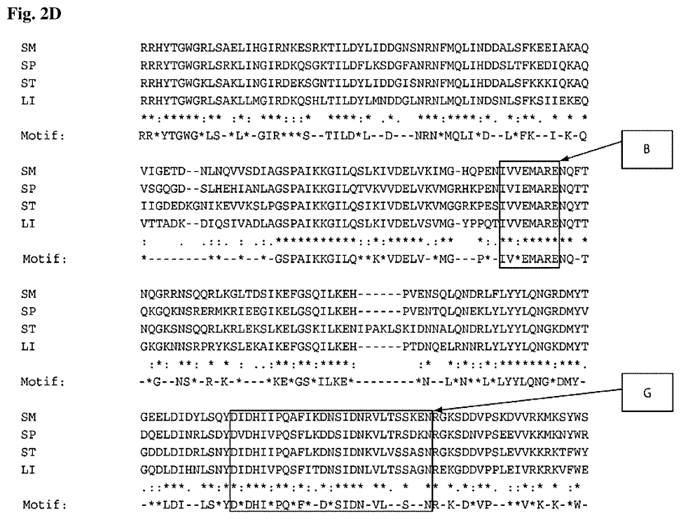

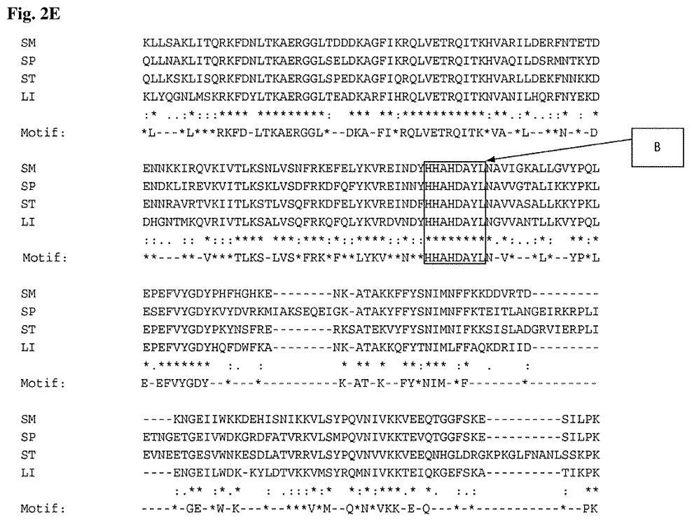

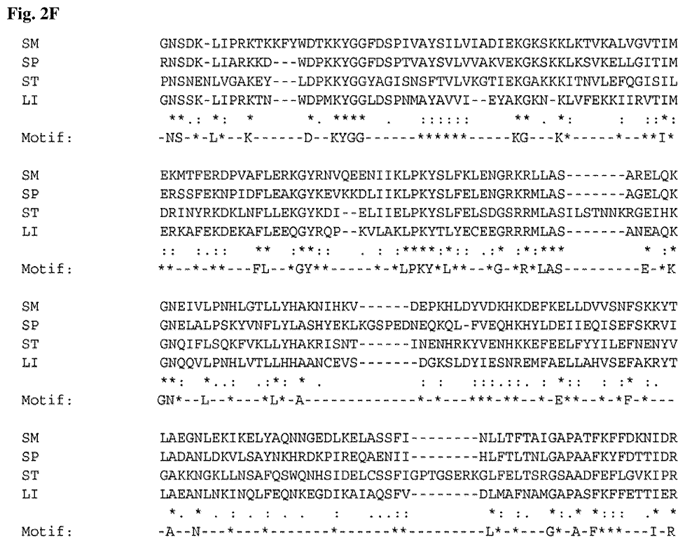

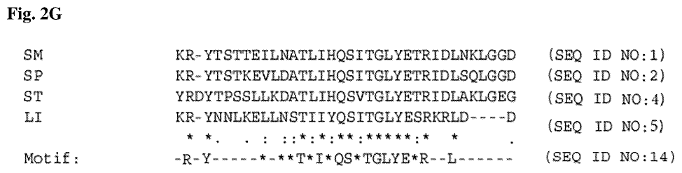

(SEQ ID NO: 23)
D-I-G-T-N-S-V-G-W-A-V-X,

wherein
X is a non-polar alkyl amino acid or a hydroxyl amino acid, e.g., X is selected from V, I, L, and T (e.g., the Cas9 molecule can comprise an N-terminal RuvC-like domain shown in FIGS. 2A-2G (depicted as Y)).

In certain embodiments, the N-terminal RuvC-like domain differs from a sequence of SEQ ID NO:23 by as many as 1 but not more than, 2, 3, 4, or 5 residues.

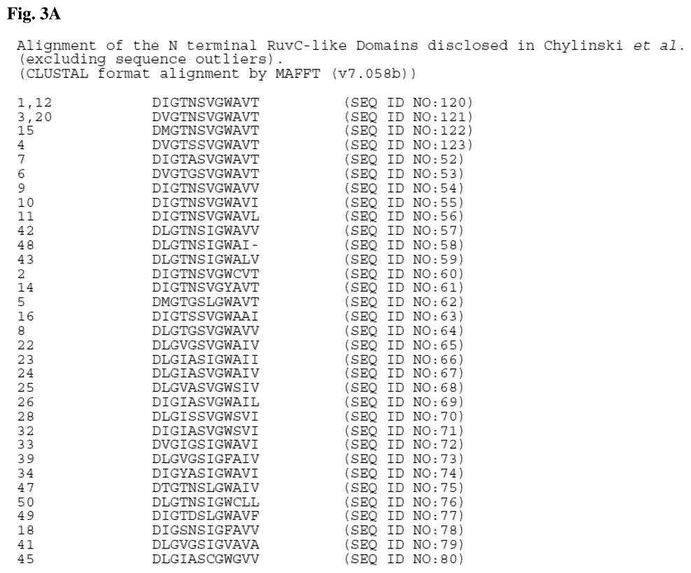

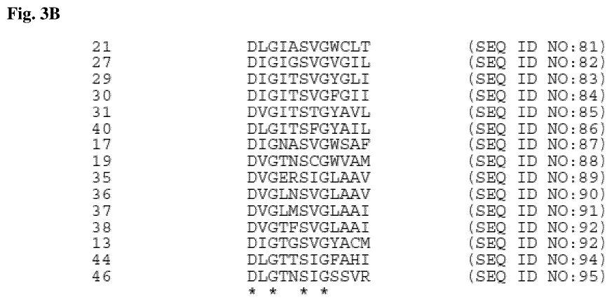

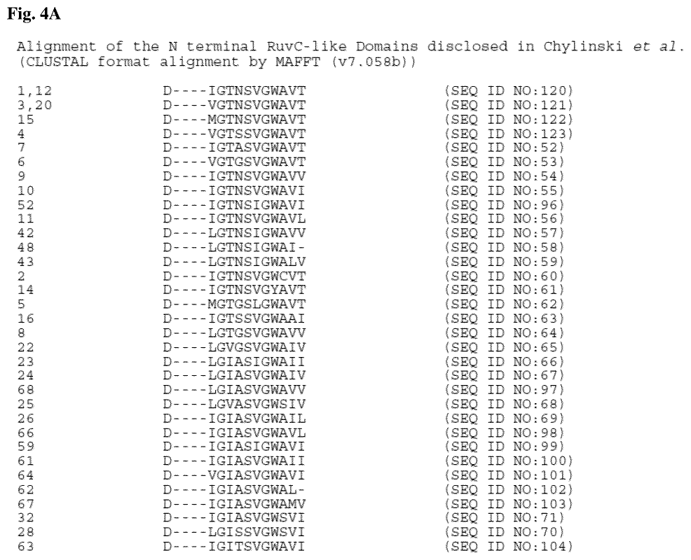

In certain embodiments, the N-terminal RuvC-like domain differs from a sequence of an N-terminal RuvC like domain disclosed herein, e.g., in FIGS. 3A-3B, as many as 1 but no more than 2, 3, 4, or 5 residues. In an embodiment, 1, 2, 3 or all of the highly conserved residues identified in FIGS. 3A-3B are present.

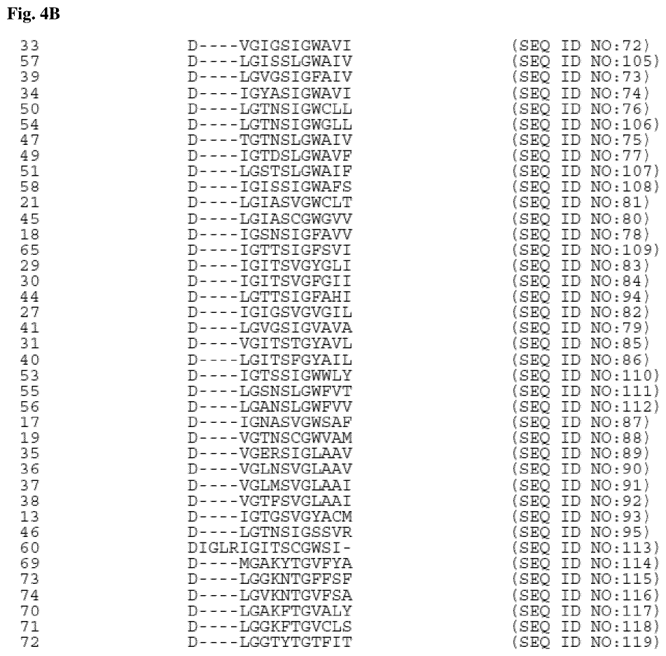

In certain embodiments, the N-terminal RuvC-like domain differs from a sequence of an N-terminal RuvC-like domain disclosed herein, e.g., in FIGS. 4A-4B, as many as 1 but no more than 2, 3, 4, or 5 residues. In an embodiment, 1, 2, or all of the highly conserved residues identified in FIGS. 4A-4B are present.

### Additional RuvC-Like Domains

In addition to the N-terminal RuvC-like domain, the Cas9 molecule or Cas9 polypeptide can comprise one or more additional RuvC-like domains. In certain embodiments, the Cas9 molecule or Cas9 polypeptide can comprise two additional RuvC-like domains. Preferably, the additional RuvC-like domain is at least 5 amino acids in length and, e.g., less than 15 amino acids in length, e.g., 5 to 10 amino acids in length, e.g., 8 amino acids in length.

An additional RuvC-like domain can comprise an amino acid sequence of Formula V:

(SEQ ID NO: 15)
I-X₁-X₂-E-X₃-A-R-E,

wherein
X₁ is V or H;
X₂ is I, L or V (e.g., I or V); and
X₃ is M or T.

In certain embodiments, the additional RuvC-like domain comprises an amino acid sequence of Formula VI:

(SEQ ID NO: 16)
I-V-X₂-E-M-A-R-E,

wherein
X₂ is I, L or V (e.g., I or V) (e.g., the Cas9 molecule or Cas9 polypeptide can comprise an additional RuvC-like domain shown in FIG. 2A-2G (depicted as B)).

An additional RuvC-like domain can comprise an amino acid sequence of Formula VII:

(SEQ ID NO: 17)
H-H-A-X₁-D-A-X₂-X₃,

wherein
X₁ is H or L;
X₂ is R or V; and
X₃ is E or V.

In certain embodiments, the additional RuvC-like domain comprises the amino acid sequence:

(SEQ ID NO: 18)
H-H-A-H-D-A-Y-L.

In certain embodiments, the additional RuvC-like domain differs from a sequence of SEQ ID NOs:15-18 by as many as 1 but not more than 2, 3, 4, or 5 residues.

In certain embodiments, the sequence flanking the N-terminal RuvC-like domain has the amino acid sequence of Formula VIII:

(SEQ ID NO: 19)
K-X₁′-Y-X₂′-X₃′-X₄′-Z-T-D-X₉′-Y,

wherein
X₁′ is selected from K and P;
X₂′ is selected from V, L, I, and F (e.g., V, I and L);
X₃′ is selected from G, A and S (e.g., G);
X₄′ is selected from L, I, V, and F (e.g., L);
X₉′ is selected from D, E, N, and Q; and
Z is an N-terminal RuvC-like domain, e.g., as described above, e.g., having 5 to 20 amino acids.

### HNH-Like Domains

In certain embodiments, an HNH-like domain cleaves a single stranded complementary domain, e.g., a complementary strand of a double stranded nucleic acid molecule. In certain embodiments, an HNH-like domain is at least 15, 20, or 25 amino acids in length but not more than 40, 35, or 30 amino acids in length, e.g., 20 to 35 amino acids in length, e.g., 25 to 30 amino acids in length. Exemplary HNH-like domains are described below.

In an embodiment, a Cas9 molecule or Cas9 polypeptide comprises an HNH-like domain having an amino acid sequence of Formula IX:

(SEQ ID NO: 25)
X₁-X₂-X₃-H-X₄-X₅-P-X₆-X₇-X₈-X⁹-X¹⁰-X¹¹-X¹²-X¹³-
X¹⁴-X¹⁵-N-X¹⁶-X¹⁷-X¹⁸-X¹⁹-X₂₀-X₂₁-X₂₂-X₂₃-N,

wherein
X₁ is selected from D, E, Q, and N (e.g., D and E);
X² is selected from L, I, R, Q, V, M, and K;
X₃ is selected from D and E;
X₄ is selected from I, V, T, A, and L (e.g., A, I, and V);
X₅ is selected from V, Y, I, L, F, and W (e.g., V, I, and L);
X₆ is selected from Q, H, R, K, Y, I, L, F, and W;
X₇ is selected from S, A, D, T, and K (e.g., S and A);
X₈ is selected from F, L, V, K, Y, M, I, R, A, E, D, and Q (e.g., F);
X₉ is selected from L, R, T, I, V, S, C, Y, K, F, and G;
X₁₀ is selected from K, Q, Y, T, F, L, W, M, A, E, G, and S;
X₁₁ is selected from D, S, N, R, L, and T (e.g., D);
X₁₂ is selected from D, N and S;
X₁₃ is selected from S, A, T, G, and R (e.g., S);
X₁₄ is selected from I, L, F, S, R, Y, Q, W, D, K, and H (e.g., I, L, and F);
X₁₅ is selected from D, S, I, N, E, A, H, F, L, Q, M, G, Y, and V;
X₁₆ is selected from K, L, R, M, T, and F (e.g., L, R, and K);
X₁₇ is selected from V, L, I, A, and T;
X₁₈ is selected from L, I, V, and A (e.g., L and I);
X₁₉ is selected from T, V, C, E, S, and A (e.g., T and V);
X₂₀ is selected from R, F, T, W, E, L, N, C, K, V, S, Q, I, Y, H, and A;
X₂₁ is selected from S, P, R, K, N, A, H, Q, G, and L;
X₂₂ is selected from D, G, T, N, S, K, A, I, E, L, Q, R, and Y; and
X₂₃ is selected from K, V, A, E, Y, I, C, L, S, T, G, K, M, D, and F.

In certain embodiments, a HNH-like domain differs from a sequence of SEQ ID NO:25 by at least one but not more than, 2, 3, 4, or 5 residues.

In certain embodiments, the HNH-like domain is cleavage competent. In other embodiments, the HNH-like domain is cleavage incompetent.

In certain embodiments, a Cas9 molecule or Cas9 polypeptide comprises an HNH-like domain comprising an amino acid sequence of Formula X:

(SEQ ID NO: 26)
X₁-X₂-X₃-H-X₄-X₅-P-X₆-S-X₈-X₉-X₁₀-D-D-S-X₁₄-X₁₅-
N-K-V-L-X₁₉-X₂₀-X₂₁-X₂₂-X₂₃-N,

wherein
X₁ is selected from D and E;
X₂ is selected from L, I, R, Q, V, M, and K;
X₃ is selected from D and E;
X₄ is selected from I, V, T, A, and L (e.g., A, I, and V);
X₅ is selected from V, Y, I, L, F, and W (e.g., V, I, and L);
X₆ is selected from Q, H, R, K, Y, I, L, F, and W;
X₈ is selected from F, L, V, K, Y, M, I, R, A, E, D, and Q (e.g., F);
X₉ is selected from L, R, T, I, V, S, C, Y, K, F, and G;
X₁₀ is selected from K, Q, Y, T, F, L, W, M, A, E, G, and S;
X₁₄ is selected from I, L, F, S, R, Y, Q, W, D, K, and H (e.g., I, L, and F);
X₁₅ is selected from D, S, I, N, E, A, H, F, L, Q, M, G, Y, and V;
X₁₉ is selected from T, V, C, E, S, and A (e.g., T and V);
X₂₀ is selected from R, F, T, W, E, L, N, C, K, V, S, Q, I, Y, H, and A;
X₂₁ is selected from S, P, R, K, N, A, H, Q, G, and L;
X₂₂ is selected from D, G, T, N, S, K, A, I, E, L, Q, R, and Y; and
X₂₃ is selected from K, V, A, E, Y, I, C, L, S, T, G, K, M, D, and F.

In certain embodiments, the HNH-like domain differs from a sequence of SEQ ID NO:26 by 1, 2, 3, 4, or 5 residues.

In certain embodiments, a Cas9 molecule or Cas9 polypeptide comprises an HNH-like domain comprising an amino acid sequence of Formula XI:

(SEQ ID NO: 27)
X₁-V-X₃-H-I-V-P-X₆-S-X₈-X₉-X₁₀-D-D-S-X₁₄-X₁₅-N-
K-V-L-T-X₂₀-X₂₁-X₂₂-X₂₃-N,

wherein
X₁ is selected from D and E;
X₃ is selected from D and E;
X₆ is selected from Q, H, R, K, Y, I, L, and W;
X₈ is selected from F, L, V, K, Y, M, I, R, A, E, D, and Q (e.g., F);
X₉ is selected from L, R, T, I, V, S, C, Y, K, F, and G;
X₁₀ is selected from K, Q, Y, T, F, L, W, M, A, E, G, and S;
X₁₄ is selected from I, L, F, S, R, Y, Q, W, D, K, and H (e.g., I, L, and F);
X₁₅ is selected from D, S, I, N, E, A, H, F, L, Q, M, G, Y, and V;
X₂₀ is selected from R, F, T, W, E, L, N, C, K, V, S, Q, I, Y, H, and A;
X₂₁ is selected from S, P, R, K, N, A, H, Q, G, and L;
X₂₂ is selected from D, G, T, N, S, K, A, I, E, L, Q, R, and Y; and
X₂₃ is selected from K, V, A, E, Y, I, C, L, S, T, G, K, M, D, and F.

In certain embodiments, the HNH-like domain differs from a sequence of SEQ ID NO:27 by 1, 2, 3, 4, or 5 residues.

In certain embodiments, a Cas9 molecule or Cas9 polypeptide comprises an HNH-like domain having an amino acid sequence of Formula XII:

(SEQ ID NO: 28)
D-X₂-D-H-I-X₅-P-Q-X₇-F-X₉-X₁₀-D-X₁₂-S-I-D-N-X₁₆-
V-L-X₁₉-X₂₀-S-X₂₂-X₂₃-N,

wherein
X₂ is selected from I and V;
X₅ is selected from I and V;
X₇ is selected from A and S;
X₉ is selected from I and L;
X₁₀ is selected from K and T;
X₁₂ is selected from D and N;
X₁₆ is selected from R, K, and L;
X₁₉ is selected from T and V;
X₂₀ is selected from S, and R;
X₂₂ is selected from K, D, and A; and
X₂₃ is selected from E, K, G, and N (e.g., the Cas9 molecule or Cas9 polypeptide can comprise an HNH-like domain as described herein).

In an embodiment, the HNH-like domain differs from a sequence of SEQ ID NO:28 by as many as 1 but no more than 2, 3, 4, or 5 residues.

In certain embodiments, a Cas9 molecule or Cas9 polypeptide comprises the amino acid sequence of Formula XIII:

(SEQ ID NO: 24)
L-Y-Y-L-Q-N-G-X₁′-D-M-Y-X₂′-X₃′-X₄′-X₅′-L-D-I-
X₆′-X₇′-L-S-X₈′-Y-Z-N-R-X₉′-K-X₁₀′-D-X₁₁′-V-P,

wherein
X₁′ is selected from K and R;
X₂′ is selected from V and T;
X₃′ is selected from G and D;
X₄′ is selected from E, Q and D;
X₅′ is selected from E and D;
X₆′ is selected from D, N, and H;
X₇′ is selected from Y, R, and N;
X₈′ is selected from Q, D, and N;
X₉′ is selected from G and E;
X₁₀′ is selected from S and G;
X₁₁′ is selected from D and N; and
Z is an HNH-like domain, e.g., as described above.

In certain embodiments, the Cas9 molecule or Cas9 polypeptide comprises an amino acid sequence that differs from a sequence of SEQ ID NO:24 by as many as 1 but not more than 2, 3, 4, or 5 residues.

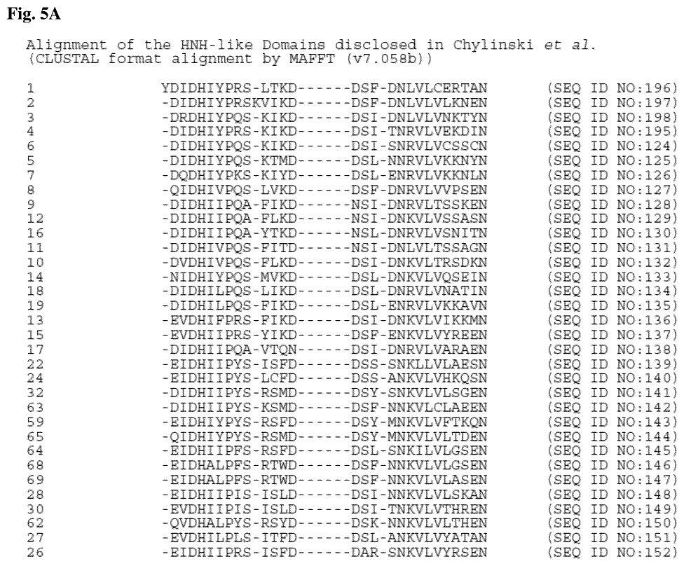

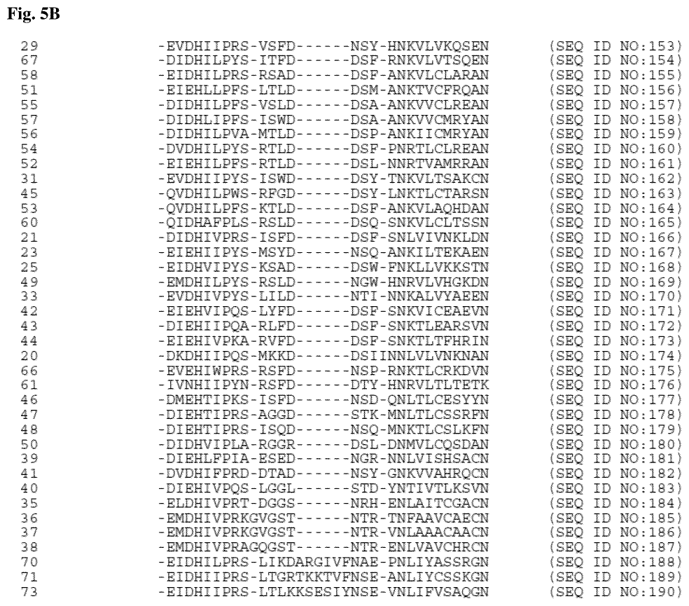

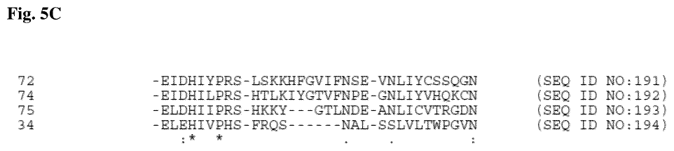

In certain embodiments, the HNH-like domain differs from a sequence of an HNH-like domain disclosed herein, e.g., in FIGS. 5A-5C, by as many as 1 but not more than 2, 3, 4, or 5 residues. In certain embodiments, 1 or both of the highly conserved residues identified in FIGS. 5A-5C are present.

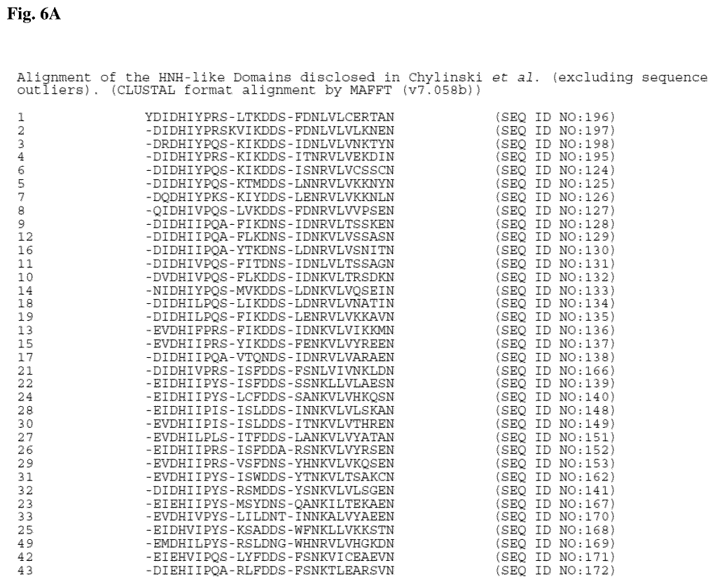

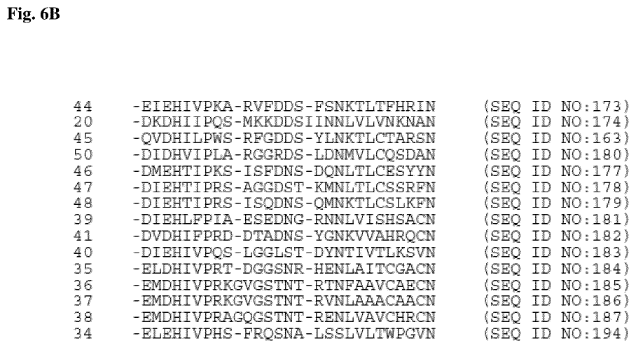

In certain embodiments, the HNH-like domain differs from a sequence of an HNH-like domain disclosed herein, e.g., in FIGS. 6A-6B, by as many as 1 but not more than 2, 3, 4, or 5 residues. In an embodiment, 1, 2, or all 3 of the highly conserved residues identified in FIGS. 6A-6B are present.

### Cas9 Activities

In certain embodiments, the Cas9 molecule or Cas9 polypeptide is capable of cleaving a target nucleic acid molecule. Typically, wild-type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 polypeptide which is a nickase, or which lacks the ability to cleave target nucleic acid. A Cas9 molecule or Cas9 polypeptide that is capable of cleaving a target nucleic acid molecule is referred to herein as an eaCas9 (an enzymatically active Cas9) molecule or eaCas9 polypeptide.

In certain embodiments, an eaCas9 molecule or eaCas9 polypeptide comprises one or more of the following enzymatic activities:
(1) nickase activity, i.e., the ability to cleave a single strand, e.g., the non-complementary strand or the complementary strand, of a nucleic acid molecule;
(2) double stranded nuclease activity, i.e., the ability to cleave both strands of a double stranded nucleic acid and create a double stranded break, which in an embodiment is the presence of two nickase activities;
(3) endonuclease activity;
(4) exonuclease activity; and
(5) helicase activity, i.e., the ability to unwind the helical structure of a double stranded nucleic acid.

In certain embodiments, an eaCas9 molecule or eaCas9 polypeptide cleaves both DNA strands and results in a double stranded break. In certain embodiments, an eaCas9 molecule or eaCas9 polypeptide cleaves only one strand, e.g., the strand to which the gRNA hybridizes to, or the strand complementary to the strand the gRNA hybridizes with. In an embodiment, an eaCas9 molecule or eaCas9 polypeptide comprises cleavage activity associated with an HNH domain. In an embodiment, an eaCas9 molecule or eaCas9 polypeptide comprises cleavage activity associated with a RuvC domain. In an embodiment, an eaCas9 molecule or eaCas9 polypeptide comprises cleavage activity associated with an HNH domain and cleavage activity associated with a RuvC domain. In an embodiment, an eaCas9 molecule or eaCas9 polypeptide comprises an active, or cleavage competent, HNH domain and an inactive, or cleavage incompetent, RuvC domain. In an embodiment, an eaCas9 molecule or eaCas9 polypeptide comprises an inactive, or cleavage incompetent, HNH domain and an active, or cleavage competent, RuvC domain.

### Targeting and PAMs

A Cas9 molecule or Cas9 polypeptide can interact with a gRNA molecule and, in concert with the gRNA molecule, localize to a site which comprises a target domain, and in certain embodiments, a PAM sequence.

In certain embodiments, the ability of an eaCas9 molecule or eaCas9 polypeptide to interact with and cleave a target nucleic acid is PAM sequence dependent. A PAM sequence is a sequence in the target nucleic acid. In an embodiment, cleavage of the target nucleic acid occurs upstream from the PAM sequence. eaCas9 molecules from different bacterial species can recognize different sequence motifs (e.g., PAM sequences). In an embodiment, an eaCas9 molecule of S. pyogenes recognizes the sequence motif NGG and directs cleavage of a target nucleic acid sequence 1 to 10, e.g., 3 to 5, bp upstream from that sequence (see, e.g., Mali 2013). In an embodiment, an eaCas9 molecule of S. thermophilus recognizes the sequence motif NGGNG (SEQ ID NO:199) and/or NNAGAAW (W=A or T) (SEQ ID NO:200) and directs cleavage of a target nucleic acid sequence 1 to 10, e.g., 3 to 5, bp upstream from these sequences (see, e.g., Horvath 2010; Deveau 2008). In an embodiment, an eaCas9 molecule of S. mutans recognizes the sequence motif NGG and/or NAAR (R=A or G) (SEQ ID NO:201) and directs cleavage of a target nucleic acid sequence 1 to 10, e.g., 3 to 5 bp, upstream from this sequence (see, e.g., Deveau 2008). In an embodiment, an eaCas9 molecule of S. aureus recognizes the sequence motif NNGRR (R=A or G) (SEQ ID NO:202) and directs cleavage of a target nucleic acid sequence 1 to 10, e.g., 3 to 5, bp upstream from that sequence. In an embodiment, an eaCas9 molecule of S. aureus recognizes the sequence motif NNGRRN (R=A or G) (SEQ ID NO:203) and directs cleavage of a target nucleic acid sequence 1 to 10, e.g., 3 to 5, bp upstream from that sequence. In an embodiment, an eaCas9 molecule of S. aureus recognizes the sequence motif NNGRRT (R=A or G) (SEQ ID NO:204) and directs cleavage of a target nucleic acid sequence 1 to 10, e.g., 3 to 5, bp upstream from that sequence. In an embodiment, an eaCas9 molecule of S. aureus recognizes the sequence motif NNGRRV (R=A or G, V=A, G, or C) (SEQ ID NO:205) and directs cleavage of a target nucleic acid sequence 1 to 10, e.g., 3 to 5, bp upstream from that sequence. The ability of a Cas9 molecule to recognize a PAM sequence can be determined, e.g., using a transformation assay as described previously (Jinek 2012). In each of the aforementioned embodiments (i.e., SEQ ID NOs:199-205), N can be any nucleotide residue, e.g., any of A, G, C, or T.

As is discussed herein, Cas9 molecules can be engineered to alter the PAM specificity of the Cas9 molecule.

Exemplary naturally occurring Cas9 molecules have been described previously (see, e.g., Chylinski 2013). Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. aureus, S. pyogenes (e.g., strains SF370, MGAS10270, MGAS10750, MGAS2096, MGAS315, MGAS5005, MGAS6180, MGAS9429, NZ131, SSI-1), S. thermophilus (e.g., strain LMD-9), S. pseudoporcinus (e.g., strain SPIN 20026), S. mutans (e.g., strains UA159, NN2025), S. macacae (e.g., strain NCTC11558), S. gallolyticus (e.g., strains UCN34, ATCC BAA-2069), S. equines (e.g., strains ATCC 9812, MGCS 124), S. dysdalactiae (e.g., strain GGS 124), S. bovis (e.g., strain ATCC 700338), S. anginosus (e.g., strain F0211), S. agalactiae (e.g., strains NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408).

In certain embodiments, a Cas9 molecule or Cas9 polypeptide comprises an amino acid sequence:
having 60%, 65%, 70%, 75%, 80%, 85%, 90%, 95%, 96%, 97%, 98%, or 99% homology with;
differs at no more than, 2, 5, 10, 15, 20, 30, or 40% of the amino acid residues when compared with;
differs by at least 1, 2, 5, 10 or 20 amino acids, but by no more than 100, 80, 70, 60, 50, 40, or 30 amino acids from; or
identical to any Cas9 molecule sequence described herein, or to a naturally occurring Cas9 molecule sequence, e.g., a Cas9 molecule from a species listed herein (e.g., SEQ ID NOs:1, 2, 4-6, or 12) or described in Chylinski 2013. In an embodiment, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to localize to a target nucleic acid.

In certain embodiments, a Cas9 molecule or Cas9 polypeptide comprises any of the amino acid sequence of the consensus sequence of FIGS. 2A-2G, wherein “*” indicates any amino acid found in the corresponding position in the amino acid sequence of a Cas9 molecule of S. pyogenes, S. thermophilus, S. mutans, or L. innocua, and “-” indicates absent. In an embodiment, a Cas9 molecule or Cas9 polypeptide differs from the sequence of the consensus sequence disclosed in FIGS. 2A-2G by at least 1, but no more than 2, 3, 4, 5, 6, 7, 8, 9, or 10 amino acid residues. In certain embodiments, a Cas9 molecule or Cas9 polypeptide comprises the amino acid sequence of SEQ ID NO:2. In other embodiments, a Cas9 molecule or Cas9 polypeptide differs from the sequence of SEQ ID NO:2 by at least 1, but no more than 2, 3, 4, 5, 6, 7, 8, 9, or 10 amino acid residues.

A comparison of the sequence of a number of Cas9 molecules indicate that certain regions are conserved. These are identified below as:
region 1 (residues 1 to 180, or in the case of region 1′ residues 120 to 180)
region 2 (residues 360 to 480);
region 3 (residues 660 to 720);
region 4 (residues 817 to 900); and
region 5 (residues 900 to 960).

In certain embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In certain embodiments, regions 1-5 each independently have 50%, 60%, 70%, 80%, 85%, 90%, 95%, 96%, 97%, 98%, or 99% homology with the corresponding residues of a Cas9 molecule or Cas9 polypeptide described herein, e.g., a sequence from FIGS. 2A-2G (SEQ ID NOs:1, 2, 4, 5, 14).

In certain embodiments, a Cas9 molecule or Cas9 polypeptide comprises an amino acid sequence referred to as region 1:
having 50%, 60%, 70%, 80%, 85%, 90%, 95%, 96%, 97%, 98%, or 99% homology with amino acids 1-180 (the numbering is according to the motif sequence in FIG. 2; 52% of residues in the four Cas9 sequences in FIGS. 2A-2G are conserved) of the amino acid sequence of Cas9 of S. pyogenes (SEQ ID NO:2);
differs by at least 1, 2, 5, 10 or 20 amino acids but by no more than 90, 80, 70, 60, 50, 40, or 30 amino acids from amino acids 1-180 of the amino acid sequence of Cas9 of S. pyogenes, S. thermophilus, S. mutans, or Listeria innocua (SEQ ID NOs:2, 4, 1, and 5, respectively); or is identical to amino acids 1-180 of the amino acid sequence of Cas9 of S. pyogenes, S. thermophilus, S. mutans, or L. innocua (SEQ ID NOs:2, 4, 1, and 5, respectively).

In certain embodiments, a Cas9 molecule or Cas9 polypeptide comprises an amino acid sequence referred to as region 1′:
having 55%, 60%, 65%, 70%, 75%, 80%, 85%, 90%, 95%, 96%, 97%, 98%, or 99% homology with amino acids 120-180 (55% of residues in the four Cas9 sequences in FIG. 2 are conserved) of the amino acid sequence of Cas9 of S. pyogenes, S. thermophilus, S. mutans, or L. innocua (SEQ ID NOs:2, 4, 1, and 5, respectively);
differs by at least 1, 2, or 5 amino acids but by no more than 35, 30, 25, 20, or 10 amino acids from amino acids 120-180 of the amino acid sequence of Cas9 of S. pyogenes, S. thermophilus, S. mutans, or L. innocua (SEQ ID NOs:2, 4, 1, and 5, respectively); or is identical to amino acids 120-180 of the amino acid sequence of Cas9 of S. pyogenes, S. thermophilus, S. mutans, or L. innocua (SEQ ID NOs:2, 4, 1, and 5, respectively).

In certain embodiments, a Cas9 molecule or Cas9 polypeptide comprises an amino acid sequence referred to as region 2:
having 50%, 55%, 60%, 65%, 70%, 75%, 80%, 85%, 90%, 95%, 96%, 97%, 98%, or 99% homology with amino acids 360-480 (52% of residues in the four Cas9 sequences in FIG. 2 are conserved) of the amino acid sequence of Cas9 of S. pyogenes, S. thermophilus, S. mutans, or L. innocua (SEQ ID NOs:2, 4, 1, and 5, respectively);
differs by at least 1, 2, or 5 amino acids but by no more than 35, 30, 25, 20, or 10 amino acids from amino acids 360-480 of the amino acid sequence of Cas9 of S. pyogenes, S. thermophilus, S. mutans, or L. innocua (SEQ ID NOs:2, 4, 1, and 5, respectively); or is identical to amino acids 360-480 of the amino acid sequence of Cas9 of S. pyogenes, S. thermophilus, S. mutans, or L. innocua (SEQ ID NOs:2, 4, 1, and 5, respectively).

In certain embodiments, a Cas9 molecule or Cas9 polypeptide comprises an amino acid sequence referred to as region 3:
having 55%, 60%, 65%, 70%, 75%, 80%, 85%, 90%, 95%, 96%, 97%, 98%, or 99% homology with amino acids 660-720 (56% of residues in the four Cas9 sequences in FIG. 2 are conserved) of the amino acid sequence of Cas9 of S. pyogenes, S. thermophilus, S. mutans, or L. innocua (SEQ ID NOs:2, 4, 1, and 5, respectively);
differs by at least 1, 2, or 5 amino acids but by no more than 35, 30, 25, 20, or 10 amino acids from amino acids 660-720 of the amino acid sequence of Cas9 of S. pyogenes, S. thermophilus, S. mutans, or L. innocua (SEQ ID NOs:2, 4, 1, and 5, respectively); or is identical to amino acids 660-720 of the amino acid sequence of Cas9 of S. pyogenes, S. thermophilus, S. mutans, or L. innocua (SEQ ID NOs:2, 4, 1, and 5, respectively).

In certain embodiments, a Cas9 molecule or Cas9 polypeptide comprises an amino acid sequence referred to as region 4:
having 50%, 55%, 60%, 65%, 70%, 75%, 80%, 85%, 90%, 95%, 96%, 97%, 98%, or 99% homology with amino acids 817-900 (55% of residues in the four Cas9 sequences in FIGS. 2A-2G are conserved) of the amino acid sequence of Cas9 of S. pyogenes, S. thermophilus, S. mutans, or L. innocua (SEQ ID NOs:2, 4, 1, and 5, respectively);
differs by at least 1, 2, or 5 amino acids but by no more than 35, 30, 25, 20, or 10 amino acids from amino acids 817-900 of the amino acid sequence of Cas9 of S. pyogenes, S. thermophilus, S. mutans, or L. innocua (SEQ ID NOs:2, 4, 1, and 5, respectively); or
is identical to amino acids 817-900 of the amino acid sequence of Cas9 of S. pyogenes, S. thermophilus, S. mutans, or L. innocua (SEQ ID NOs:2, 4, 1, and 5, respectively).

In certain embodiments, a Cas9 molecule or Cas9 polypeptide comprises an amino acid sequence referred to as region 5:
having 50%, 55%, 60%, 65%, 70%, 75%, 80%, 85%, 90%, 95%, 96%, 97%, 98%, or 99% homology with amino acids 900-960 (60% of residues in the four Cas9 sequences in FIGS. 2A-2G are conserved) of the amino acid sequence of Cas9 of S. pyogenes, S. thermophilus, S. mutans, or L. innocua (SEQ ID NOs:2, 4, 1, and 5, respectively);
differs by at least 1, 2, or 5 amino acids but by no more than 35, 30, 25, 20, or 10 amino acids from amino acids 900-960 of the amino acid sequence of Cas9 of S. pyogenes, S. thermophilus, S. mutans, or L. innocua (SEQ ID NOs:2, 4, 1, and 5, respectively); or
is identical to amino acids 900-960 of the amino acid sequence of Cas9 of S. pyogenes, S. thermophilus, S. mutans, or L. innocua (SEQ ID NOs:2, 4, 1, and 5, respectively).

### Engineered or Altered Cas9

Cas9 molecules and Cas9 polypeptides described herein can possess any of a number of properties, including nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In certain embodiments, a Cas9 molecule or Cas9 polypeptide can include all or a subset of these properties. In a typical embodiment, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (engineered, as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity (as compared with a naturally occurring or other reference Cas9 molecule) or altered helicase activity. As discussed herein, an engineered Cas9 molecule or Cas9 polypeptide can have nickase activity (as opposed to double strand nuclease activity). In certain embodiments, an engineered Cas9 molecule or Cas9 polypeptide can have an alteration that alters its size, e.g., a deletion of amino acid sequence that reduces its size, e.g., without significant effect on one or more Cas9 activities. In certain embodiments, an engineered Cas9 molecule or Cas9 polypeptide can comprise an alteration that affects PAM recognition, e.g., an engineered Cas9 molecule can be altered to recognize a PAM sequence other than that recognized by the endogenous wild-type PI domain. In certain embodiments, a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In an embodiment, a Cas9 molecule or Cas9 polypeptide can comprises one or more mutations or differences, e.g., at least 1, 2, 3, 4, 5, 10, 15, 20, 30, 40, or 50 mutations but less than 200, 100, or 80 mutations relative to a reference, e.g., a parental, Cas9 molecule.

In certain embodiments, a mutation or mutations do not have a substantial effect on a Cas9 activity, e.g., a Cas9 activity described herein. In other embodiments, a mutation or mutations have a substantial effect on a Cas9 activity, e.g., a Cas9 activity described herein.

### Non-Cleaving and Modified-Cleavage Cas9

In an embodiment, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

In certain embodiments, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In certain embodiments, an eaCas9 molecule or eaCas9 polypeptide comprises an active, or cleavage competent, HNH-like domain (e.g., an HNH-like domain described herein, e.g., SEQ ID NOs:24-28) and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence disclosed in FIGS. 2A-2G or an aspartic acid at position 10 of SEQ ID NO:2, e.g., can be substituted with an alanine. In an embodiment, the eaCas9 molecule or eaCas9 polypeptide differs from wild-type in the N-terminal RuvC-like domain and does not cleave the target nucleic acid, or cleaves with significantly less efficiency, e.g., less than 20, 10, 5, 1, or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. aureus, or S. thermophilus. In an embodiment, the reference Cas9 molecule is the naturally occurring Cas9 molecule having the closest sequence identity or homology.

In an embodiment, an eaCas9 molecule or eaCas9 polypeptide comprises an inactive, or cleavage incompetent, HNH domain and an active, or cleavage competent, N-terminal RuvC-like domain (e.g., a RuvC-like domain described herein, e.g., SEQ ID NOs:15-23). Exemplary inactive, or cleavage incompetent HNH-like domains can have a mutation at one or more of: a histidine in an HNH-like domain, e.g., a histidine shown at position 856 of the consensus sequence disclosed in FIGS. 2A-2G, e.g., can be substituted with an alanine; and one or more asparagines in an HNH-like domain, e.g., an asparagine shown at position 870 of the consensus sequence disclosed in FIGS. 2A-2G and/or at position 879 of the consensus sequence disclosed in FIGS. 2A-2G, e.g., can be substituted with an alanine. In an embodiment, the eaCas9 differs from wild-type in the HNH-like domain and does not cleave the target nucleic acid, or cleaves with significantly less efficiency, e.g., less than 20, 10, 5, 1, or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. aureus, or S. thermophilus. In an embodiment, the reference Cas9 molecule is the naturally occurring Cas9 molecule having the closest sequence identity or homology.

In certain embodiments, exemplary Cas9 activities comprise one or more of PAM specificity, cleavage activity, and helicase activity. A mutation(s) can be present, e.g., in: one or more RuvC domains, e.g., an N-terminal RuvC domain; an HNH domain; a region outside the RuvC domains and the HNH domain. In an embodiment, a mutation(s) is present in a RuvC domain. In an embodiment, a mutation(s) is present in an HNH domain. In an embodiment, mutations are present in both a RuvC domain and an HNH domain.

Exemplary mutations that may be made in the RuvC domain or HNH domain with reference to the S. pyogenes Cas9 sequence include: D10A, E762A, H840A, N854A, N863A, and/or D986A. Exemplary mutations that may be made in the RuvC domain with reference to the S. aureus Cas9 sequence include N580A (see, e.g., SEQ ID NO:11).

Whether or not a particular sequence, e.g., a substitution, may affect one or more activity, such as targeting activity, cleavage activity, etc., can be evaluated or predicted, e.g., by evaluating whether the mutation is conservative. In an embodiment, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In an embodiment, a Cas9 molecule comprises a cleavage property that differs from naturally occurring Cas9 molecules, e.g., that differs from the naturally occurring Cas9 molecule having the closest homology. For example, a Cas9 molecule can differ from naturally occurring Cas9 molecules, e.g., a Cas9 molecule of S. aureus or S. pyogenes, as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S. aureus or S. pyogenes); its ability to modulate, e.g., decreased or increased, cleavage of a single strand of a nucleic acid, e.g., a non-complimentary strand of a nucleic acid molecule or a complementary strand of a nucleic acid molecule (nickase activity), e.g., as compared to a naturally occurring Cas9 molecule (e.g., a Cas9 molecule of S. aureus or S. pyogenes); or the ability to cleave a nucleic acid molecule, e.g., a double stranded or single stranded nucleic acid molecule, can be eliminated. In certain embodiments, the nickase is S. aureus Cas9-derived nickase comprising the sequence of SEQ ID NO:10 (D10A) or SEQ ID NO:11 (N580A) (Friedland 2015).

In an embodiment, the altered Cas9 molecule is an eaCas9 molecule comprising one or more of the following activities: cleavage activity associated with a RuvC domain; cleavage activity associated with an HNH domain; cleavage activity associated with an HNH domain and cleavage activity associated with a RuvC domain.

In certain embodiments, the altered Cas9 molecule or Cas9 polypeptide comprises a sequence in which:
the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G; and
the sequence corresponding to the residues identified by “*” in the consensus sequence disclosed in FIGS. 2A-2G differs at no more than 1, 2, 3, 4, 5, 10, 15, 20, 25, 30, 35, or 40% of the “*” residues from the corresponding sequence of naturally occurring Cas9 molecule, e.g., an S. pyogenes, S. thermophilus, S. mutans, or L. inocua Cas9 molecule.

In an embodiment, the altered Cas9 molecule or Cas9 polypeptide is an eaCas9 molecule or eaCas9 polypeptide comprising the amino acid sequence of S. pyogenes Cas9 disclosed in FIGS. 2A-2G (SEQ ID NO:2) with one or more amino acids that differ from the sequence of S. pyogenes (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G (SEQ ID NO:14).

In an embodiment, the altered Cas9 molecule or Cas9 polypeptide is an eaCas9 molecule or eaCas9 polypeptide comprising the amino acid sequence of S. thermophilus Cas9 disclosed in FIGS. 2A-2G (SEQ ID NO:4) with one or more amino acids that differ from the sequence of S. thermophilus (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G (SEQ ID NO:14).

In an embodiment, the altered Cas9 molecule or Cas9 polypeptide is an eaCas9 molecule or eaCas9 polypeptide comprising the amino acid sequence of S. mutans Cas9 disclosed in FIGS. 2A-2G (SEQ ID NO:1) with one or more amino acids that differ from the sequence of S. mutans (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G (SEQ ID NO:14).

In an embodiment, the altered Cas9 molecule or Cas9 polypeptide is an eaCas9 molecule or eaCas9 polypeptide comprising the amino acid sequence of L. inocua Cas9 disclosed in FIGS. 2A-2G (SEQ ID NO:5) with one or more amino acids that differ from the sequence of L. inocua (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G (SEQ ID NO:14).

In certain embodiments, the altered Cas9 molecule or Cas9 polypeptide, e.g., an eaCas9 molecule or eaCas9 polypeptide, can be a fusion, e.g., of two of more different Cas9 molecules, e.g., of two or more naturally occurring Cas9 molecules of different species. For example, a fragment of a naturally occurring Cas9 molecule of one species can be fused to a fragment of a Cas9 molecule of a second species. As an example, a fragment of a Cas9 molecule of S. pyogenes comprising an N-terminal RuvC-like domain can be fused to a fragment of Cas9 molecule of a species other than S. pyogenes (e.g., S. thermophilus) comprising an HNH-like domain.

Cas9 with Altered or No PAM Recognition

Naturally occurring Cas9 molecules can recognize specific PAM sequences, for example the PAM recognition sequences described above for, e.g., S. pyogenes, S. thermophilus, S. mutans, and S. aureus.

In certain embodiments, a Cas9 molecule or Cas9 polypeptide has the same PAM specificities as a naturally occurring Cas9 molecule. In other embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule or Cas9 polypeptide recognizes in order to decrease off-target sites and/or improve specificity; or eliminate a PAM recognition requirement. In certain embodiments, a Cas9 molecule or Cas9 polypeptide can be altered, e.g., to increase length of PAM recognition sequence and/or improve Cas9 specificity to high level of identity (e.g., 98%, 99%, or 100% match between gRNA and a PAM sequence), e.g., to decrease off-target sites and/or increase specificity. In certain embodiments, the length of the PAM recognition sequence is at least 4, 5, 6, 7, 8, 9, 10, or 15 amino acids in length. In an embodiment, the Cas9 specificity requires at least 90%, 95%, 96%, 97%, 98%, 99% or more homology between the gRNA and the PAM sequence. Cas9 molecules or Cas9 polypeptides that recognize different PAM sequences and/or have reduced off-target activity can be generated using directed evolution. Exemplary methods and systems that can be used for directed evolution of Cas9 molecules are described (see, e.g., Esvelt 2011). Candidate Cas9 molecules can be evaluated, e.g., by methods described below.

### Size-Optimized Cas9

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. Provided herein are Cas9 molecules or Cas9 polypeptides comprising one or more deletions and optionally one or more linkers, wherein a linker is disposed between the amino acid residues that flank the deletion. Methods for identifying suitable deletions in a reference Cas9 molecule, methods for generating Cas9 molecules with a deletion and a linker, and methods for using such Cas9 molecules will be apparent to one of ordinary skill in the art upon review of this document.

A Cas9 molecule, e.g., a S. aureus or S. pyogenes Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome-editing. A Cas9 molecule can comprise one or more deletions that do not substantially affect or decrease the activity of the resultant Cas9 molecules described herein. Activities that are retained in the Cas9 molecules comprising a deletion as described herein include one or more of the following:
a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in an embodiment is the presence of two nickase activities;
an endonuclease activity;
an exonuclease activity;
a helicase activity, i.e., the ability to unwind the helical structure of a double stranded nucleic acid;
and recognition activity of a nucleic acid molecule, e.g., a target nucleic acid or a gRNA.

Activity of the Cas9 molecules described herein can be assessed using the activity assays described herein or in the art.

### Identifying Regions Suitable for Deletion

Suitable regions of Cas9 molecules for deletion can be identified by a variety of methods. Naturally-occurring orthologous Cas9 molecules from various bacterial species, e.g., any one of those listed in Table 1, can be modeled onto the crystal structure of S. pyogenes Cas9 (Nishimasu 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

### Nucleic Acids Encoding Cas9 Molecules

Nucleic acids encoding the Cas9 molecules or Cas9 polypeptides, e.g., an eaCas9 molecule or eaCas9 polypeptides are provided herein. Exemplary nucleic acids encoding Cas9 molecules or Cas9 polypeptides have been described previously (see, e.g., Cong 2013; Wang 2013; Mali 2013; Jinek 2012).

In an embodiment, a nucleic acid encoding a Cas9 molecule or Cas9 polypeptide can be a synthetic nucleic acid sequence. For example, the synthetic nucleic acid molecule can be chemically modified, e.g., as described herein. In an embodiment, the Cas9 mRNA has one or more (e.g., all of the following properties: it is capped, polyadenylated, substituted with 5-methylcytidine and/or pseudouridine.

In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

In addition, or alternatively, a nucleic acid encoding a Cas9 molecule or Cas9 polypeptide may comprise a nuclear localization sequence (NLS). Nuclear localization sequences are known in the art.

An exemplary codon optimized nucleic acid sequence encoding a Cas9 molecule of S. pyogenes is set forth in SEQ ID NO:3. The corresponding amino acid sequence of an S. pyogenes Cas9 molecule is set forth in SEQ ID NO:2.

Exemplary codon optimized nucleic acid sequences encoding a Cas9 molecule of S. aureus are set forth in SEQ ID NOs:7-9. An amino acid sequence of an S. aureus Cas9 molecule is set forth in SEQ ID NO:6.

If any of the above Cas9 sequences are fused with a peptide or polypeptide at the C-terminus, it is understood that the stop codon will be removed.

### Other Cas Molecules and Cas Polypeptides

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) have been described previously (see, e.g., Haft 2005 and Makarova 2011). Exemplary Cas molecules (and Cas systems) are also shown in Table 2.

TABLE 2
Cas systems

| | | | Structure of | Families (and | |
|---|---|---|---|---|---|
| | | | encoded | superfamily) of | |
| Gene | System type | Name from | protein (PDB | encoded | |
| name^{‡} | or subtype | Haft 2005^{§} | accessions)^{¶} | protein^{#}** | Representatives |
| cas1 | Type I | cas1 | 3GOD, 3LFX | COG1518 | SERP2463, SPy1047 |
| | Type II | | and 2YZS | | and ygbT |
| | Type III | | | | |
| cas2 | Type I | cas2 | 2IVY, 2I8E and | COG1343 and | SERP2462, SPy1048, |
| | Type II | | 3EXC | COG3512 | SPy1723 (N-terminal |
| | Type III | | | | domain) and ygbF |
| cas3′ | Type I^{‡‡} | cas3 | NA | COG1203 | APE1232 and ygcB |
| cas3″ | Subtype I-A | NA | NA | COG2254 | APE1231 and |
| | Subtype I-B | | | | BH0336 |
| cas4 | Subtype I-A | cas4 and csa1 | NA | COG1468 | APE1239 and |
| | Subtype I-B | | | | BH0340 |
| | Subtype I-C | | | | |
| | Subtype I-D | | | | |
| | Subtype II- | | | | |
| | B | | | | |
| cas5 | Subtype I-A | cas5a, cas5d, | 3KG4 | COG1688 | APE1234, BH0337, |
| | Subtype I-B | cas5e, cas5h, | | (RAMP) | devS and ygcI |
| | Subtype I-C | cas5p, cas5t | | | |
| | Subtype I-E | and cmx5 | | | |
| cas6 | Subtype I-A | cas6 and cmx6 | 3I4H | COG1583 and | PF1131 and s1r7014 |
| | Subtype I-B | | | COG5551 | |
| | Subtype I-D | | | (RAMP) | |
| | Subtype III- | | | | |
| | A• Subtype | | | | |
| | III-B | | | | |
| cas6e | Subtype I-E | cse3 | 1WJ9 | (RAMP) | ygcH |
| cas6f | Subtype I-F | csy4 | 2XLJ | (RAMP) | y1727 |
| cas7 | Subtype I-A | csa2, csd2, | NA | COG1857 and | devR and ygcJ |
| | Subtype I-B | cse4, csh2, | | COG3649 | |
| | Subtype I-C | csp1 and cst2 | | (RAMP) | |
| | Subtype I-E | | | | |
| cas8a1 | Subtype I- | cmx1, cst1, | NA | BH0338-like | LA3191^{§§} and |
| | A^{‡‡} | csx8, csx13 | | | PG2018^{§§} |
| | | and CXXC-CXXC | | | |
| cas8a2 | Subtype I- | csa4 and csx9 | NA | PH0918 | AF0070, AF1873, |
| | A^{‡‡} | | | | MJ0385, PF0637, |
| | | | | | PH0918 and |
| | | | | | SSO1401 |
| cas8b | Subtype I- | csh1 and | NA | BH0338-like | MTH1090 and |
| | B^{‡‡} | TM1802 | | | TM1802 |
| cas8c | Subtype I- | csd1 and csp2 | NA | BH0338-like | BH0338 |
| | C^{‡‡} | | | | |
| cas9 | Type II^{‡‡} | csn1 and csx12 | NA | COG3513 | FTN_0757 and |
| | | | | | SPy1046 |
| cas10 | Type III^{‡‡} | cmr2, csm1 | NA | COG1353 | MTH326, Rv2823c^{§§} |
| | | and csx11 | | | and TM1794^{§§} |
| cas10d | Subtype I- | csc3 | NA | COG1353 | slr7011 |
| | D^{‡‡} | | | | |
| csy1 | Subtype I- | csy1 | NA | y1724-like | y1724 |
| | F^{‡‡} | | | | |
| csy2 | Subtype I-F | csy2 | NA | (RAMP) | y1725 |
| csy3 | Subtype I-F | csy3 | NA | (RAMP) | y1726 |
| cse1 | Subtype I- | cse1 | NA | YgcL-like | ygcL |
| | E^{‡‡} | | | | |
| cse2 | Subtype I-E | cse2 | 2ZCA | YgcK-like | ygcK |
| csc1 | Subtype I-D | csc1 | NA | alr1563-like | alr1563 |
| | | | | (RAMP) | |
| csc2 | Subtype I-D | csc1 and csc2 | NA | COG1337 | slr7012 |
| | | | | (RAMP) | |
| csa5 | Subtype I-A | csa5 | NA | AF1870 | AF1870, MJ0380, |
| | | | | | PF0643 and |
| | | | | | SSO1398 |
| csn2 | Subtype II- | csn2 | NA | SPy1049-like | SPy1049 |
| | A | | | | |
| csm2 | Subtype III- | csm2 | NA | COG1421 | MTH1081 and |
| | A^{‡‡} | | | | SERP2460 |
| csm3 | Subtype III- | csc2 and csm3 | NA | COG1337 | MTH1080 and |
| | A | | | (RAMP) | SERP2459 |
| csm4 | Subtype III- | csm4 | NA | COG1567 | MTH1079 and |
| | A | | | (RAMP) | SERP2458 |
| csm5 | Subtype III- | csm5 | NA | COG1332 | MTH1078 and |
| | A | | | (RAMP) | SERP2457 |
| csm6 | Subtype III- | APE2256 and | 2WTE | COG1517 | APE2256 and |
| | A | csm6 | | | SSO1445 |
| cmr1 | Subtype III- | cmr1 | NA | COG1367 | PF1130 |
| | B | | | (RAMP) | |
| cmr3 | Subtype III- | cmr3 | NA | COG1769 | PF1128 |
| | B | | | (RAMP) | |
| cmr4 | Subtype III- | cmr4 | NA | COG1336 | PF1126 |
| | B | | | (RAMP) | |
| cmr5 | Subtype III- | cmr5 | 2ZOP and | COG3337 | MTH324 and PF1125 |
| | B^{‡‡} | | 2OEB | | |
| cmr6 | Subtype III- | cmr6 | NA | COG1604 | PF1124 |
| | B | | | (RAMP) | |
| csb1 | Subtype I-U | GSU0053 | NA | (RAMP) | Balac_1306 and |
| | | | | | GSU0053 |
| csb2 | Subtype I- | NA | NA | (RAMP) | Balac_1305 and |
| | U^{§§} | | | | GSU0054 |
| csb3 | Subtype I-U | NA | NA | (RAMP) | Balac_1303^{§§} |
| csx17 | Subtype I-U | NA | NA | NA | Btus_2683 |
| csx14 | Subtype I-U | NA | NA | NA | GSU0052 |
| csx10 | Subtype I-U | csx10 | NA | (RAMP) | Caur_2274 |
| csx16 | Subtype III- | VVA1548 | NA | NA | VVA1548 |
| | U | | | | |
| csaX | Subtype III- | csaX | NA | NA | SSO1438 |
| | U | | | | |
| csx3 | Subtype III- | csx3 | NA | NA | AF1864 |
| | U | | | | |
| csx1 | Subtype III- | csa3, csx1, | 1XMX and 2I71 | COG1517 and | MJ1666, NE0113, |
| | U | csx2,DXTHG, | | COG4006 | PF1127 and TM1812 |
| | | NE0113 and | | | |
| | | TIGR02710 | | | |
| csx15 | Unknown | NA | NA | TTE2665 | TTE2665 |
| csf1 | Type U | csf1 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

### Cpf1 Molecules

The crystal structure of Acidaminococcus sp. Cpf1 in complex with crRNA and a double-stranded (ds) DNA target including a TTTN PAM sequence has been solved by Yamano 2016, incorporated by reference herein. Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:anti-repeat duplex in Cas9 gRNAs.

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran 2013 and Yamano 2016, as well as in Cotta-Ramusino 2016. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand as shown below (where C denotes the site of cleavage):

5′-------------------[protospacer]--[C]---------------------3′
3′----------------------------------------------------------5′

On the other hand, inactivation of a Cas9 HNH domain results in a nickase that cleaves the bottom or non-complementary strand:

5′-------------------[protospacer]---------------------------3′
3′-----------------------------------[C]---------------------5′

Modifications of PAM specificity relative to naturally occurring Cas9 reference molecules has been described by Kleinstiver 2015a for both S. pyogenes and S. aureus (Kleinstiver 2015b). Kleinstiver et al. have also described modifications that improve the targeting fidelity of Cas9 (Kleinstiver 2016). Each of these references is incorporated by reference herein.

RNA-guided nucleases have been split into two or more parts, as described by Zetsche 2015, incorporated by reference, and by Fine 2015, incorporated by reference.

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger 2014, which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder 2015 and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino 2016.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate Cas9 molecules, candidate gRNA molecules, candidate Cas9 molecule/gRNA molecule complexes, can be evaluated by art-known methods or as described herein. For example, exemplary methods for evaluating the endonuclease activity of Cas9 molecule have been described previously (Jinek 2012).

### Binding and Cleavage Assay: Testing the Endonuclease Activity of Cas9 Molecules

The ability of a Cas9 molecule/gRNA molecule complex to bind to and cleave a target nucleic acid can be evaluated in a plasmid cleavage assay. In this assay, a synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 minutes at 37° C. with purified Cas9 protein molecule (50-500 nM) and gRNA (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl₂. The reactions are stopped with 5×DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands, while nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 μmol) are radiolabeled by incubating with 5 units T4 polynucleotide kinase and ˜3-6 pmol (˜20-40 mCi) [γ-³²P]-ATP in 1×T4 polynucleotide kinase reaction buffer at 37° C. for 30 minutes, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 minutes, followed by slow cooling to room temperature. For cleavage assays, gRNA molecules are annealed by heating to 95° C. for 30 seconds, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgCl2, 1 mM DTT, 5% glycerol) in a total volume of 9 L. Reactions are initiated by the addition of 1 μL target DNA (10 nM) and incubated for 1 hour at 37° C. Reactions are quenched by the addition of 20 μL of loading dye (5 mM EDTA, 0.025% SDS, 5% glycerol in formamide) and heated to 95° C. for 5 minutes. Cleavage products are resolved on 12% denaturing polyacrylamide gels containing 7 M urea and visualized by phosphorimaging. The resulting cleavage products indicate that whether the complementary strand, the non-complementary strand, or both are cleaved.

One or both of these assays can be used to evaluate the suitability of a candidate gRNA molecule or candidate Cas9 molecule.

### Binding Assay: Testing the Binding of Cas9 Molecules to Target DNA

Exemplary methods for evaluating the binding of Cas9 molecules to target DNA have been described previously (Jinek 2012).

For example, in an electrophoretic mobility shift assay, target DNA duplexes are formed by mixing of each strand (10 nmol) in deionized water, heating to 95° C. for 3 minutes, and slow cooling to room temperature. All DNAs are purified on 8% native gels containing 1×TBE. DNA bands are visualized by UV shadowing, excised, and eluted by soaking gel pieces in DEPC-treated H₂O. Eluted DNA is ethanol precipitated and dissolved in DEPC-treated H₂O. DNA samples are 5′ end labeled with [γ-³²P]-ATP using T4 polynucleotide kinase for 30 minutes at 37° C. Polynucleotide kinase is heat denatured at 65° C. for 20 minutes, and unincorporated radiolabel is removed using a column. Binding assays are performed in buffer containing 20 mM HEPES pH 7.5, 100 mM KCl, 5 mM MgCl₂, 1 mM DTT, and 10% glycerol in a total volume of 10 μL. Cas9 protein molecules are programmed with equimolar amounts of pre-annealed gRNA molecule and titrated from 100 pM to 1 μM. Radiolabeled DNA is added to a final concentration of 20 pM. Samples are incubated for 1 hour at 37° C. and resolved at 4° C. on an 8% native polyacrylamide gel containing 1×TBE and 5 mM MgCl₂. Gels are dried and DNA visualized by phosphorimaging.

### Differential Scanning Flourimetry (DSF)

The thermostability of Cas9-gRNA ribonucleoprotein (RNP) complexes can be measured via DSF. This technique measures the thermostability of a protein, which can increase under favorable conditions such as the addition of a binding RNA molecule, e.g., a gRNA.

The assay can be performed using two different protocols, one to test the best stoichiometric ratio of gRNA:Cas9 protein and another to determine the best solution conditions for RNP formation.

To determine the best solution conditions for forming RNP complexes, a 2 M solution of Cas9 is made in water with 10× SYPRO Orange® (Life Technologies cat #S-6650) and dispensed into a 384 well plate. An equimolar amount of gRNA diluted in solutions with varied pH and salt is then added. After incubating at room temperature for 10 minutes and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with 2 M Cas9 in optimal buffer from assay 1 above, and incubating at room temperature for 10 minutes in a 384 well plate. An equal volume of optimal buffer and 10× SYPRO Orange® (Life Technologies cat #S-6650) is added and the plate is sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### NHEJ Approaches for Gene Targeting

In certain embodiments of the methods provided herein, NHEJ-mediated deletion is used to delete all or a portion of a γ-globin gene (e.g., HBG1, HBG2) negative regulatory element (e.g., silencer). As described herein, nuclease-induced NHEJ can be used to knock out all or a portion of a regulatory element in a target-specific manner. In other embodiments, NHEJ-mediated insertion is used to insert a sequence into a γ-globin gene negative regulatory element, resulting in inactivation of the regulatory element.

While not wishing to be bound by theory, it is believed that, in certain embodiments, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein.

The indel mutations generated by NHEJ are unpredictable in nature; however, at a given break site certain indel sequences are favored and are over represented in the population, likely due to small regions of microhomology. The lengths of deletions can vary widely; they are most commonly in the 1-50 bp range, but can reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs (e.g., motifs less than or equal to 50 nucleotides in length) as long as the generation of a specific final sequence is not required. If a double-strand break is targeted near to a target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. In this way, DNA segments as large as several hundred kilobases can be deleted. Both of these approaches can be used to delete specific DNA sequences; however, the error-prone nature of NHEJ may still produce indel mutations at the site of repair.

Both double strand cleaving eaCas9 molecules and single strand, or nickase, eaCas9 molecules can be used in the methods and compositions described herein to generate NHEJ-mediated indels. NHEJ-mediated indels targeted to a regulatory region of interest can be used to disrupt or delete a target regulatory element.

### Placement of Double Strand or Single Strand Breaks Relative to the Target Position

In certain embodiments in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In an embodiment, the cleavage site is between 0-30 bp away from the target position (e.g., less than 30, 25, 20, 15, 10, 9, 8, 7, 6, 5, 4, 3, 2, or 1 bp from the target position).

In certain embodiments in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In certain embodiments, the gRNAs are configured to position cuts at the same position, or within a few nucleotides of one another, on different strands, essentially mimicking a double strand break. In certain embodiments, the closer nick is between 0-30 bp away from the target position (e.g., less than 30, 25, 20, 15, 10, 9, 8, 7, 6, 5, 4, 3, 2, or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20, or 10 bp). In certain embodiments, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks in deleted). In certain embodiments, two gRNAs, e.g., independently, unimolecular (or chimeric) or modular gRNA, are configured to position a double-strand break on both sides of a target position. In other embodiments, three gRNAs, e.g., independently, unimolecular (or chimeric) or modular gRNA, are configured to position a double strand break (i.e., one gRNA complexes with a cas9 nuclease) and two single strand breaks or paired single strand breaks (i.e., two gRNAs complex with Cas9 nickases) on either side of the target position. In still other embodiments, four gRNAs, e.g., independently, unimolecular (or chimeric) or modular gRNA, are configured to generate two pairs of single strand breaks (i.e., two pairs of two gRNAs complex with Cas9 nickases) on either side of the target position. The double strand break(s) or the closer of the two single strand nicks in a pair will ideally be within 0-500 bp of the target position (e.g., no more than 450, 400, 350, 300, 250, 200, 150, 100, 50, or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20, or 10 bp).

### HDR Repair, HDR-Mediated Knock-In, Knock-Out, or Deletion, and Template Nucleic Acids

In certain embodiments of the methods provided herein, HDR-mediated sequence alteration is used to alter (e.g., delete, disrupt, or modify) the sequence of one or more nucleotides in a γ-globin gene (e.g., HBG1, HBG2) regulatory region using an exogenously provided template nucleic acid (also referred to herein as a donor construct). While not wishing to be bound by theory, it is believed that HDR-mediated alteration of an HBG target position within a γ-globin gene regulatory region occurs by HDR with an exogenously provided donor template or template nucleic acid. For example, the donor construct or template nucleic acid provides for alteration of an HBG target position. It is contemplated that a plasmid donor can be used as a template for homologous recombination. It is further contemplated that a single stranded donor template can be used as a template for alteration of the HBG target position by alternate methods of HDR (e.g., single strand annealing) between the target sequence and the donor template. Donor template-effected alteration of an HBG target position depends on cleavage by a Cas9 molecule. Cleavage by Cas9 can comprise a double-strand break or two single-strand breaks.

In certain embodiments of the methods provided herein, HDR-mediated alteration is used to knock out or delete all or a portion of a γ-globin gene (e.g., HBG1, HBG2) negative regulatory element (e.g., silencer). As described herein, HDR can be used to knock out or delete all or a portion of a regulatory element in a target-specific manner.

In other embodiments, HDR-mediated sequence alteration is used to alter the sequence of one or more nucleotides in a γ-globin gene (e.g., HBG1, HBG2) regulatory region without using an exogenously provided template nucleic acid. While not wishing to be bound by theory, it is believed that alteration of an HBG target position occurs by HDR with an endogenous genomic donor sequence. For example, the endogenous genomic donor sequence provides for alteration of the HBG target position. It is contemplated that in an embodiment the endogenous genomic donor sequence is located on the same chromosome as the target sequence. It is further contemplated that in another embodiment the endogenous genomic donor sequence is located on a different chromosome from the target sequence. Alteration of an HBG target position by endogenous genomic donor sequence depends on cleavage by a Cas9 molecule. Cleavage by Cas9 can comprise a double-strand break or two single-strand breaks.

In certain embodiments of the methods provided herein, HDR-mediated alteration is used to alter a single nucleotide in a γ-globin gene regulatory region. These embodiments may utilize either one double-strand break or two single-strand breaks. In certain embodiments, a single nucleotide alteration is incorporated using (1) one double-strand break, (2) two single-strand breaks, (3) two double-strand breaks with a break occurring on each side of the target position, (4) one double-strand break and two single strand breaks with the double strand break and two single strand breaks occurring on each side of the target position, (5) four single-strand breaks with a pair of single-strand breaks occurring on each side of the target position, or (6) one single-strand break.

In certain embodiments wherein a single-stranded template nucleic acid is used, the target position can be altered by alternative HDR.

In certain embodiments of the methods provided herein, HDR-mediated alteration is used to introduce an alteration (e.g., deletion) of one or more nucleotides in a γ-globin gene regulatory region. In certain embodiments, the γ-globin gene regulatory region may be a HBG target position. In certain embodiments, the alteration (e.g., deletion) may be introduced at a target site within the HBG target position. In certain embodiments, the alteration (e.g., deletion) may be selected from one or more of HBG1 13 bp del c.−114 to −102, HBG1 4 bp del c.−225 to −222, and HBG1 13 bp del c.−114 to −102. In certain embodiments, the target site may be selected from one or more of HBG1 c.−114 to −102 (e.g., nucleotides 2824-2836 of SEQ ID NO:902 (HBG1)), HBG1 c.−225 to −222 (e.g., nucleotides 2716-2719 of SEQ ID NO:902 (HBG1)), and HBG2 c.−114 to −102 (e.g., nucleotides 2748-2760 of SEQ ID NO:903 (HBG2)).

Donor template-effected alteration of an HBG target position depends on cleavage by a Cas9 molecule. Cleavage by Cas9 can comprise a nick, a double-strand break, or two single-strand breaks, e.g., one on each strand of the target nucleic acid. After introduction of the breaks on the target nucleic acid, resection occurs at the break ends resulting in single stranded overhanging DNA regions.

In canonical HDR, a double-stranded donor template is introduced, comprising homologous sequence to the target nucleic acid that will either be directly incorporated into the target nucleic acid or used as a template to change the sequence of the target nucleic acid. After resection at the break, repair can progress by different pathways, e.g., by the double Holliday junction model (or double-strand break repair, DSBR, pathway) or the synthesis-dependent strand annealing (SDSA) pathway. In the double Holliday junction model, strand invasion by the two single stranded overhangs of the target nucleic acid to the homologous sequences in the donor template occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in alteration of the target nucleic acid, e.g., incorporation of an HPFH mutant sequence of the donor template at the corresponding HBG target position. Crossover with the donor template may occur upon resolution of the junctions. In the SDSA pathway, only one single stranded overhang invades the donor template and new DNA is synthesized from the end of the invading strand to fill the gap resulting from resection. The newly synthesized DNA then anneals to the remaining single stranded overhang, new DNA is synthesized to fill in the gap, and the strands are ligated to produce the altered DNA duplex.

In alternative HDR, a single strand donor template, e.g., template nucleic acid, is introduced. A nick, single-strand break, or double-strand break at the target nucleic acid, for altering a desired HBG target position, is mediated by a Cas9 molecule, e.g., described herein, and resection at the break occurs to reveal single stranded overhangs. Incorporation of the sequence of the template nucleic acid to alter an HBG target position typically occurs by the SDSA pathway, as described above.

Additional details on template nucleic acids are provided in Section IV entitled “Template nucleic acids” in International Application PCT/US2014/057905.

In certain embodiments, double-strand cleavage is effected by a Cas9 molecule having cleavage activity associated with an HNH-like domain and cleavage activity associated with a RuvC-like domain, e.g., an N-terminal RuvC-like domain, e.g., a wild-type Cas9. Such embodiments require only a single gRNA.

In certain embodiments, one single-strand break, or nick, is effected by a Cas9 molecule having nickase activity, e.g., a Cas9 nickase as described herein. A nicked target nucleic acid can be a substrate for alt-HDR.

In other embodiments, two single-strand breaks, or nicks, are effected by a Cas9 molecule having nickase activity, e.g., cleavage activity associated with an HNH-like domain or cleavage activity associated with an N-terminal RuvC-like domain. Such embodiments usually require two gRNAs, one for placement of each single-strand break. In an embodiment, the Cas9 molecule having nickase activity cleaves the strand to which the gRNA hybridizes, but not the strand that is complementary to the strand to which the gRNA hybridizes. In an embodiment, the Cas9 molecule having nickase activity does not cleave the strand to which the gRNA hybridizes, but rather cleaves the strand that is complementary to the strand to which the gRNA hybridizes.

In certain embodiments, the nickase has HNH activity, e.g., a Cas9 molecule having the RuvC activity inactivated, e.g., a Cas9 molecule having a mutation at D10, e.g., the D10A mutation (see, e.g., SEQ ID NO:10). D10A inactivates RuvC; therefore, the Cas9 nickase has (only) HNH activity and will cut on the strand to which the gRNA hybridizes (e.g., the complementary strand, which does not have the NGG PAM on it). In other embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In other embodiments, a Cas9 molecule having an N863 mutation, e.g., the N863A mutation, mutation can be used as a nickase. N863A inactivates HNH therefore the Cas9 nickase has (only) RuvC activity and cuts on the non-complementary strand (the strand that has the NGG PAM and whose sequence is identical to the gRNA).

In certain embodiments in which a nickase and two gRNAs are used to position two single strand nicks, one nick is on the + strand and one nick is on the − strand of the target nucleic acid. The PAMs can be outwardly facing. The gRNAs can be selected such that the gRNAs are separated by, from about 0-50, 0-100, or 0-200 nucleotides. In an embodiment, there is no overlap between the target sequences that are complementary to the targeting domains of the two gRNAs. In an embodiment, the gRNAs do not overlap and are separated by as much as 50, 100, or 200 nucleotides. In an embodiment, the use of two gRNAs can increase specificity, e.g., by decreasing off-target binding (Ran 2013).

In certain embodiments, a single nick can be used to induce HDR, e.g., alt-HDR. It is contemplated herein that a single nick can be used to increase the ratio of HR to NHEJ at a given cleavage site. In an embodiment, a single-strand break is formed in the strand of the target nucleic acid to which the targeting domain of said gRNA is complementary. In other embodiments, a single-strand break is formed in the strand of the target nucleic acid other than the strand to which the targeting domain of said gRNA is complementary.

### Placement of Double-Strand or Single-Strand Breaks Relative to the Target Position

A double-strand break or single-strand break in one of the strands should be sufficiently close to an HBG target position that an alteration is produced in the desired region, e.g., incorporation of an HPFH mutation. In certain embodiments, the distance is not more than 50, 100, 200, 300, 350, or 400 nucleotides from the HBG target position. While not wishing to be bound by theory, in certain embodiments it is believed that the break should be sufficiently close to the HBG target position that the target position is within the region that is subject to exonuclease-mediated removal during end resection. If the distance between the HBG target position and a break is too great, the sequence desired to be altered may not be included in the end resection and, therefore, may not be altered, as donor sequence, either exogenously provided donor sequence or endogenous genomic donor sequence, in some embodiments is only used to alter sequence within the end resection region.

In certain embodiments, the methods described herein introduce one or more breaks near a γ-globin gene regulatory region(s), e.g., enhancer region(s), e.g., silencer region(s), e.g., promoter region(s) of the HGB1 and/or HGB2 gene(s). In certain of these embodiments, two or more breaks are introduced that flank at least a portion of the regulatory region(s), e.g., enhancer region(s), e.g., silencer region(s), of the HGB1 and/or HGB2 gene(s). The two or more breaks remove (e.g., delete) a genomic sequence including at least a portion of the γ-globin gene regulatory region(s), e.g., enhancer region(s), e.g., silencer region(s), of the HGB1 and/or HGB2 gene(s). All methods described herein result in altering the regulatory region(s), e.g., enhancer region(s), e.g., silencer region(s), of the HGB1 and/or HGB2 gene(s).

In certain embodiments, the gRNA targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, or 200 nucleotides of the region desired to be altered, e.g., a mutation. The break, e.g., a double-strand or single-strand break, can be positioned upstream or downstream of the region desired to be altered, e.g., a mutation. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of a mutation.

In certain embodiments, a single-strand break is accompanied by an additional single-strand break, positioned by a second gRNA molecule, as discussed below. For example, the targeting domains bind configured such that a cleavage event, e.g., the two single strand breaks, are positioned within 1, 2, 3, 4, 5, 10, 15, 20, 25, 30, 35, 40, 45, 50, 60, 70, 80, 90, 100, 150, or 200 nucleotides of an HBG target position. In an embodiment, the first and second gRNA molecules are configured such that, when guiding a Cas9 nickase, a single-strand break will be accompanied by an additional single strand break, positioned by a second gRNA, sufficiently close to one another to result in alteration of the desired region. In an embodiment, the first and second gRNA molecules are configured such that a single-strand break positioned by said second gRNA is within 10, 20, 30, 40, or 50 nucleotides of the break positioned by said first gRNA molecule, e.g., when the Cas9 is a nickase. In an embodiment, the two gRNA molecules are configured to position cuts at the same position, or within a few nucleotides of one another, on different strands, e.g., essentially mimicking a double-strand break.

In certain embodiments in which a gRNA (unimolecular (or chimeric) or modular gRNA) and Cas9 nuclease induce a double-strand break for the purpose of inducing HDR-mediated sequence alteration, the cleavage site is 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the HBG target position. In certain embodiments, the cleavage site is 0-100 bp (e.g., 0 to 75, 0 to 50, 0 to 25, 25 to 100, 25 to 75, 25 to 50, 50 to 100, 50 to 75 or 75 to 100 bp) away from the HBG target position.

In certain embodiments, one can promote HDR by using nickases to generate a break with overhangs. While not wishing to be bound by theory, the single stranded nature of the overhangs can enhance the likelihood of a cell repairing the break by HDR as opposed to, e.g., NHEJ. Specifically, in some embodiments, HDR is promoted by selecting a first gRNA that targets a first nickase to a first target sequence, and a second gRNA that targets a second nickase to a second target sequence which is on the opposite DNA strand from the first target sequence and offset from the first nick.

In certain embodiments, the targeting domain of a gRNA molecule is configured to position a cleavage event sufficiently far from a preselected nucleotide that the nucleotide is not altered. In certain embodiments, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

### Placement of a First Break and a Second Break Relative to Each Other

In certain embodiments, a double-strand break can be accompanied by an additional double-strand break, positioned by a second gRNA molecule, as is discussed below.

In certain embodiments, a double-strand break can be accompanied by two additional single-strand breaks, positioned by a second gRNA molecule and a third gRNA molecule.

In certain embodiments, first and second single-strand breaks can be accompanied by two additional single-strand breaks positioned by a third gRNA molecule and a fourth gRNA molecule.

When two or more gRNAs are used to position two or more cleavage events, e.g., double-strand or single-strand breaks, in a target nucleic acid, it is contemplated that the two or more cleavage events may be made by the same or different Cas9 proteins. For example, when two gRNAs are used to position two double-strand breaks, a single Cas9 nuclease may be used to create both double-strand breaks. When two or more gRNAs are used to position two or more single-strand breaks (nicks), a single Cas9 nickase may be used to create the two or more nicks. When two or more gRNAs are used to position at least one double-strand break and at least one single-strand break, two Cas9 proteins may be used, e.g., one Cas9 nuclease and one Cas9 nickase. It is contemplated that when two or more Cas9 proteins are used that the two or more Cas9 proteins may be delivered sequentially to control specificity of a double-strand versus a single-strand break at the desired position in the target nucleic acid.

In some embodiments, the targeting domain of the first gRNA molecule and the targeting domain of the second gRNA molecules are complementary to opposite strands of the target nucleic acid molecule. In some embodiments, the gRNA molecule and the second gRNA molecule are configured such that the PAMs are oriented outward.

In certain embodiments, two gRNA are selected to direct Cas9-mediated cleavage at two positions that are a preselected distance from each other. In certain embodiments, the two points of cleavage are on opposite strands of the target nucleic acid. In some embodiments, the two cleavage points form a blunt ended break, and in other embodiments, they are offset so that the DNA ends comprise one or two overhangs (e.g., one or more 5′ overhangs and/or one or more 3′ overhangs). In some embodiments, each cleavage event is a nick. In certain embodiments, the nicks are close enough together that they form a break that is recognized by the double stranded break machinery (as opposed to being recognized by, e.g., the SSBr machinery). In certain embodiments, the nicks are far enough apart that they create an overhang that is a substrate for HDR, i.e., the placement of the breaks mimics a DNA substrate that has experienced some resection. For instance, in some embodiments the nicks are spaced to create an overhang that is a substrate for processive resection. In some embodiments, the two breaks are spaced within 25-65 nucleotides of each other. The two breaks may be, e.g., about 25, 30, 35, 40, 45, 50, 55, 60, or 65 nucleotides of each other. The two breaks may be, e.g., at least about 25, 30, 35, 40, 45, 50, 55, 60, or 65 nucleotides of each other. The two breaks may be, e.g., at most about 30, 35, 40, 45, 50, 55, 60, or 65 nucleotides of each other. In certain embodiments, the two breaks are about 25-30, 30-35, 35-40, 40-45, 45-50, 50-55, 55-60, or 60-65 nucleotides of each other.

In some embodiments, the break that mimics a resected break comprises a 3′ overhang (e.g., generated by a DSB and a nick, where the nick leaves a 3′ overhang), a 5′ overhang (e.g., generated by a DSB and a nick, where the nick leaves a 5′ overhang), a 3′ and a 5′ overhang (e.g., generated by three cuts), two 3′ overhangs (e.g., generated by two nicks that are offset from each other), or two 5′ overhangs (e.g., generated by two nicks that are offset from each other).

In certain embodiments in which two gRNAs (independently, unimolecular (or chimeric) or modular gRNA) complexing with Cas9 nickases induce two single-strand breaks for the purpose of inducing HDR-mediated alteration, the closer nick is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, or 75 to 100 bp) away from the HBG target position and the two nicks will ideally be within 25-65 bp of each other (e.g., 25 to 50, 25 to 45, 25 to 40, 25 to 35, 25 to 30, 30 to 55, 30 to 50, 30 to 45, 30 to 40, 30 to 35, 35 to 55, 35 to 50, 35 to 45, 35 to 40, 40 to 55, 40 to 50, 40 to 45 bp, 45 to 50 bp, 50 to 55 bp, 55 to 60 bp, or 60 to 65 bp) and no more than 100 bp away from each other (e.g., no more than 90, 80, 70, 60, 50, 40, 30, 20, 10, or 5 bp away from each other). In certain embodiments, the cleavage site is between 0-100 bp (e.g., 0 to 75, 0 to 50, 0 to 25, 25 to 100, 25 to 75, 25 to 50, 50 to 100, 50 to 75, or 75 to 100 bp) away from the HBG target position.

In some embodiments, two gRNAs, e.g., independently, unimolecular (or chimeric) or modular gRNA, are configured to position a double-strand break on both sides of a target position. In other embodiments, three gRNAs, e.g., independently, unimolecular (or chimeric) or modular gRNA, are configured to position a double-strand break (i.e., one gRNA complexes with a cas9 nuclease) and two single-strand breaks or paired single-strand breaks (i.e., two gRNAs complex with Cas9 nickases) on either side of the target position. In other embodiments, four gRNAs, e.g., independently, unimolecular (or chimeric) or modular gRNA, are configured to generate two pairs of single-strand breaks (i.e., two pairs of two gRNAs complex with Cas9 nickases) on either side of the target position. The double-strand break(s) or the closer of the two single-strand nicks in a pair will ideally be within 0-500 bp of the HBG target position (e.g., no more than 450, 400, 350, 300, 250, 200, 150, 100, 50 or 25 bp from the target position). When nickases are used, the two nicks in a pair are, in certain embodiments, within 25-65 bp of each other (e.g., between 25 to 55, 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, 40 to 45 bp, 45 to 50 bp, 50 to 55 bp, 55 to 60 bp, or 60 to 65 bp) and no more than 100 bp away from each other (e.g., no more than 90, 80, 70, 60, 50, 40, 30, or 20 or 10 bp).

When two gRNAs are used to target Cas9 molecules to breaks, different combinations of Cas9 molecules are envisioned. In some embodiments, a first gRNA is used to target a first Cas9 molecule to a first target position, and a second gRNA is used to target a second Cas9 molecule to a second target position. In some embodiments, the first Cas9 molecule creates a nick on the first strand of the target nucleic acid, and the second Cas9 molecule creates a nick on the opposite strand, resulting in a double-strand break (e.g., a blunt ended cut or a cut with overhangs).

Different combinations of nickases can be chosen to target one single-strand break to one strand and a second single-strand break to the opposite strand. When choosing a combination, one can take into account that there are nickases having one active RuvC-like domain, and nickases having one active HNH domain. In certain embodiments, a RuvC-like domain cleaves the non-complementary strand of the target nucleic acid molecule. In certain embodiments, an HNH-like domain cleaves a single stranded complementary domain, e.g., a complementary strand of a double stranded nucleic acid molecule. Generally, if both Cas9 molecules have the same active domain (e.g., both have an active RuvC domain or both have an active HNH domain), one will choose two gRNAs that bind to opposite strands of the target. In more detail, in some embodiments a first gRNA is complementary with a first strand of the target nucleic acid and binds a nickase having an active RuvC-like domain and causes that nickase to cleave the strand that is non-complementary to that first gRNA, i.e., a second strand of the target nucleic acid; and a second gRNA is complementary with a second strand of the target nucleic acid and binds a nickase having an active RuvC-like domain and causes that nickase to cleave the strand that is non-complementary to that second gRNA, i.e., the first strand of the target nucleic acid. Conversely, in some embodiments, a first gRNA is complementary with a first strand of the target nucleic acid and binds a nickase having an active HNH domain and causes that nickase to cleave the strand that is complementary to that first gRNA, i.e., a first strand of the target nucleic acid; and a second gRNA is complementary with a second strand of the target nucleic acid and binds a nickase having an active HNH domain and causes that nickase to cleave the strand that is complementary to that second gRNA, i.e., the second strand of the target nucleic acid. In another arrangement, if one Cas9 molecule has an active RuvC-like domain and the other Cas9 molecule has an active HNH domain, the gRNAs for both Cas9 molecules can be complementary to the same strand of the target nucleic acid, so that the Cas9 molecule with the active RuvC-like domain will cleave the non-complementary strand and the Cas9 molecule with the HNH domain will cleave the complementary strand, resulting in a double stranded break.

### Homology Arms of the Donor Template

A homology arm should extend at least as far as the region in which end resection may occur, e.g., in order to allow the resected single stranded overhang to find a complementary region within the donor template. The overall length could be limited by parameters such as plasmid size or viral packaging limits. In an embodiment, a homology arm does not extend into repeated elements, e.g., Alu repeats or LINE repeats.

Exemplary homology arm lengths include at least 50, 100, 250, 500, 750, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides.

A template nucleic acid, as that term is used herein, refers to a nucleic acid sequence which can be used in conjunction with a Cas9 molecule and a gRNA molecule to alter (e.g., delete, disrupt, or modify) the structure of an HBG target position. In certain embodiments, the HBG target position can be a site between two nucleotides, e.g., adjacent nucleotides, on the target nucleic acid into which one or more nucleotides is added. Alternatively, the HBG target position may comprise one or more nucleotides that are altered by a template nucleic acid. In certain embodiments, an alteration (e.g., deletion) may be introduced at a target site within the HBG target position. In certain embodiments, the alteration (e.g., deletion) may be selected from one or more of HBG1 13 bp del c.−114 to −102, HBG1 4 bp del c.−225 to −222, and HBG1 13 bp del c.−114 to −102. In certain embodiments, the target site may be selected from one or more of HBG1 c.−114 to −102 (e.g., nucleotides 2824-2836 of SEQ ID NO:902 (HBG1)), HBG1 c.−225 to −222 (e.g., nucleotides 2716-2719 of SEQ ID NO:902 (HBG1)), and HBG2 c.−114 to −102 (e.g., nucleotides 2748-2760 of SEQ ID NO:903 (HBG2)).

In certain embodiments, the target nucleic acid is modified to have some or all of the sequence of the template nucleic acid, typically at or near cleavage site(s). In certain embodiments, the template nucleic acid is single stranded. In other embodiments, the template nucleic acid is double stranded. In certain embodiments, the template nucleic acid is DNA, e.g., double stranded DNA. In other embodiments, the template nucleic acid is single stranded DNA. In an embodiment, the template nucleic acid is encoded on the same vector backbone, e.g., AAV genome, plasmid DNA, as the Cas9 and gRNA. In certain embodiments, the template nucleic acid is excised from a vector backbone in vivo, e.g., it is flanked by gRNA recognition sequences. In certain embodiments, the template nucleic acid comprises endogenous genomic sequence.

In certain embodiments, the template nucleic acid alters the structure of the target position by participating in an HDR event. In certain embodiments, the template nucleic acid alters the sequence of the target position. In certain embodiments, the template nucleic acid results in the incorporation of a modified, or non-naturally occurring base into the target nucleic acid.

In certain embodiments, the template nucleic acid results in a deletion of one or more nucleotides of the target nucleic acid. In certain embodiments, the template nucleic acid results in deletion of one or more nucleotides of a HBG target position. In certain embodiments, an alteration (e.g., deletion) may be introduced at a target site within the HBG target position. In certain embodiments, the alteration (e.g., deletion) may be selected from one or more of HBG1 13 bp del c.−114 to −102, HBG1 4 bp del c.−225 to −222, and HBG1 13 bp del c.−114 to −102. In certain embodiments, the target site may be selected from one or more of HBG1 c.−114 to −102 (e.g., nucleotides 2824-2836 of SEQ ID NO:902 (HBG1)), HBG1 c.−225 to −222 (e.g., nucleotides 2716-2719 of SEQ ID NO:902 (HBG1)), and HBG2 c.−114 to −102 (e.g., nucleotides 2748-2760 of SEQ ID NO:903 (HBG2)).

Typically, the template sequence undergoes a breakage mediated or catalyzed recombination with the target sequence. In certain embodiments, the template nucleic acid includes sequence that corresponds to a site on the target sequence that is cleaved by an eaCas9 mediated cleavage event. In certain embodiments, the template nucleic acid includes sequence that corresponds to both a first site on the target sequence that is cleaved in a first Cas9 mediated event, and a second site on the target sequence that is cleaved in a second Cas9 mediated event.

A template nucleic acid having homology with an HBG target position in a γ-globin gene regulatory region can be used to alter the structure of the regulatory region. For example, a template nucleic acid having homology with the region 5′ and 3′ of an HBG target position in a γ-globin gene regulatory region can be used to delete one or more nucleotides of an HBG target position.

A template nucleic acid typically comprises the following components:

[5′ homology arm]-[replacement sequence]-[3′ homology arm].

The homology arms provide for recombination into the chromosome, thus replacing the undesired element, e.g., a mutation or signature, with the replacement sequence. The homology arms are regions that are homologous to regions of DNA within or near (e.g., flanking or adjoining) a target nucleic acid to be cleaved. In certain embodiments, the homology arms flank the most distal cleavage sites.

In certain embodiments, a template nucleic acid may be used to remove (e.g., delete) a genomic sequence including at least a portion of the γ-globin gene regulatory region(s), e.g., enhancer region(s), e.g., silencer region(s), of the HGB1 and/or HGB2 gene(s). In certain embodiments, a template nucleic acid may be used to delete one or more nucleotides of an HBG target position, i.e., introduce an alteration (e.g., deletion) into an HBG target position. In certain embodiments, an alteration (e.g., deletion) may be introduced at a target site within the HBG target position. In certain embodiments, the alteration (e.g., deletion) may be selected from one or more of HBG1 13 bp del c.−114 to −102, HBG1 4 bp del c.−225 to −222, and HBG1 13 bp del c.−114 to −102. In certain embodiments, the target site may be selected from one or more of HBG1 c.−114 to −102 (e.g., nucleotides 2824-2836 of SEQ ID NO:902 (HBG1)), HBG1 c.−225 to −222 (e.g., nucleotides 2716-2719 of SEQ ID NO:902 (HBG1)), and HBG2 c.−114 to −102 (e.g., nucleotides 2748-2760 of SEQ ID NO:903 (HBG2)).

Replacement sequences in donor templates have been described elsewhere, including in Cotta-Ramusino 2016, which is incorporated by reference herein. A replacement sequence can be any suitable length. In certain embodiments, a replacement sequence may include 1, 2, 3, 4, 5, 6, 7, 8, 9, 10, 11, 12, 13, 14, 15, 16, 17, 18, 19, or 20 or more sequence modifications relative to the naturally-occurring sequence within a cell in which editing is desired.

In certain embodiments, where the desired repair outcome is a deletion of the target nucleic acid, a replacement sequence may be 0 nucleotides or 0 bp. In certain embodiments, the template nucleic acid omits the sequence that is homologous to the target nucleic acid sequence to be deleted. If the replacement sequence is 0 nucleotides or 0 bp, then the sequence of the target nucleic acid that is positioned between where the 5′ homology arm and 3′ homology arm anneal to the template nucleic acid will be deleted.

In certain embodiments, the 3′ end of the 5′ homology arm is the position next to the 5′ end of the replacement sequence. In certain embodiments, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the replacement sequence. In certain embodiments, when the replacement sequence is 0 nucleotides or 0 bp, the 3′ end of the 5′ homology arm is the position next to the 5′ end of the 3′ homology arm. In certain embodiments where the replacement sequence is 0 nucleotides or 0 bp, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the 3′ homology arm.

In certain embodiments, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the replacement sequence. In certain embodiments where the replacement sequence is 0 nucleotides or 0 bp, the 5′ end of the 3′ homology arm is the position next to the 3′ end of the 5′ homology arm. In an embodiment, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the 5′ homology arm.

In certain embodiments, to alter one or more nucleotides at an HBG target position, the homology arms, e.g., the 5′ and 3′ homology arms, may each comprise about 1000 bp of sequence flanking the most distal gRNAs (e.g., 1000 bp of sequence on either side of the HBG target position).

It is contemplated herein that one or both homology arms may be shortened to avoid including certain sequence repeat elements, e.g., Alu repeats or LINE elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

It is contemplated herein that template nucleic acids for altering the sequence of an HBG target position may be designed for use as a single-stranded oligonucleotide, e.g., a single-stranded oligodeoxynucleotide (ssODN). When using a ssODN, 5′ and 3′ homology arms may range up to about 200 nucleotides in length, e.g., at least 25, 50, 75, 100, 125, 150, 175, or 200 bp in length. Longer homology arms are also contemplated for ssODNs as improvements in oligonucleotide synthesis continue to be made. In some embodiments, a longer homology arm is made by a method other than chemical synthesis, e.g., by denaturing a long double stranded nucleic acid and purifying one of the strands, e.g., by affinity for a strand-specific sequence anchored to a solid substrate.

While not wishing to be bound by theory, in certain embodiments alt-HDR proceeds more efficiently when the template nucleic acid has extended homology 5′ to the nick (i.e., in the 5′ direction of the nicked strand) or target site (i.e., in the 5′ direction of the target site). Accordingly, in some embodiments, the template nucleic acid has a longer homology arm and a shorter homology arm, wherein the longer homology arm can anneal 5′ of the nick or target site. In some embodiments, the arm that can anneal 5′ to the nick or target site is at least 25, 50, 75, 100, 125, 150, 175, or 200, 300, 400, 500, 600, 700, 800, 900, 1000, 1500, 2000, 3000, 4000, or 5000 nucleotides from the nick or target site or the 5′ or 3′ end of the replacement sequence. In some embodiments, the arm that can anneal 5′ to the nick or target site is at least 10%, 20%, 30%, 40%, or 50% longer than the arm that can anneal 3′ to the nick or target site. In some embodiments, the arm that can anneal 5′ to the nick or target site is at least 2×, 3×, 4×, or 5× longer than the arm that can anneal 3′ to the nick or target site. Depending on whether a ssDNA template can anneal to the intact strand or the nicked strand, the homology arm that anneals 5′ to the nick or target site may be at the 5′ end of the ssDNA template or the 3′ end of the ssDNA template, respectively.

Similarly, in some embodiments, the template nucleic acid has a 5′ homology arm, a replacement sequence, and a 3′ homology arm, such that the template nucleic acid has extended homology to the 5′ of the nick. For example, the 5′ homology arm and 3′ homology arm may be substantially the same length, but the replacement sequence may extend farther 5′ of the nick than 3′ of the nick. In some embodiments, the replacement sequence extends at least 10%, 20%, 30%, 40%, 50%, 2×, 3×, 4×, or 5× further to the 5′ end of the nick than the 3′ end of the nick.

While not wishing to be bound by theory, in some embodiments alt-HDR proceeds more efficiently when the template nucleic acid is centered on the nick or target site. Accordingly, in some embodiments, the template nucleic acid has two homology arms that are essentially the same size. For instance, the first homology arm of a template nucleic acid may have a length that is within 10%, 9%, 8%, 7%, 6%, 5%, 4%, 3%, 2%, or 1% of the second homology arm of the template nucleic acid.

Similarly, in some embodiments, the template nucleic acid has a 5′ homology arm, a replacement sequence, and a 3′ homology arm, such that the template nucleic acid extends substantially the same distance on either side of the nick or target site. For example, the homology arms may have different lengths, but the replacement sequence may be selected to compensate for this. For example, the replacement sequence may extend further 5′ from the nick than it does 3′ of the nick, but the homology arm 5′ of the nick is shorter than the homology arm 3′ of the nick, to compensate. The converse is also possible, e.g., that the replacement sequence may extend further 3′ from the nick than it does 5′ of the nick, but the homology arm 3′ of the nick is shorter than the homology arm 5′ of the nick, to compensate.

### Exemplary Template Nucleic Acids

In certain embodiments, the template nucleic acid is double stranded. In other embodiments, the template nucleic acid is single stranded. In certain embodiments, the template nucleic acid comprises a single stranded portion and a double stranded portion. In certain embodiments, the template nucleic acid comprises about 50 to 100 bp, e.g., 55 to 95, 60 to 90, 65 to 85, or 70 to 80 bp, homology on either side of the nick, target site, and/or replacement sequence. In certain embodiments, the template nucleic acid comprises about 50, 55, 60, 65, 70, 75, 80, 85, 90, 95, or 100 bp homology 5′ of the nick, target site, or replacement sequence, 3′ of the nick, target site, or replacement sequence, or both 5′ and 3′ of the nick, target site, or replacement sequences.

In certain embodiments, the template nucleic acid comprises about 150 to 200 bp, e.g., 155 to 195, 160 to 190, 165 to 185, or 170 to 180 bp, homology 3′ of the nick, target site, and/or replacement sequence. In certain embodiments, the template nucleic acid comprises about 150, 155, 160, 165, 170, 175, 180, 185, 190, 195, or 200 bp homology 3′ of the nick, target site, or replacement sequence. In certain embodiments, the template nucleic acid comprises less than about 100, 90, 80, 70, 60, 50, 40, 30, 20, 15, or 10 bp homology 5′ of the nick, target site, or replacement sequence.

In certain embodiments, the template nucleic acid comprises about 150 to 200 bp, e.g., 155 to 195, 160 to 190, 165 to 185, or 170 to 180 bp, homology 5′ of the nick, target site, and/or replacement sequence. In certain embodiments, the template nucleic acid comprises about 150, 155, 160, 165, 170, 175, 180, 185, 190, 195, or 200 bp homology 5′ of the nick, target site, or replacement sequence. In certain embodiments, the template nucleic acid comprises less than about 100, 90, 80, 70, 60, 50, 40, 30, 20, 15, or 10 bp homology 3′ of the nick, target site, or replacement sequence.

In certain embodiments, the template nucleic acid comprises a nucleotide sequence, e.g., of one or more nucleotides, that will be added to or will template a change in the target nucleic acid. In other embodiments, the template nucleic acid comprises a nucleotide sequence that may be used to modify the target position. In other embodiments, the template nucleic acid comprises a nucleotide sequence that may be used to delete one or more nucleotides of a HBG target position.

The template nucleic acid may comprise a replacement sequence. In some embodiments, the template nucleic acid comprises a 5′ homology arm. In other embodiments, the template nucleic acid comprises a 3′ homology arm.

The template nucleic acid may comprise a 5′ homology arm, replacement sequence that is 0 nucleotides or 0 bp, and a 3′ homology arm.

In certain embodiments, the template nucleic acid is linear double stranded DNA. The length may be, e.g., about 150-200 bp, e.g., about 150, 160, 170, 180, 190, or 200 bp. The length may be, e.g., at least 150, 160, 170, 180, 190, or 200 bp. In some embodiments, the length is no greater than 150, 160, 170, 180, 190, or 200 bp. In some embodiments, a double stranded template nucleic acid has a length of about 160 bp, e.g., about 155-165, 150-170, 140-180, 130-190, 120-200, 110-210, 100-220, 90-230, or 80-240 bp.

The template nucleic acid can be linear single stranded DNA. In certain embodiments, the template nucleic acid is (i) linear single stranded DNA that can anneal to the nicked strand of the target nucleic acid, (ii) linear single stranded DNA that can anneal to the intact strand of the target nucleic acid, (iii) linear single stranded DNA that can anneal to the plus strand of the target nucleic acid, (iv) linear single stranded DNA that can anneal to the minus strand of the target nucleic acid, or more than one of the preceding. The length may be, e.g., about 150-200 nucleotides, e.g., about 150, 160, 170, 180, 190, or 200 nucleotides. The length may be, e.g., at least 150, 160, 170, 180, 190, or 200 nucleotides. In some embodiments, the length is no greater than 150, 160, 170, 180, 190, or 200 nucleotides. In some embodiments, a single stranded template nucleic acid has a length of about 160 nucleotides, e.g., about 155-165, 150-170, 140-180, 130-190, 120-200, 110-210, 100-220, 90-230, or 80-240 nucleotides.

In some embodiments, the template nucleic acid is circular double stranded DNA, e.g., a plasmid. In some embodiments, the template nucleic acid comprises about 500 to 1000 bp of homology on either side of the replacement sequence, target site, and/or the nick. In some embodiments, the template nucleic acid comprises about 300, 400, 500, 600, 700, 800, 900, 1000, 1500, or 2000 bp of homology 5′ of the nick, target site, or replacement sequence, 3′ of the nick, target site, or replacement sequence, or both 5′ and 3′ of the nick, target site, or replacement sequence. In some embodiments, the template nucleic acid comprises at least 300, 400, 500, 600, 700, 800, 900, 1000, 1500, or 2000 bp of homology 5′ of the nick, target site, or replacement sequence, 3′ of the nick, target site, or replacement sequence, or both 5′ and 3′ of the nick, target site, or replacement sequence. In some embodiments, the template nucleic acid comprises no more than 300, 400, 500, 600, 700, 800, 900, 1000, 1500, or 2000 bp of homology 5′ of the nick, target site, or replacement sequence, 3′ of the nick, target site, or replacement sequence, or both 5′ and 3′ of the nick, target site, or replacement sequence.

In certain embodiments, one or both homology arms may be shortened to avoid including certain sequence repeat elements, e.g., Alu repeats, LINE elements. For example, a 5′ homology arm may be shortened to avoid a sequence repeat element, while a 3′ homology arm may be shortened to avoid a sequence repeat element. In some embodiments, both the 5′ and the 3′ homology arms may be shortened to avoid including certain sequence repeat elements.

In some embodiments, the template nucleic acid is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In some embodiments, the template nucleic acid comprises about 150 to 1000 nucleotides of homology on either side of the replacement sequence, target site, and/or the nick. In some embodiments, the template nucleic acid comprises about 100, 150, 200, 300, 400, 500, 600, 700, 800, 900, 1000, 1500, or 2000 nucleotides 5′ of the nick, target site, or replacement sequence, 3′ of the nick, target site, or replacement sequence, or both 5′ and 3′ of the nick, target site, or replacement sequence. In some embodiments, the template nucleic acid comprises at least 100, 150, 200, 300, 400, 500, 600, 700, 800, 900, 1000, 1500, or 2000 nucleotides 5′ of the nick, target site, or replacement sequence, 3′ of the nick, target site, or replacement sequence, or both 5′ and 3′ of the nick, target site, or replacement sequence. In some embodiments, the template nucleic acid comprises at most 100, 150, 200, 300, 400, 500, 600, 700, 800, 900, 1000, 1500, or 2000 nucleotides 5′ of the nick, target site, or replacement sequence, 3′ of the nick, target site, or replacement sequence, or both 5′ and 3′ of the nick, target site, or replacement sequence.

In some embodiments, the template nucleic acid is a lentiviral vector, e.g., an IDLV (integration deficiency lentivirus). In some embodiments, the template nucleic acid comprises about 500 to 1000 bp of homology on either side of the replacement sequence, target site, and/or the nick. In some embodiments, the template nucleic acid comprises about 300, 400, 500, 600, 700, 800, 900, 1000, 1500, or 2000 bp of homology 5′ of the nick, target site, or replacement sequence, 3′ of the nick, target site, or replacement sequence, or both 5′ and 3′ of the nick, target site, or replacement sequence. In some embodiments, the template nucleic acid comprises at least 300, 400, 500, 600, 700, 800, 900, 1000, 1500, or 2000 bp of homology 5′ of the nick, target site, or replacement sequence, 3′ of the nick, target site, or replacement sequence, or both 5′ and 3′ of the nick or replacement sequence. In some embodiments, the template nucleic acid comprises no more than 300, 400, 500, 600, 700, 800, 900, 1000, 1500, or 2000 bp of homology 5′ of the nick, target site, or replacement sequence, 3′ of the nick, target site, or replacement sequence, or both 5′ and 3′ of the nick, target site, or replacement sequence.

In an embodiment, the template nucleic acid comprises one or more mutations, e.g., silent mutations, that prevent Cas9 from recognizing and cleaving the template nucleic acid. The template nucleic acid may comprise, e.g., at least 1, 2, 3, 4, 5, 10, 20, or 30 silent mutations relative to the corresponding sequence in the genome of the cell to be altered. In certain embodiments, the template nucleic acid comprises at most 2, 3, 4, 5, 10, 20, 30, or 50 silent mutations relative to the corresponding sequence in the genome of the cell to be altered. In an embodiment, the cDNA comprises one or more mutations, e.g., silent mutations that prevent Cas9 from recognizing and cleaving the template nucleic acid. The template nucleic acid may comprise, e.g., at least 1, 2, 3, 4, 5, 10, 20, or 30 silent mutations relative to the corresponding sequence in the genome of the cell to be altered. In certain embodiments, the template nucleic acid comprises at most 2, 3, 4, 5, 10, 20, 30, or 50 silent mutations relative to the corresponding sequence in the genome of the cell to be altered.

In certain embodiments of the methods provided herein, HDR-mediated alteration is used to introduce an alteration (e.g., deletion) of one or more nucleotides in a γ-globin gene regulatory region. In certain embodiments, the γ-globin gene regulatory region may be a HBG target position. In certain embodiments, an alteration (e.g., deletion) may be introduced at a target site within the HBG target position. In certain embodiments, the alteration (e.g., deletion) may be selected from one or more of HBG1 13 bp del c.−114 to −102, HBG1 4 bp del c.−225 to −222, and HBG1 13 bp del c.−114 to −102. In certain embodiments, the target site may be selected from one or more of HBG1 c.−114 to −102 (e.g., nucleotides 2824-2836 of SEQ ID NO:902 (HBG1)), HBG1 c.−225 to −222 (e.g., nucleotides 2716-2719 of SEQ ID NO:902 (HBG1)), and HBG2 c.−114 to −102 (e.g., nucleotides 2748-2760 of SEQ ID NO:903 (HBG2)).

In certain embodiments, a template nucleic acid for introducing an alteration (e.g., deletion) at a target site within an HBG target position (i.e., an HBG1 or HBG2 regulatory region) comprises, from the 5′ to 3′ direction, a 5′ homology arm, a replacement sequence, and a 3′ homology arm, wherein the replacement sequence is 0 nucleotides or 0 bp. In certain embodiments, the template nucleic acid may be a single stranded oligodeoxynucleotide (ssODN). In certain embodiments, the 5′ homology arm may be any of the 5′ homology arms described herein. In certain embodiments, the 3′ homology arms may be any of the 3′ homology arms described herein. In certain embodiments, an alteration (e.g., deletion) may be introduced at a target site within the HBG target position. In certain embodiments, the alteration (e.g., deletion) may be selected from one or more of HBG1 13 bp del c.−114 to −102, HBG1 4 bp del c.−225 to −222, and HBG1 13 bp del c.−114 to −102. In certain embodiments, the target site may be selected from one or more of HBG1 c.−114 to −102 (e.g., nucleotides 2824-2836 of SEQ ID NO:902 (HBG1)), HBG1 c.−225 to −222 (e.g., nucleotides 2716-2719 of SEQ ID NO:902 (HBG1)), and HBG2 c.−114 to −102 (e.g., nucleotides 2748-2760 of SEQ ID NO:903 (HBG2)).

For example, a template nucleic acid for introducing the alteration HBG1 13 bp del c.−114 to −102 at the target site HBG1 c.−114 to −102 (e.g., nucleotides 2824-2836 of SEQ ID NO:902 (HBG1)) may comprise a 5′ homology arm, a replacement sequence, and a 3′ homology arm, where the replacement sequence is 0 nucleotides or 0 bp. In certain embodiments, the 5′ homology arm comprises about 200 nucleotides in length, e.g., at least 25, 50, 75, 100, 125, 150, 175, or 200 nucleotides in length. In certain embodiments, the 5′ homology arm comprises about 50 to 100 bp, e.g., 55 to 95, 60 to 90, 70 to 90, or 80 to 90 bp, homology 5′ of the target site HBG1 c.−114 to −102 (e.g., nucleotides 2824-2836 of SEQ ID NO:902 (HBG1)). In certain embodiments, the 5′ homology arm comprises, consists essentially of, or consists of SEQ ID NO:904 (ssODN1 5′ homology arm). In certain embodiments, the 5′ homology arm comprises, consists essentially of, or consists of SEQ ID NO:907 (PhTx ssODN1 5′homology arm). In certain embodiments, the 3′ homology arm comprises about 200 nucleotides in length, e.g., at least 25, 50, 75, 100, 125, 150, 175, or 200 nucleotides in length. In certain embodiments, the 3′ homology arm comprises about 50 to 100 bp, e.g., 55 to 95, 60 to 90, 70 to 90, or 80 to 90 bp, homology 3′ of the target site HBG1 c.−114 to −102 (e.g., nucleotides 2824-2836 of SEQ ID NO:902 (HBG1)). In certain embodiments, the 3′ homology arm comprises, consists essentially of, or consists of SEQ ID NO:905 (ssODN1 3′ homology arm). In certain embodiments, the 3′ homology arm comprises, consists essentially of, or consists of SEQ ID NO:908 (PhTx ssODN1 3′homology arm). In certain embodiments, the template nucleic acid comprises, consists essentially of, or consists of SEQ ID NO:906. In certain embodiments, the template nucleic acid comprises, consists essentially of, or consists of SEQ ID NO:909 (PhTx ssODN1).

In another example, a template nucleic acid for introducing the alteration HBG2 13 bp del c.−114 to −102 at the target site HBG2 c.−114 to −102 (e.g., nucleotides 2748-2760 of SEQ ID NO:903 (HBG2)) may comprise a 5′ homology arm, a replacement sequence, and a 3′ homology arm, where the replacement sequence is 0 nucleotides or 0 bp. In certain embodiments, the 5′ homology arm comprises about 200 nucleotides in length, e.g., at least 25, 50, 75, 100, 125, 150, 175, or 200 nucleotides in length. In certain embodiments, the 5′ homology arm comprises about 50 to 100 bp, e.g., 55 to 95, 60 to 90, 70 to 90, or 80 to 90 bp, homology 5′ of the target site HBG2 c.−114 to −102 (e.g., nucleotides 2748-2760 of SEQ ID NO:903 (HBG2)). In certain embodiments, the 5′ homology arm comprises, consists essentially of, or consists of SEQ ID NO:904 (ssODN1 5′ homology arm). In certain embodiments, the 5′ homology arm comprises, consists essentially of, or consists of SEQ ID NO:907 (PhTx ssODN1 5′ homology arm). In certain embodiments, the 3′ homology arm comprises about 200 nucleotides in length, e.g., at least 25, 50, 75, 100, 125, 150, 175, or 200 nucleotides in length. In certain embodiments, the 3′ homology arm comprises about 50 to 100 bp, e.g., 55 to 95, 60 to 90, 70 to 90, or 80 to 90 bp, homology 3′ of the target site HBG2 c.−114 to −102 (e.g., nucleotides 2748-2760 of SEQ ID NO:903 (HBG2)). In certain embodiments, the 3′ homology arm comprises, consists essentially of, or consists of SEQ ID NO:905 (ssODN1 3′ homology arm). In certain embodiments, the 3′ homology arm comprises, consists essentially of, or consists of SEQ ID NO:908 (PhTx ssODN1 3′homology arm). In certain embodiments, the template nucleic acid comprises, consists essentially of, or consists of SEQ ID NO:906. In certain embodiments, the template nucleic acid comprises, consists essentially of, or consists of SEQ ID NO:909 (PhTx ssODN1).

In another example, a template nucleic acid for introducing the alteration HBG1 4 bp del c.−225 to −222 at the target site HBG1 c.−225 to −222 (e.g., nucleotides 2716-2719 of SEQ ID NO:902 (HBG1)) may comprise a 5′ homology arm, a replacement sequence, and a 3′ homology arm, where the replacement sequence is 0 nucleotides or 0 bp. In certain embodiments, the 5′ homology arm comprises about 200 nucleotides in length, e.g., at least 25, 50, 75, 100, 125, 150, 175, or 200 nucleotides in length. In certain embodiments, the 5′ homology arm comprises about 50 to 100 bp, e.g., 55 to 95, 60 to 90, 70 to 90, or 80 to 90 bp, homology 5′ of the target site HBG1 c.−225 to −222 (e.g., nucleotides 2716-2719 of SEQ ID NO:902 (HBG1)). In certain embodiments, the 3′ homology arm comprises about 200 nucleotides in length, e.g., at least 25, 50, 75, 100, 125, 150, 175, or 200 nucleotides in length. In certain embodiments, the 3′ homology arm comprises about 50 to 100 bp, e.g., 55 to 95, 60 to 90, 70 to 90, or 80 to 90 bp, homology 3′ of the target site HBG1 c.−225 to −222 (e.g., nucleotides 2716-2719 of SEQ ID NO:902 (HBG1)).

In certain embodiments, the 5′ homology arm comprises a 5′ phosphorothionate (PhTx) modification. In certain embodiments, the 3′ homology arm comprises a 3′ PhTx modification.

In certain embodiments, the template nucleic acid comprises a 5′ and 3′ PhTx modification. In certain embodiments, a template nucleic acid for altering a single nucleotide in a 7-globin gene (e.g., HBG1, HBG2) regulatory region comprises, from the 5′ to 3′ direction, a 5′ homology arm, a replacement sequence, and a 3′ homology arm, wherein the replacement is designed to incorporate the single nucleotide alteration. For example, where the alteration being incorporated is HBG1 c.−114 C>T; c.−158 C>T; c.−167 C>T; c.−196 C>T; or c.−201 C>T or HBG2 c.−109 G>T; c.−114 C>T; c.−157 C>T; c.−158 C>T; c.−167 C>T; c.−211 C>T, the replacement sequence may comprise the single nucleotide T and, optionally, one more nucleotides on one or both sides of this T. Similarly, where the alteration being incorporated is HBG1 c.−117 G>A; c.−170 G>A; or c.−499 T>A or HBG2 c.−114 C>A or c.−167 C>A, the replacement sequence may comprise the single nucleotide A and, optionally, one more nucleotides on one or both sides of this A; where the alteration being incorporated is HBG1 c.−175 T>G or c.−195 C>G or HBG2 c.−202 C>G; c.−255 C>G; c.−309 A>G; c.−369 C>G; or c.−567 T>G, the replacement sequence may comprise the single nucleotide G and, optionally, one more nucleotides on one or both sides of this G; and where the alteration being incorporated is HBG1 c.−175 T>C; c.−198 T>C; or c.−251 T>C or HBG2 c.−175 T>C or c.−228 T>C, the replacement sequence may comprise the single nucleotide C and, optionally, one more nucleotides on one or both sides of this C.

In certain embodiments, the 5′ and 3′ homology arms each comprise a length of sequence flanking the nucleotides corresponding to the replacement sequence. In certain embodiments, a template nucleic acid comprises a replacement sequence flanked by a 5′ homology arm and a 3′ homology arm each independently comprising 10 or more, 20 or more, 50 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 1000 or more, 1100 or more, 1200 or more, 1300 or more, 1400 or more, 1500 or more, 1600 or more, 1700 or more, 1800 or more, 1900 or more, or 2000 or more nucleotides. In certain embodiments, a template nucleic acid comprises a replacement sequence flanked by a 5′ homology arm and a 3′ homology arm each independently comprising at least 50, 100, or 150 nucleotides, but not long enough to include a repeated element. In certain embodiments, a template nucleic acid comprises a replacement sequence flanked by a 5′ homology arm and a 3′ homology arm each independently comprising 5 to 100, 10 to 150, or 20 to 150 nucleotides. In certain embodiments, the replacement sequence optionally comprises a promoter and/or polyA signal.

### Single-Strand Annealing

Single strand annealing (SSA) is another DNA repair process that repairs a double-strand break between two repeat sequences present in a target nucleic acid. Repeat sequences utilized by the SSA pathway are generally greater than 30 nucleotides in length. Resection at the break ends occurs to reveal repeat sequences on both strands of the target nucleic acid. After resection, single strand overhangs containing the repeat sequences are coated with RPA protein to prevent the repeats sequences from inappropriate annealing, e.g., to themselves. RAD52 binds to and each of the repeat sequences on the overhangs and aligns the sequences to enable the annealing of the complementary repeat sequences. After annealing, the single-strand flaps of the overhangs are cleaved. New DNA synthesis fills in any gaps, and ligation restores the DNA duplex. As a result of the processing, the DNA sequence between the two repeats is deleted. The length of the deletion can depend on many factors including the location of the two repeats utilized, and the pathway or processivity of the resection.

In contrast to HDR pathways, SSA does not require a template nucleic acid to alter a target nucleic acid sequence. Instead, the complementary repeat sequence is utilized.

### Other DNA Repair Pathways

### SSBR (Single Strand Break Repair)

Single-stranded breaks (SSB) in the genome are repaired by the SSBR pathway, which is a distinct mechanism from the DSB repair mechanisms discussed above. The SSBR pathway has four major stages: SSB detection, DNA end processing, DNA gap filling, and DNA ligation. A more detailed explanation is given in Caldecott 2008, and a summary is given here.

In the first stage, when a SSB forms, PARP1 and/or PARP2 recognize the break and recruit repair machinery. The binding and activity of PARP1 at DNA breaks is transient and it seems to accelerate SSBr by promoting the focal accumulation or stability of SSBr protein complexes at the lesion. Arguably the most important of these SSBr proteins is XRCC1, which functions as a molecular scaffold that interacts with, stabilizes, and stimulates multiple enzymatic components of the SSBr process including the protein responsible for cleaning the DNA 3′ and 5′ ends. For instance, XRCC1 interacts with several proteins (DNA polymerase beta, PNK, and three nucleases, APE1, APTX, and APLF) that promote end processing. APE1 has endonuclease activity. APLF exhibits endonuclease and 3′ to 5′ exonuclease activities. APTX has endonuclease and 3′ to 5′ exonuclease activity.

This end processing is an important stage of SSBR since the 3′- and/or 5′-termini of most, if not all, SSBs are ‘damaged.’ End processing generally involves restoring a damaged 3′-end to a hydroxylated state and and/or a damaged 5′ end to a phosphate moiety, so that the ends become ligation-competent. Enzymes that can process damaged 3′ termini include PNKP, APE1, and TDP1. Enzymes that can process damaged 5′ termini include PNKP, DNA polymerase beta, and APTX. LIG3 (DNA ligase III) can also participate in end processing. Once the ends are cleaned, gap filling can occur.

At the DNA gap filling stage, the proteins typically present are PARP1, DNA polymerase beta, XRCC1, FEN1 (flap endonuclease 1), DNA polymerase delta/epsilon, PCNA, and LIG1. There are two ways of gap filling, the short patch repair and the long patch repair. Short patch repair involves the insertion of a single nucleotide that is missing. At some SSBs, “gap filling” might continue displacing two or more nucleotides (displacement of up to 12 bases have been reported). FEN1 is an endonuclease that removes the displaced 5′-residues. Multiple DNA polymerases, including Polo, are involved in the repair of SSBs, with the choice of DNA polymerase influenced by the source and type of SSB.

In the fourth stage, a DNA ligase such as LIG1 (Ligase I) or LIG3 (Ligase III) catalyzes joining of the ends. Short patch repair uses Ligase III and long patch repair uses Ligase I.

Sometimes, SSBR is replication-coupled. This pathway can involve one or more of CtIP, MRN, ERCC1, and FEN1. Additional factors that may promote SSBR include: aPARP, PARP1, PARP2, PARG, XRCC1, DNA polymerase b, DNA polymerase d, DNA polymerase e, PCNA, LIG1, PNK, PNKP, APE1, APTX, APLF, TDP1, LIG3, FEN1, CtIP, MRN, and ERCC1.

### MMR (Mismatch Repair)

Cells contain three excision repair pathways: MMR, BER, and NER. The excision repair pathways have a common feature in that they typically recognize a lesion on one strand of the DNA, then exo/endonucleases remove the lesion and leave a 1-30 nucleotide gap that is sub-sequentially filled in by DNA polymerase and finally sealed with ligase. A more complete picture is given in Li 2008, and a summary is provided here.

Mismatch repair (MMR) operates on mispaired DNA bases.

The MSH2/6 or MSH2/3 complexes both have ATPases activity that plays an important role in mismatch recognition and the initiation of repair. MSH2/6 preferentially recognizes base-base mismatches and identifies mispairs of 1 or 2 nucleotides, while MSH2/3 preferentially recognizes larger ID mispairs.

hMLH1 heterodimerizes with hPMS2 to form hMutLa which possesses an ATPase activity and is important for multiple steps of MMR. It possesses a PCNA/replication factor C (RFC)-dependent endonuclease activity which plays an important role in Y nick-directed MMR involving EXO1 (EXO1 is a participant in both HR and MMR.) It regulates termination of mismatch-provoked excision. Ligase I is the relevant ligase for this pathway. Additional factors that may promote MMR include: EXO1, MSH2, MSH3, MSH6, MLH1, PMS2, MLH3, DNA Pol d, RPA, HMGB1, RFC, and DNA ligase I.

### Base Excision Repair (BER)

The base excision repair (BER) pathway is active throughout the cell cycle; it is responsible primarily for removing small, non-helix-distorting base lesions from the genome. In contrast, the related Nucleotide Excision Repair pathway (discussed in the next section) repairs bulky helix-distorting lesions. A more detailed explanation is given in Caldecott 2008, and a summary is given here.

Upon DNA base damage, base excision repair (BER) is initiated and the process can be simplified into five major steps: (a) removal of the damaged DNA base; (b) incision of the subsequent a basic site; (c) clean-up of the DNA ends; (d) insertion of the desired nucleotide (e.g., HPFH mutant) into the repair gap; and (e) ligation of the remaining nick in the DNA backbone. These last steps are similar to the SSBR.

In the first step, a damage-specific DNA glycosylase excises the damaged base through cleavage of the N-glycosidic bond linking the base to the sugar phosphate backbone. Then AP endonuclease-1 (APE1) or bifunctional DNA glycosylases with an associated lyase activity incised the phosphodiester backbone to create a DNA single strand break (SSB). The third step of BER involves cleaning-up of the DNA ends. The fourth step in BER is conducted by Polo that adds a new complementary nucleotide into the repair gap and in the final step XRCC1/Ligase III seals the remaining nick in the DNA backbone. This completes the short-patch BER pathway in which the majority (˜80%) of damaged DNA bases are repaired. However, if the 5′ ends in step 3 are resistant to end processing activity, following one nucleotide insertion by Pol β there is then a polymerase switch to the replicative DNA polymerases, Pol δ/ε, which then add ˜2-8 more nucleotides into the DNA repair gap. This creates a 5′ flap structure, which is recognized and excised by flap endonuclease-1 (FEN-1) in association with the processivity factor proliferating cell nuclear antigen (PCNA). DNA ligase I then seals the remaining nick in the DNA backbone and completes long-patch BER. Additional factors that may promote the BER pathway include: DNA glycosylase, APE1, Polb, Pold, Pole, XRCC1, Ligase III, FEN-1, PCNA, RECQL4, WRN, MYH, PNKP, and APTX.

### Nucleotide Excision Repair (NER)

Nucleotide excision repair (NER) is an important excision mechanism that removes bulky helix-distorting lesions from DNA. Additional details about NER are given in Marteijn 2014, and a summary is given here. NER a broad pathway encompassing two smaller pathways: global genomic NER (GG-NER) and transcription coupled repair NER (TC-NER). GG-NER and TC-NER use different factors for recognizing DNA damage. However, they utilize the same machinery for lesion incision, repair, and ligation.

Once damage is recognized, the cell removes a short single-stranded DNA segment that contains the lesion. Endonucleases XPF/ERCC1 and XPG (encoded by ERCC5) remove the lesion by cutting the damaged strand on either side of the lesion, resulting in a single-strand gap of 22-30 nucleotides. Next, the cell performs DNA gap filling synthesis and ligation. Involved in this process are: PCNA, RFC, DNA Pol δ, DNA Pol ε or DNA Pol κ, and DNA ligase I or XRCC1/Ligase III. Replicating cells tend to use DNA pol F and DNA ligase I, while non-replicating cells tend to use DNA Pol δ, DNA Pol κ, and the XRCC1/Ligase III complex to perform the ligation step.

NER can involve the following factors: XPA-G, POLH, XPF, ERCC1, XPA-G, and LIG1. Transcription-coupled NER (TC-NER) can involve the following factors: CSA, CSB, XPB, XPD, XPG, ERCC1, and TTDA. Additional factors that may promote the NER repair pathway include XPA-G, POLH, XPF, ERCC1, XPA-G, LIG1, CSA, CSB, XPA, XPB, XPC, XPD, XPF, XPG, TTDA, UVSSA, USP7, CETN2, RAD23B, UV-DDB, CAK subcomplex, RPA, and PCNA.

### Interstrand Crosslink (ICL)

A dedicated pathway called the ICL repair pathway repairs interstrand crosslinks. Interstrand crosslinks, or covalent crosslinks between bases in different DNA strand, can occur during replication or transcription. ICL repair involves the coordination of multiple repair processes, in particular, nucleolytic activity, translesion synthesis (TLS), and HDR. Nucleases are recruited to excise the ICL on either side of the crosslinked bases, while TLS and HDR are coordinated to repair the cut strands. ICL repair can involve the following factors: endonucleases, e.g., XPF and RAD51C, endonucleases such as RAD51, translesion polymerases, e.g., DNA polymerase zeta and Rev 1), and the Fanconi anemia (FA) proteins, e.g., FancJ.

### Other Pathways

Several other DNA repair pathways exist in mammals.

Translesion synthesis (TLS) is a pathway for repairing a single stranded break left after a defective replication event and involves translesion polymerases, e.g., DNA polo and Rev 1.

Error-free postreplication repair (PRR) is another pathway for repairing a single stranded break left after a defective replication event.

Examples of gRNAs in Genome Editing Methods gRNA molecules as described herein can be used with Cas9 molecules that generate a double strand break or a single strand break to alter the sequence of a target nucleic acid, e.g., a target position or target genetic signature. gRNA molecules useful in these methods are described below.

In certain embodiments, the gRNA, e.g., a chimeric gRNA, is configured such that it comprises one or more of the following properties:
(a) it can position, e.g., when targeting a Cas9 molecule that makes double strand breaks, a double strand break (i) within 50, 100, 150, 200, 250, 300, 350, 400, 450, or 500 nucleotides of a target position, or (ii) sufficiently close that the target position is within the region of end resection;
(b) it has a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and
(c)(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes or S. aureus tail and proximal domain, or a sequence that differs by no more than 1, 2, 3, 4, 5, 6, 7, 8, 9, or 10 nucleotides therefrom;
(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes or S. aureus gRNA, or a sequence that differs by no more than 1, 2, 3, 4, 5, 6, 7, 8, 9 or 10 nucleotides therefrom;
(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes or S. aureus gRNA, or a sequence that differs by no more than 1, 2, 3, 4, 5, 6, 7, 8, 9 or 10 nucleotides therefrom;
(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes or S. aureus tail domain, or a sequence that differs by no more than 1, 2, 3, 4, 5, 6, 7, 8, 9, or 10 nucleotides therefrom; or
(c)(v) the tail domain comprises 15, 20, 25, 30, 35, 40 nucleotides or all of the corresponding portions of a naturally occurring tail domain, e.g., a naturally occurring S. pyogenes or S. aureus tail domain.

In certain embodiments, the gRNA is configured such that it comprises properties a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(ii), and c(i); a(i), b(ii), and c(ii); a(i), b(iii), and c(i); a(i), b(iii), and c(ii); a(i), b(iv), and c(i); a(i), b(iv), and c(ii); a(i), b(v), and c(i); a(i), b(v), and c(ii); a(i), b(vi), and c(i); a(i), b(vi), and c(ii); a(i), b(vii), and c(i); a(i), b(vii), and c(ii); a(i), b(viii), and c(i); a(i), b(viii), and c(ii); a(i), b(ix), and c(i); a(i), b(ix), and c(ii); a(i), b(x), and c(i); a(i), b(x), and c(ii); a(i), b(xi), or c(i); a(i), b(xi), and c(ii).

In certain embodiments, the gRNA, e.g., a chimeric gRNA, is configured such that it comprises one or more of the following properties:
(a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;
(b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and
(c)(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes or S. aureus tail and proximal domain, or a sequence that differs by no more than 1, 2, 3, 4, 5, 6, 7, 8, 9 or 10 nucleotides therefrom;
(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, or S. aureus gRNA, or a sequence that differs by no more than 1, 2, 3, 4, 5, 6, 7, 8, 9 or 10 nucleotides therefrom;
(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes or S. aureus gRNA, or a sequence that differs by no more than 1, 2, 3, 4, 5, 6, 7, 8, 9, or 10 nucleotides therefrom;
(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes or S. aureus tail domain, or a sequence that differs by no more than 1, 2, 3, 4, 5, 6, 7, 8, 9 or 10 nucleotides therefrom; or
(c)(v) the tail domain comprises 15, 20, 25, 30, 35, or 40 nucleotides or all of the corresponding portions of a naturally occurring tail domain, e.g., a naturally occurring S. pyogenes or S. aureus tail domain.

In certain embodiments, the gRNA is configured such that it comprises properties a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(ii), and c(i); a(i), b(ii), and c(ii); a(i), b(iii), and c(i); a(i), b(iii), and c(ii); a(i), b(iv), and c(i); a(i), b(iv), and c(ii); a(i), b(v), and c(i); a(i), b(v), and c(ii); a(i), b(vi), and c(i); a(i), b(vi), and c(ii); a(i), b(vii), and c(i); a(i), b(vii), and c(ii); a(i), b(viii), and c(i); a(i), b(viii), and c(ii); a(i), b(ix), and c(i); a(i), b(ix), and c(ii); a(i), b(x), and c(i); a(i), b(x), and c(ii); a(i), b(xi), and c(i); a(i), b(xi), and c(ii).

In certain embodiments, the gRNA is used with a Cas9 nickase molecule having HNH activity, e.g., a Cas9 molecule having the RuvC activity inactivated, e.g., a Cas9 molecule having a mutation at D10, e.g., the D10A mutation.

In an embodiment, the gRNA is used with a Cas9 nickase molecule having RuvC activity, e.g., a Cas9 molecule having the HNH activity inactivated, e.g., a Cas9 molecule having a mutation at 840, e.g., the H840A.

In an embodiment, the gRNAs are used with a Cas9 nickase molecule having RuvC activity, e.g., a Cas9 molecule having the HNH activity inactivated, e.g., a Cas9 molecule having a mutation at N863, e.g., the N863A mutation.

In an embodiment, a pair of gRNAs, e.g., a pair of chimeric gRNAs, comprising a first and a second gRNA, is configured such that they comprises one or more of the following properties:
(a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;
(b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides;
(c)(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes or S. aureus tail and proximal domain, or a sequence that differs by no more than 1, 2, 3, 4, 5, 6, 7, 8, 9, or 10 nucleotides therefrom;
(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes or S. aureus gRNA, or a sequence that differs by no more than 1, 2, 3, 4, 5, 6, 7, 8, 9, or 10 nucleotides therefrom;
(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes or S. aureus gRNA, or a sequence that differs by no more than 1, 2, 3, 4, 5, 6, 7, 8, 9 or 10 nucleotides therefrom;
(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes or S. aureus tail domain; or, or a sequence that differs by no more than 1, 2, 3, 4, 5, 6, 7, 8, 9, or 10 nucleotides therefrom; or
(c)(v) the tail domain comprises 15, 20, 25, 30, 35, or 40 nucleotides or all of the corresponding portions of a naturally occurring tail domain, e.g., a naturally occurring S. pyogenes or S. aureus tail domain;
(d) the gRNAs are configured such that, when hybridized to target nucleic acid, they are separated by 0-50, 0-100, 0-200, at least 10, at least 20, at least 30 or at least 50 nucleotides; (e) the breaks made by the first gRNA and second gRNA are on different strands; and (f) the PAMs are facing outwards.

In certain embodiments, one or both of the gRNAs is configured such that it comprises properties a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(i), c, and d; a(i), b(i), c, and e; a(i), b(i), c, d, and e; a(i), b(ii), and c(i); a(i), b(ii), and c(ii); a(i), b(ii), c, and d; a(i), b(ii), c, and e; a(i), b(ii), c, d, and e; a(i), b(iii), and c(i); a(i), b(iii), and c(ii); a(i), b(iii), c, and d; a(i), b(iii), c, and e; a(i), b(iii), c, d, and e; a(i), b(iv), and c(i); a(i), b(iv), and c(ii); a(i), b(iv), c, and d; a(i), b(iv), c, and e; a(i), b(iv), c, d, and e; a(i), b(v), and c(i); a(i), b(v), and c(ii); a(i), b(v), c, and d; a(i), b(v), c, and e; a(i), b(v), c, d, and e; a(i), b(vi), and c(i); a(i), b(vi), and c(ii); a(i), b(vi), c, and d; a(i), b(vi), c, and e; a(i), b(vi), c, d, and e; a(i), b(vii), and c(i); a(i), b(vii), and c(ii); a(i), b(vii), c, and d; a(i), b(vii), c, and e; a(i), b(vii), c, d, and e; a(i), b(viii), and c(i); a(i), b(viii), and c(ii); a(i), b(viii), c, and d; a(i), b(viii), c, and e; a(i), b(viii), c, d, and e; a(i), b(ix), and c(i); a(i), b(ix), and c(ii); a(i), b(ix), c, and d; a(i), b(ix), c, and e; a(i), b(ix), c, d, and e; a(i), b(x), and c(i); a(i), b(x), and c(ii); a(i), b(x), c, and d; a(i), b(x), c, and e; a(i), b(x), c, d, and e; a(i), b(xi), and c(i); a(i), b(xi), and c(ii); a(i), b(xi), c, and d; a(i), b(xi), c, and e; a(i), b(xi), c, d, and e.

In certain embodiments, the gRNAs are used with a Cas9 nickase molecule having HNH activity, e.g., a Cas9 molecule having the RuvC activity inactivated, e.g., a Cas9 molecule having a mutation at D10, e.g., the D10A mutation.

In certain embodiments, the gRNAs are used with a Cas9 nickase molecule having RuvC activity, e.g., a Cas9 molecule having the HNH activity inactivated, e.g., a Cas9 molecule having a mutation at H840, e.g., the H840A mutation.

In certain embodiments, the gRNAs are used with a Cas9 nickase molecule having RuvC activity, e.g., a Cas9 molecule having the HNH activity inactivated, e.g., a Cas9 molecule having a mutation at N863, e.g., the N863A mutation.

### Target Cells

Cas9 molecules and gRNA molecules, e.g., a Cas9 molecule/gRNA molecule complex, can be used to alter (e.g., introduce a mutation or deletion in) a target nucleic acid, e.g., a T-globin gene (e.g., HBG1, HBG2) regulatory region, in a wide variety of cells. In certain embodiments, alteration of a target nucleic acid in a target cell may be performed in vitro, ex vivo or in vivo.

The Cas9 and gRNA molecules described herein can be delivered to a target cell. In certain embodiments, the target cell is an erythroid cell, e.g., an erythroblast. In certain embodiments, erythroid cells are preferentially targeted, e.g., at least about 90%, 95%, 96%, 97%, 98%, 99%, or 100% of the targeted cells are erythroid cells. For example, in the case of in vivo delivery, erythroid cells are preferentially targeted, and if cells are treated ex vivo and returned to the subject, erythroid cells are preferentially modified.

In certain embodiments, the target cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In certain embodiments, the target cell is a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In certain embodiments, the target cell is a myeloid progenitor cell (e.g., a common myeloid progenitor (CMP) cell or granulocyte macrophage progenitor (GMP) cell). In certain embodiments, the target cell is a lymphoid progenitor cell, e.g., a common lymphoid progenitor (CLP) cell. In certain embodiments, the target cell is an erythroid progenitor cell (e.g., an MEP cell). In certain embodiments, the target cell is a hematopoietic stem/progenitor cell (e.g., a long term HSC (LT-HSC), short term HSC (ST-HSC), MPP cell, or lineage restricted progenitor (LRP) cell). In certain embodiments, the target cell is a CD34⁺ cell, CD34⁺CD90⁺ cell, CD34⁺CD38⁻ cell, CD34⁺CD90⁺CD49f⁺CD38⁻ CD45RA⁻ cell, CD105⁺ cell, CD31⁺, or CD133⁺ cell, or a CD34⁺CD90⁺CD133⁺ cell. In certain embodiments, the target cell is an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In certain embodiments, the target cell is a mobilized peripheral blood hematopoietic CD34⁺ cell (after the patient is treated with a mobilization agent, e.g., G-CSF or Plerixafor). In certain embodiments, the target cell is a peripheral blood endothelial cell.

In certain embodiments, a target cell is manipulated ex vivo by editing a γ-globin gene regulatory region, then the target cell is administered to the subject. Sources of target cells for ex vivo manipulation may include, for example, the subject's blood, bone marrow, or cord blood. Other sources of target cells for ex vivo manipulation may include, for example, heterologous donor blood, cord blood, or bone marrow. In certain embodiments, an erythrocyte is removed from the subject, manipulated ex vivo as described above, and returned to the subject. In certain embodiments, a hematopoietic stem cell is removed from the subject, manipulated ex vivo as described above, and returned to the subject. In certain embodiments, an erythroid progenitor cell is removed from a subject, manipulated ex vivo as described above, and returned to the subject. In certain embodiments, a myeloid progenitor cell is removed from a subject, manipulated ex vivo as described above, and returned to the subject. In certain embodiments, a multipotent progenitor (MPP) cell is removed from a subject, manipulated ex vivo as described above, and returned to the subject. In certain embodiments, a hematopoietic stem/progenitor cell (HSC) is removed from a subject, manipulated ex vivo as described above, and returned to the subject. In certain embodiments, a CD34⁺ HSC is removed from a subject, manipulated ex vivo as described above, and returned to the subject.

In certain embodiments, modified HSCs generated ex vivo are administered to a subject without myeloablative pre-conditioning. In other embodiments, the modified HSCs are administered after mild myeloablative conditioning such that, followed engraftment, some of the hematopoietic cells are derived from the modified HSCs. In still other embodiments, the modified HSCs are administered after full myeloablation such that, following engraftment, 100% of the hematopoietic cells are derived from the modified HSCs.

A suitable cell can also include a stem cell such as, e.g., an embryonic stem cell, induced pluripotent stem cell, hematopoietic stem cell, or hemogenic endothelial (HE) cell (precursor to both hematopoietic stem cells and endothelial cells). In certain embodiments, the cell is an induced pluripotent stem (iPS) cell or a cell derived from an iPS cell, e.g., an iPS cell generated from the subject, modified using the methods disclosed herein, and differentiated into a clinically relevant cell such as e.g., an erythrocyte. In certain embodiments, AAV is used to transduce the target cells.

In certain embodiments, stem cells used for gene editing as described herein may be prepared for use in accordance with the methods described in the examples in Gori 2016 at, e.g., pages 219-223, 223-224, 227-231, 231-236, 235-238, 240-241, 242-244, which is incorporated herein by reference. Stem cells can be cultured and expanded in any manner that is suitable and known by the skilled artisan.

Cells produced by the methods described herein may be used immediately. Alternatively, the cells may be frozen (e.g., in liquid nitrogen) and stored for later use. The cells will usually be frozen in 10% dimethylsulfoxide (DMSO), 50% serum, 40% buffered medium, or some other such solution as is commonly used in the art to preserve cells at such freezing temperature, and thawed in such a manner as commonly known in the art for thawing frozen cultured cells. Cells may also be thermostabilized for prolonged storage at 4° C.

### Delivery, Formulations, and Routes of Administration

Genome editing system components, e.g., RNA-guided nuclease molecules, e.g., a Cas9 molecule, gRNA molecule (e.g., a Cas9 molecule/gRNA molecule complex), and a donor template nucleic acid, or all three, can be delivered, formulated, or administered in a variety of forms, see, e.g., Tables 3 and 4.

In certain embodiments, one Cas9 molecule and two or more (e.g., 2, 3, 4, or more) different gRNA molecules are delivered, e.g., by an AAV vector. In certain embodiments, the sequence encoding the Cas9 molecule and the sequence(s) encoding the two or more (e.g., 2, 3, 4, or more) different gRNA molecules are present on the same nucleic acid molecule, e.g., an AAV vector. When a Cas9 or gRNA component is delivered encoded in DNA the DNA will typically include a control region, e.g., comprising a promoter, to effect expression. Useful promoters for Cas9 molecule sequences include CMV, SFFV, EFS, EF-1a, PGK, CAG, and CBH promoters or a blood cell specific promoter. In an embodiment, the promoter is a constitutive promoter. In another embodiment, the promoter is a tissue specific promoter. Useful promoters for gRNAs include T7.H1, EF-1a, U6, U1, and tRNA promoters. Promoters with similar or dissimilar strengths can be selected to tune the expression of components. Sequences encoding a Cas9 molecule can comprise a nuclear localization signal (NLS), e.g., an SV40 NLS. In an embodiment, the sequence encoding a Cas9 molecule comprises at least two nuclear localization signals. In an embodiment, a promoter for a Cas9 molecule or a gRNA molecule can be, independently, inducible, tissue specific, or cell specific.

Table 3 provides examples of how the components can be formulated, delivered, or administered.

TABLE 3
Elements

| | | Donor | |
|---|---|---|---|
| | | Template | |
| Cas9 | gRNA | Nucleic | |
| Molecule(s) | Molecule(s) | Acid | Comments |
| DNA | DNA | DNA | In this embodiment, a Cas9 |
| | | | molecule, typically an eaCas9 |
| | | | molecule, and a gRNA are |
| | | | transcribed from DNA. In this |
| | | | embodiment, they are encoded |
| | | | on separate molecules. In |
| | | | this embodiment, the donor |
| | | | template is provided as |
| | | | a separate DNA molecule. |
| DNA | DNA | | In this embodiment, a Cas9 |
| | | | molecule, typically an eaCas9 |
| | | | molecule, and a gRNA are |
| | | | transcribed from DNA. In this |
| | | | embodiment, they are encoded |
| | | | on separate molecules. In |
| | | | this embodiment, the donor |
| | | | template is provided on |
| | | | the same DNA molecule that |
| | | | encodes the gRNA. |
| DNA | | DNA | In this embodiment, a Cas9 |
| | | | molecule, typically an eaCas9 |
| | | | molecule, and a gRNA are |
| | | | transcribed from DNA, here |
| | | | from a single molecule. In this |
| | | | embodiment, the donor |
| | | | template is provided as a |
| | | | separate DNA molecule. |
| DNA | DNA | | In this embodiment, a Cas9 |
| | | | molecule, typically an eaCas9 |
| | | | molecule, and a gRNA are |
| | | | transcribed from DNA. In this |
| | | | embodiment, they are encoded |
| | | | on separate molecules. In |
| | | | this embodiment, the donor |
| | | | template is provided on the |
| | | | same DNA molecule that |
| | | | encodes the Cas9. |
| DNA | RNA | DNA | In this embodiment, a |
| | | | Cas9 molecule, |
| | | | typically an eaCas9 molecule, |
| | | | is transcribed from DNA, and |
| | | | a gRNA is provided as in vitro |
| | | | transcribed or synthesized RNA. |
| | | | In this embodiment, the donor |
| | | | template is provided as |
| | | | a separate DNA molecule. |
| DNA | RNA | | In this embodiment, a Cas9 |
| | | | molecule, typically an eaCas9 |
| | | | molecule, is transcribed |
| | | | from DNA, and a gRNA |
| | | | is provided as in vitro |
| | | | transcribed or synthesized |
| | | | RNA. In this embodiment, |
| | | | the donor template is |
| | | | provided on the same DNA |
| | | | molecule that encodes the Cas9. |
| mRNA | RNA | DNA | In this embodiment, |
| | | | a Cas9 molecule, |
| | | | typically an eaCas9 molecule, |
| | | | is translated from in vitro |
| | | | transcribed mRNA, and a gRNA |
| | | | is provided as in vitro transcribed |
| | | | or synthesized RNA. In this |
| | | | embodiment, the |
| | | | donor template is provided |
| | | | as a DNA molecule. |
| mRNA | DNA | DNA | In this embodiment, |
| | | | a Cas9 molecule, |
| | | | typically an eaCas9 |
| | | | molecule, is translated |
| | | | from in vitro transcribed mRNA, |
| | | | and a gRNA is transcribed from |
| | | | DNA. In this embodiment, |
| | | | the donor template is |
| | | | provided as a separate |
| | | | DNA molecule. |
| mRNA | DNA | | In this embodiment, a Cas9 |
| | | | molecule, typically an eaCas9 |
| | | | molecule, is translated |
| | | | from in vitro transcribed mRNA, |
| | | | and a gRNA is transcribed from |
| | | | DNA. In this embodiment, |
| | | | the donor template is |
| | | | provided on the same |
| | | | DNA molecule that encodes |
| | | | the gRNA. |
| Protein | DNA | DNA | In this embodiment, a Cas9 |
| | | | molecule, typically an eaCas9 |
| | | | molecule, is provided as a |
| | | | protein, and a gRNA is |
| | | | transcribed from DNA. In this |
| | | | embodiment, the donor template |
| | | | is provided as a separate |
| | | | DNA molecule. |
| Protein | DNA | | In this embodiment, a Cas9 |
| | | | molecule, typically an eaCas9 |
| | | | molecule, is provided as a |
| | | | protein, and a gRNA is |
| | | | transcribed from |
| | | | DNA. In this embodiment, |
| | | | the donor template |
| | | | is provided on the same |
| | | | DNA molecule that |
| | | | encodes the gRNA. |
| Protein | RNA | DNA | In this embodiment, an |
| | | | eaCas9 molecule is |
| | | | provided as a protein, and |
| | | | a gRNA is provided |
| | | | as transcribed or synthesized |
| | | | RNA. In this embodiment, the |
| | | | donor template is provided as |
| | | | a DNA molecule. |

Table 4 summarizes various delivery methods for the components of a Cas system, e.g., the Cas9 molecule component and the gRNA molecule component, as described herein.

TABLE 4

| | | Delivery | | | |
|---|---|---|---|---|---|
| | | into | | | |
| | | Non- | Duration | | Type of |
| | | Dividing | of | Genome | Molecule |
| Delivery Vector/Mode | | Cells | Expression | Integration | Delivered |
| Physical (e.g., | | YES | Transient | NO | Nucleic |
| electroporation, | | | | | Acids |
| particle gun, | | | | | and |
| Calcium Phosphate | | | | | Proteins |
| transfection, cell | | | | | |
| compression | | | | | |
| or squeezing) | | | | | |
| Viral | Retrovirus | NO | Stable | YES | RNA |
| | Lentivirus | YES | Stable | YES/NO | RNA |
| | | | | with | |
| | | | | modifi- | |
| | | | | cations | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes | YES | Stable | NO | DNA |
| | Simplex | | | | |
| | Virus | | | | |
| Non-Viral | Cationic | YES | Transient | Depends | Nucleic |
| | Liposomes | | | on what is | Acids |
| | | | | delivered | and |
| | | | | | Proteins |
| | Polymeric | YES | Transient | Depends | Nucleic |
| | Nanoparticles | | | on what is | Acids |
| | | | | delivered | and |
| | | | | | Proteins |
| Biological | Attenuated | YES | Transient | NO | Nucleic |
| Non-Viral | Bacteria | | | | Acids |
| Delivery | Engineered | YES | Transient | NO | Nucleic |
| Vehicles | Bacteriophages | | | | Acids |
| | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

DNA-Based Delivery of an RNA-Guided Nuclease and or One or More gRNA Molecules

Nucleic acids encoding RNA-guided nucleases, e.g., Cas9 molecules (e.g., eaCas9 molecules), gRNA molecules, a donor template nucleic acid, or any combination (e.g., two or all) thereof can be administered to subjects or delivered into cells by art-known methods or as described herein. For example, Cas9-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding Cas9 molecules (e.g., eaCas9 molecules) and/or gRNA molecules can be conjugated to molecules (e.g., N-acetylgalactosamine) promoting uptake by the target cells (e.g., erythrocytes, HSCs). Donor template molecules can likewise be conjugated to molecules (e.g., N-acetylgalactosamine) promoting uptake by the target cells (e.g., erythrocytes, HSCs).

In some embodiments, the Cas9- and/or gRNA-encoding DNA is delivered by a vector (e.g., viral vector/virus or plasmid).

Vectors can comprise a sequence that encodes a Cas9 molecule and/or a gRNA molecule and/or a donor template with high homology to the region (e.g., target sequence) being targeted. In certain embodiments, the donor template comprises all or part of a target sequence. Exemplary donor templates are a repair template, e.g., a gene correction template, or a gene mutation template, e.g., point mutation (e.g., single nucleotide (nt) substitution) template). A vector can also comprise a sequence encoding a signal peptide (e.g., for nuclear localization, nucleolar localization, or mitochondrial localization), fused, e.g., to a Cas9 molecule sequence. For example, the vectors can comprise a nuclear localization sequence (e.g., from SV40) fused to the sequence encoding the Cas9 molecule.

One or more regulatory/control elements, e.g., promoters, enhancers, introns, polyadenylation signals, Kozak consensus sequences, or internal ribosome entry sites (IRES), can be included in the vectors. In some embodiments, the promoter is recognized by RNA polymerase II (e.g., a CMV promoter). In other embodiments, the promoter is recognized by RNA polymerase III (e.g., a U6 promoter). In some embodiments, the promoter is a regulated promoter (e.g., inducible promoter). In other embodiments, the promoter is a constitutive promoter. In some embodiments, the promoter is a tissue specific promoter. In some embodiments, the promoter is a viral promoter. In other embodiments, the promoter is a non-viral promoter.

In some embodiments, the vector is a viral vector (e.g., for generation of recombinant viruses). In some embodiments, the virus is a DNA virus (e.g., dsDNA or ssDNA virus). In other embodiments, the virus is an RNA virus (e.g., an ssRNA virus). In some embodiments, the virus infects dividing cells. In other embodiments, the virus infects non-dividing cells. Exemplary viral vectors/viruses include, e.g., retroviruses, lentiviruses, adenovirus, adeno-associated virus (AAV), vaccinia viruses, poxviruses, and herpes simplex viruses.

In some embodiments, the virus infects both dividing and non-dividing cells. In some embodiments, the virus can integrate into the host genome. In some embodiments, the virus is engineered to have reduced immunity, e.g., in human. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the Cas9 molecule and/or the gRNA molecule. In other embodiments, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In an embodiment, the viral vector recognizes a specific cell type or tissue. For example, the viral vector can be pseudotyped with a different/alternative viral envelope glycoprotein; engineered with a cell type-specific receptor (e.g., genetic modification(s) of one or more viral envelope glycoproteins to incorporate a targeting ligand such as a peptide ligand, a single chain antibody, or a growth factor); and/or engineered to have a molecular bridge with dual specificities with one end recognizing a viral glycoprotein and the other end recognizing a moiety of the target cell surface (e.g., a ligand-receptor, monoclonal antibody, avidin-biotin and chemical conjugation).

In some embodiments, the Cas9- and/or gRNA-encoding nucleic acid sequence is delivered by a recombinant retrovirus. In some embodiments, the retrovirus (e.g., Moloney murine leukemia virus) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the Cas9- and/or gRNA-encoding nucleic acid sequence is delivered by a recombinant lentivirus. In an embodiment, the donor template nucleic acid is delivered by a recombinant retrovirus. For example, the lentivirus is replication-defective, e.g., does not comprise one or more genes required for viral replication.

In an embodiment, the Cas9- and/or gRNA-encoding nucleic acid sequence is delivered by a recombinant lentivirus. In an embodiment, the donor template nucleic acid is delivered by a recombinant lentivirus. For example, the lentivirus is replication-defective, e.g., does not comprise one or more genes required for viral replication.

In some embodiments, the Cas9- and/or gRNA-encoding nucleic acid sequence is delivered by a recombinant adenovirus. In an embodiment, the donor template nucleic acid is delivered by a recombinant adenovirus. In some embodiments, the adenovirus is engineered to have reduced immunity in human.

In some embodiments, the Cas9- and/or gRNA-encoding nucleic acid sequence is delivered by a recombinant AAV. In an embodiment, the donor template nucleic acid is delivered by a recombinant AAV. In some embodiments, the AAV does not incorporate its genome into that of a host cell, e.g., a target cell as describe herein. In some embodiments, the AAV can incorporate its genome into that of the host cell. In some embodiments, the AAV is a self-complementary adeno-associated virus (scAAV), e.g., a scAAV that packages both strands which anneal together to form double stranded DNA.

In an embodiment, an AAV capsid that can be used in the methods described herein is a capsid sequence from serotype AAV1, AAV2, AAV3, AAV4, AAV5, AAV6, AAV7, AAV8, AAV9, AAV.rh8, AAV.rh10, AAV.rh32/33, AAV.rh43, AAV.rh64R1, or AAV7m8.

In an embodiment, the Cas9- and/or gRNA-encoding DNA is delivered in a re-engineered AAV capsid, e.g., with 50% or greater, e.g., 60% or greater, 70% or greater, 80% or greater, 90% or greater, or 95% or greater, sequence homology with a capsid sequence from serotypes AAV1, AAV2, AAV3, AAV4, AAV5, AAV6, AAV7, AAV8, AAV9, AAV.rh8, AAV.rh10, AAV.rh32/33, AAV.rh43, or AAV.rh64R1.

In an embodiment, the Cas9- and/or gRNA-encoding DNA is delivered by a chimeric AAV capsid. In an embodiment, the donor template nucleic acid is delivered by a chimeric AAV capsid. Exemplary chimeric AAV capsids include, but are not limited to, AAV9i1, AAV2i8, AAV-DJ, AAV2G9, AAV2i8G9, or AAV8G9.

In an embodiment, the AAV is a self-complementary adeno-associated virus (scAAV), e.g., a scAAV that packages both strands which anneal together to form double stranded DNA.

In some embodiments, the Cas9- and/or gRNA-encoding DNA is delivered by a hybrid virus, e.g., a hybrid of one or more of the viruses described herein. In an embodiment, the hybrid virus is hybrid of an AAV (e.g., of any AAV serotype), with a Bocavirus, B19 virus, porcine AAV, goose AAV, feline AAV, canine AAV, or MVM.

A packaging cell is used to form a virus particle that is capable of infecting a target cell. Exemplary packaging cells include 293 cells, which can package adenovirus, and ψ2 or PA317 cells, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., Cas9. For example, an AAV vector used in gene therapy typically only possesses inverted terminal repeat (ITR) sequences from the AAV genome which are required for packaging and gene expression in the host or target cell. The missing viral functions can be supplied in trans by the packaging cell line and/or plasmid containing E2A, E4, and VA genes from adenovirus, and plasmid encoding Rep and Cap genes from AAV, as described in “Triple Transfection Protocol.” Henceforth, the viral DNA is packaged in a cell line, which contains a helper plasmid encoding the other AAV genes, namely rep and cap, but lacking ITR sequences. In certain embodiments, the viral DNA is packaged in a producer cell line, which contains E1A and/or E1B genes from adenovirus. The cell line is also infected with adenovirus as a helper. The helper virus (e.g., adenovirus or HSV) or helper plasmid promotes replication of the AAV vector and expression of AAV genes from the helper plasmid with ITRs. The helper plasmid is not packaged in significant amounts due to a lack of ITR sequences. Contamination with adenovirus can be reduced by, e.g., heat treatment to which adenovirus is more sensitive than AAV.

In certain embodiments, the viral vector is capable of cell type and/or tissue type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, single chain antibody, or growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In certain embodiments, the viral vector achieves cell type specific expression. For example, a tissue-specific promoter can be constructed to restrict expression of the transgene (Cas9 and gRNA) to only the target cell. The specificity of the vector can also be mediated by microRNA-dependent control of transgene expression. In an embodiment, the viral vector has increased efficiency of fusion of the viral vector and a target cell membrane. For example, a fusion protein such as fusion-competent hemagglutin (HA) can be incorporated to increase viral uptake into cells. In an embodiment, the viral vector has the ability of nuclear localization. For example, a virus that requires the breakdown of the nuclear envelope (during cell division) and therefore will not infect a non-diving cell can be altered to incorporate a nuclear localization peptide in the matrix protein of the virus thereby enabling the transduction of non-proliferating cells.

In some embodiments, the Cas9- and/or gRNA-encoding DNA is delivered by a non-vector based method (e.g., using naked DNA or DNA complexes). For example, the DNA can be delivered, e.g., by organically modified silica or silicate (Ormosil), electroporation, transient cell compression or squeezing (see, e.g., Lee 2012), gene gun, sonoporation, magnetofection, lipid-mediated transfection, dendrimers, inorganic nanoparticles, calcium phosphates, or a combination thereof.

In an embodiment, delivery via electroporation comprises mixing the cells with the Cas9- and/or gRNA-encoding DNA in a cartridge, chamber or cuvette and applying one or more electrical impulses of defined duration and amplitude. In an embodiment, delivery via electroporation is performed using a system in which cells are mixed with the Cas9- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the Cas9- and/or gRNA-encoding DNA is delivered by a combination of a vector and a non-vector based method. In an embodiment, the donor template nucleic acid is delivered by a combination of a vector and a non-vector based method. For example, virosomes combine liposomes with an inactivated virus (e.g., HIV or influenza virus), which can result in more efficient gene transfer, e.g., in respiratory epithelial cells than either viral or liposomal methods alone.

In certain embodiments, the delivery vehicle is a non-viral vector, and in certain of these embodiments the non-viral vector is an inorganic nanoparticle. Exemplary inorganic nanoparticles include, e.g., magnetic nanoparticles (e.g., Fe₃MnO₂) or silica. The outer surface of the nanoparticle can be conjugated with a positively charged polymer (e.g., polyethylenimine, polylysine, polyserine) which allows for attachment (e.g., conjugation or entrapment) of payload. In an embodiment, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids for gene transfer are shown below in Table 1.

TABLE 1
Lipids Used for Gene Transfer

| Lipid | Abbreviation | Feature |
|---|---|---|
| 1,2-Dioleoyl-sn-glycero-3- | DOPC | Helper |
| phosphatidylcholine | | |
| 1,2-Dioleoyl-sn-glycero-3- | DOPE | Helper |
| phosphatidylethanolamine | | |
| Cholesterol | Helper | |
| N-[1-(2,3-Dioleyloxy)propyl]N,N,N- | DOTMA | Cationic |
| trimethylammonium chloride | | |
| 1,2-Dioleoyloxy-3- | DOTAP | Cationic |
| trimethylammonium-propane | | |
| Dioctadecylamidoglycylspermine | DOGS | Cationic |
| N-(3-Aminopropyl)-N,N- | GAP-DLRIE | Cationic |
| dimethyl-2,3-bis(dodecyloxy)-1- | | |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)- | 2Oc | Cationic |
| 2,4,6-trimethylpyridinium | | |
| 2,3-Dioleyloxy-N-[2(sperminecarboxamido- | DOSPA | Cationic |
| ethyl]-N,N-dimethyl- | | |
| 1-propanaminium trifluoroacetate | | |
| 1,2-Dioleyl-3-trimethylammonium-propane | DOPA | Cationic |
| N-(2-Hydroxyethyl)-N,N-dimethyl- | MDRIE | Cationic |
| 2,3-bis(tetradecyloxy)-1- | | |
| propanaminium bromide | | |
| Dimyristooxypropyl dimethyl | DMRI | Cationic |
| hydroxyethyl ammonium bromide | | |
| 3β-[N-(N',N'-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]- | | |
| dimethylammonium chloride | | |
| rac-[2(2,3-Dihexadecyloxypropyl- | CLIP-6 | Cationic |
| oxymethyloxy)ethyl]trimethylammonium | | |
| bromide | | |
| Ethyldimyristoylphosphatidylcholine | EDMPC | Cationic |
| 1,2-Distearyloxy-N,N-dimethyl- | DSDMA | Cationic |
| 3-aminopropane | | |
| 1,2-Dimyristoyl-trimethylammonium | DMTAP | Cationic |
| propane | | |
| O,O'-Dimyristyl-N-lysyl aspartate | DMKE | Cationic |
| 1,2-Distearoyl-sn-glycero-3- | DSEPC | Cationic |
| ethylphosphocholine | | |
| N-Palmitoyl D-erythro-sphingosyl | CCS | Cationic |
| carbamoyl-spermine | | |
| N-t-Butyl-N0-tetradecyl-3- | diC14-amidine | Cationic |
| tetradecylaminopropionamidine | | |
| Octadecenolyoxy[ethyl-2- | DOTIM | Cationic |
| heptadecenyl-3 hydroxyethyl] | | |
| imidazolinium chloride | | |
| N1-Cholesteryloxycarbonyl-3,7- | CDAN | Cationic |
| diazanonane-1,9-diamine | | |
| 2-(3-[Bis(3-amino-propyl)- | RPR209120 | Cationic |
| amino]propylamino)-N- | | |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl- | DLin-KC2- | Cationic |
| [1,3]-dioxolane | DMA | |
| dilinoleyl-methyl-4- | DLin-MC3- | Cationic |
| dimethylaminobutyrate | DMA | |

Exemplary polymers for gene transfer are shown below in Table 5.

TABLE 5
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3'-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In an embodiment, the vehicle has targeting modifications to increase target cell update of nanoparticles and liposomes, e.g., cell specific antigens, monoclonal antibodies, single chain antibodies, aptamers, polymers, sugars (e.g., N-acetylgalactosamine (GalNAc)), and cell penetrating peptides. In an embodiment, the vehicle uses fusogenic and endosome-destabilizing peptides/polymers. In an embodiment, the vehicle undergoes acid-triggered conformational changes (e.g., to accelerate endosomal escape of the cargo). In an embodiment, a stimuli-cleavable polymer is used, e.g., for release in a cellular compartment. For example, disulfide-based cationic polymers that are cleaved in the reducing cellular environment can be used.

In an embodiment, the delivery vehicle is a biological non-viral delivery vehicle. In an embodiment, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In an embodiment, the vehicle is a genetically modified bacteriophage (e.g., engineered phages having large packaging capacity, less immunogenic, containing mammalian plasmid maintenance sequences and having incorporated targeting ligands). In an embodiment, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In an embodiment, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject (i.e., patient) derived membrane-bound nanovesicle (30-100 nm) of endocytic origin (e.g., can be produced from various cell types and can therefore be taken up by cells without the need of for targeting ligands).

In an embodiment, one or more nucleic acid molecules (e.g., DNA molecules) other than the components of a Cas system, e.g., the Cas9 molecule component and/or the gRNA molecule component described herein, are delivered. In an embodiment, the nucleic acid molecule is delivered at the same time as one or more of the components of the Cas system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In an embodiment, the nucleic acid molecule is delivered by a different means than one or more of the components of the Cas system, e.g., the Cas9 molecule component and/or the gRNA molecule component, are delivered. The nucleic acid molecule can be delivered by any of the delivery methods described herein. For example, the nucleic acid molecule can be delivered by a viral vector, e.g., an integration-deficient lentivirus, and the Cas9 molecule component and/or the gRNA molecule component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

### Delivery of RNA Encoding an RNA-Guided Nuclease

RNA encoding RNA-guided nucleases, e.g., Cas9 molecules, and/or gRNA molecules, can be delivered into cells, e.g., target cells described herein, by art-known methods or as described herein. For example, Cas9-encoding and/or gRNA-encoding RNA can be delivered, e.g., by microinjection, electroporation, transient cell compression or squeezing (see, e.g., Lee 2012), lipid-mediated transfection, peptide-mediated delivery, or a combination thereof. Cas9-encoding and/or gRNA-encoding RNA can be conjugated to molecules) promoting uptake by the target cells (e.g., target cells described herein).

In an embodiment, delivery via electroporation comprises mixing the cells with the RNA encoding Cas9 molecules and/or gRNA molecules, with or without donor template nucleic acid molecules, in a cartridge, chamber or cuvette and applying one or more electrical impulses of defined duration and amplitude. In an embodiment, delivery via electroporation is performed using a system in which cells are mixed with the RNA encoding Cas9 molecules and/or gRNA molecules, with or without donor template nucleic acid molecules in a vessel connected to a device (e.g., a pump) which feeds the mixture into a cartridge, chamber or cuvette wherein one or more electrical impulses of defined duration and amplitude are applied, after which the cells are delivered to a second vessel. Cas9-encoding and/or gRNA-encoding RNA can be conjugated to molecules to promote uptake by the target cells (e.g., target cells described herein).

### Delivery of RNA-Guided Nucleases

RNA-guided nucleases, e.g., Cas9 molecules, can be delivered into cells by art-known methods or as described herein. For example, Cas9 protein molecules can be delivered, e.g., by microinjection, electroporation, transient cell compression or squeezing (see, e.g., Lee 2012), lipid-mediated transfection, peptide-mediated delivery, or a combination thereof. Delivery can be accompanied by DNA encoding a gRNA or by a gRNA. Cas9 protein can be conjugated to molecules promoting uptake by the target cells (e.g., target cells described herein).

In an embodiment, delivery via electroporation comprises mixing the cells with the Cas9 molecules and/or gRNA molecules, with or without donor nucleic acid, in a cartridge, chamber or cuvette and applying one or more electrical impulses of defined duration and amplitude. In an embodiment, delivery via electroporation is performed using a system in which cells are mixed with the Cas9 molecules and/or gRNA molecules, with or without donor nucleic acid in a vessel connected to a device (e.g., a pump) which feeds the mixture into a cartridge, chamber or cuvette wherein one or more electrical impulses of defined duration and amplitude are applied, after which the cells are delivered to a second vessel. Cas9-encoding and/or gRNA-encoding RNA can be conjugated to molecules to promote uptake by the target cells (e.g., target cells described herein).

### Route of Administration of Genome Editing System Components

Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically may be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In an embodiment, significantly smaller amounts of the components (compared with systemic approaches) may exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration may be provided as a periodic bolus (for example, intravenously) or as continuous infusion from an internal reservoir or from an external reservoir (for example, from an intravenous bag or implantable pump). Components may be administered locally, for example, by continuous release from a sustained release drug delivery device.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, with the choice of the appropriate system depending on the required rate of release for a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

### Bi-Modal or Differential Delivery of Genome Editing System Components

Separate delivery of Cas system components, e.g., the Cas9 molecule component and the gRNA molecule component, and more particularly, delivery of the components by differing modes, can enhance performance, e.g., by improving tissue specificity and safety.

In certain embodiments, the Cas9 molecule and the gRNA molecule are delivered by different modes, or as sometimes referred to herein as differential modes. Different or differential modes as used herein refer modes of delivery that confer different pharmacodynamic or pharmacokinetic properties on the subject component molecule, e.g., a Cas9 molecule, gRNA molecule, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components, e.g., a Cas9 molecule and a gRNA molecule, can be delivered by modes that differ in terms of resulting half-life or persistent of the delivered component the body, or in a particular compartment, tissue or organ. In an embodiment, a gRNA molecule can be delivered by such modes. The Cas9 molecule component can be delivered by a mode which results in less persistence or less exposure to the body or a particular compartment or tissue or organ.

More generally, in an embodiment, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a Cas9 molecule, is delivered in a transient manner, for example as mRNA or as protein, ensuring that the full Cas9 molecule/gRNA molecule complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in an embodiment, a first component, e.g., a gRNA molecule is delivered by a first delivery mode that results in a first spatial, e.g., tissue, distribution. A second component, e.g., a Cas9 molecule is delivered by a second delivery mode that results in a second spatial, e.g., tissue, distribution. In an embodiment the first mode comprises a first element selected from a liposome, nanoparticle, e.g., polymeric nanoparticle, and a nucleic acid, e.g., viral vector. The second mode comprises a second element selected from the group. In an embodiment, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the Cas9 molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA molecule and the Cas9 molecule are packaged in separated delivery vehicles with distinct but overlapping tissue tropism, the fully functional complex is only be formed in the tissue that is targeted by both vectors.

### Ex Vivo Delivery of Cas System Components

In certain embodiments, Cas system components described in Table 3 are introduced into cells which are then introduced into the subject. Methods of introducing the components can include, e.g., any of the delivery methods described in Table 4.

### Modified Nucleosides, Nucleotides, and Nucleic Acids

Modified nucleosides and modified nucleotides can be present in nucleic acids, e.g., particularly gRNA, but also other forms of RNA, e.g., mRNA, RNAi, or siRNA. As described herein, “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside further comprising a phosphate group.

Modified nucleosides and nucleotides can include one or more of:
(i) alteration, e.g., replacement, of one or both of the non-linking phosphate oxygens and/or of one or more of the linking phosphate oxygens in the phosphodiester backbone linkage;
(ii) alteration, e.g., replacement, of a constituent of the ribose sugar, e.g., of the 2′ hydroxyl on the ribose sugar;
(iii) wholesale replacement of the phosphate moiety with “dephospho” linkers;
(iv) modification or replacement of a naturally occurring nucleobase;
(v) replacement or modification of the ribose-phosphate backbone;
(vi) modification of the 3′ end or 5′ end of the oligonucleotide, e.g., removal, modification or replacement of a terminal phosphate group or conjugation of a moiety; and
(vii) modification of the sugar.

The modifications listed above can be combined to provide modified nucleosides and nucleotides that can have two, three, four, or more modifications. For example, a modified nucleoside or nucleotide can have a modified sugar and a modified nucleobase. In an embodiment, every base of a gRNA is modified, e.g., all bases have a modified phosphate group, e.g., all are phosphorothioate groups. In an embodiment, all, or substantially all, of the phosphate groups of a unimolecular (or chimeric) or modular gRNA molecule are replaced with phosphorothioate groups.

In an embodiment, modified nucleotides, e.g., nucleotides having modifications as described herein, can be incorporated into a nucleic acid, e.g., a “modified nucleic acid.” In an embodiment, the modified nucleic acids comprise one, two, three or more modified nucleotides. In an embodiment, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are a modified nucleotides.

Unmodified nucleic acids can be prone to degradation by, e.g., cellular nucleases. For example, nucleases can hydrolyze nucleic acid phosphodiester bonds. Accordingly, in one aspect the modified nucleic acids described herein can contain one or more modified nucleosides or nucleotides, e.g., to introduce stability toward nucleases.

In an embodiment, the modified nucleosides, modified nucleotides, and modified nucleic acids described herein can exhibit a reduced innate immune response when introduced into a population of cells, both in vivo and ex vivo. The term “innate immune response” includes a cellular response to exogenous nucleic acids, including single stranded nucleic acids, generally of viral or bacterial origin, which involves the induction of cytokine expression and release, particularly the interferons, and cell death. In an embodiment, the modified nucleosides, modified nucleotides, and modified nucleic acids described herein can disrupt binding of a major groove interacting partner with the nucleic acid. In an embodiment, the modified nucleosides, modified nucleotides, and modified nucleic acids described herein can exhibit a reduced innate immune response when introduced into a population of cells, both in vivo and ex vivo, and also disrupt binding of a major groove interacting partner with the nucleic acid.

### Definitions of Chemical Groups

As used herein, “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 12, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In an embodiment, aryl groups have from 6 to about 20 carbon atoms.

As used herein, “alkenyl” refers to an aliphatic group containing at least one double bond.

As used herein, “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl.

As used herein, “arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

As used herein, “cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl.

As used herein, “heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl.

As used herein, “heteroaryl” refers to a monovalent radical of a heteroaromatic ring system. Examples of heteroaryl moieties include, but are not limited to, imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

### Phosphate Backbone Modifications

### Phosphate Group

In an embodiment, the phosphate group of a modified nucleotide can be modified by replacing one or more of the oxygens with a different substituent. Further, the modified nucleotide, e.g., modified nucleotide present in a modified nucleic acid, can include the wholesale replacement of an unmodified phosphate moiety with a modified phosphate as described herein. In an embodiment, the modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. In an embodiment, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotide diastereomers. In an embodiment, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of the Phosphate Group

The phosphate group can be replaced by non-phosphorus containing connectors. In an embodiment, the charge phosphate group can be replaced by a neutral moiety.

Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, and methyleneoxymethylimino.

Replacement of the ribophosphate backbone Scaffolds that can mimic nucleic acids can also be constructed wherein the phosphate linker and ribose sugar are replaced by nuclease resistant nucleoside or nucleotide surrogates. In an embodiment, the nucleobases can be tethered by a surrogate backbone. Examples can include, without limitation, the morpholino, cyclobutyl, pyrrolidine and peptide nucleic acid (PNA) nucleoside surrogates.

Sugar modifications The modified nucleosides and modified nucleotides can include one or more modifications to the sugar group. For example, the 2′ hydroxyl group (OH) can be modified or replaced with a number of different “oxy” or “deoxy” substituents. In an embodiment, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom.

Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In an embodiment, the “oxy”-2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In an embodiment, the “oxy”-2′ hydroxyl group modification can include the methoxyethyl group (MOE), (OCH₂CH₂OCH₃, e.g., a PEG derivative).

“Deoxy” modifications can include hydrogen (i.e., deoxyribose sugars, e.g., at the overhang portions of partially ds RNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the 1′ position on the sugar, e.g., alpha-nucleosides. The modified nucleic acids can also include “abasic” sugars, which lack a nucleobase at C-1′. These abasic sugars can also be further modified at one or more of the constituent sugar atoms. The modified nucleic acids can also include one or more sugars that are in the L form, e.g., L-nucleosides.

Generally, RNA includes the sugar group ribose, which is a 5-membered ring having an oxygen. Exemplary modified nucleosides and modified nucleotides can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In an embodiment, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds) or threose nucleic acid (TNA, where ribose is replaced with α-L-threofuranosyl-(3′→2′)).

### Modifications on the Nucleobase

The modified nucleosides and modified nucleotides described herein, which can be incorporated into a modified nucleic acid, can include a modified nucleobase. Examples of nucleobases include, but are not limited to, adenine (A), guanine (G), cytosine (C), and uracil (U). These nucleobases can be modified or wholly replaced to provide modified nucleosides and modified nucleotides that can be incorporated into modified nucleic acids. The nucleobase of the nucleotide can be independently selected from a purine, a pyrimidine, a purine or pyrimidine analog. In an embodiment, the nucleobase can include, for example, naturally-occurring and synthetic derivatives of a base.

### Uracil

In an embodiment, the modified nucleobase is a modified uracil. Exemplary nucleobases and nucleosides having a modified uracil include without limitation pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmoSU), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (TCm⁵U), 1-taurinomethyl-pseudouridine, 5-taurinomethyl-2-thio-uridine(τm⁵s2U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s2U), 1-methyl-4-thio-pseudouridine (m¹s4ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s2U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s2Um), 5-methoxycarbonylmethyl-2′-O-methyl-uridine (mem ⁵Um), 5-carbamoylmethyl-2′-O-methyl-uridine (ncm ⁵Um), 5-carboxymethylaminomethyl-2′-O-methyl-uridine (cmnm ⁵Um), 3,2′-O-dimethyl-uridine (m³Um), 5-(isopentenylaminomethyl)-2′-O-methyl-uridine (inm ⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

### Cytosine

In an embodiment, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m³C), N4-acetyl-cytidine (act), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k²C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f ⁵Cm), N4,N4,2′-O-trimethyl-cytidine (m⁴2Cm), 1-thio-cytidine, 2′-F-ara-cytidine, 2′-F-cytidine, and 2′-OH-ara-cytidine.

### Adenine

In an embodiment, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenosine, 7-deaza-8-aza-adenosine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenosine (m²A), N6-methyl-adenosine (m⁶A), 2-methylthio-N6-methyl-adenosine (ms2 m⁶A), N6-isopentenyl-adenosine (i⁶A), 2-methylthio-N6-isopentenyl-adenosine (ms²i6A), N6-(cis-hydroxyisopentenyl)adenosine (io⁶A), 2-methylthio-N6-(cis-hydroxyisopentenyl)adenosine (ms2io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g6A), N6,N6-dimethyl-adenosine (m⁶2A), N6-hydroxynorvalylcarbamoyl-adenosine (hn⁶A), 2-methylthio-N6-hydroxynorvalylcarbamoyl-adenosine (ms2hn⁶A), N6-acetyl-adenosine (ac⁶A), 7-methyl-adenosine, 2-methylthio-adenosine, 2-methoxy-adenosine, α-thio-adenosine, 2′-O-methyl-adenosine (Am), N⁶,2′—O-dimethyl-adenosine (m⁶Am), N⁶-Methyl-2′-deoxyadenosine, N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

### Guanine

In an embodiment, the modified nucleobase is a modified guanine. Exemplary nucleobases and nucleosides having a modified guanine include without limitation inosine (I), 1-methyl-inosine (m¹I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQi), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m⁷G), N2-methyl-guanosine (m²G), N2,N2-dimethyl-guanosine (m² 2G), N2,7-dimethyl-guanosine (m²,7G), N2, N2,7-dimethyl-guanosine (m²,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m² 2Gm), 1-methyl-2′-O-methyl-guanosine (m′Gm), N2,7-dimethyl-2′-O-methyl-guanosine (m²,7Gm), 2′-O-methyl-inosine (Im), 1,2′-O-dimethyl-inosine (m′Im), O⁶-phenyl-2′-deoxyinosine, 2′-O-ribosylguanosine (phosphate) (Gr(p)), 1-thio-guanosine, O⁶-methyl-guanosine, O⁶-Methyl-2′-deoxyguanosine, 2′-F-ara-guanosine, and 2′-F-guanosine.

Exemplary Modified gRNAs

In some embodiments, the modified nucleic acids can be modified gRNAs. It is to be understood that any of the gRNAs described herein can be modified in accordance with this section, including any gRNA that comprises a targeting domain from SEQ ID NOs:251-901.

As discussed above, transiently expressed or delivered nucleic acids can be prone to degradation by, e.g., cellular nucleases. Accordingly, in one aspect the modified gRNAs described herein can contain one or more modified nucleosides or nucleotides which introduce stability toward nucleases. While not wishing to be bound by theory it is also believed that certain modified gRNAs described herein can exhibit a reduced innate immune response when introduced into a population of cells, particularly the cells of the present invention. As noted above, the term “innate immune response” includes a cellular response to exogenous nucleic acids, including single stranded nucleic acids, generally of viral or bacterial origin, which involves the induction of cytokine expression and release, particularly the interferons, and cell death.

While some of the exemplary modification discussed in this section may be included at any position within the gRNA sequence, in some embodiments, a gRNA comprises a modification at or near its 5′ end (e.g., within 1-10, 1-5, or 1-2 nucleotides of its 5′ end). In some embodiments, a gRNA comprises a modification at or near its 3′ end (e.g., within 1-10, 1-5, or 1-2 nucleotides of its 3′ end). In some embodiments, a gRNA comprises both a modification at or near its 5′ end and a modification at or near its 3′ end.

In an embodiment, the 5′ end of a gRNA is modified by the inclusion of a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)). The cap or cap analog can be included during either chemical synthesis or in vitro transcription of the gRNA.

In an embodiment, an in vitro transcribed gRNA is modified by treatment with a phosphatase (e.g., calf intestinal alkaline phosphatase) to remove the 5′ triphosphate group.

In an embodiment, the 3′ end of a gRNA is modified by the addition of one or more (e.g., 25-200) adenine (A) residues. The polyA tract can be contained in the nucleic acid (e.g., plasmid, PCR product, viral genome) encoding the gRNA, or can be added to the gRNA during chemical synthesis, or following in vitro transcription using a polyadenosine polymerase (e.g., E. coli Poly(A)Polymerase).

In an embodiment, in vitro transcribed gRNA contains both a 5′ cap structure or cap analog and a 3′ polyA tract. In an embodiment, an in vitro transcribed gRNA is modified by treatment with a phosphatase (e.g., calf intestinal alkaline phosphatase) to remove the 5′ triphosphate group and comprises a 3′ polyA tract.

In some embodiments, gRNAs can be modified at a 3′ terminal U ribose. For example, the two terminal hydroxyl groups of the U ribose can be oxidized to aldehyde groups and a concomitant opening of the ribose ring to afford a modified nucleoside as shown below:

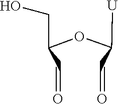

wherein “U” can be an unmodified or modified uridine.

In another embodiment, the 3′ terminal U can be modified with a 2′3′ cyclic phosphate as shown below:

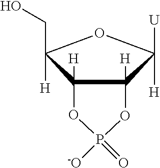

wherein “U” can be an unmodified or modified uridine.

In some embodiments, the gRNA molecules may contain 3′ nucleotides which can be stabilized against degradation, e.g., by incorporating one or more of the modified nucleotides described herein. In this embodiment, e.g., uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In some embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphorothioate group. In some embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

In some embodiments, a gRNA can include “locked” nucleic acids (LNA) in which the 2′ OH-group can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In some embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNA molecules include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In an embodiment, a gRNA comprises a 4′-S, 4′-Se or a 4′-C-aminomethyl-2′-O-Me modification.

In some embodiments, deaza nucleotides, e.g., 7-deaza-adenosine, can be incorporated into the gRNA. In some embodiments, O- and N-alkylated nucleotides, e.g., N6-methyl adenosine, can be incorporated into the gRNA. In some embodiments, one or more or all of the nucleotides in a gRNA molecule are deoxynucleotides.

miRNA Binding Sites

microRNAs (or miRNAs) are naturally occurring cellular 19-25 nucleotide long noncoding RNAs. They bind to nucleic acid molecules having an appropriate miRNA binding site, e.g., in the 3′ UTR of an mRNA, and down-regulate gene expression. While not wishing to be bound by theory, it is believed that this down regulation occurs by either reducing nucleic acid molecule stability or inhibiting translation. An RNA species disclosed herein, e.g., an mRNA encoding Cas9, can comprise an miRNA binding site, e.g., in its 3′UTR. The miRNA binding site can be selected to promote down regulation of expression is a selected cell type.

### EXAMPLES

The following Examples are merely illustrative and are not intended to limit the scope or content of the invention in any way.

### Example 1: Screening of S. pyogenes gRNAs for Use in Inserting 13 bp Del c.−114 to −102 into HBG1 and HBG2 Regulatory Regions

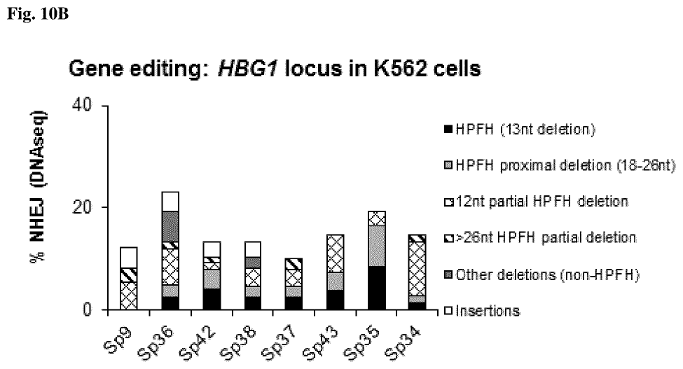

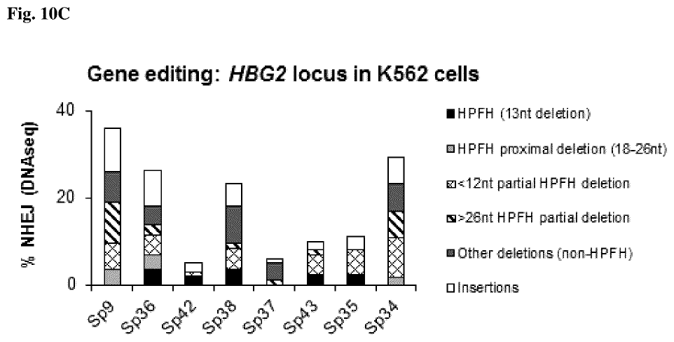

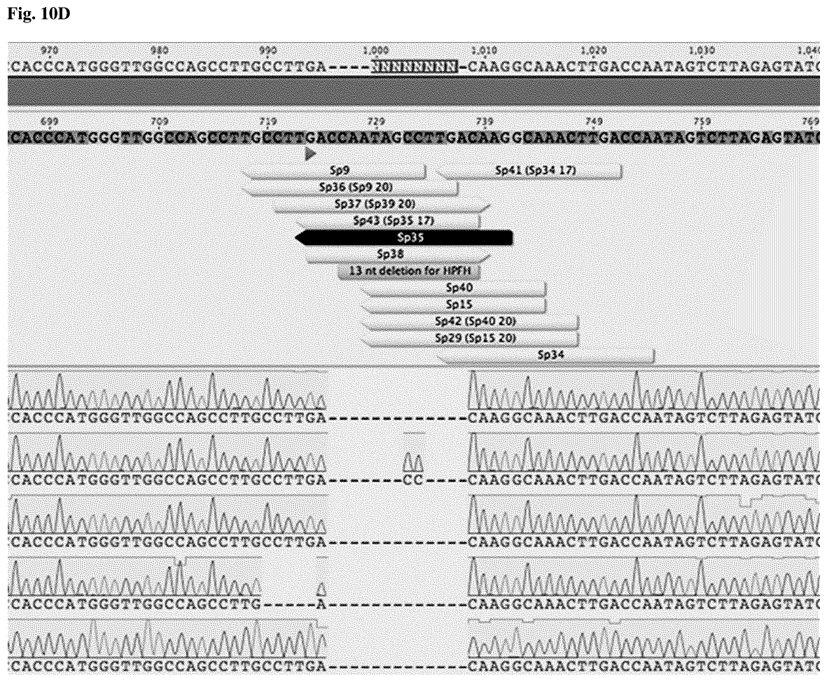

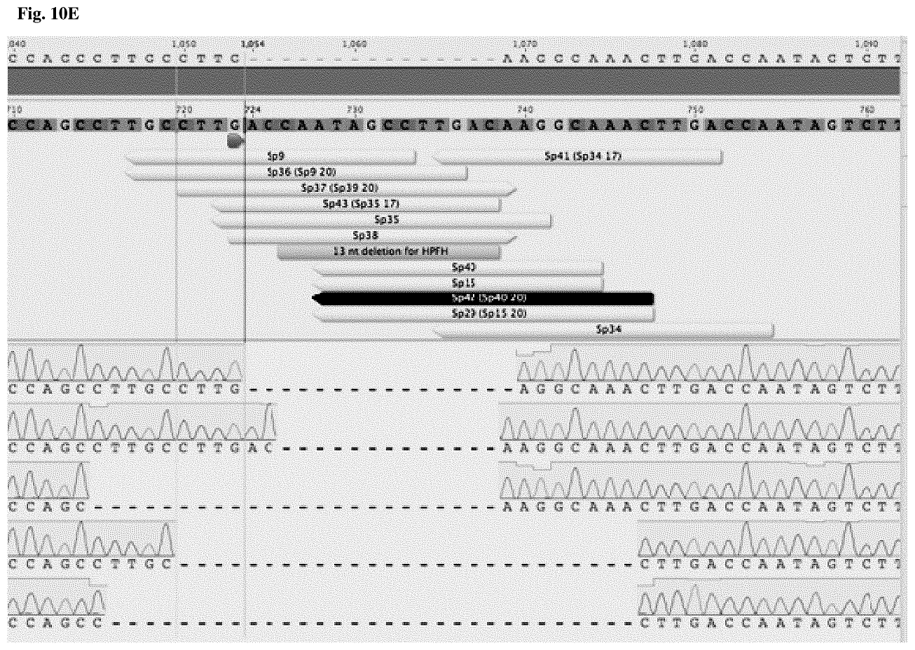

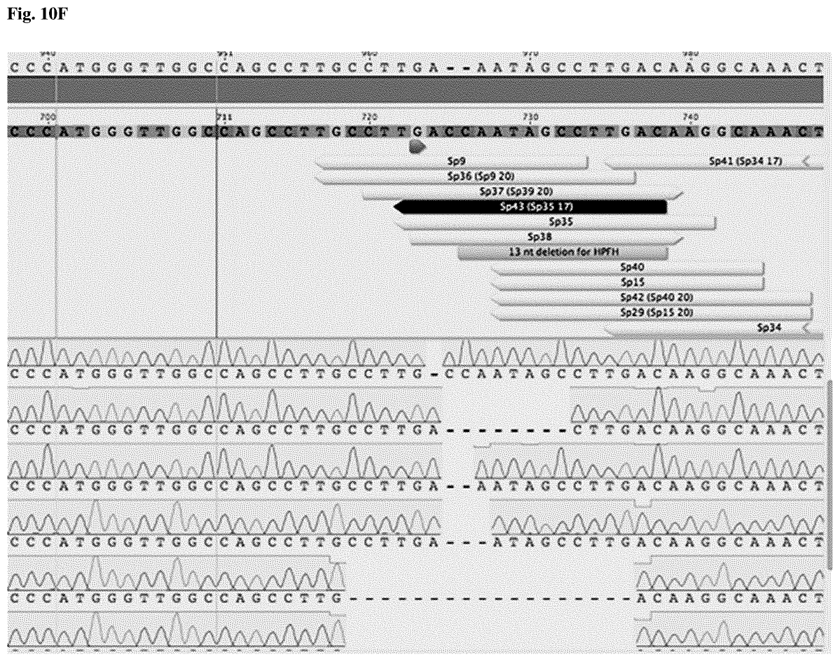

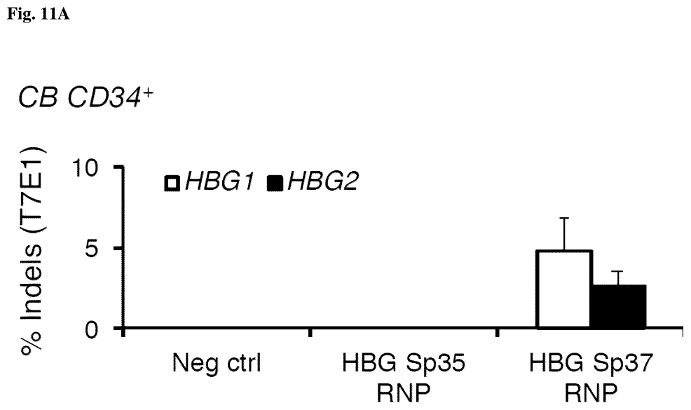

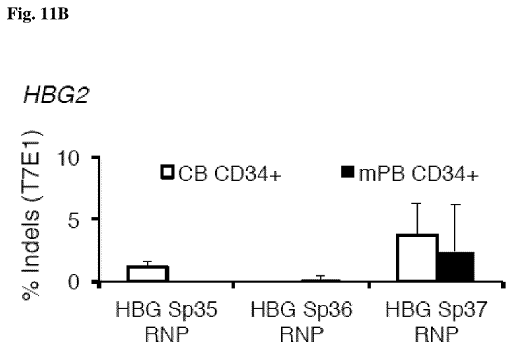

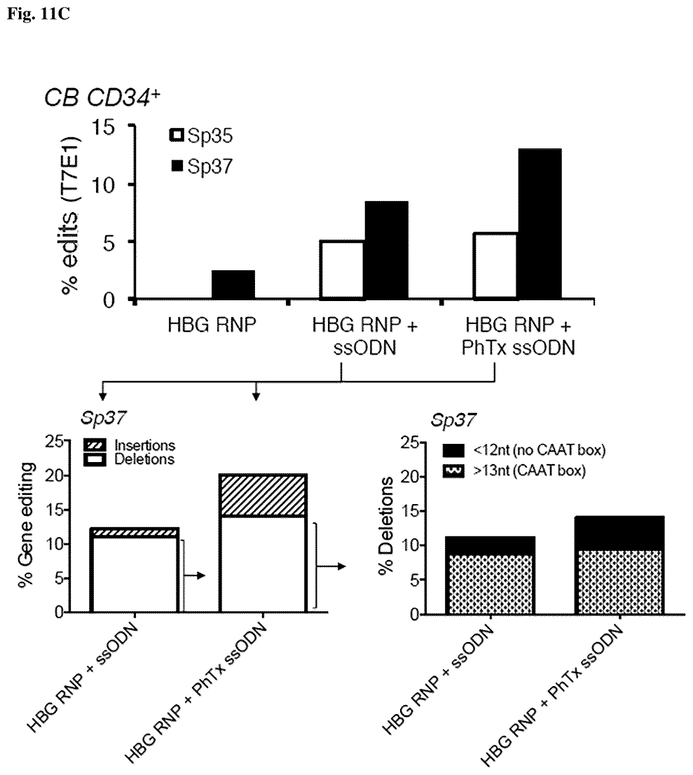

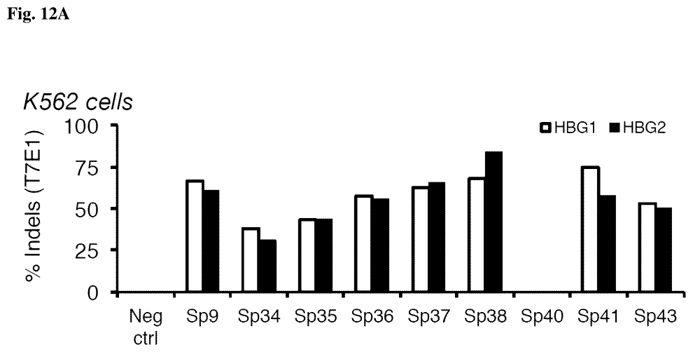

S. pyogenes gRNAs targeting a 26 nt fragment spanning and including the 13 nucleotides at c.−114 to −102 of HBG1 (e.g., nucleotides 2824-2836 of SEQ ID NO:902 (HBG1)), resulting in the alteration HBG1 13 bp del c.−114 to −102) and HBG2 (e.g., nucleotides 2748-2760 of SEQ ID NO:903 (HBG2), resulting in the alteration HBG1 13 bp del c.−114 to −102) were designed as set forth herein. After gRNAs were designed in silico and tiered, a subset of the gRNAs were selected and screened for activity and specificity in human K562 cells. The gRNAs selected for screening contain the targeting domain sequences as set forth in Table 8. The DNA encoding a U6 promoter and each of the S. pyogenes gRNAs were co-electroporated (Amaxa Nucleofector) with plasmid DNA encoding S. pyogenes Cas9 into human K562 cells. The experimental conditions were generally in accordance with those known in the art (e.g., Gori 2016, which is hereby incorporated by reference herein). Three days after electroporation, gDNA was extracted from K562 cells and then the HBG1 and HBG2 loci were PCR amplified from the gDNA. Gene editing was evaluated in the PCR products by T7E1 endonuclease assay analysis. Of the ten sgRNAs screened, eight cut in both the HBG1 and HBG2 targeted regions in the promoter sequences (FIG. 10A).

The HBG1 and HBG2 PCR products for the K562 cells that were targeted with the eight active sgRNAs were then analyzed by DNA sequencing analysis and scored for insertions and deletions detected. The deletions were subdivided into precise 13 nt deletions at the HPFH site, HPFH inclusive and proximal small deletions (18-26 nt), 12 nt deletions (i.e., partial deletion) of the HPFH target site, >26 nt deletions that span a portion of the HPFH target site, and other deletions, e.g., deletions proximal to but outside the HPFH target site. Seven of the eight sgRNAs targeted deletion of the 13 nt (HPFH mutation induction) (FIG. 10B) for HBG1. At least five of the eight sgRNAs also supported targeted deletion of the 13 nt (HPFH mutation induction) in HBG2 promoter region (FIG. 10C). Note that DNA sequence results for HBG2 in cells treated with HBG Sp34 sgRNA were not available. These data indicate that Cas9 and sgRNA support precise induction of the 13 bp del c.−114 to −102 HPFH mutation. FIGS. 10D-10F depict examples of the types of deletions observed in target sequences in HBG1. The gRNA used in each particular example is shown in black, and the other gRNAs not targeted in each panel's example are shown in white.

TABLE 8
Select list of gRNAs for screening in K562 cells

| | | | Targeting | Targeting | | |
|---|---|---|---|---|---|---|
| | | | domain sequence | domain sequence | | |
| | | | plus 3 NT S. | plus 3 NT S. | | |
| | Targeting | Targeting | pyogenes PAM | pyogenes PAM | | |
| gRNA | domain sequence | domain sequence | sequence (NGG) | sequence (NGG) | | Guide |
| ID | (RNA) | (DNA) | (RNA) | (DNA) | Sense | category |
| Sp9 | GGCUAUUGGU | GGCTATTGGTC | GGCUAUUGGU | GGCTATTGGTC | Anti- | spy17 |
| | CAAGGCA | AAGGCA | CAAGGCAAGG | AAGGCAAGG | sense | |
| | (SEQ ID NO: 277) | (SEQ ID NO: 910) | (SEQ ID NO: 920) | (SEQ ID NO: 930) | | |
| Sp36 | CAAGGCUAUU | CAAGGCTATT | CAAGGCUAUU | CAAGGCTATT | Anti- | spy20 |
| | GGUCAAGGCA | GGTCAAGGCA | GGUCAAGGCA | GGTCAAGGCA | sense | |
| | (SEQ ID NO: 338) | (SEQ ID NO: 911) | AGG | AGG | | |
| | | | (SEQ ID NO: 921) | (SEQ ID NO: 931) | | |
| Sp40 | UGCCUUGUCA | TGCCTTGTCAA | UGCCUUGUCA | TGCCTTGTCAA | Anti- | spy17 |
| | AGGCUAU | GGCTAT | AGGCUAUUGG | GGCTATTGG | sense | |
| | (SEQ ID NO: 327) | (SEQ ID NO: 912) | (SEQ ID NO: 922) | (SEQ ID NO: 932) | | |
| Sp42 | GUUUGCCUUG | GTTTGCCTTGT | GUUUGCCUUG | GTTTGCCTTGT | Anti- | spy20 |
| | UCAAGGCUAU | CAAGGCTAT | UCAAGGCUAU | CAAGGCTATT | sense | |
| | (SEQ ID NO: 299) | (SEQ ID NO: 913) | UGG | GG | | |
| | | | (SEQ ID NO: 923) | (SEQ ID NO: 933) | | |
| Sp38 | GACCAAUAGC | GACCAATAGC | GACCAAUAGC | GACCAATAGC | Sense | spy17 |
| | CUUGACA | CTTGACA | CUUGACAAGG | CTTGACAAGG | | |
| | (SEQ ID NO: 276) | (SEQ ID NO: 914) | (SEQ ID NO: 924) | (SEQ ID NO: 934) | | |
| Sp37 | CUUGACCAAU | CTTGACCAATA | CUUGACCAAU | CTTGACCAATA | Sense | spy20 |
| | AGCCUUGACA | GCCTTGACA | AGCCUUGACA | GCCTTGACAA | | |
| | (SEQ ID NO: 333) | (SEQ ID NO: 915) | AGG | GG | | |
| | | | (SEQ ID NO: 925) | (SEQ ID NO: 935) | | |
| Sp43 | GUCAAGGCUA | GTCAAGGCTA | GUCAAGGCUA | GTCAAGGCTA | Anti- | spy17 |
| | UUGGUCA | TTGGTCA | UUGGUCAAGG | TTGGTCAAGG | sense | |
| | (SEQ ID NO: 278) | (SEQ ID NO: 916) | (SEQ ID NO: 926) | (SEQ ID NO: 936) | | |
| Sp35 | CUUGUCAAGG | CTTGTCAAGGC | CUUGUCAAGG | CTTGTCAAGGC | Anti- | spy20 |
| | CUAUUGGUCA | TATTGGTCA | CUAUUGGUCA | TATTGGTCAAG | sense | |
| | (SEQ ID NO: 339) | (SEQ ID NO: 917) | AGG | G | | |
| | | | (SEQ ID NO: 927) | (SEQ ID NO: 937) | | |
| Sp41 | UCAAGUUUGC | TCAAGTTTGCC | UCAAGUUUGC | TCAAGTTTGCC | Anti- | spy17 |
| | CUUGUCA | TTGTCA | CUUGUCAAGG | TTGTCAAGG | sense | |
| | (SEQ ID NO: 310) | (SEQ ID NO: 918) | (SEQ ID NO: 928) | (SEQ ID NO: 938) | | |
| Sp34 | UGGUCAAGUU | TGGTCAAGTTT | UGGUCAAGUU | TGGTCAAGTTT | Anti- | spy20 |
| | UGCCUUGUCA | GCCTTGTCA | UGCCUUGUCA | GCCTTGTCAAG | sense | |
| | (SEQ ID NO: 340) | (SEQ ID NO: 919) | AGG | G | | |
| | | | (SEQ ID NO: 929) | (SEQ ID NO: 939) | | |

### Example 2: Cas9 RNP Containing gRNA Targeting the HPFH Mutation Supports Gene Editing i Human Hematopoietic Stem/Progenitor Cells

Human cord blood (CB) CD34⁺ cells were prestimulated with human cytokines (stem cell factor (SCF), thrombopoietin (TPO), Flt3 ligand (FL)) and small molecules (prostaglandin E2 (PGE2), StemRegenin 1 (SR1)) for two days. The experimental conditions were generally in accordance with the methods provided in Gori 2016 at pages 240-241, which is hereby incorporated by reference herein. The CB CD34⁺ cells were electroporated (Amaxa Nucleofector) with S. pyogenes Cas9 RNP containing (e.g., 5′ ARCA capped and 3′ polyA (20A) tail) sgRNAs (Table 8) that target the HBG1 and HBG2 regulatory regions. Three days after electroporation, gDNA was extracted from the RNP-treated CB CD34⁺ cells, and gene editing was analyzed by T7E1 assay and DNA sequencing.

Of the RNPs containing different gRNAs tested in CB CD34⁺ cells, only Sp37 gRNA (comprising SEQ ID NO:333) resulted in detectable editing at the target site in the HBG1 and HBG2 promoters as determined by T7E1 analysis of indels in HBG1 and HBG2 specific PCR products amplified from gDNA extracted from electroporated CB CD34⁺ cells from a three cord blood donors (FIG. 11A). The average level of editing detected in cells electroporated with Cas9 protein complexed to Sp37 was 5±2% indels at HBG1 and 3±1% indels detected at HBG2 (three separate experiments, and CB donors).

Next, three S. pyogenes gRNAs whose target sites are within the HBG promoter (Sp35 (comprising SEQ ID NO:339), Sp36 (comprising SEQ ID NO:338), Sp37 (comprising SEQ ID NO:333)) were complexed to wild-type S. pyogenes Cas9 protein to form ribonucleoprotein complexes. These HBG targeted RNPS were electroporated into CB CD34⁺ cells (n=3 donors) and adult mobilized peripheral blood (mPB) CD34⁺ cell donors (n=3 donors). CB CD34⁺ cells were prepared in accordance with the methods described above and as in Gori 2016 at pages 240-241. Adult mPB CD34⁺ cells were prepared in substantially the same way as the CB CD34⁺ cells, except without the addition of SR1. Approximately three days after Cas9 RNP delivery, the level of insertions/deletions at the target site was analyzed by T7E1 endonuclease analysis of the HBG2 PCR products amplified from genomic DNA extracted from the samples. Each of these RNPs supported only low level gene editing in both the CB and adult CD34⁺ cells across three donors and three separate experiments (FIG. 11B).

In order to increase gene editing at the target site and to increase the occurrence of the 13 bp deletion at the target site, single strand deoxynucleotide donor repair templates (ssODNs) that encode 87 bp and 89 bp of homology on the 5′ and 3′ side of the targeted deletion site of HBG1 and HBG2 were generated. The construct ssODN1 (SEQ ID NO:906, Table 9), including the 5′ and 3′ homology arm, was designed to ‘encode’ the 13 bp deletion with sequence homology arms engineered flanking this absent sequence to create a perfect deletion. The 5′ homology arm (SEQ ID NO:904, Table 9) includes nucleotides homologous to the sequence 5′ of c.−114 to −102 of HBG1 and HBG2 (i.e., nucleotides homologous to the sequence 5′ of nucleotides 2824-2836 of SEQ ID NO:902 (HBG1) and nucleotides homologous to the sequence 5′ of nucleotides 2748-2760 of SEQ ID NO:903 (HBG2)). The 3′ homology arm (SEQ ID NO:905, Table 9) includes nucleotides homologous to the 3′ region of c.−114 to −102 of HBG1 and HBG2 (i.e., nucleotides homologous to the sequence 3′ of nucleotides 2824-2836 of SEQ ID NO:902 (HBG1) and nucleotides homologous to the sequence 3′ of nucleotides 2748-2760 of SEQ ID NO:903 (HBG2)). The ssODN1 construct was modified at the ends to contain phosphorothioates (PhTx) at the 5′ and 3′ ends (SEQ ID NO:909, Table 9) to form PhTx ssODN1.

TABLE 9
Single strand deoxynucleotide donor repair templates (ssODN)

| | SEQ | |
|---|---|---|
| ssODN ID | ID NO | Sequence |
| ssODN1 | 904 | GGGTGCTTCCTTTTATTCTTCATCCCTAGCCAGCCGCCGGCCCCTG |
| 5′ homology arm | | GCCTCACTGGATACTCTAAGACTATTGGTCAAGTTTGCCTT |
| ssODN1 | 905 | GTCAAGGCAAGGCTGGCCAACCCATGGGTGGAGTTTAGCCAGGGACCG |
| 3′ homology arm | | TTTCAGACAGATATTTGCATTGAGATAGTGTGGGGAAGGGG |
| ssODN1 | 906 | GGGTGCTTCCTTTTATTCTTCATCCCTAGCCAGCCGCCGGCCCCTG |
| | | GCCTCACTGGATACTCTAAGACTATTGGTCAAGTTTGCCTTGTCAAG |
| | | GCAAGGCTGGCCAACCCATGGGTGGAGTTTAGCCAGGGACCGTTTCAG |
| | | ACAGATATTTGCATTGAGATAGTGTGGGGAAGGGG |
| PhTx ssODN1 | 907 | *GGGTGCTTCCTTTTATTCTTCATCCCTAGCCAGCCGCCGGCCCCTG |
| 5′ homology arm | | GCCTCACTGGATACTCTAAGACTATTGGTCAAGTTTGCCTT |
| PhTx ssODN1 | 908 | GTCAAGGCAAGGCTGGCCAACCCATGGGTGGAGTTTAGCCAGGGACCG |
| 3′ homology arm | | TTTCAGACAGATATTTGCATTGAGATAGTGTGGGGAAGGGG* |
| PhTx ssODN1 | 909 | *GGGTGCTTCCTTTTATTCTTCATCCCTAGCCAGCCGCCGGCCCCTG |
| | | GCCTCACTGGATACTCTAAGACTATTGGTCAAGTTTGCCTTGTCAAG |
| | | GCAAGGCTGGCCAACCCATGGGTGGAGTTTAGCCAGGGACCGTTTCAG |
| | | ACAGATATTTGCATTGAGATAGTGTGGGGAAGGGG* |

The homology arms flanking the deletion are indicated by bold [5′ homology arm] and underline [3′ homology arm]). Note the absence of the 13 bp sequence in ssODNl and PhTx ssODN1.
*Represents modification by phosphothioate.
CB CD34⁺ cells were prepared in accordance with the methods described above and as in Gori 2016 at pages 240-241. The ssODNs (ie., ssODN1 and PhTx ssODN1) were co-delivered with RNP targeting HBG containing the Sp37 gRNA (HBG Sp37 RNP) or HBG Sp35 (HBG Sp35 RNP) to CB CD34⁺ cells.

Co-delivery of the ssODN1 and PhTx ssODN1 donor templates encoding the 13 bp deletion with RNP containing Sp35 gRNA (i.e., HBG Sp35 RNP) or RNP containing Sp37 gRNA (i.e., HBG Sp37 RNP) led to a 6-fold and 5-fold increase in gene editing of the target site, respectively, as determined by T7E1 analysis of the HBG2 PCR product (FIG. 11C). DNA sequencing analysis (Sanger sequencing) of the HBG2 PCR product indicated that 20% gene editing in cells that were treated with HBG Sp37 RNP and PhTx ssODN1, with 15% deletions and 5% insertions (FIG. 11C, lower left panel). Further analysis of the specific type and size of deletions at the target site revealed that ¾ of 75% of the total deletions detected contained the HPFH 13 bp deletion (which included deletion of the CAAT box in the proximal promoter), the absence of which is associated with elevation of HbF expression (FIG. 11C, lower right panel). The remaining ¼ of deletions were partial deletions that did not span the full 13 bp deletion. These data indicate that co-delivery of a homologous ssODN that is engineered to have a deletion supported precise gene editing (deletion) at HBG in human CD34⁺ cells.

### Example 3: Screening of S. pyogenes gRNAs Delivered to K562 Cells as Ribonucleoprotein Complexes for Use in Causing 13 bp Del c.−114 to −102 into HBG1 and HBG2 Regulatory Regions

Guide RNAs screened by electroporation of Cas9 and gRNA DNA into K562 cells as described in Example 1 (FIG. 10) were in vitro transcribed and then complexed with S. pyogenes Wt Cas9 protein to form ribonucleoprotein complexes (RNPs). To compare the activity of these RNPs to what was observed by DNA delivery of Cas9 and gRNAs into K562 cells (i.e., Example 1) and by RNP delivery to human CD34⁺ cells (i.e., Example 2), here RNPs were delivered to K562 cells by electroporation (Amaxa Nucleofector). The gRNAs complexed to S. pyogenes Cas9 protein were modified gRNAs ((e.g., 5′ ARCA capped and 3′ polyA (20A) tail; Table 8) and target the HBG1 and HBG2 regulatory regions.

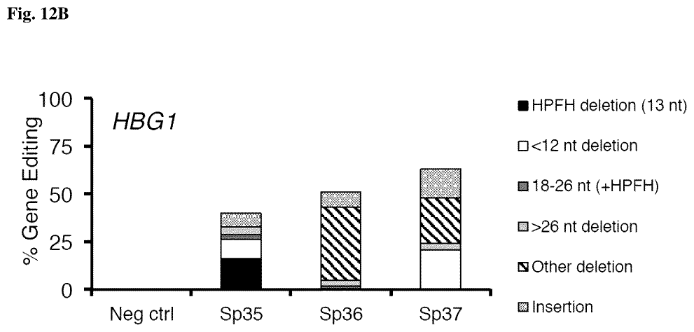

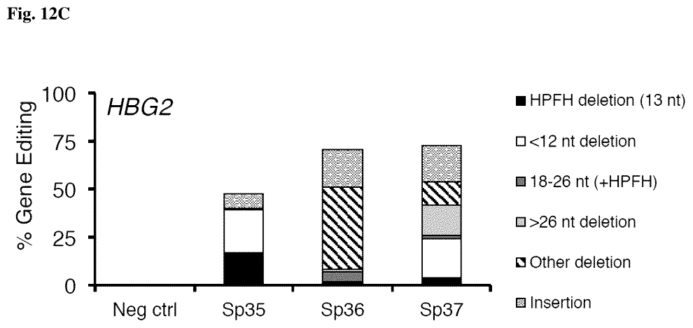

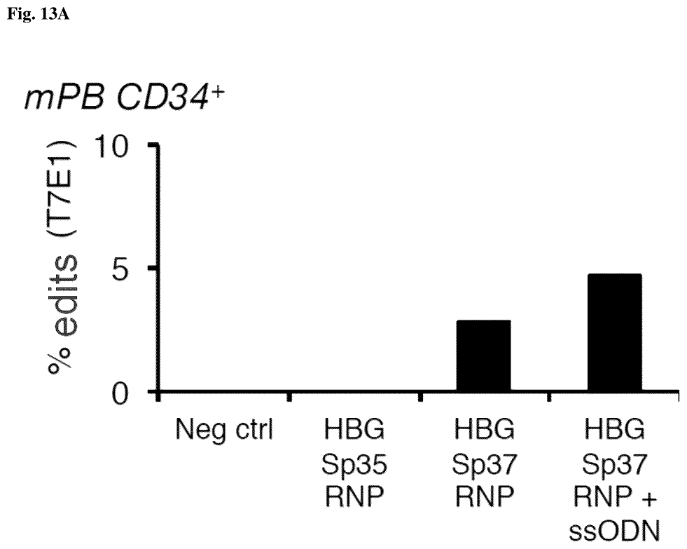

Three days after electroporation, gDNA was extracted from K562 cells and then the HBG1 and HBG2 promoter regions amplified by PCR, followed by T7E1 analysis of the PCR products. (FIG. 12A). Eight out of nine RNPs supported a high percentage of NHEJ. Sp37 RNP, the only gRNA shown to be active in human CD34+ cells (<10% editing in CD34⁺ cells), was highly active in K562 cells, with >60% indels detected at both HBG1 and HBG2 (FIG. 12A). The other gRNA that targets the HPFH deletion mutation site, Sp35, supported 43% editing at HBG1 and HBG2 (FIG. 12A).

DNA sequencing analysis was performed on a subset of PCR products from the gDNA from cells that were treated with Cas9 complexed to gRNAs closest to the targeted HPFH site. DNA sequences were scored for insertions and deletions detected. The deletions were subdivided into precise 13 nt deletions at the HPFH site, HPFH inclusive and proximal small deletions (18-26 nt), 12 nt deletions (i.e., partial deletion) of the HPFH target site, >26 nt deletions that span a portion of the HPFH target site, and other deletions, e.g., deletions proximal to but outside the HPFH target site. The 13 nt deletion was detected in cells treated with RNP complexed to gRNAs Sp35 and 37 (HPFH mutation induction) (FIG. 12B) for HBG1/HBG2 These data indicate that Cas9 and sgRNAs (Sp35 and Sp37) delivered to hematopoietic cells as ribonucleoprotein complexes caused the c.−114 to −102 HPFH mutation.

### Example 4: Cas9 RNP Targeting the HPFH Mutation Supports Gene Editing in Human Adult Mobilized Peripheral Blood Hematopoietic Stem/Progenitor Cells with Increased HBG Expression in Erythroblast Progeny

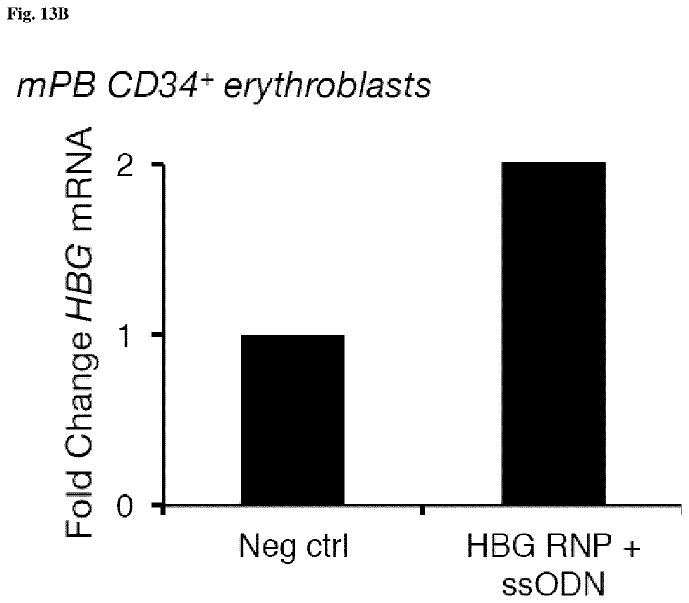

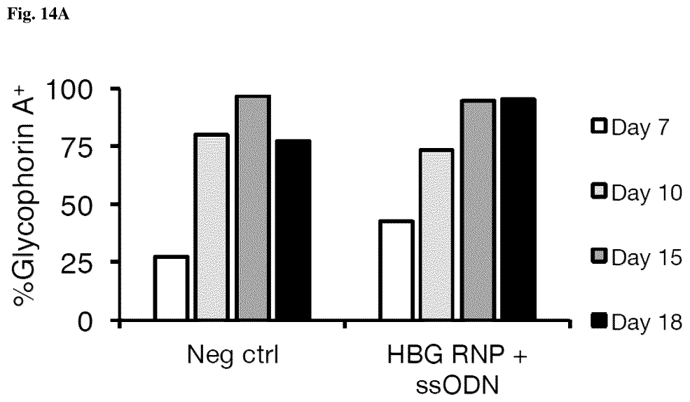

To determine whether editing HBG with Cas9 RNP complexed to Sp37 gRNA or Sp35 gRNA (i.e., the gRNAs that target the 13 bp deletion that is associated with HPFH) in the promoter of HBG supports an increase in HBG expression in erythroid progeny of edited CD34⁺ cells, human adult CD34⁺ cells from mobilized peripheral blood (mPB) were electroporated with the RNPs. Briefly, mPB CD34⁺ cells were prestimulated for 2 days with human cytokines and PGE2 in StemSpan Serum-Free Expansion Medium (SFEM) and then electroporated with Cas9 protein precomplexed to Sp35 and Sp37, respectively. See Gori 2016. T7E1 analysis of HBG PCR product indicated ˜3% indels detected for mPB CD34⁺ cells treated with RNP complexed to Sp37 while no editing was detected for cells that were treated with RNP complexed to Sp35 (FIG. 13A).

In order to increase gene editing at the target site and to increase the occurrence of the 13 bp deletion at the target site, PhTx ssODN1 was co-delivered with the precomplexed RNP targeting HBG containing the Sp37 gRNA. Co-delivery of the PhTx ssODN1 donor encoding the 13 bp deletion led to a nearly 2-fold increase in gene editing of the target site (FIG. 13A).

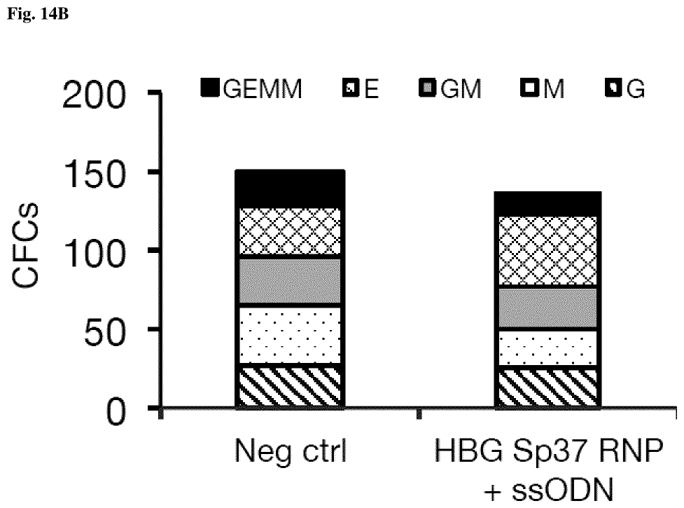

To determine whether editing HBG increases production of fetal hemoglobin in erythroid progeny of edited adult CD34⁺ cells, the cells were differentiated into erythroblasts by culture for up to 18 days in the presence of human cytokines (erythropoietin, SCF, IL3), human plasma (Octoplas), and other supplements (hydrocortisone, heparin, transferrin). Over the time course of differentiation, mRNA was collected to evaluate HBG gene expression in the erythroid progeny of RNP treated mPB CD34⁺ cells and donor matched negative (untreated) controls. By day seven of differentiation, erythroblast progeny of human CD34⁺ cells that were treated with HBG Sp37 RNP and 13 bp HPFH deletion encoding ssODN1 (˜5% indels detected in gDNA from the bulk cell population by T7E1 analysis) exhibited a 2-fold increase in HBG mRNA production (FIG. 13B). Furthermore, the erythroblasts differentiated from RNP treated CD34+ cells maintained the kinetics of differentiation observed for donor matched untreated control cells as determined by flow analysis for acquisition of erythroid phenotype (% Glycophorin A+ cells) (FIG. 14A). Importantly, CD34⁺ cells that were electroporated with HBG Sp37 RNP and ssODN1 maintained their ex vivo hematopoietic activity (i.e., no difference in the quantity or diversity of erythroid and myeloid colonies compared to untreated donor matched CD34⁺ cell negative control), as determined in hematopoietic colony forming cell (CFC) assays (FIG. 14B). These data indicate that targeted disruption of HBG1/HBG2 proximal promoter region supported an increase in HBG expression in erythroid progeny of RNP treated adult hematopoietic stem/progenitor cells without altering differentiation potential.

### Sequences

Genome editing system components according to the present disclosure (including without limitation, RNA-guided nucleases, guide RNAs, donor template nucleic acids, nucleic acids encoding nucleases or guide RNAs, and portions or fragments of any of the foregoing), are exemplified by the nucleotide and amino acid sequences presented in the Sequence Listing. The sequences presented in the Sequence Listing are not intended to be limiting, but rather illustrative of certain principles of genome editing systems and their component parts, which, in combination with the instant disclosure, will inform those of skill in the art about additional implementations and modifications that are within the scope of this disclosure. A list of the sequences presented is provided in the following Table 10.

TABLE 10
Sequences presented in the Sequence Listing:

| SEQ ID NOS: | DESCRIPTION |
|---|---|
| 1-2, 4-6, | Cas9 polypeptides |
| 12, 14 | |
| 3, 7-11, 13 | Cas9 coding sequences |
| 15-23, | Cas9 RuvC-like domains |
| 52-123 | |
| 24-28, | Cas9 HNH-like domains |
| 124-198 | |
| 29-31, 38-51 | Full-length modular and |
| | unimolecular gRNAs |
| 32-37 | gRNA proximal and tail domains |
| 199-205 | PAM sequences |
| 251-901 | gRNA targeting domains (RNA)- |
| | see Tables 6-8 |
| 910-919 | gRNA targeting domains (DNA)- |
| | see Table 8 |
| 920-929 | gRNA targeting domains plus |
| | PAM (NGG) (RNA)-see Table 8 |
| 930-939 | gRNA targeting domains plus |
| | PAM (NGG) (DNA)-see Table 8 |
| 902, 903 | Human HBG1, 2 promoter |
| | sequences including HPFH |
| | deletion site |
| 904-909 | Oligonucleotide donor sequences |
| | and homology arms-see Table 9 |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

Ahern et al., Br J Haematol 25(4):437-444 (1973)
Akinbami Hemoglobin 40:64-65 (2016)
Aliyu et al. Am J Hematol 83:63-70 (2008)
Anders et al. Nature 513(7519):569-573 (2014)
Angastiniotis & Modell Ann N Y Acad Sci 850:251-269 (1998)
Bae et al. Bioinformatics 30(10):1473-1475 (2014)
Barbosa et al. Braz J Med Bio Res 43(8):705-711 (2010)
Bouva Hematologica 91(1):129-132 (2006)
Brousseau Am J Hematol 85(1):77-78 (2010)
Caldecott Nat Rev Genet 9(8):619-631 (2008)
Chassanidis Ann Hematol 88(6):549-555 (2009)
Chylinski et al. RNA Biol 10(5):726-737 (2013)
Cong et al. Science 399(6121):819-823 (2013)
Costa et al., Cad Saude Publica 18(5):1469-1471 (2002)
Cotta-Ramusino et al., Int'l Patent Publication No. WO 2016/073990 (2016)
Fine et al. Sci Rep. 5:10777 (2015)
Friedland et al. Genome Biol 16:257 (2015)
Fu et al. Nat Biotechnol 32:279-284 (2014)
Gori et al, Int'l Patent Publication No. WO 2016/182959 A1 (2016)
Guilinger et al. Nat Biotechnol 32:577-582 (2014)
Jinek et al. Science 337(6096):816-821 (2012)
Jinek et al. Science 343(6176):1247997 (2014)
Kleinstiver et al. Nature 523(7561):481-485 (2015a)
Kleinstiver et al. Nat Biotechnol 33(12):1293-1298 (2015b)
Kleinstiver et al. Nature 529(7587):490-495 (2016)
Lee et al. Nano Lett 12(12):6322-6327 (2012)
Lewis “Medical-Surgical Nursing: Assessment and Management of Clinical Problems” (2014)
Li Cell Res 18(1):85-98 (2008)
Maeder et al. Int'l Patent Publication No. WO 2015/138510 (2015)
Mali et al. Science 339(6121):823-826 (2013)
Mantovani et al. Nucleic Acids Res 16(16):7783-7797 (1988)
Marteijn et al. Nat Rev Mol Cell Biol 15(7):465-481 (2014)
Nishimasu et al. Cell 156(5):935-949 (2014)
Ran et al. Cell 154(6):1380-1389 (2013)
Shmakov et al. Molecular Cell 60(3):385-397 (2015)
Sternberg et al. Nature 507(7490):62-67 (2014)
Superti-Furga et al. EMBO J 7(10):3099-3107 (1988)
Thein Hum Mol Genet 18(R2):R216-223 (2009)
Waber et al. Blood 67(2):551-554 (1986)
Wang et al. Cell 153(4):910-918 (2013)
Xu et al. Genes Dev 24(8):783-798 (2010)
Yamano et al. Cell 165(4): 949-962 (2016)
Zetsche et al. Nat Biotechnol 33(2):139-42 (2015)

1-369. (canceled)

370. A genome editing system comprising:
(a) an RNA-guided nuclease; and
(b) a guide RNA (gRNA) comprising a targeting domain comprising a nucleotide sequence that is identical to, or differs by no more than 1, 2, 3, 4, or 5 nucleotides, from a nucleotide sequence set forth in any of SEQ ID NOs:251-901, wherein the targeting domain is complementary to a target domain located within a region selected from the group consisting of an HBG1, HBG2, and HBG1 and HBG2 regulatory region.

371. The genome editing system of claim 370, wherein the gRNA comprises one or more modifications selected from the group consisting of a 2′-acetylation, a 2′-methylation, and a phosphorothioate modification.

372. The genome editing system of claim 371, wherein the RNA-guided nuclease is a Cas9 molecule.

373. The genome editing system of claim 372, wherein the Cas9 molecule is selected from the group consisting of a S. pyogenes, S. aureus, and S. thermophilus Cas9 molecule.

374. The genome editing system of claim 373, wherein the gRNA is a modular gRNA or unimolecular gRNA.

375. A cell modified by a genome editing system comprising:
(a) an RNA-guided nuclease; and
(b) a guide RNA (gRNA) comprising a targeting domain comprising a nucleotide sequence that is identical to, or differs by no more than 1, 2, 3, 4, or 5 nucleotides, from a nucleotide sequence set forth in any of SEQ ID NOs:251-901, wherein the targeting domain is complementary to a target domain located within a region selected from the group consisting of an HBG1, HBG2, and HBG1 and HBG2 regulatory region.

376. The cell of claim 375, wherein the gRNA comprises one or more modifications selected from the group consisting of a 2′-acetylation, a 2′-methylation, and a phosphorothioate modification.

377. The cell of claim 376, wherein the RNA-guided nuclease is a Cas9 molecule.

378. The cell of claim 377, wherein the Cas9 molecule is selected from the group consisting of a S. pyogenes, S. aureus, and S. thermophilus Cas9 molecule.

379. The cell of claim 378, wherein the gRNA is a modular gRNA or unimolecular gRNA.

380. The cell of claim 379, wherein the cell is selected from one or more cells selected from the group consisting of (1) a cell capable of differentiating into an erythroblast, (2) a cell capable of differentiating into an erythrocyte, (3) a precursor of an erythroblast, (4) a precursor of an erythrocyte, and (5) a long-term hematopoietic stem cell (LT-HSC).

381. The cell of claim 380, wherein the cell is a CD34+ cell.

382. A method of increasing the level of fetal hemoglobin in a human cell by genome editing, the method comprising the step of introducing into the human cell
a ribonucleoprotein (RNP) complex comprising:
(a) an RNA-guided nuclease; and
(b) a guide RNA (gRNA) comprising a targeting domain comprising a nucleotide sequence that is identical to, or differs by no more than 1, 2, 3, 4, or 5 nucleotides, from a nucleotide sequence set forth in any of SEQ ID NOs:251-901, wherein the targeting domain is complementary to a target domain located within a region selected from the group consisting of an HBG1, HBG2, and HBG1 and HBG2 regulatory region.

383. The method of claim 382, wherein the gRNA comprises one or more modifications selected from the group consisting of a 2′-acetylation, a 2′-methylation, and a phosphorothioate modification.

384. The method of claim 383, wherein the RNA-guided nuclease is a Cas9 molecule.

385. The method of claim 384, wherein the Cas9 molecule is selected from the group consisting of a S. pyogenes, S. aureus, and S. thermophilus Cas9 molecule.

386. The method of claim 385, wherein the gRNA is a modular gRNA or unimolecular gRNA.

387. The method of claim 386, wherein the cell is selected from one or more cells selected from the group consisting of (1) a cell capable of differentiating into an erythroblast, (2) a cell capable of differentiating into an erythrocyte, (3) a precursor of an erythroblast, (4) a precursor of an erythrocyte, and (5) a long-term hematopoietic stem cell (LT-HSC).

388. The method of claim 387, wherein the cell is a CD34+ cell.

389. The method of claim 388, wherein the step of introducing into the human cell is performed via electroporation.
